(19) United States
(12) Patent Application Publication
(10) Pub. No.: US 2023/0125667 A1
(43) Pub. Date: Apr. 27, 2023

(54) NOVEL TDZD ANALOGS AS AGENTS THAT DELAY, PREVENT, OR REVERSE AGE-ASSOCIATED DISEASES AND AS ANTI-CANCER AND ANTILEUKEMIC AGENTS

(71) Applicants: United States Government as represented by the Department of Veterans Affairs, Washington (US); BIOVENTURES, LLC, Little Rock, AR (US)

(72) Inventors: Suresh Kuarm Bowroju, Little Rock, AR (US); Peter Crooks, Little Rock, AR (US); Narsimha Penthalla, Little Rock, AR (US); Srinivas Ayyadevara, Little Rock, AR (US); Monica Guzman, New York, NY (US); Robert J. J. Shmokler Reis, Little Rock, AR (US); Eloisi Lopes, New York, NY (US); Samuel Kakraba, Little Rock, AR (US)

(21) Appl. No.: 17/799,638

(22) PCT Filed: Feb. 12, 2021
(86) PCT No.: PCT/US2021/017970

§ 371 Date: Aug. 12, 2022

Related U.S. Application Data
(60) Provisional application No. 62/976,604, filed on Feb. 14, 2020.

(51) Int. Cl. C07D 285/08; C07D 493/04; C07D 417/12; A61P 25/28; A61P 35/02

(52) U.S. Cl. CPC C07D 285/08; C07D 493/04; C07D 417/12; A61P 25/28; A61P 35/02

(57) ABSTRACT

The present disclosure is concerned with TDZD analogs for the treatment of various neurodegenerative diseases such as sarcopenia, supranuclear palsy, Alzheimer's disease, Parkinson's disease, Huntington's disease, and dementia, and various cancers such as, for example, sarcomas, carcinomas, hematological cancers, solid tumors, breast cancer, cervical cancer, gastrointestinal cancer, colorectal cancer, brain cancer, skin cancer, prostate cancer, ovarian cancer, bladder cancer, thyroid cancer, testicular cancer, pancreatic cancer, endometrial cancer, melanomas, gliomas, leukemias, lymphomas, chronic myeloproliferative disorders, myelodysplastic syndromes, myeloproliferative neoplasms, and plasma cell neoplasms (myelomas). This abstract is intended as a scanning tool for purposes of searching in the particular art and is not intended to be limiting of the present invention.

### CROSS-REFERENCE TO RELATED APPLICATIONS

This Application claims the benefit of U.S. Application No. 62/976,604, filed on Feb. 14, 2020, the contents of which are hereby incorporated by reference in their entirety.

### FIELD OF THE DISCLOSURE

The present disclosure relates to the use of a series of 2,4-disubstituted thiadiazolidinone (TDZD) analogs to prevent and/or treat Alzheimer's disease and progressive supranuclear palsy; to inhibit protein aggregation believed to drive numerous age-associated diseases; and to serve as potent anti-cancer and specifically, anti-leukemic agents. This disclosure provides compounds having a structure represented by a formula:

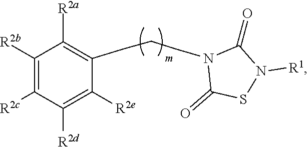

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

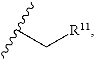

provided that one of R^{2a}, R^{2b}, R^{2c}, R^{2d}, and R^{2e} is Ar¹ or

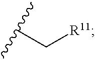

wherein R¹¹, when present, is a carboxylate residue of a chemotherapeutic agent or a carbamide residue of a chemotherapeutic agent; and wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R²⁰, when present, is selected from hydrogen, —OH, C1-C10 alkyl, C1-C10 alkoxy, C1-C10 alkylamino, and (C1-C10)(C1-C10) dialkylamino, provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is

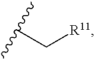

then R¹¹ is not —OC(O)₂(C1-C8 alkyl), —NHC(O)₂(C1-C8 alkyl), or —N(C1-C4 alkyl)C(O)₂(C1-C8 alkyl), or a pharmaceutically acceptable salt thereof, and methods of making and using same. This disclosure also provides compounds having a structure represented by a formula:

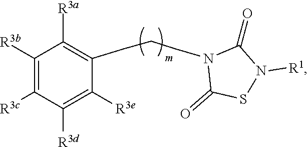

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH, or a pharmaceutically acceptable salt thereof, and methods of making and using same. This disclosure also provides compounds of the following general formula.

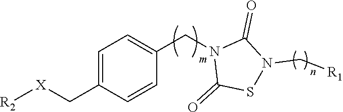

X=NH, O
m=0-3 [carbons]; n=0-10 [carbons],
   R₁ refers to H or different straight or branched chain hydrocarbyl groups or substituted hydrocarbyl with carbon numbers ranging from 0 to 10; or alkyl halides; or alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thiol or diverse hetero-aromatic or other aromatic ring. Halides refers to F, Cl, Br, or I (atoms of fluorine, chlorine, bromine or iodine). R₂ refers to:

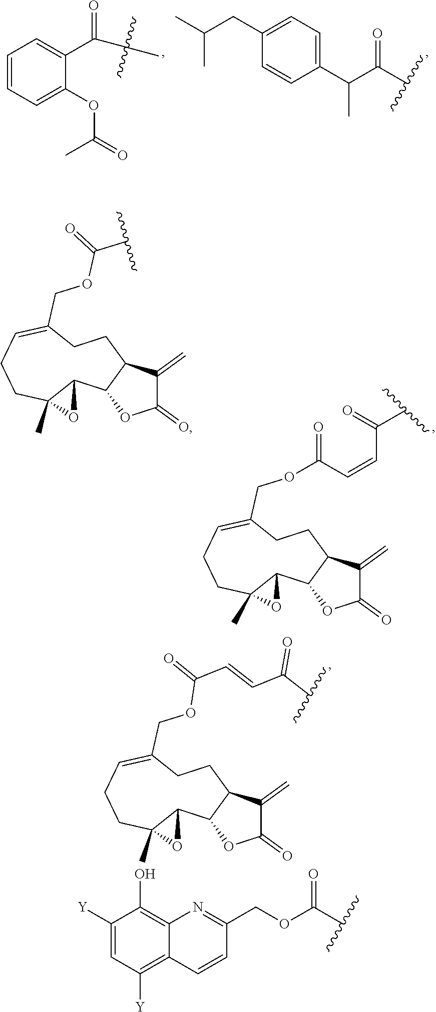

where Y refers to only H or F or Cl or Br or I; or R₂ refers to aromatic or hetero-aromatic ring systems; 1-naphthyl or 2-napthyl group; different simple and substituted 2/3-furyl; 2 or 3 or 4 or 5 or 6 or 7-benzofuryl; 2 or 4 or 5-oxazolyl; 3 or 4 or 5-isoxazolyl; 4 or 5-oxadiazolyl, 2 or 4 or 5 or 6 or 7-benzoxazolyl; 4 or 5 or 6 or 7-benzoxadiazolyl; 2 or 3-pyrrolyl; 3 or 4 or 5-pyrazolyl; 2 or 4 or 5-imidazolyl; 2 or 3 or 4 or 5 or 6 or 7-quinolyl; 2 or 4 or 5 or 6 or 7-quinazolyl; 3 or 4 or 5 or 6 or 7-indazolyl; 4 or 5-triazolyl; 5-tetrazolyl; 2 or 3 or 4 pyridyl; 2 or 4 or 5 or 6-pyrimidyl; 2 or 3-pyrazinyl; 3 or 4 or 5 or 6-pyridazinyl; 2 or 3 or 4 or 5 or 6 or 7-indolyl; 1 or 3 or 4 or 5 or 6 or 7-isoindolyl; 1 or 2 or 3 or 5 or 6 or 7 or 8-indolizinyl; 2 or 4 or 5 or 6 or 7-benzimidazolyl; 3 or 4 or 5 or 6 or 7-indazolyl; 4 or 5 or 6 or 7-benzotriazolyl; 5 or 6 or 7 or 8-tetrazolopyridazinyl; 1 or 2 or 3 or 5 or 6 or 7 or 8-carbazolyl; purinyl; 1 or 3 or 4 or 5 or 6 or 7-isoquinolinyl; 2 or 3 or 5 or 6 or 7 or 8-imidazopyridyl, and the like, and methods of making and using same.

### BACKGROUND

The thiadiazolidinone (TDZD) ring system 1 (FIG. 1) possesses several interesting pharmacological properties, including inhibition of acetylcholinesterase activity (Martinez et al. (2000) Eur J Med Chem. 35(10): 913-922), inhibition of glycogen synthase kinase 3β (GSK3β) (Martinez et al. (2002) Med Res Rev. 22(4): 373-384; Martinez et al. (2002) J Med Chem. 45(6): 1292-1299; Martinez et al. (2005) J Med Chem. 48(23): 7103-7112), opening of potassium channels (Martinez et al. (1997) Bioorganic & Medicinal Chemistry. 5(7): 1275-1283), and agonism to muscarinic receptors (Martinez et al. (1999) Arch Pharm (Weinheim). 332(6): 191-194). GSK-3β has been shown to be involved in several important cellular functions, and inhibition of this enzyme is believed to have therapeutic potential in the treatment of disorders such as type-II diabetes and bipolar disorder (Martinez et al. (2000) Eur J Med Chem. 35(10): 913-922; Martinez et al. (2002) Med Res Rev. 22(4): 373-384; Martinez et al. (2002) J Med Chem. 45(6): 1292-1299; Martinez et al. (2005) J Med Chem. 48(23): 7103-7112). Sustained oral administration of the TDZD compounds decreases hyperphosphorylation of the microtubule-associated protein tau and lowers brain amyloid plaque accrual (both processes implicated in Alzheimer's disease), improves learning and memory, and prevents neuronal loss (Dominguez et al. (2012) J Biol Chem. 287(2): 893-904). TDZD analogs were reported as irreversible inhibitors of GSK-3β, based on the lack of recovery in enzyme function after the removal of unbound drug from the reaction medium, and this irreversibility explained the non-competitive inhibition pattern of TDZD analogs with respect to ATP (Dominguez et al. (2012) J Biol Chem. 287(2): 893-904). The irreversible inhibition of GSK-3β by TDZD analogs and the observation of very low protein turnover rate for the enzyme are mainly relevant from a pharmacological perspective and may have significant implications for its therapeutic potential against age associated diseases like Alzheimer disease (Dominguez et al. (2012) J Biol Chem. 287(2): 893-904).

Aspirin (acetylsalicylic acid), an anti-inflammatory drug, reduces risk for many age-dependent diseases, including cardiovascular disease (CVD), cancers, Alzheimer's disease, and type-2 diabetes (Bartolucci et al. (2011) Am J Cardiol. 107(12): 1796-1801; Ong et al. (2010) Diabetes Care. 33(2): 317-321; Szekely et al. (2008) Neurology. 70(24): 2291-2298). Several anti-inflammatory drugs reduce protein oxidation and misfolding, and thus inhibit protein aggregation and delay age-related diseases and conditions such as sarcopenia (Ayyadevara et al. (2017) Antioxid Redox Signal. 27(17): 1383-1396; Taylor and Brown (1974) Proc Soc Exp Biol Med. 145(1): 32-36). To improve the prevention or delay of age-dependent physiological declines with anti-inflammatory drugs (Halicka et al. (2012) Aging (Albany N.Y.). 4(12): 952-965; Pomponi et al. (2011) Ageing Res Rev. 10(1): 124-131), novel TDZD conjugates were designed with aspirin, ibuprofen, and parthenolide, which may work additively or synergistically to promote long-term survival. Recently, it was reported that progressive increase in protein aggregation, known to accompany aging of the nematode C. elegans, is ameliorated by aspirin, which also extends nematode lifespan by 22% (Ayyadevara et al. (2013) Antioxid Redox Signal. 18(5): 481-490). Without wishing to be bound by theory, these results are consistent with previous reports of significant life extension by aspirin in male mice (Strong et al. (2008) Aging Cell. 7(5): 641-650) and in diabetic humans (Ong et al. (2010) Diabetes Care. 33(2): 317-321; Pignone et al. (2010) Diabetes Care. 33(6): 1395-1402). Celecoxib, another anti-inflammatory drug designed as a selective COX-2 inhibitor, also moderately extends C. elegans life span while reducing protein aggregation (Ching et al. (2011) Aging Cell. 10(3): 506-519). Based on the above findings, it is postulated that anti-inflammatory, nonselective cyclo-oxygenase inhibitors such as aspirin, parthenolides, TDZD analogs, and the selective COX-2 inhibitor ibuprofen, may all share the ability to relieve diverse age-associated conditions by reducing protein aggregation. The TDZD ring system was also modified by introducing diverse alkyl and heterocyclic amine groups of varying chain length. More importantly from a drug-design perspective, —NH₂ and/or —OH groups were introduced to the thiadiazolidinone (TDZD) moiety in order to create drugs with improved water solubility, bioavailability, and tissue targeting.

Recently, it was reported that TDZD analogs produce rapid cell-death kinetics in leukemia cells but not in normal bone marrow cells, and several TDZD-8 analogs are reported as potent anti-leukemic agents (Guzman et al. (2007) Blood. 110(13): 4436-4444; Nasim et al. (2011) BioorgMed Chem Lett. 21(16): 4879-4883).

Sesquiterpene lactones have been isolated from many species of medicinal plants (Chaturvedi D. Sesquiterpene lactones: structural diversity and their biological activities, In-Opportunity, Challanges and Scope of Natural Products in Medicinal Chemistry. ISBN: 978-81-308-0448-4, Research Signpost, Trivandrum. 2011: 313-334), and possess a wide variety of biological activities (Chadwick et al. (2013) Int J Mol Sci. 14(6): 12780-12805; Irmgard (2011) Current Drug Targets. 12(11): 1560-1573). One such sesquiterpene lactone, parthenolide (PTL, FIG. 2), originally isolated from the medicinal herb feverfew (Tanacetum parthenium) (Orofino Kreuger et al. (2012) Anti-Cancer Drugs. 23(9): 883-896), has been identified as an anticancer agent that is particularly effective against both hematologic and solid tumors (Knight D. W. (1995) Nat Prod Rep. 12(3): 271-276). PTL and its derivatives promote apoptosis by inhibiting the activity of the NF-κB transcription factor complex, thereby downregulating anti-apoptotic genes under NF-κB control and also increasing reactive oxygen species (ROS) through inhibition of the glutathione pathway (Bork et al. (1997) FEBS Lett. 402(1): 85-90; Wen et al. (2002) J Biol Chem. 277(41): 38954-38964; Hehner et al. (1998) J Biol Chem. 273(3): 1288-1297; Sweeney et al. (2004) Clin Cancer Res. 10(16): 5501-5507; Yip-Schneider et al. (2005) Mol Cancer Ther. 4(4): 587-594; Nozaki et al. (2001) Oncogene. 20(17): 2178-2185).

It was previously demonstrated that PTL induces robust apoptosis of primary acute myeloid leukemia (AML) stem cells in culture (Guzman et al. (2005) Blood. 105(11): 4163-4169; Guzman and Jordan (2005) Expert Opin Biol Ther. 5(9): 1147-1152; Dai et al. (2010) Br J Haematol. 151(1): 70-83; Kim et al. (2010) Journal of Pharmacology and Experimental Therapeutics. 335(2): 389-400). AML is a clonal malignancy of the hematopoietic system characterized by accumulation of immature cell populations in the bone marrow or peripheral blood (Deschler and Lubbert (2006) Cancer. 107(9): 2099-2107). AML is the most common leukemia in adults and has the lowest survival rate of all leukemias (Estey and Dohner (2006) The Lancet. 368(9550): 1894-1907; Löwenberg et al. (1998) Journal of Clinical Oncology. 16(3): 872-881; Tazzari et al. (2007) Leukemia. 21(3): 427-438). The poor water-solubility of PTL was recently overcome without loss of its anti-leukemic activity, by derivatizing PTL into several alkylamino analogs, which can then be converted into water-soluble organic salts (Nasim and Crooks (2008) Bioorganic & Medicinal Chemistry Letters. 18(14): 3870-3873). PTL has also been the source of several novel antileukemic compounds over the past decade. For instance, melampomagnolide B (MMB) (FIG. 3) a melampolide originally isolated from Magnolia grandiflora (S. El-Feraly (1984) Phytochemistry. 23(10): 2372-2374), has been identified as a new antileukemic sesquiterpene with properties similar to PTL. MMB was synthesized utilizing a modification of the method of Macias et al. via selenium oxide oxidation of the C10 methyl group of PTL, which also results in concomitant conversion of the geometry of the C9-C10 double bond from trans to cis orientation (Macias et al. (1992) Phytochemistry. 31(6): 1969-1977).

A biotin-conjugated derivative of MMB was designed and synthesized in order to elucidate its mechanism of action (Nasim et al. (2011) BioorgMed Chem. 19(4): 1515-1519). More importantly from a drug-design point of view, MMB is a more interesting molecule because it contains a primary —OH group, which provides the means to design prodrugs with improved water solubility, bioavailability, and tissue targeting. However, MMB itself is less potent against leukemia cell lines than its parent molecule PTL and also against solid tumor cell lines. More recently the anti-cancer activity of MMB was enhanced by synthesizing a variety of carbamate (Janganati et al. (2014) Bioorg Med Chem Lett. 24(15): 3499-3502; Janganati et al. (2015) J Med Chem. 58(22): 8896-8906; Albayati et al. (2017) Bioorg Med Chem. 25(3): 1235-1241), carbonate (Janganati et al. (2015) J Med Chem. 58(22): 8896-8906), ester (Bommagani et al. (2017) Eur J Med Chem. 136: 393-405), amide (Janganati et al. (2017) BioorgMed Chem. 25(14): 3694-3705), and triazole (click chemistry) (Janganati et al. (2018) Eur J Med Chem. 157: 562-581) conjugated derivatives. To further enhance the potency of MMB and TDZD, MMB-TDZD conjugated analogs have also been designed and synthesized as potent anti-cancer and anti-aging compounds.

In sum, despite the widespread utility of TDZD-MMB conjugated ring systems, the design and synthesis of novel conjugated analogs having improved potency and selectivity continues to remain elusive. Thus, there remains a need for conjugated TDZD analogs, compositions containing the analogs, and methods of making and using same.

### SUMMARY

In accordance with the purpose(s) of the invention, as embodied and broadly described herein, the invention, in one aspect, relates to compounds and compositions for use in the prevention and treatment of neurodegenerative diseases (e.g., sarcopenia, supranuclear palsy, Alzheimer's disease, dementia) and disorders of uncontrolled cellular proliferation such as, for example, cancer (e.g., sarcomas, carcinomas, hematological cancers, solid tumors, breast cancer, cervical cancer, gastrointestinal cancer, colorectal cancer, brain cancer, skin cancer, prostate cancer, ovarian cancer, bladder cancer, thyroid cancer, testicular cancer, pancreatic cancer, endometrial cancer, melanomas, gliomas, leukemias, lymphomas, chronic myeloproliferative disorders, myelodysplastic syndromes, myeloproliferative neoplasms, and plasma cell neoplasms (myelomas)).

Thus, disclosed are compounds having a structure represented by a formula:

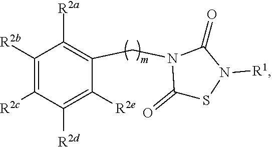

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

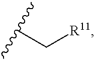

provided that one of R^{2a}, R^{2b}, R^{2c}, R^{2d}, and R^{2e} is Ar¹ or

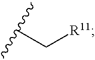

wherein R¹¹, when present, is a carboxylate residue of a chemotherapeutic agent or a carbamide residue of a chemotherapeutic agent; and wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R²⁰, when present, is selected from hydrogen, —OH, C1-C10 alkyl, C1-C10 alkoxy, C1-C10 alkylamino, and (C1-C10)(C1-C10) dialkylamino, provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is

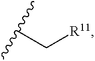

then R¹¹ is not —OC(O)₂(C1-C8 alkyl), —NHC(O)₂(C1-C8 alkyl), or —N(C1-C4 alkyl)C(O)₂(C1-C8 alkyl), or a pharmaceutically acceptable salt thereof.

Also disclosed are compounds having a structure represented by a formula:

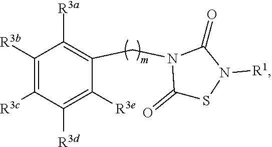

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH, or a pharmaceutically acceptable salt thereof.

Also disclosed are compounds selected from:

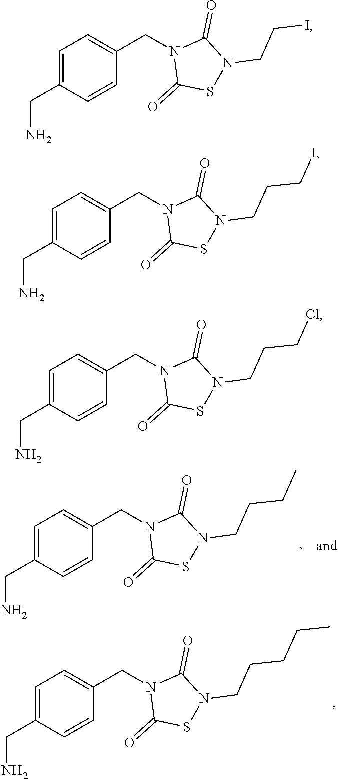 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof.

Also disclosed is a compound having a structure:

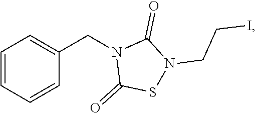 [SMILES: O=c1sn(CCI)c(=O)n1Cc1ccccc1]

or a pharmaceutically acceptable salt thereof.

Also disclosed are compounds of the following structural design:

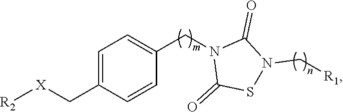

in which m=0-3 [carbons]; n=0-10 [carbons], where carbons are indicated by an oblique angle in parentheses, X=O or NH, R₁ refers to H or different straight or branched chain hydrocarbyl groups or substituted hydrocarbyl with carbon numbers ranging from 0 to 10; or alkyl halides; or alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thiol or diverse hetero-aromatic or other aromatic ring, where halogen refers to F, Cl, Br, or I (atoms of fluorine, chlorine, bromine or iodine), R₂ refers to:

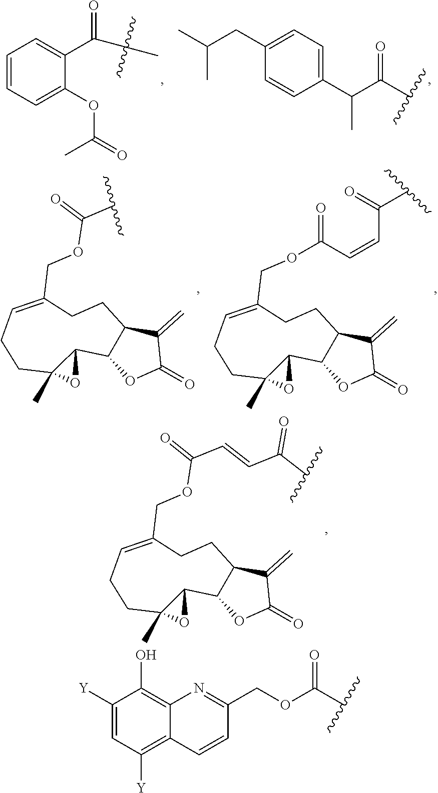

where Y refers to only H or Cl or Br, or I, or R₂ also refers to different aromatic or hetero-aromatic ring systems like 1-naphthyl or 2-naphthyl group; different simple and substituted 2/3-furyl; 2 or 3 or 4 or 5 or 6 or 7-benzofuryl; 2 or 4 or 5-oxazolyl; 3 or 4 or 5-isoxazolyl; 4 or 5-oxadiazolyl, 2 or 4 or 5 or 6 or 7-benzoxazolyl; 4 or 5 or 6 or 7-benzoxadiazolyl; 2 or 3-pyrrolyl; 3 or 4 or 5-pyrazolyl; 2 or 4 or 5-imidazolyl; 2 or 3 or 4 or 5 or 6 or 7-quinolyl; 2 or 4 or 5 or 6 or 7-quinazolyl; 3 or 4 or 5 or 6 or 7-indazolyl; 4 or 5-triazolyl; 5-tetrazolyl; 2 or 3 or 4 pyridyl; 2 or 4 or 5 or 6-pyrimidyl; 2 or 3-pyrazinyl; 3 or 4 or 5 or 6-pyridazinyl; 2 or 3 or 4 or 5 or 6 or 7-indolyl; 1 or 3 or 4 or 5 or 6 or 7-isoindolyl; 1 or 2 or 3 or 5 or 6 or 7 or 8-indolizinyl; 2 or 4 or 5 or 6 or 7-benzimidazolyl; 3 or 4 or 5 or 6 or 7-indazolyl; 4 or 5 or 6 or 7-benzotriazolyl; 5 or 6 or 7 or 8-tetrazolopyridazinyl; 1 or 2 or 3 or 5 or 6 or 7 or 8-carbazolyl; purinyl; 1 or 3 or 4 or 5 or 6 or 7-isoquinolinyl; 2 or 3 or 5 or 6 or 7 or 8-imidazopyridyl, and the like moieties.

Also disclosed are pharmaceutical compositions comprising a therapeutically effective amount of a disclosed compound and a pharmaceutically acceptable carrier.

Also disclosed are methods for treating a disorder of uncontrolled cellular proliferation in a subject, the method comprising administering to the subject an effective amount of a disclosed compound.

Also disclosed are methods for treating a neurological disorder in a subject, the method comprising administering to the subject an effective amount of a disclosed compound.

Also disclosed are methods for treating a neurological disorder in a subject, the method comprising administering to the subject an effective amount of a compound selected from:

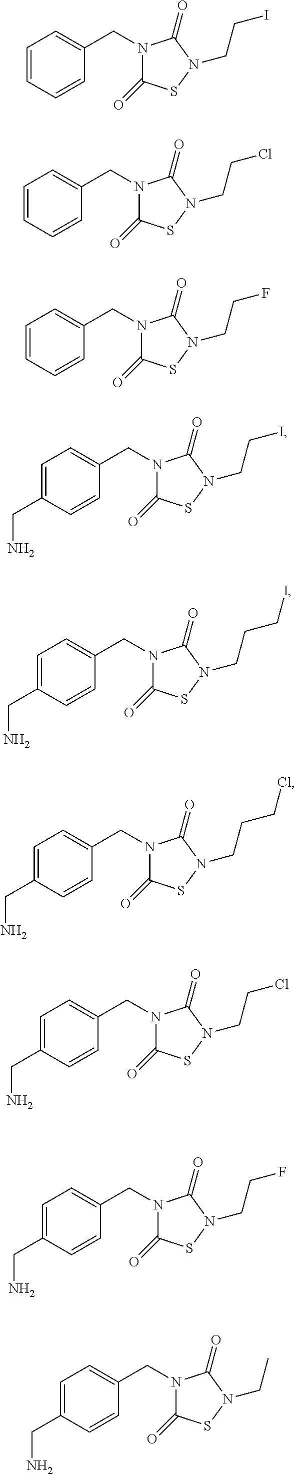

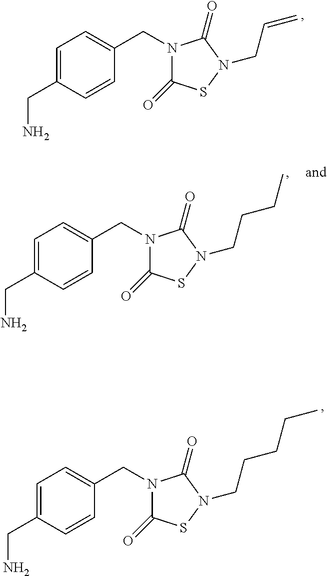 [SMILES: C=CCn1sc(=O)n(Cc2ccc(CN)cc2)c1=O ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof.

Also disclosed are kits comprising a disclosed compound, and one or more of: (a) at least one agent associated with the treatment of a disorder of uncontrolled cellular proliferation; (b) at least one agent associated with the treatment of a neurological disorder; (c) instructions for administering the compound in connection with treating a disorder of uncontrolled cellular proliferation; (d) instructions for administering the compound in connection with treating a neurological disorder; (e) instructions for treating a disorder of uncontrolled cellular proliferation; and (f) instructions for treating a neurological disorder.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a generic chemical structure of 2,4-substituted thiadiazolidinones (TDSDs).

FIG. 2 shows the chemical structure of parthenolide (PTL).

FIG. 3 shows the chemical structure of melampomagnolide B (MMB).

FIG. 4 shows representative data illustrating that PNR-962 (5 μM) reduces protein aggregation in human neuroblastoma cells (SH-SY5Y-APP_{Sw}) that express APP_{Sw}, an aggregation-prone mutant form of amyloid precursor protein.

FIG. 5 shows representative data illustrating that a GSK-3 inhibitor reduces paralysis >75% in C. elegans adults with muscle expression of Aβ₁₋₄₂. Specifically, the percent of worms paralyzed 48 hr after induction of Aβ₁₋₄₂ synthesis, by upshift to 25° C. at the L3/L4 transition, is shown. Significance, as shown, was assessed by a 2-tailed heteroscedastic t test.

FIG. 6 shows representative data demonstrating that PNR-962, a TDZD analogue, reduces huntingtin-like aggregates in a C. elegans Huntington's disease-model strain Specifically, adult worms expressing Q40::YFP were imaged and YFP⁺ muscle aggregates were quantified after 3 days of exposure to 5-μM PNR-962. Treated worms differ from controls by 2-tailed t test (P<0.0001).

FIG. 7 shows representative data demonstrating that the number of aggregation foci decreases with exposure to PNR-962 (a GSK-3β inhibitor), or to an (aspirin+GSK-3β inhibitor) combo drug (BSK-179) in a C. elegans model of Huntington's Disease.

FIG. 8 shows representative data demonstrating that the lifespan of C. elegans is increased by aspirin, PNR-962, or BSK-179

FIG. 9 shows representative data illustrating that BSK-179 reduces amyloid accumulation in human neuroblastoma cells expressing mutant amyloid precursor protein (APP_{Sw}). Amyloid foci were stained with thioflavin T in SH-SY5Y-APP_{Sw} cells treated for 2 days.

FIG. 10 shows the chemical structures of aspirin and BSK-179.

FIG. 11 shows a representative image illustrating a proposed binding conformation of TDZD analogues to GSK3β.

FIG. 12 shows representative data generated from a virtual screen against GSK3β.

FIG. 13 shows representative data pertaining to a SeeSAR structure-activity analysis of TDZD-GSK33.

FIG. 14 shows representative data pertaining to the in vitro screening of TDZD analogues in HEK-TAU cells (by thioflavin-T staining).

FIG. 15 shows representative data demonstrating the potency of TDZD analogues.

FIG. 16 shows a representative image generated via computational modeling of TDZD analogues.

FIG. 17 shows further representative data demonstrating the potency of TDZD analogues.

FIG. 18A-G shows representative data illustrating a computer prediction that PNR886 and PNR962 bind stably to the same allosteric hydrophobic pocket in GSK3β.

FIG. 19A-I show representative snapshots of full-length GSK3β bound to TDZD-8 (FIG. 19A-C) and its analogs PNR962 (FIG. 19D-F) and PNR886 (FIG. 19G-I).

FIG. 19J-L show representative data illustrating the root mean square deviation (RMSD) of protein-ligand complexes during 200-ns simulations of GSK3β binding to TDZD-8 (FIG. 19J), PNR962 (FIG. 19K) and PNR886 (FIG. 19L).

FIG. 20 shows representative data illustrating that TDZD analogs bind to GSK3β in MM/GBSA assay.

FIG. 21A-C show representative dose-response curves for in vitro inhibition of GSK3β activity by TDZ-8 (FIG. 21A), PNR962 (FIG. 21B), and PNR886 (FIG. 21C).

FIG. 22 shows representative data illustrating the superimposition of drugs for QSAR modeling and prediction.

FIG. 23 shows representative data illustrating the suppression of tau hyperphosphorylation by PNR962, a TDZD analogue.

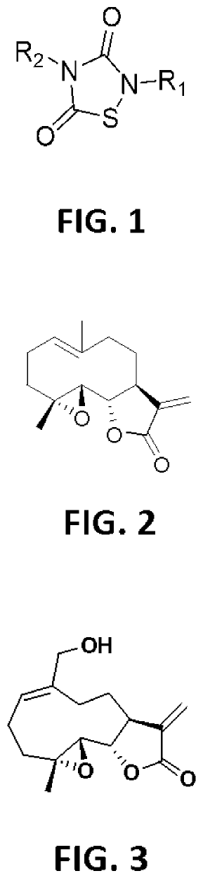

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (TLs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents.

Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic.

The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A′, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for Ste. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “R°,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₄Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄(O)₂R^{∘}; —(CH₂)₀₋₄(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂R^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}2, —C(S)NR^{†}2, —N(N^{†})NR^{†}₂, or —N(R^{†})S(O)₂R^{†}; wherein each R^{†} is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

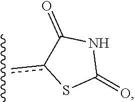

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (F/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, is ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

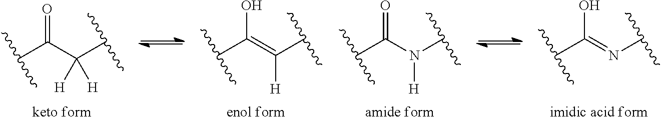 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

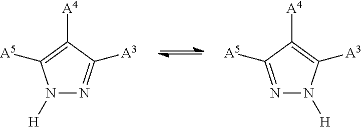

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

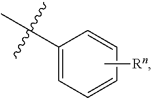

which is understood to be equivalent to a formula:

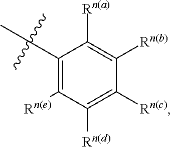

wherein n is typically an integer. That is, Rⁿ is understood to represent five independent substituents, R^{n(a)}, R^{(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to TDZD analogs useful in treating neurodegenerative diseases (e.g., sarcopenia, supranuclear palsy, Alzheimer's disease, dementia) and disorders of uncontrolled cellular proliferation such as, for example, cancer (e.g., sarcomas, carcinomas, hematological cancers, solid tumors, breast cancer, cervical cancer, gastrointestinal cancer, colorectal cancer, brain cancer, skin cancer, prostate cancer, ovarian cancer, bladder cancer, thyroid cancer, testicular cancer, pancreatic cancer, endometrial cancer, melanomas, gliomas, leukemias, lymphomas, chronic myeloproliferative disorders, myelodysplastic syndromes, myeloproliferative neoplasms, and plasma cell neoplasms (myelomas)).

In one aspect, the compounds are useful in the treatment of neurodegenerative diseases, as further described below.

In one aspect, the compounds are useful in the treatment of disorders of uncontrolled cellular proliferation, as further described herein.

The compound may be a free form or a salt form. When the compound is in a salt form, the salt is preferably a pharmaceutically acceptable salt. Pharmaceutically acceptable salts may include, without limitation, hydrochloride, hydrobromide, phosphate, sulfate, methane-sulfonate, acetate, formate, tartrate, bitartrate, stearate, phthalate, hydroiodide, lactate, monohydrate, mucate, nitrate, phosphate, salicylate, phenylpropionate, isobutyrate, hypophosphite, maleic acid, malic acid, citrate, isocitrate, succinate, lactate, gluconate, glucuronate, pyruvate, oxalate, fumarate, propionate, aspartate, glutamate, benzoate, terephthalate, and the like. In other embodiments, the pharmaceutical acceptable salt includes an alkaline or alkaline earth metal ion salt. In particular, sodium, potassium or other pharmaceutically acceptable inorganic salts are used.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are compounds having a structure represented by a formula:

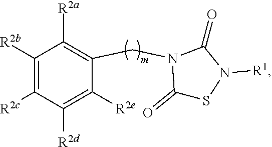

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

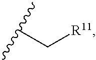

provided that one of R^{2a}, R^{2b}, R^{2c}, R^{2d}, and R^{2e} is Ar¹ or

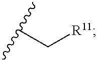

wherein R¹¹, when present, is a carboxylate residue of a chemotherapeutic agent or a carbamide residue of a chemotherapeutic agent; and wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R²⁰, when present, is selected from hydrogen, —OH, C1-C10 alkyl, C1-C10 alkoxy, C1-C10 alkylamino, and (C1-C10)(C1-C10) dialkylamino, provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is

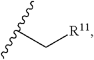

then R¹¹ is not —OC(O)₂(C1-C8 alkyl), —NHC(O)₂(C1-C8 alkyl), or —N(C1-C4 alkyl)C(O)₂(C1-C8 alkyl), or a pharmaceutically acceptable salt thereof.

In one aspect, disclosed are compounds having a structure represented by a formula:

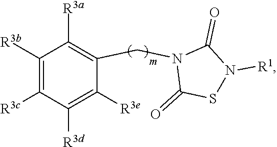

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH, or a pharmaceutically acceptable salt thereof.

In one aspect, disclosed are compounds selected from:

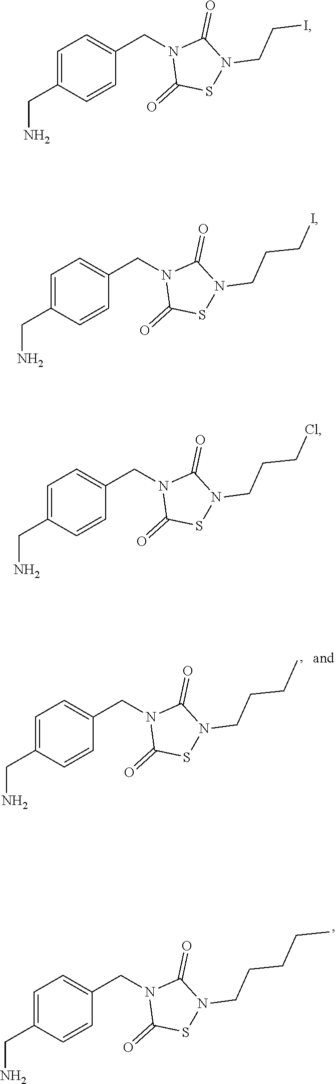 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof.

In one aspect, disclosed is a compound having a structure:

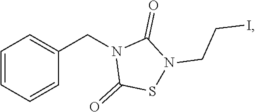 [SMILES: O=c1sn(CCI)c(=O)n1Cc1ccccc1]

or a pharmaceutically acceptable salt thereof.

In various aspects, the compound has a structure represented by a formula:

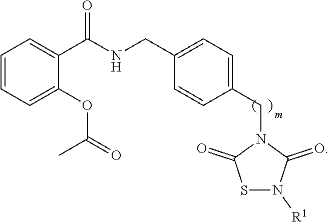

In a further aspect, m is 1 and R¹ is selected from C1-C10 alkyl, C1-C10 haloalkyl, and Cy¹.

In various aspects, the compound has a structure represented by a formula:

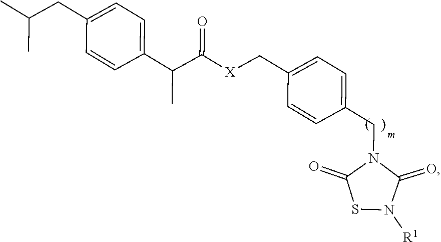

wherein X is NH or O. In a further aspect, m is 1 and R¹ is selected from C1-C10 alkyl, C1-C10 haloalkyl, and Cy¹.

In various aspects, the compound has a structure represented by a formula:

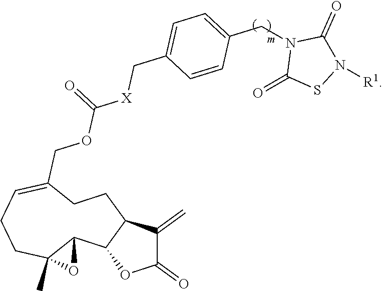

In a further aspect, m is 1, X is NH, and R¹ is selected from C1-C10 alkyl, C1-C10 haloalkyl, and Cy¹.

In various aspects, the compound has a structure represented by a formula:

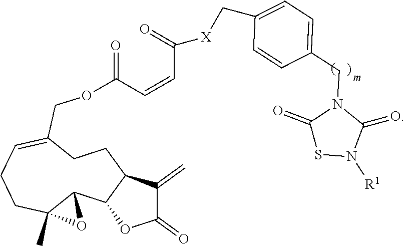

In a further aspect, m is 1, X is NH, and R¹ is selected from C1-C10 alkyl, C1-C10 haloalkyl, and Cy¹.

In various aspects, the compound has a structure represented by a formula:

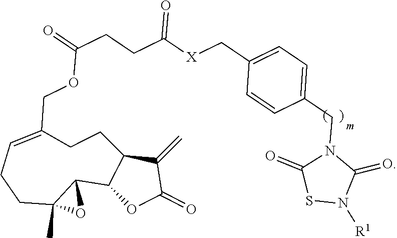

In a further aspect, m is 1, X is NH, and R¹ is selected from C1-C10 alkyl, C1-C10 haloalkyl, and Cy¹.

In various aspects, the compound has a structure represented by a formula:

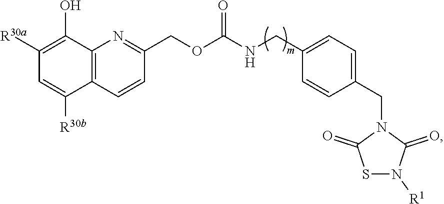

wherein each of R^{30a} and R^{30b} is independently selected from hydrogen and halogen. In a further aspect, m is 1, X is NH, and R¹ is selected from C1-C10 alkyl, C1-C10 haloalkyl, and Cy¹.

In various aspects, the compound has a structure represented by a formula:

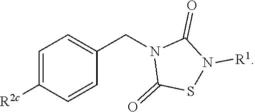

In a further aspect, R¹ is selected from C1-C10 alkyl, C1-C10 haloalkyl, and Cy¹. In a still further aspect, R^{2c} is

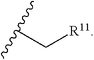

In yet a further aspect, R¹¹ is selected from:

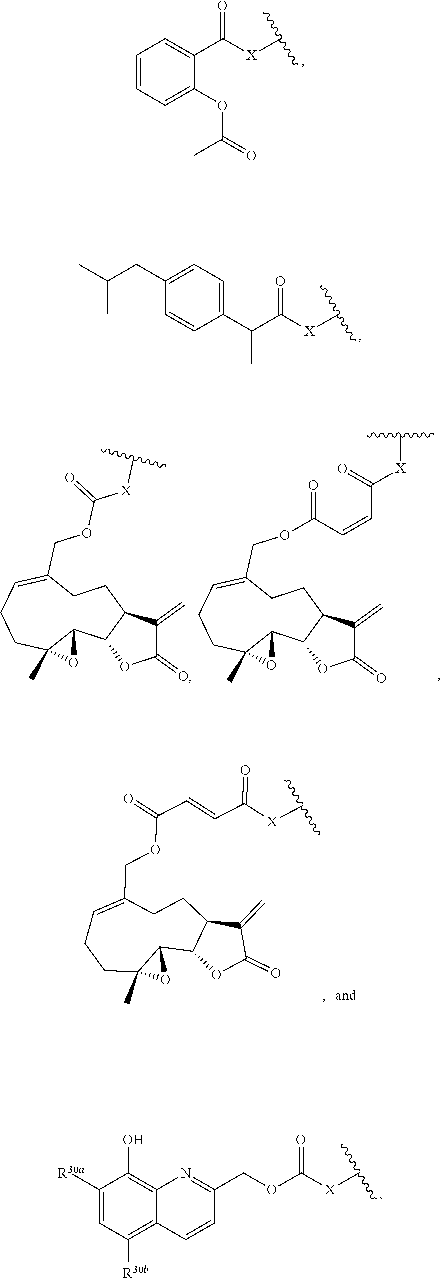

wherein X is selected from NH and O; and wherein each of R^{30a} and R^{30b}, when present, is independently selected from hydrogen and halogen. In an even further aspect, R¹ is selected from C1-C10 alkyl, C1-C10 haloalkyl, and Cy¹, R^{2c} is

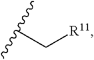

and R¹¹ is selected from:

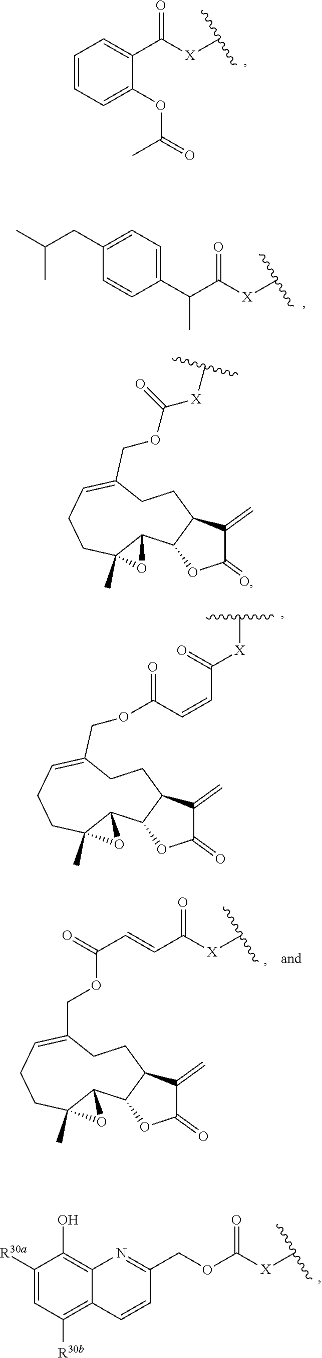

wherein X is selected from NH and O; wherein each of R^{30a} and R^{30b} is independently selected from hydrogen and halogen.

In various aspects, the compound is selected from:

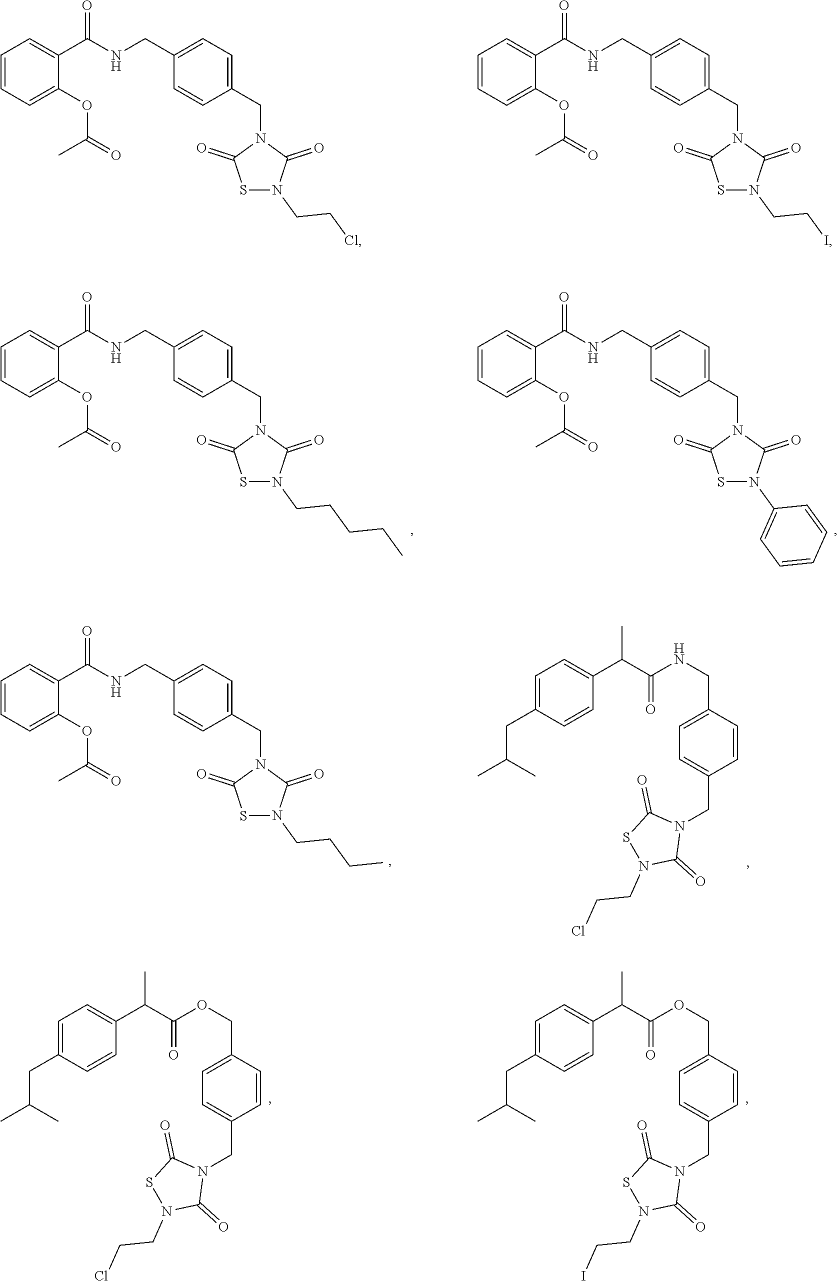

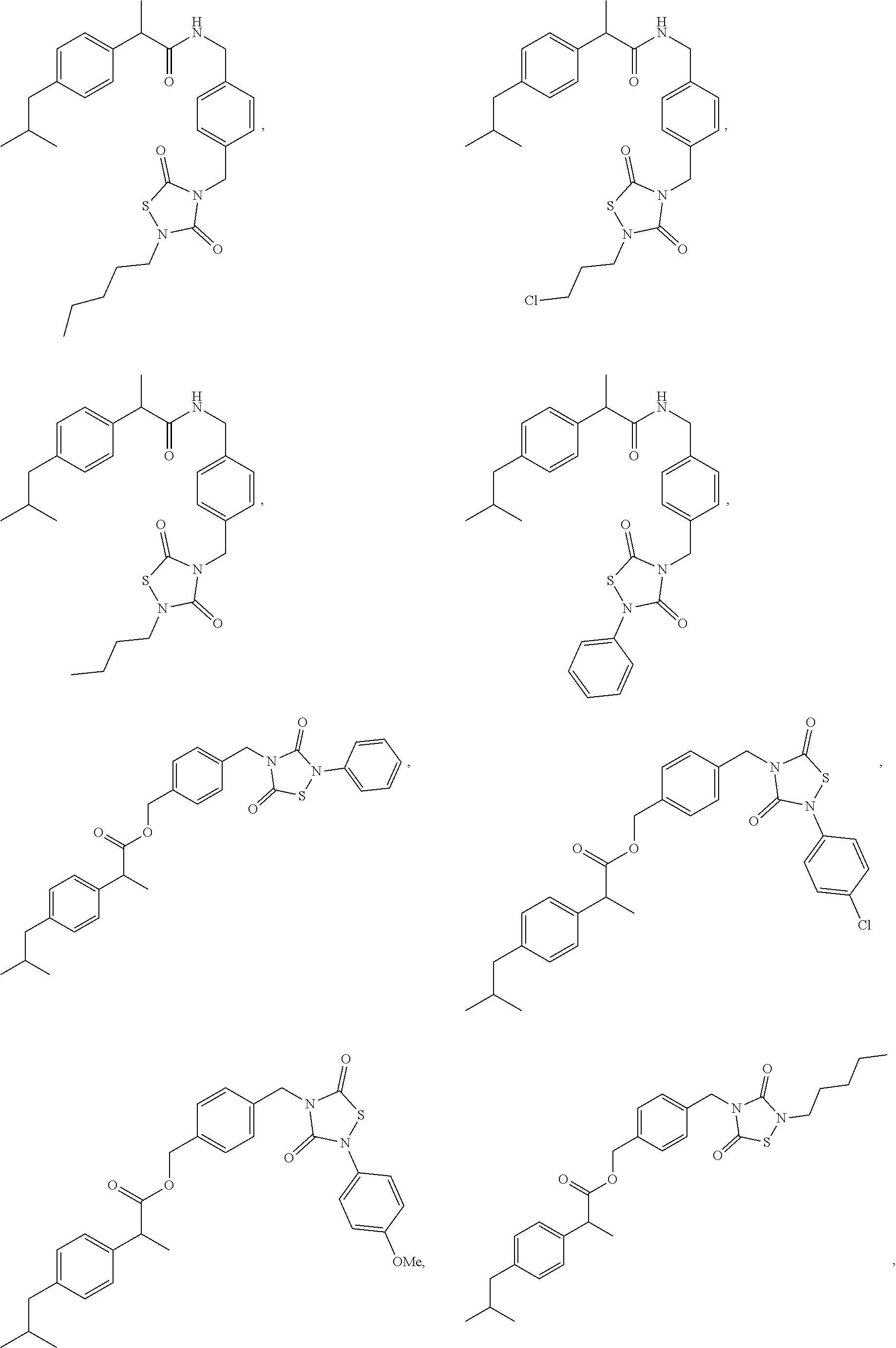

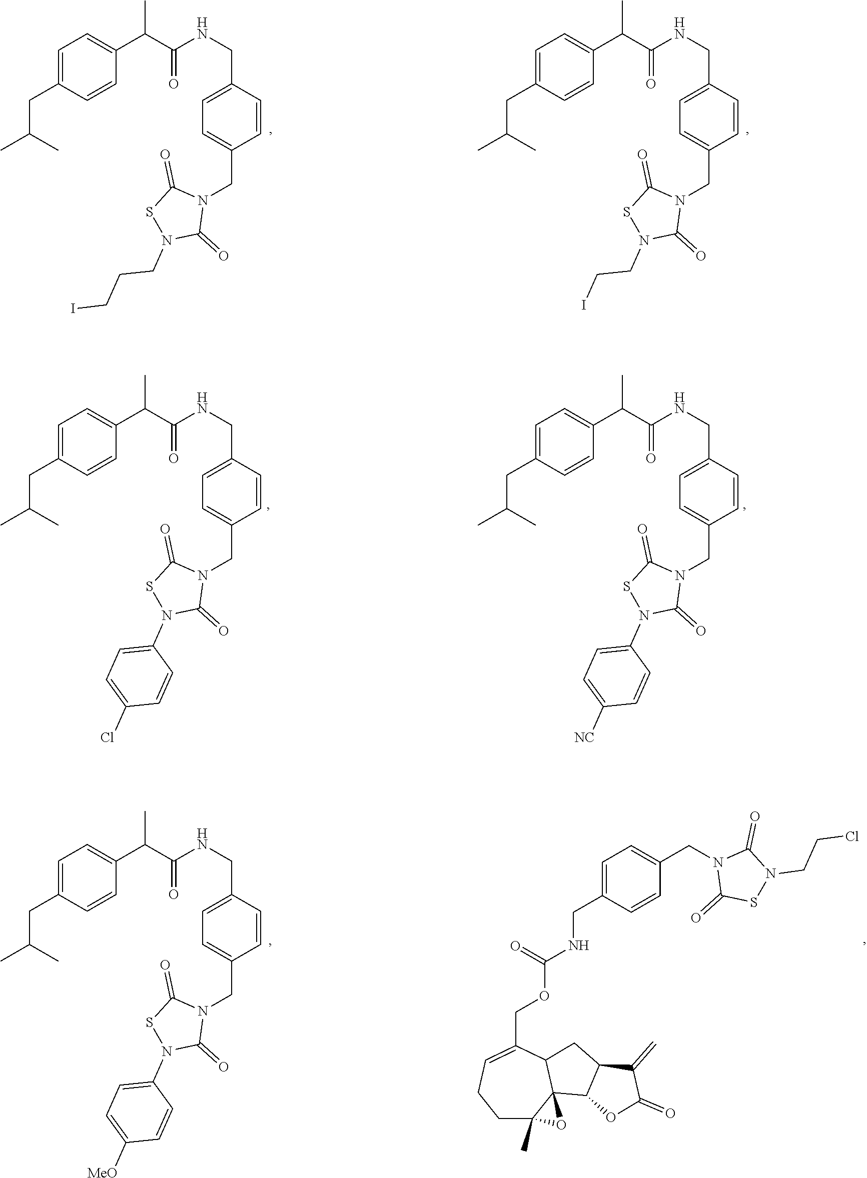 [SMILES: CC(C)Cc1ccc(C(C)C(=O)NCc2ccc(Cn3c(=O)sn(CCCI)c3=O)cc2)cc1 ; CC(C)Cc1ccc(C(C)C(=O)NCc2ccc(Cn3c(=O)sn(CCI)c3=O)cc2)cc1 ; CC(C)Cc1ccc(C(C)C(=O)NCc2ccc(Cn3c(=O)sn(-c4ccc(Cl)cc4)c3=O)cc2)cc1 ; CC(C)Cc1ccc(C(C)C(=O)NCc2ccc(Cn3c(=O)sn(-c4ccc(C#N)cc4)c3=O)cc2)cc1 ; COc1ccc(-n2sc(=O)n(Cc3ccc(CNC(=O)C(C)c4ccc(CC(C)C)cc4)cc3)c2=O)cc1 ; C=C1C(=O)O[C@H]2[C@H]1CC1C(COC(=O)NCc3ccc(Cn4c(=O)sn(CCCl)c4=O)cc3)=CCC[C@@]3(C)O[C@@]123]

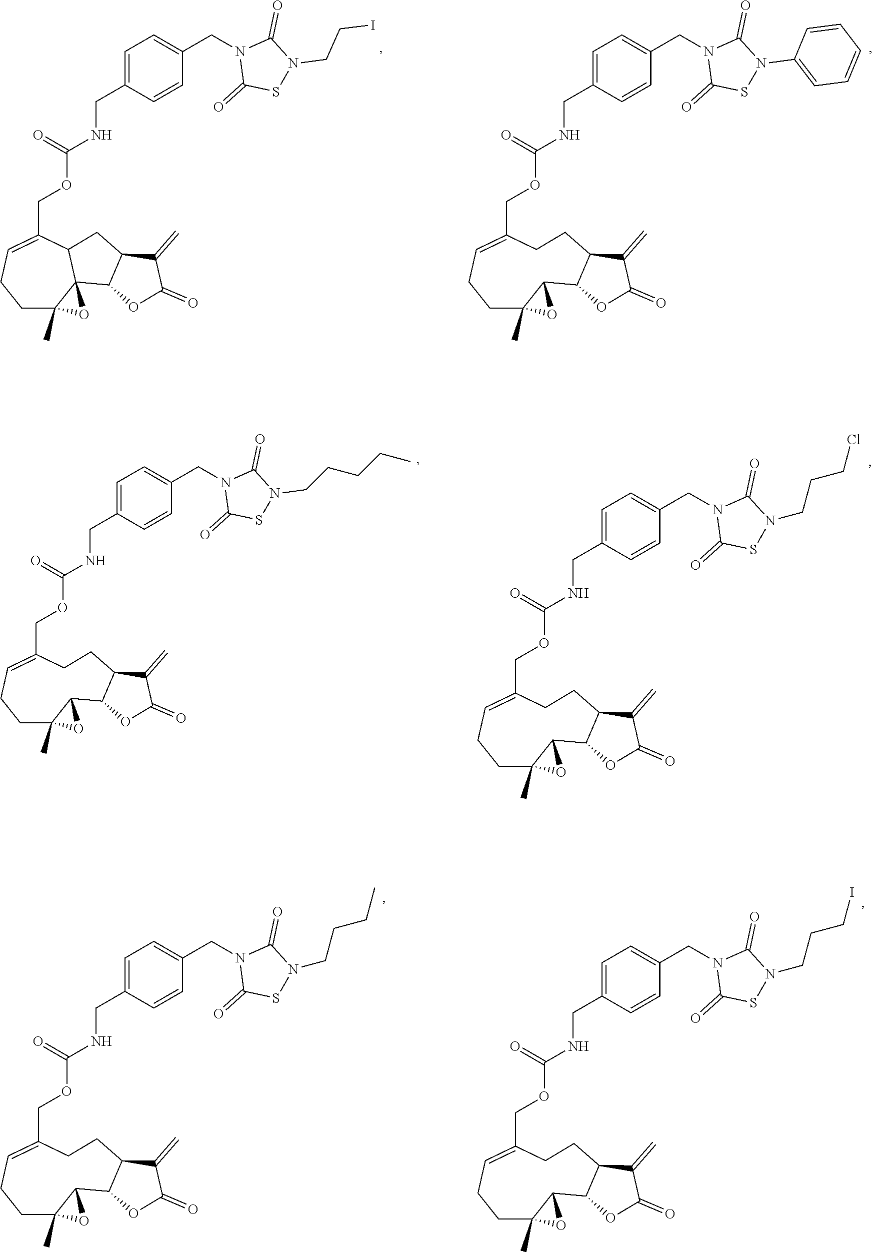

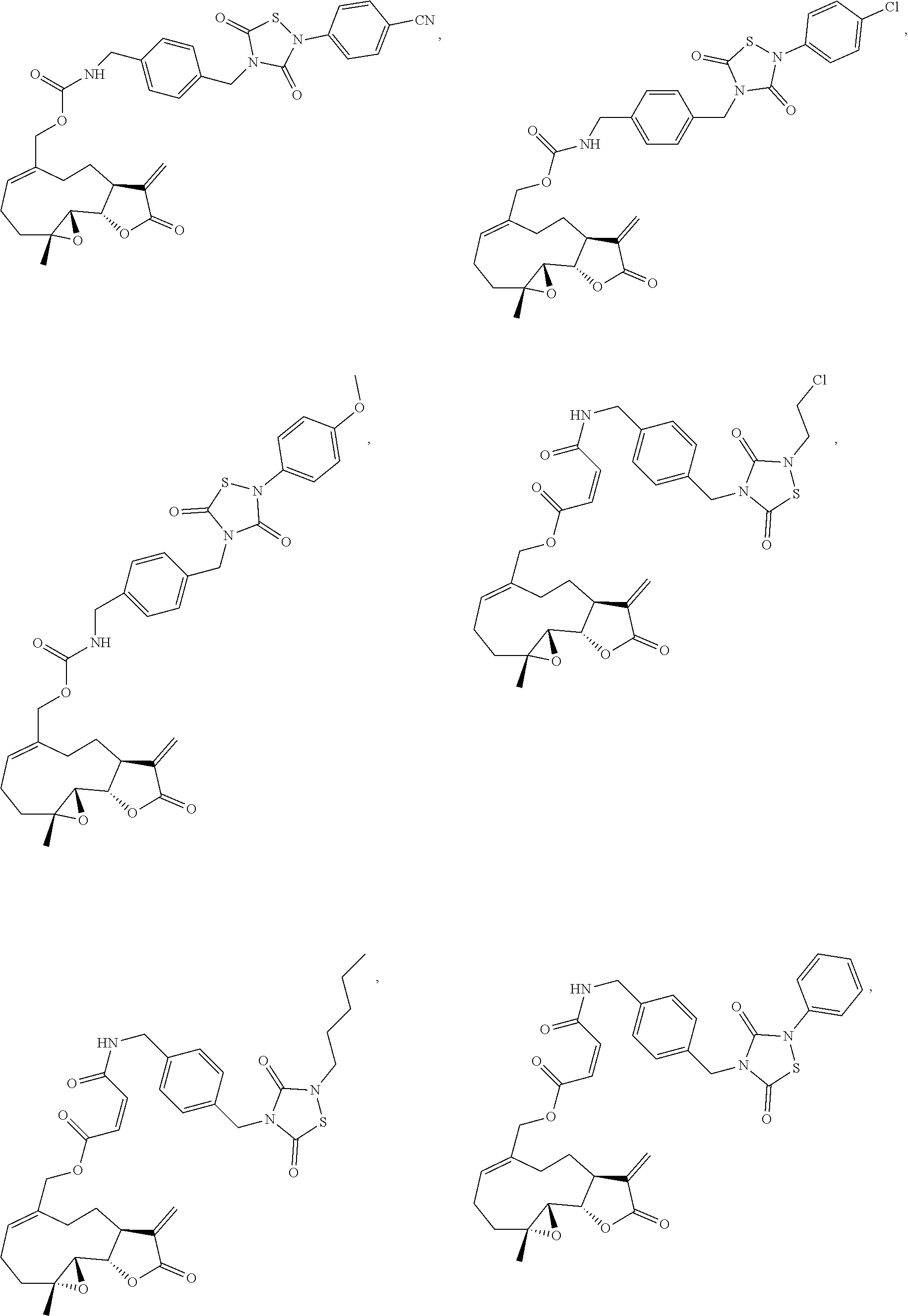

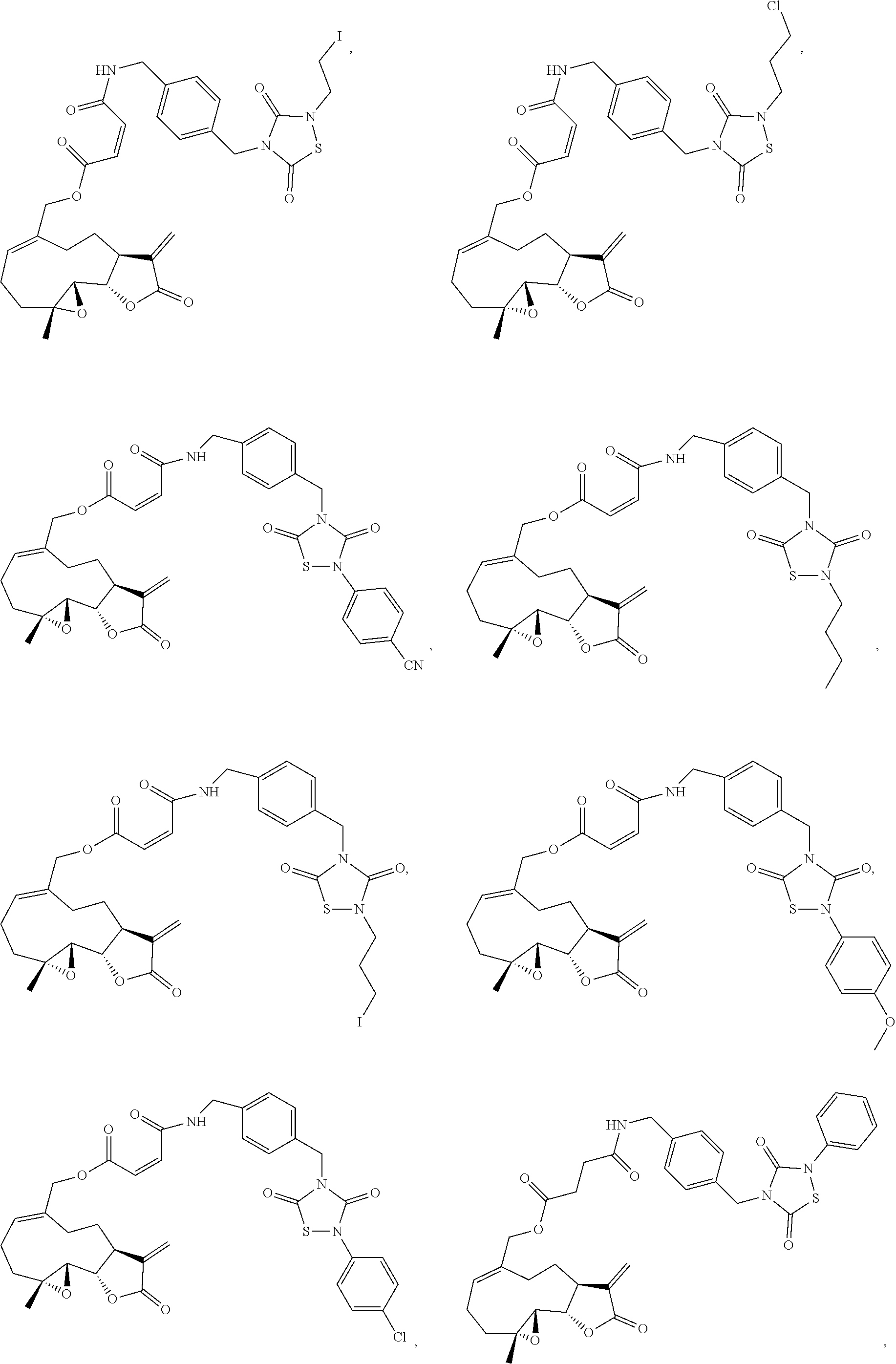

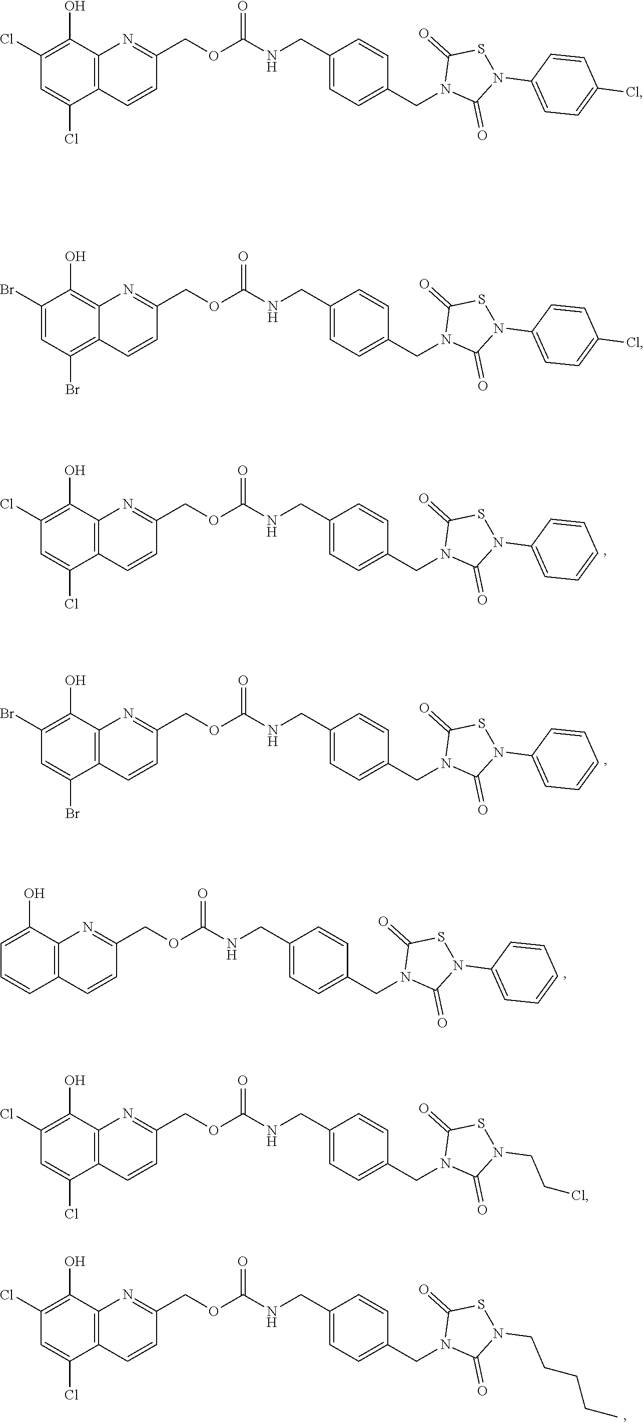

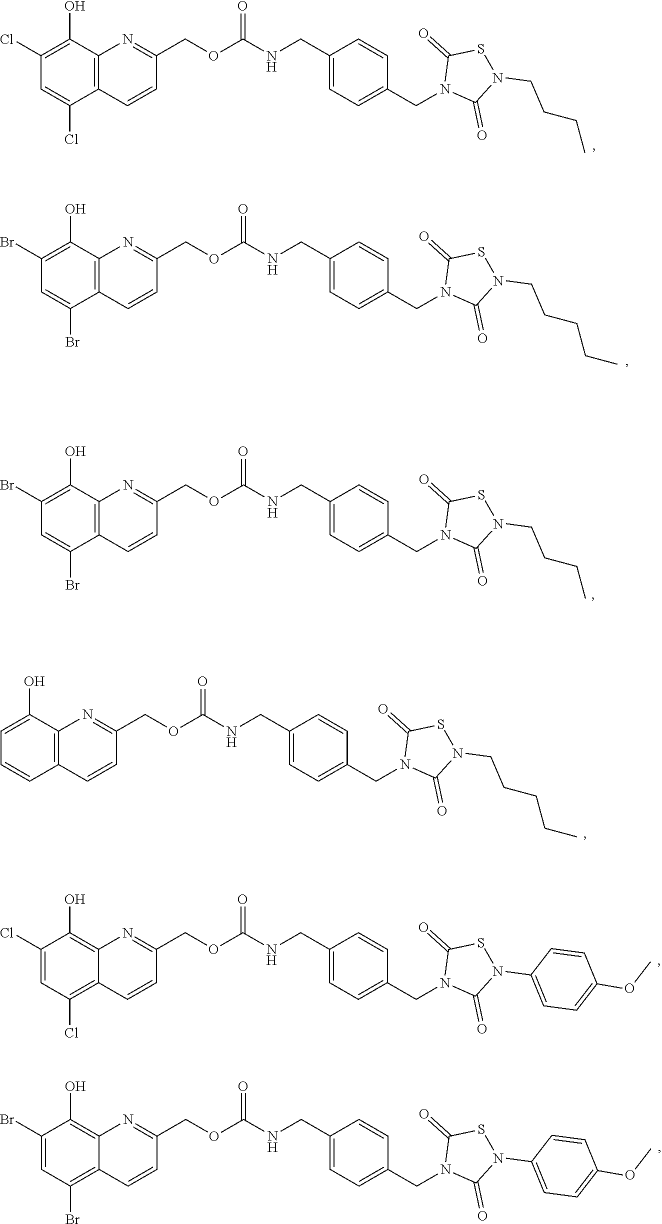 [SMILES: CCCCn1sc(=O)n(Cc2ccc(CNC(=O)OCc3ccc4c(Cl)cc(Cl)c(O)c4n3)cc2)c1=O ; CCCCCn1sc(=O)n(Cc2ccc(CNC(=O)OCc3ccc4c(Br)cc(Br)c(O)c4n3)cc2)c1=O ; CCCCn1sc(=O)n(Cc2ccc(CNC(=O)OCc3ccc4c(Br)cc(Br)c(O)c4n3)cc2)c1=O ; CCCCCn1sc(=O)n(Cc2ccc(CNC(=O)OCc3ccc4cccc(O)c4n3)cc2)c1=O ; COc1ccc(-n2sc(=O)n(Cc3ccc(CNC(=O)OCc4ccc5c(Cl)cc(Cl)c(O)c5n4)cc3)c2=O)cc1 ; COc1ccc(-n2sc(=O)n(Cc3ccc(CNC(=O)OCc4ccc5c(Br)cc(Br)c(O)c5n4)cc3)c2=O)cc1]

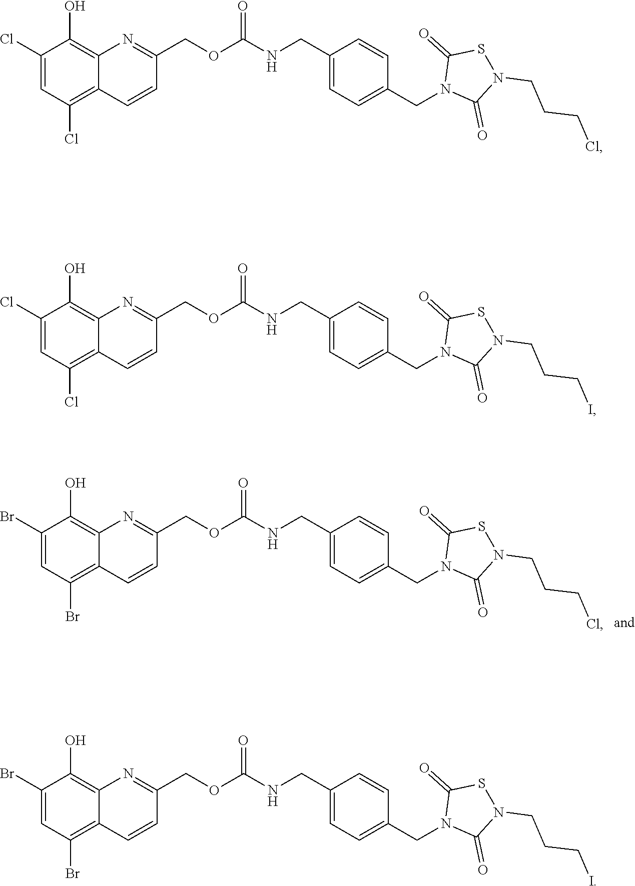 [SMILES: O=C(NCc1ccc(Cn2c(=O)sn(CCCCl)c2=O)cc1)OCc1ccc2c(Cl)cc(Cl)c(O)c2n1 ; O=C(NCc1ccc(Cn2c(=O)sn(CCCI)c2=O)cc1)OCc1ccc2c(Cl)cc(Cl)c(O)c2n1 ; O=C(NCc1ccc(Cn2c(=O)sn(CCCCl)c2=O)cc1)OCc1ccc2c(Br)cc(Br)c(O)c2n1 ; O=C(NCc1ccc(Cn2c(=O)sn(CCCI)c2=O)cc1)OCc1ccc2c(Br)cc(Br)c(O)c2n1]

In various aspects, the compound is selected from:

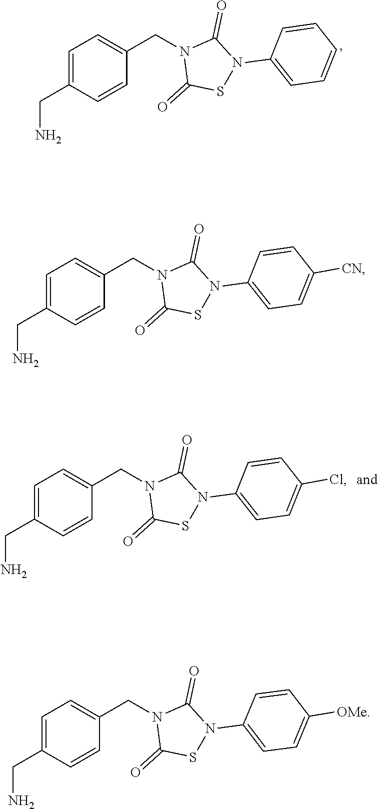 [SMILES: NCc1ccc(Cn2c(=O)sn(-c3ccccc3)c2=O)cc1 ; N#Cc1ccc(-n2sc(=O)n(Cc3ccc(CN)cc3)c2=O)cc1 ; NCc1ccc(Cn2c(=O)sn(-c3ccc(Cl)cc3)c2=O)cc1 ; COc1ccc(-n2sc(=O)n(Cc3ccc(CN)cc3)c2=O)cc1]

In one aspect, m is 0, 1, 2, or 3. In a further aspect, m is 0, 1, or 2. In a still further aspect, m is 0 or 1. In yet a further aspect, m is 1 or 2. In an even further aspect, m is 2 or 3. In a still further aspect, m is 1, 2, or 3. In yet a further aspect, m is 3. In an even further aspect, m is 2. In a still further aspect, m is 1. In yet a further aspect, m is 0.

a. X Groups

In one aspect, X is selected from NH and O. In a further aspect, X is NH. In a still further aspect, X is O.

b. R¹ Groups

In one aspect, R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹. In a further aspect, R¹ is selected from C1-C8 alkyl, C2-C8 alkenyl, C1-C8 haloalkyl, C1-C8 cyanoalkyl, C1-C8 nitroalkyl, C1-C8 hydroxyalkyl, C1-C8 alkoxy, C1-C8 alkenoxy, C1-C8 thioalkyl, C1-C8 alkylthiol, C1-C8 alkylamino, (C1-C8)(C1-C8) dialkylamino, C1-C8 aminoalkyl, —(C1-C8 alkyl)-O—(C1-C8 alkyl), —(C1-C8 alkyl)C(O)R¹⁰, —(C1-C8 alkyl)OC(O)(C1-C8 alkyl), —(C1-C8 alkyl)NHC(O)(C1-C8 alkyl), —(C1-C8 alkyl)N(C1-C8 alkyl)C(O)(C1-C8 alkyl), —(C1-C8)Cy¹, and Cy¹. In a still further aspect, R¹ is selected from C1-C4 alkyl, C2-C4 alkenyl, C1-C4 haloalkyl, C1-C4 cyanoalkyl, C1-C4 nitroalkyl, C1-C4 hydroxyalkyl, C1-C4 alkoxy, C1-C4 alkenoxy, C1-C4 thioalkyl, C1-C4 alkylthiol, C1-C4 alkylamino, (C1-C4)(C1-C4) dialkylamino, C1-C4 aminoalkyl, —(C1-C4 alkyl)-O—(C1-C4 alkyl), —(C1-C4 alkyl)C(O)R¹⁰, —(C1-C4 alkyl)OC(O)(C1-C4 alkyl), —(C1-C4 alkyl)NHC(O)(C1-C4 alkyl), —(C1-C4 alkyl)N(C1-C4 alkyl)C(O)(C1-C4 alkyl), —(C1-C4)Cy¹, and Cy¹.

In various aspects, R¹ is selected from C1-C10 alkyl, C2-C10 alkenyl, C1-C10 haloalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹. In a further aspect, R¹ is selected from C1-C8 alkyl, C2-C8 alkenyl, C1-C8 haloalkyl, —(C1-C8 alkyl)-O—(C1-C8 alkyl), —(C1-C8 alkyl)C(O)R¹⁰, —(C1-C8 alkyl)OC(O)(C1-C8 alkyl), —(C1-C8 alkyl)NHC(O)(C1-C8 alkyl), —(C1-C8 alkyl)N(C1-C8 alkyl)C(O)(C1-C8 alkyl), —(C1-C8)Cy¹, and Cy¹. In a still further aspect, R¹ is selected from C1-C4 alkyl, C2-C4 alkenyl, C1-C4 haloalkyl, —(C1-C4 alkyl)—O—(C1-C4 alkyl), —(C1-C4 alkyl)C(O)R¹⁰, —(C1-C4 alkyl)OC(O)(C1-C4 alkyl), —(C1-C4 alkyl)NHC(O)(C1-C4 alkyl), —(C1-C4 alkyl)N(C1-C4 alkyl)C(O)(C1-C4 alkyl), —(C1-C4)Cy¹, and Cy¹. In yet a further aspect, R¹ is selected from methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)₂Cl, —CH(CH₃)₂F, —CH₂OCH₃, —CH₂CH₂OCH₃, —CH₂OCH₂CH₃, —CH₂CH₂CH₂OCH₂CH₃, —CH₂CH₂OCH(CH₃)₂, —CH₂C(O)R¹⁰, —CH₂CH₂C(O)R¹⁰, —CH₂CH₂CH₂C(O)R¹⁰, —CH(CH₃)CH₂C(O)R¹⁰, —CH₂OC(O)CH₃, —CH₂CH₂O(CO)CH₃, —CH₂OC(O)CH₂CH₃, —CH₂CH₂CH₂OC(O)CH₂CH₃, —CH₂CH₂OC(O)CH(CH₃)₂, —CH₂NHC(O)CH₃, —CH₂CH₂NHC(O)CH₃, —CH₂NHC(O)CH₂CH₃, —CH₂CH₂CH₂NHC(O)CH₂CH₃, —CH₂CH₂NHC(O)CH(CH₃)₂, —CH₂N(CH₃)C(O)CH₃, —CH₂CH₂N(CH₂CH₃)C(O)CH₃, —CH₂N(CH₂CH₂CH₃)C(O)CH₂CH₃, —CH₂CH₂CH₂N(CH(CH₃)₂)C(O)CH₂CH₃, —CH₂CH₂N(CH₃)C(O)CH(CH₃)₂, —CH₂Cy¹, —CH₂CH₂Cy¹, —CH₂CH₂CH₂Cy¹, —CH(CH₃)₂Cy¹, and Cy¹. In an even further aspect, R¹ is selected from methyl, ethyl, ethenyl, —CH₂Cl, —CH₂F, —CH₂CH₂Cl, —CH₂CH₂F, —CH₂OCH₃, —CH₂CH₂OCH₃, —CH₂OCH₂CH₃, —CH₂C(O)R¹⁰, —CH₂CH₂C(O)R¹⁰, —CH₂OC(O)CH₃, —CH₂CH₂O(CO)CH₃, —CH₂OC(O)CH₂CH₃, —CH₂NHC(O)CH₃, —CH₂CH₂NHC(O)CH₃, —CH₂NHC(O)CH₂CH₃, —CH₂N(CH₃)C(O)CH₃, —CH₂CH₂N(CH₂CH₃)C(O)CH₃, —CH₂Cy¹, —CH₂CH₂Cy¹, and Cy¹. In a still further aspect, R¹ is selected from methyl, —CH₂Cl, —CH₂F, —CH₂OCH₃, —CH₂C(O)R¹⁰, —CH₂OC(O)CH₃, —CH₂NHC(O)CH₃, —CH₂N(CH₃)C(O)CH₃, —CH₂Cy¹, and Cy¹.

In various aspects, R¹ is selected from C1-C10 alkyl, C1-C10 cyanoalkyl, C1-C10 nitroalkyl, —(C1-C10)Cy¹, and Cy¹. In a further aspect, R¹ is selected from C1-C8 alkyl, C1-C8 cyanoalkyl, C1-C8 nitroalkyl, —(C1-C8)Cy¹, and Cy¹. In a still further aspect, R¹ is selected from C1-C4 alkyl, C1-C4 cyanoalkyl, C1-C4 nitroalkyl, —(C1-C4)Cy¹, and Cy¹. In yet a further aspect, R¹ is selected from methyl, ethyl, n-propyl, isopropyl, —CH₂CN, —CH₂CH₂CN, —CH₂CH₂CH₂CN, —CH(CH₃)₂CN, —CH₂NO₂, —CH₂CH₂NO₂, —CH₂CH₂CH₂NO₂, —CH(CH₃)₂NO₂, —CH₂Cy¹, —CH₂CH₂Cy¹, —CH₂CH₂CH₂Cy¹, —CH(CH₃)₂Cy¹, and Cy¹. In an even further aspect, R¹ is selected from methyl, ethyl, —CH₂CN, —CH₂CH₂CN, —CH₂NO₂, —CH₂CH₂NO₂, —CH₂Cy¹, —CH₂CH₂Cy¹, and Cy¹. In a still further aspect, R¹ is selected from methyl, —CH₂CN, —CH₂NO₂, —CH₂Cy¹, and Cy¹.

In various aspects, R¹ is selected from C1-C10 alkyl, C1-C10 hydroxyalkyl, C1-C10 alkoxy, C1-C10 alkenoxy, —(C1-C10)Cy¹, and Cy¹. In a further aspect, R¹ is selected from C1-C8 alkyl, C1-C8 hydroxyalkyl, C1-C8 alkoxy, C1-C8 alkenoxy, —(C1-C8)Cy¹, and Cy¹. In a still further aspect, R¹ is selected from C1-C4 alkyl, C1-C4 hydroxyalkyl, C1-C4 alkoxy, C1-C4 alkenoxy, —(C1-C4)Cy¹, and Cy¹. In yet a further aspect, R¹ is selected from methyl, ethyl, n-propyl, isopropyl, —CH₂OH, —CH₂CH₂OH, —CH₂CH₂CH₂OH, —CH(CH₃)₂OH, —OCH₃, —OCH₂CH₃, —OCH₂CH₂CH₃, —OCH₂(CH₃)₂, —OCH═CH₂, —OCH═CHCH₃, —OC(CH₃)₂CH₂, —CH₂Cy¹, —CH₂CH₂Cy¹, —CH₂CH₂CH₂Cy¹, —CH(CH₃)₂Cy¹, and Cy¹. In an even further aspect, R¹ is selected from methyl, ethyl, —CH₂OH, —CH₂CH₂OH, —OCH₃, —OCH₂CH₃, —OCH═CH₂, —CH₂Cy¹, —CH₂CH₂Cy¹, and Cy¹. In a still further aspect, R¹ is selected from methyl, —CH₂OH, —OCH₃, —CH₂Cy¹, and Cy¹.

In various aspects, R¹ is selected from C1-C10 alkyl, C1-C10 thioalkyl, C1-C10 alkylthiol, —(C1-C10)Cy¹, and Cy¹. In a further aspect, R¹ is selected from C1-C8 alkyl, C1-C8 thioalkyl, C1-C8 alkylthiol, —(C1-C8)Cy¹, and Cy¹. In a still further aspect, R¹ is selected from C1-C4 alkyl, C1-C4 thioalkyl, C1-C4 alkylthiol, —(C1-C4)Cy¹, and Cy¹. In yet a further aspect, R¹ is selected from methyl, ethyl, n-propyl, isopropyl, —CH₂SH, —CH₂CH₂SH, —CH₂CH₂CH₂SH, —CH(CH₃)₂SH, —SCH₃, —SCH₂CH₃, —SCH₂CH₂CH₃, —SCH₂(CH₃)₂, —CH₂Cy¹, —CH₂CH₂Cy¹, —CH₂CH₂CH₂Cy¹, —CH(CH₃)₂Cy¹, and Cy¹. In an even further aspect, R¹ is selected from methyl, ethyl, —CH₂SH, —CH₂CH₂SH, —SCH₃, —SCH₂CH₃, —CH₂Cy¹, —CH₂CH₂Cy¹, and Cy¹. In a still further aspect, R¹ is selected from methyl, —CH₂SH, —SCH₃, —CH₂Cy¹, and Cy¹.

In various aspects, R¹ is selected from C1-C10 alkyl, C1-C10 alkylamino, (C1-C10)(C1-C10) dialkylamino, C1-C10 aminoalkyl, —(C1-C10)Cy¹, and Cy¹. In a further aspect, R¹ is selected from C1-C8 alkyl, C1-C8 alkylamino, (C1-C8)(C1-C8) dialkylamino, C1-C8 aminoalkyl, —(C1-C8)Cy¹, and Cy¹. In a still further aspect, R¹ is selected from C1-C4 alkyl, C1-C4 alkylamino, (C1-C4)(C1-C4) dialkylamino, C1-C4 aminoalkyl, —(C1-C4)Cy¹, and Cy¹. In yet a further aspect, R¹ is selected from methyl, ethyl, n-propyl, isopropyl, —CH₂NH₂, —CH₂CH₂NH₂, —CH₂CH₂CH₂NH₂, —CH(CH₃)₂NH₂, —NHCH₃, —NHCH₂CH₃, —NHCH₂CH₂CH₃, —NHCH₂(CH₃)₂, —N(CH₃)₂, —N(CH₃)CH₂CH₃, —N(CH₃)CH₂CH₂CH₃, —N(CH₃)CH₂(CH₃)₂, —CH₂Cy¹, —CH₂CH₂Cy¹, —CH₂CH₂CH₂Cy¹, —CH(CH₃)₂Cy¹, and Cy¹. In an even further aspect, R¹ is selected from methyl, ethyl, —CH₂NH₂, —CH₂CH₂NH₂, —NHCH₃, —NHCH₂CH₃, —NHCH₂(CH₃)₂, —N(CH₃)₂, —N(CH₃)CH₂CH₃, —CH₂Cy¹, —CH₂CH₂Cy¹, and Cy¹. In a still further aspect, R¹ is selected from methyl, —CH₂NH₂, —NHCH₃, —NHCH₂(CH₃)₂, —N(CH₃)₂, —CH₂Cy¹, and Cy¹.

In various aspects, R¹ is selected from C1-C10 alkyl, —(C1-C10)Cy¹, and Cy¹. In a further aspect, R¹ is selected from C1-C8 alkyl, —(C1-C8)Cy¹, and Cy¹. In a still further aspect, R¹ is selected from C1-C4 alkyl, —(C1-C4)Cy¹, and Cy¹. In yet a further aspect, R¹ is selected from methyl, ethyl, n-propyl, isopropyl, —CH₂Cy¹, —CH₂CH₂Cy¹, —CH₂CH₂CH₂Cy¹, —CH(CH₃)₂Cy¹, and Cy¹. In an even further aspect, R¹ is selected from methyl, ethyl, —CH₂Cy¹, —CH₂CH₂Cy¹, and Cy¹. In a still further aspect, R¹ is selected from methyl, —CH₂Cy¹, and Cy¹.

In various aspects, R¹ is selected from C1-C10 alkyl, C1-C10 haloalkyl, —(C1-C10)Cy¹, and Cy¹. In a further aspect, R¹ is selected from C1-C8 alkyl, C1-C8 haloalkyl, —(C1-C8)Cy¹, and Cy¹. In a further aspect, R¹ is selected from C1-C4 alkyl, C1-C4 haloalkyl, —(C1-C8)Cy¹, and Cy¹. In a still further aspect, R¹ is selected from methyl, ethyl, n-propyl, isopropyl, CH₂Cl, —CH₂F, —CH₂CH₂Cl, —CH₂CH₂F, —CH₂CH₂CH₂Cl, —CH₂CH₂CH₂F, —CH(CH₃)₂Cl, —CH(CH₃)₂F, —CH₂Cy¹, —CH₂CH₂Cy¹, —CH₂CH₂CH₂Cy¹, —CH(CH₃)₂Cy¹, and Cy¹. In yet a further aspect, R¹ is selected from methyl, ethyl, —CH₂Cl, —CH₂F, —CH₂CH₂Cl, —CH₂CH₂F, —CH₂Cy¹, —CH₂CH₂Cy¹, and Cy¹. In an even further aspects, R¹ is selected from methyl, —CH₂Cl, —CH₂F, —CH₂Cy¹, and Cy¹.

In various aspects, R¹ is selected from C1-C10 alkyl, C1-C10 haloalkyl, and Cy¹. In a further aspect, R¹ is selected from C1-C8 alkyl, C1-C8 haloalkyl, —(C1-C8)Cy¹, and Cy¹. In a further aspect, R¹ is selected from C1-C4 alkyl, C1-C4 haloalkyl, and Cy¹. In a still further aspect, R¹ is selected from methyl, ethyl, n-propyl, isopropyl, —CH₂Cl, —CH₂F, —CH₂CH₂Cl, —CH₂CH₂F, —CH₂CH₂CH₂Cl, —CH₂CH₂CH₂F, —CH(CH₃)₂Cl, —CH(CH₃)₂I, —CH(CH₃)₂F, and Cy¹. In yet a further aspect, R¹ is selected from methyl, ethyl, —CH₂Cl, —CH₂F, —CH₂CH₂Cl, —CH₂CH₂F, and Cy¹. In an even further aspects, R¹ is selected from methyl, —CH₂Cl, —CH₂F, and Cy¹.

In various aspects, R¹ is Cy¹.

c. R^{2a}, R^{2b}, R^{2c}, R^{2d}, and R^{2E} Groups

In one aspect, each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

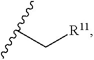

provided that one of R^{2a}, R^{2b}, R^{2c}, R^{2d}, and R^{2e} is Ar¹ or

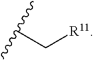

In a further aspect, each of R^{2a}, R^{2b}, R^{2c}, R^{2d}, and R^{2e} is independently selected from hydrogen, —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, Ar¹, and

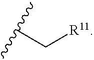

In a still further aspect, each of R^{2a}, R^{2b}, R^{2c}, R^{2d}, and R^{2e} is independently selected from hydrogen, —F, —Cl, —CN, —NH₂, —OH, —NO₂, —OC(O)CH₃, —OC(O)CH₂CH₃, methyl, ethyl, ethenyl, —CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, —N(CH₃)CH₂CH₃, Ar¹, and

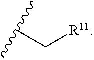

In yet a further aspect, each of R^{2a}, R^{2b}, R^{2c}, R^{2d}, and R^{2e} is independently selected from hydrogen, —F, —Cl, —CN, —NH₂, —OH, —NO₂, —OC(O)CH₃, methyl, —CH₂Cl, —CH₂F, —CH₂CN, —CH₂OH, —OCH₂Cl, —OCH₂F, —OCH₃, —CH₂NH₂, —NHCH₃, —N(CH₃)₂, Ar¹, and

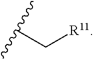

In various aspects, each of R^{2a}, R^{2b}, R^{2c}, R^{2d}, and R^{2e} is independently selected from hydrogen, halogen, —CN, —NH₂, —OH, —NO₂, C1-C4 alkyl, Ar¹, and a structure having a formula:

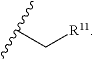

In a further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, n-propyl, isopropyl, Ar¹, and a structure having a formula:

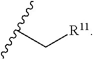

In a still further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, Ar¹, and a structure having a formula:

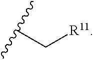

In yet a further aspect, each of R^{2a}, R^{2b}, R^{2c}, R^{2d}, and R^{2e} is independently selected from hydrogen, —F, —Cl, —CN, —NH₂, —OH, —NO₂, methyl, Ar¹, and a structure having a formula:

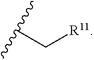

In various aspects, each of R^{2a}, R^{2b}, R^{2c}, R^{2d}, and R^{2e} is independently selected from hydrogen, halogen, Ar¹, and a structure having a formula:

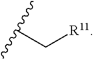

In a further aspect, each of R^{2a}, R^{2b}, R^{2c}, R^{2d}, and R^{2e} is independently selected from hydrogen, —Br, —Cl, —F, Ar¹, and a structure having a formula:

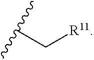

In a still further aspect, each of R^{2a}, R^{2b}, R^{2c}, R^{2d}, and R^{2e} is independently selected from hydrogen, —Cl, —F, Ar¹, and a structure having a formula:

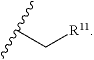

In yet a further aspect, each of R^{2a}, R^{2b}, R^{2c}, R^{2d}, and R^{2e} is independently selected from hydrogen, —F, Ar¹, and a structure having a formula:

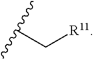

In various aspects, each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), Ar¹, and a structure having formula

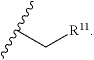

In a further aspect, each of R^{2a}, R^{2b}, R^{2c}, R^{2d}, and R^{2e} is independently selected from —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, Ar¹, and a structure having a formula:

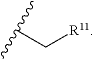

In a still further aspect, each of R^{2a}, R^{2b}, R^{2c}, R^{2d}, and R^{2e} is independently selected from —F, —Cl, —CN, —NH₂, —OH, —NO₂, —OC(O)CH₃, —OC(O)CH₂CH₃, Ar¹, and a structure having a formula:

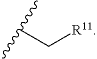

In yet a further aspect, each of R^{2a}, R^{2b}, R^{2c}, R^{2d}, and R^{2e} is independently selected from —F, —Cl, —CN, —NH₂, —OH, —NO₂, —OC(O)CH₃, Ar¹, and a structure having a formula:

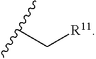

In various aspects, each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, Ar¹, and a structure having a formula:

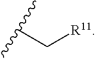

In a further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, n-propyl, isopropyl, ethenyl, n-propenyl, isopropenyl, Ar¹, and a structure having a formula:

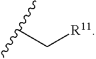

In a still further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, ethenyl, Ar¹, and a structure having a formula:

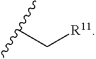

In yet a further aspect, each of R^{2a}, R^{2b}, R^{2c}, R^{2d}, and R^{2e} is independently selected from hydrogen —F, —Cl, —CN, —NH₂, —OH, —NO₂, methyl, Ar¹, and a structure having a formula:

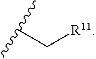

In various aspects, each of R^{2a}, R^{2b}, R^{2c}, R^{2d}, and R^{2e} is independently selected from hydrogen, halogen, —CN, —NH₂, —OH, —NO₂, C1-C4 haloalkyl, C1-C4 cyanoalkyl, Ar¹, and a structure having a formula:

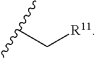

In a further aspect, each of R^{2a}, R^{2b}, R^{2c}, R^{2d}, and R^{2e} is independently selected from hydrogen, —F, —Cl, —CN, —NH₂, —OH, —NO₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, Ar¹, and a structure having a formula:

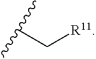

In a still further aspect, each of R^{2a}, R^{2b}, R^{2c}, R^{2d}, and R^{2e} is independently selected from hydrogen, —F, —Cl, —CN, —NH₂, —OH, —NO₂, —CH₂Cl, —CH₂F, —CH₂CH₂Cl, —CH₂CH₂F, —CH₂CN, —CH₂CH₂CN, Ar¹, and a structure having a formula:

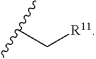

In yet a further aspect, each of R^{2a}, R^{2b}, R^{2c}, R^{2d}, and R^{2e} is independently selected from hydrogen, —F, —Cl, —CN, —NH₂, —OH, —NO₂, —CH₂Cl, —CH₂F, —CH₂CN, Ar¹, and a structure having a formula:

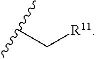

In various aspects, each of R^{2a}, R^{2b}, R^{2c}, R^{2d}, and R^{2e} is independently selected from hydrogen, halogen, —CN, —NH₂, —OH, —NO₂, C1-C4 hydroxyalkyl, C1-C4 haloalkoxy, C1-C4 alkoxy, Ar¹, and a structure having a formula:

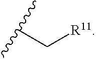

In a further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, OCH(CH₃)CH₂Cl, —OCH(CH₃)CH₂F, —OCH₃, —OCH₂CH₃, —OCH₂CH₂CH₃, —OCH(CH₃)₂, Ar¹, and a structure having a formula:

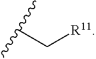

In a still further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —CH₂CH₂OH, —OCH₂Cl, —OCH₂F, —OCH₂CCH₂Cl, —OCH₂CH₂F, —OCH₃, —OCH₂CH₃, Ar¹, and a structure having a formula:

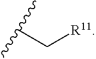

In yet a further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —OCH₂Cl, —OCH₂F, —OCH₃, Ar¹, and a structure having a formula:

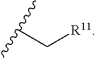

In various aspects, each of R^{2a}, R^{2b}, R^{2c}, R^{2d}, and R^{2e} is independently selected from hydrogen, halogen, —CN, —NH₂, —OH, —NO₂, C1-C4 alkylamino, (C1-C4)(C1-C4) dialkylamino, C1-C4 aminoalkyl, Ar¹, and a structure having a formula:

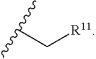

In a further aspect, each of R^{2a}, R^{2b}, R^{2c}, R^{2d}, and R^{2e} is independently selected from hydrogen, —F, —Cl, —CN, —NH₂, —OH, —NO₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, Ar¹, and a structure having a formula:

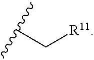

In a still further aspect, each of R^{2a}, R^{2b}, R^{2c}, R^{2d}, and R^{2e} is independently selected from hydrogen, —F, —Cl, —CN, —NH₂, —OH, —NO₂, —CH₂NH₂, —CH₂CH₂NH₂, —NHCH₃, —NHCH₂CH₃, —N(CH₃)₂, —N(CH₃)CH₂CH₃, Ar¹, and a structure having a formula:

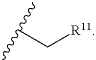

In yet a further aspect, each of R^{2a}, R^{2b}, R^{2c}, R^{2d}, and R^{2e} is independently selected from hydrogen, —F, —Cl, —CN, —NH₂, —OH, —NO₂, —CH₂NH₂, —NHCH₃, —N(CH₃)₂, Ar¹, and a structure having a formula:

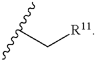

In various aspects, one of R^{2a}, R^{2b}, R^{2c}, R^{2d}, and R^{2e} is selected from Ar¹ and

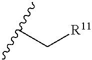

and four of R^{2a}, R^{2b}, R^{2c}, R^{2d}, and R^{2e} are independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, one of R^{2a}, R^{2b}, R^{2c}, R^{2d}, and R^{2e} is selected from Ar¹ and

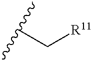

and four of R^{2a}, R^{2b}, R^{2c}, R^{2d}, and R^{2e} are independently selected from hydrogen, —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₃)CH(CH₃)₂. In a still further aspect, one of R^{2a}, R^{2b}, R^{2c}, R^{2d}, and R^{2e} is selected from Ar¹ and

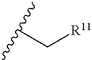

and four of R^{2a}, R^{2b}, R^{2c}, R^{2d}, and R^{2e} are independently selected from hydrogen, —F, —Cl, —CN, —NH₂, —OH, —NO₂, —OC(O)CH₃, —OC(O)CH₂CH₃, methyl, ethyl, ethenyl, —CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, and —N(CH₃)CH₂CH₃. In yet a further aspect, one of R^{2a}, R^{2b}, R^{2c}, R^{2d}, and R^{2e} is selected from Ar¹ and

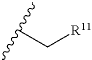

and four of R^{2a}, R^{2b}, R^{2c}, R^{2d}, and R^{2e} are independently selected from hydrogen, —F, —Cl, —CN, —NH₂, —OH, —NO₂, —OC(O)CH₃, methyl, —CH₂Cl, —CH₂F, —CH₂CN, —CH₂OH, —OCH₂Cl, —OCH₂F, —OCH₃, —CH₂NH₂, —NHCH₃, and —N(CH₃)₂.

In various aspects, R^{2c} is selected from Ar¹ and

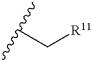

and R^{2a}, R^{2b}, R^{2d}, and R^{2e} are independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, R^{2c} is selected from Ar¹ and

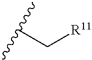

and R^{2a}, R^{2b}, R^{2d}, and R^{2e} are independently selected from hydrogen, —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₃)CH(CH₃)₂. In a still further aspect, R^{2c} is selected from Ar¹ and

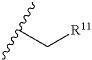

and R^{2a}, R^{2b}, R^{2d}, and R^{2e} are independently selected from hydrogen, —F, —Cl, —CN, —NH₂, —OH, —NO₂, —OC(O)CH₃, —OC(O)CH₂CH₃, methyl, ethyl, ethenyl, —CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, and —N(CH₃)CH₂CH₃. In yet a further aspect, R^{2c} is selected from Ar¹ and

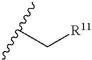

and R^{2a}, R^{2b}, R^{2d}, and R^{2e} are independently selected from hydrogen, —F, —Cl, —CN, —NH₂, —OH, —NO₂, —OC(O)CH₃, methyl, —CH₂Cl, —CH₂F, —CH₂CN, —CH₂OH, —OCH₂Cl, —OCH₂F, —OCH₃, —CH₂NH₂, —NHCH₃, and —N(CH₃)₂.

In various aspects, one of R^{2a}, R^{2b}, R^{2c}, R^{2d}, and R^{2e} is

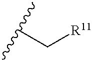

and four of R^{2a}, R^{2b}, R^{2c}, R^{2d}, and R^{2e} are independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, one of R^{2a}, R^{2b}, R^{2c}, R^{2d}, and R^{2e} is

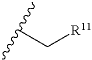

and four of R^{2a}, R^{2b}, R^{2c}, R^{2d}, and R^{2e} are independently selected from hydrogen, —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₃)CH(CH₃)₂. In a still further aspect, one of R^{2a}, R^{2b}, R^{2c}, R^{2d}, and R^{2e} is

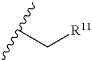

and four of R^{2a}, R^{2b}, R^{2c}, R^{2d}, and R^{2e} are independently selected from hydrogen, —F, —Cl, —CN, —NH₂, —OH, —NO₂, —OC(O)CH₃, —OC(O)CH₂CH₃, methyl, ethyl, ethenyl, —CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, and —N(CH₃)CH₂CH₃. In yet a further aspect, one of R^{2a}, R^{2b}, R^{2c}, R^{2d}, and R^{2e} is

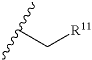

and four of R^{2a}, R^{2b}, R^{2c}, R^{2d}, and R^{2e} are independently selected from hydrogen, —F, —Cl, —CN, —NH₂, —OH, —NO₂, —OC(O)CH₃, methyl, —CH₂Cl, —CH₂F, —CH₂CN, —CH₂OH, —OCH₂Cl, —OCH₂F, —OCH₃, —CH₂NH₂, —NHCH₃, and —N(CH₃)₂.

In various aspects, R^{2c} is

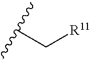

and R^{2a}, R^{2b}, R^{2d}, and R^{2e} are independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, R^{2c} is

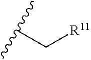

and R^{2a}, R^{2b}, R^{2d}, and R^{2e} are independently selected from hydrogen, —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₃)CH(CH₃)₂. In a still further aspect, R^{2c} is

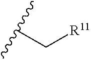

and R^{2a}, R^{2b}, R^{2d}, and R^{2e} are independently selected from hydrogen, —F, —Cl, —CN, —NH₂, —OH, —NO₂, —OC(O)CH₃, —OC(O)CH₂CH₃, methyl, ethyl, ethenyl, —CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, and —N(CH₃)CH₂CH₃. In yet a further aspect, R^{2c} is

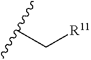

and R^{2a}, R^{2b}, R^{2d}, and R^{2e} are independently selected from hydrogen, —F, —Cl, —CN, —NH₂, —OH, —NO₂, —OC(O)CH₃, methyl, —CH₂Cl, —CH₂F, —CH₂CN, —CH₂OH, —OCH₂Cl, —OCH₂F, —OCH₃, —CH₂NH₂, —NHCH₃, and —N(CH₃)₂.

In various aspects, one of R^{2a}, R^{2b}, R^{2c}, R^{2d}, and R^{2e} is Ar¹ and four of R^{2a}, R^{2b}, R^{2c}, R^{2d}, and R^{2e} are independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, one of R^{2a}, R^{2b}, R^{2c}, R^{2d}, and R^{2e} is Ar¹ and four of R^{2a}, R^{2b}, R^{2c}, R^{2d}, and R^{2e} are independently selected from hydrogen, —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₃)CH(CH₃)₂. In a still further aspect, one of R^{2a}, R^{2b}, R^{2c}, R^{2d}, and R^{2e} is Ar¹ and four of R^{2a}, R^{2b}, R^{2c}, R^{2d}, and R^{2e} are independently selected from hydrogen, —F, —Cl, —CN, —NH₂, —OH, —NO₂, —OC(O)CH₃, —OC(O)CH₂CH₃, methyl, ethyl, ethenyl, —CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, and —N(CH₃)CH₂CH₃. In yet a further aspect, one of R^{2a}, R^{2b}, R^{2c}, R^{2d}, and R^{2e} is Ar¹ and four of R^{2a}, R^{2b}, R^{2c}, R^{2d}, and R^{2e} are independently selected from hydrogen, —F, —Cl, —CN, —NH₂, —OH, —NO₂, —OC(O)CH₃, methyl, —CH₂Cl, —CH₂F, —CH₂CN, —CH₂OH, —OCH₂Cl, —OCH₂F, —OCH₃, —CH₂NH₂, —NHCH₃, and —N(CH₃)₂.

In various aspects, R^{2c} is Ar¹ and R^{2a}, R^{2b}, R^{2d}, and R^{2e} are independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, R^{2c} is Ar¹ and R^{2a}, R^{2b}, R^{2d}, and R^{2e} are independently selected from hydrogen, —F, —Cl, —CN, —NH₂, —OH, —NO₂, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)CH₂OH, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₃)CH(CH₃)₂. In a still further aspect, R^{2c} is Ar¹ and R^{2a}, R^{2b}, R^{2c}, and R^{2e} are independently selected from hydrogen, —F, —Cl, —CN, —NH₂, —OH, —NO₂, —OC(O)CH₃, —OC(O)CH₂CH₃, methyl, ethyl, ethenyl, —CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, and —N(CH₃)CH₂CH₃. In yet a further aspect, R^{2c} is Ar¹ and R^{2a}, R^{2b}, R^{2d}, and R^{2e} are independently selected from hydrogen, —F, —Cl, —CN, —NH₂, —OH, —NO₂, —OC(O)CH₃, methyl, —CH₂Cl, —CH₂F, —CH₂CN, —CH₂OH, —OCH₂Cl, —OCH₂F, —OCH₃, —CH₂NH₂, —NHCH₃, and —N(CH₃)₂.

In various aspects, R^{2c} is selected from Ar¹ and

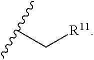

In a further aspect, R^{2a} is selected from Ar¹ and

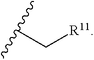

In a still further aspect, R^{2b} is selected from Ar¹ and

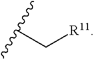

In yet a further aspect, R^{2d} is selected from Ar¹ and

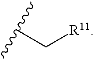

In yet a further aspect, R^{2e} is selected from Ar¹ and

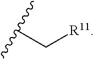

In various aspects, at least one of R^{2a}, R^{2b}, R^{2c}, R^{2d}, and R^{2e} is hydrogen. In a further aspect, at least two of R^{2a}, R^{2b}, R^{2c}, R^{2d}, and R^{2e} is hydrogen. In a still further aspect, at least three of R^{2a}, R^{2b}, R^{2c}, R^{2d}, and R^{2e} is hydrogen. In yet a further aspect, four of R^{2a}, R^{2b}, R^{2c}, R^{2d}, and R^{2e} are hydrogen.

In various aspects, three of R^{2a}, R^{2b}, R^{2c}, R^{2d}, and R^{2e} are hydrogen and one of R^{2a}, R^{2b}, R^{2c}, R^{2d}, and R^{2e} is selected from Ar¹ and

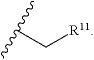

In various aspects, R^{2c} is selected from Ar¹ and

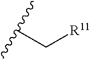

and each of R^{2a}, R^{2b}, R^{2d}, and R^{2e} is hydrogen.

In various aspects, one of R^{2a}, R^{2b}, R^{2c}, R^{2d}, and R^{2e} is

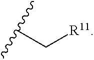

In various aspects, R^{2c} is

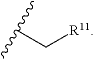

In a further aspect, R^{2a} is

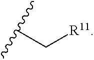

In a still further aspect, R^{2b} is

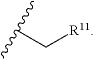

In yet a further aspect, R^{2d} is

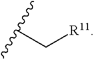

In an even further aspect, R^{2e} is

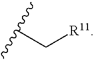

In various aspects, one of R^{2a}, R^{2b}, R^{2c}, R^{2d}, and R^{2e} is Ar¹.

In various aspects, R^{2c} is Ar¹. In a further aspect, R^{2a} is Ar¹. In a still further aspect, R^{2b} is Ar¹. In yet a further aspect, R^{2d} is Ar¹. In an even further aspect, R^{2e} is Ar¹.

d. R^{3a}, R^{3b}, R^{3c}, R^{3d}, AND R^{3e} Groups

In one aspect, each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂. In a further aspect, each of R^{3a}, R^{3b}, R^{3c}, R^{3d}, and R^{3e} is independently selected from hydrogen, halogen, —CN, —NH₂, —OH, —NO₂, —CO₂H, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂Cl, —OCH₂F, —OCHCl₂, —OCHF₂, —OCCl₃, —OCF₃, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, and Ar¹. In a still further aspect, each of R^{3a}, R^{3b}, R^{3c}, R^{3d}, and R^{3e} is independently selected from hydrogen, halogen, —CN, —NH₂, —OH, —NO₂, —CO₂H, —OC(O)CH₃, —OC(O)CH₂CH₃, methyl, ethyl, ethenyl, CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCHCl₂, —OCHF₂, —OCCl₃, —OCF₃, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, —N(CH₃)CH₂CH₃, and Ar¹. In yet a further aspect, each of R^{3a}, R^{3b}, R^{3c}, R^{3d}, and R^{3e} is independently selected from hydrogen, halogen, —CN, —NH₂, —OH, —NO₂, —CO₂H, —OC(O)CH₃, —OC(O)CH₂CH₃, methyl, CH₂Cl, —CH₂F, —CH₂CN, —CH₂OH, —OCH₂Cl, —OCH₂F, —OCHCl₂, —OCHF₂, —OCCl₃, —OCF₃, —OCH₃, —CH₂NH₂, —NHCH₃, —N(CH₃)₂, and Ar¹.

In various aspects, each of R^{3a}, R^{3b}, R^{3c}, R^{3d}, and R^{3e} is independently selected from hydrogen, halogen, —CN, —NH₂, —OH, —NO₂, —CO₂H, C1-C4 hydroxyalkyl, C1-C4 alkylamino, and Ar¹. In a further aspect, each of R^{3a}, R^{3b}, R^{3c}, R^{3d}, and R^{3e} is independently selected from hydrogen, —Br, —Cl, —F, —CN, —NH₂, —OH, —NO₂, —CO₂H, —CH₂OH, —CH₂CH₂OH, —CH₂CH₂CH₂OH, —CH(CH₃)CH₂OH, —CH₂NH₂, —CH₂CH₂NH₂, —CH₂CH₂CH₂NH₂, —CH(CH₃)CH₂NH₂, and Ar¹. In a still further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂OH, —CH₂CH₂OH, —CH₂NH₂, —CH₂CH₂NH₂, and Ar¹. In yet a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂OH, —CH₂NH₂, and Ar¹.

In various aspects, each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 hydroxyalkyl, C1-C4 alkylamino, and Ar¹. In a further aspect, each of R^{3a}, R^{3b}, R^{3c}, R^{3d}, and R^{3e} is independently selected from hydrogen, —Br, —Cl, —F, —CN, —NH₂, —OH, —NO₂, —CO₂H, —OC(O)CH₃, —OC(O)CH₂CH₃, —OC(O)CH(CH₃)₂, —OC(O)CH₂CH₂CH₃, —CH₂OH, —CH₂CH₂OH, —CH₂CH₂CH₂OH, —CH(CH₃)CH₂OH, —CH₂NH₂, —CH₂CH₂NH₂, —CH₂CH₂CH₂NH₂, —CH(CH₃)CH₂NH₂, and Ar¹. In a still further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —OC(O)CH₃, —OC(O)CH₂CH₃, —CH₂OH, —CH₂CH₂OH, —CH₂NH₂, —CH₂CH₂NH₂, and Ar¹. In yet a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —OC(O)CH₃, —CH₂OH, —CH₂NH₂, and Ar¹.

In various aspects, each of R^{3a}, R^{3b}, R^{3c}, R^{3d}, and R^{3e} is independently selected from hydrogen, halogen, —CN, —NH₂, —OH, —NO₂, —CO₂H, C1-C4 alkyl, C2-C4 alkenyl, C1-C4 hydroxyalkyl, C1-C4 alkylamino, and Ar¹. In a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, methyl, ethyl, n-propyl, isopropyl, ethenyl, n-propenyl, isopropenyl, —CH₂OH, —CH₂CH₂OH, —CH₂CH₂CH₂OH, —CH(CH₃)CH₂OH, —CH₂NH₂, —CH₂CH₂NH₂, —CH₂CH₂CH₂NH₂, —CH(CH₃)CH₂NH₂, and Ar¹. In a still further aspect, each of R^{3a}, R^{3b}, R^{3c}, R^{3d}, and R^{3e} is independently selected from hydrogen, —Cl, —F, —CN, —NH₂, —OH, —NO₂, —CO₂H, methyl, ethyl, ethenyl, —CH₂OH, —CH₂CH₂OH, —CH₂NH₂, —CH₂CH₂NH₂, and Ar¹. In yet a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, methyl, —CH₂OH, —CH₂NH₂, and Ar¹.

In various aspects, each of R^{3a}, R^{3b}, R^{3c}, R^{3d}, and R^{3e} is independently selected from hydrogen, halogen, —CN, —NH₂, —OH, —NO₂, —CO₂H, C1-C4 haloalkyl, C1-C4 cyanoalkyl, C1-C4 hydroxyalkyl, C1-C4 alkylamino, and Ar¹. In a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —CH₂NH₂, —CH₂CH₂NH₂, —CH₂CH₂CH₂NH₂, —CH(CH₃)CH₂NH₂, and Ar¹. In a still further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂Cl, —CH₂F, —CH₂CH₂Cl, —CH₂CH₂F, —CH₂CN, —CH₂CH₂CN, —CH₂OH, —CH₂CH₂OH, —CH₂NH₂, —CH₂CH₂NH₂, and Ar¹. In yet a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂Cl, —CH₂F, —CH₂CN, —CH₂OH, —CH₂NH₂, and Ar¹.

In various aspects, each of R^{3a}, R^{3b}, R^{3c}, R^{3d}, and R^{3e} is independently selected from hydrogen, halogen, —CN, —NH₂, —OH, —NO₂, —CO₂H, C1-C4 hydroxyalkyl, C1-C4 haloalkoxy, C1-C4 alkoxy, and Ar¹. In a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂OH, —CH₂CH₂OH, —CH₂CH₂CH₂OH, —CH(CH₃)CH₂OH, —OCH₂Cl, —OCH₂F, —OCHCl₂, —OCHF₂, —OCCl₃, —OCF₃, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, and Ar¹. In a still further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂OH, —CH₂CH₂OH, —OCH₂Cl, —OCH₂F, —OCHCl₂, —OCHF₂, —OCCl₃, —OCF₃, —OCH₂CH₂Cl, —OCH₂CH₂F, —OCH₃, —OCH₂CH₃, and Ar¹. In yet a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂OH, —OCH₂Cl, —OCH₂F, —OCHCl₂, —OCHF₂, —OCCl₃, —OCF₃, —OCH₃, and Ar¹.

In various aspects, each of R^{3a}, R^{3b}, R^{3c}, R^{3d}, and R^{3e} is independently selected from hydrogen, halogen, —CN, —NH₂, —OH, —NO₂, —CO₂H, C1-C4 alkylamino, (C1-C4)(C1-C4) dialkylamino, C1-C4 aminoalkyl, and Ar¹. In a further aspect, each of R^{3a}, R^{3b}, R^{3c}, R^{3d}, and R^{ae} is independently selected from hydrogen, —Cl, —F, —CN, —NH₂, —OH, —NO₂, —CO₂H, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, and Ar¹. In a still further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂NH₂, —CH₂CH₂NH₂, —NHCH₃, —NHCH₂CH₃, —N(CH₃)₂, —N(CH₃)CH₂CH₃, and Ar¹. In yet a further aspect, each of R^{3a}, R^{3b}, R^{3c}, R^{3d}, and R^{3e} is independently selected from hydrogen, —Cl, —F, —CN, —NH₂, —OH, —NO₂, —CO₂H, —CH₂NH₂, —NHCH₃, —N(CH₃)₂, and Ar¹.

In various aspects, one of R^{3a}, R^{3b}, R^{3c}, R^{3d}, and R^{3e} is —CO₂H, —CH₂OH, or —CH₂NH₂ and four of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹. In a further aspect, one of R^{3a}, R^{3b}, R^{3c}, R^{3d}, and R^{3e} is —CO₂H, —CH₂OH, or —CH₂NH₂ and four of R^{3a}, R^{3b}, R^{3c}, R^{3d}, and R^{3e} is independently selected from hydrogen, halogen, —CN, —NH₂, —OH, —NO₂, —CO₂H, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂Cl, —OCH₂F, —OCHCl₂, —OCHF₂, —OCCl₃, —OCF₃, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, and Ar¹. In a still further aspect, one of R^{3a}, R^{3b}, R^{3c}, R^{3d}, and R^{3e} is —CO₂H, —CH₂OH, or —CH₂NH₂ and four of R^{3a}, R^{3b}, R^{3c}, R^{3d}, and R^{3e} is independently selected from hydrogen, halogen, —CN, —NH₂, —OH, —NO₂, —CO₂H, —OC(O)CH₃, —OC(O)CH₂CH₃, methyl, ethyl, ethenyl, CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCHCl₂, —OCHF₂, —OCCl₃, —OCF₃, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, —N(CH₃)CH₂CH₃, and Ar¹. In yet a further aspect, one of R^{3a}, R^{3b}, R^{3c}, R^{3d}, and R^{3e} is —CO₂H, —CH₂OH, or —CH₂NH₂ and four of R^{3a}, R^{3b}, R^{3c}, R^{3d}, and R^{3e} is independently selected from hydrogen, halogen, —CN, —NH₂, —OH, —NO₂, —CO₂H, —OC(O)CH₃, —OC(O)CH₂CH₃, methyl, CH₂Cl, —CH₂F, —CH₂CN, —CH₂OH, —OCH₂Cl, —OCH₂F, —OCHCl₂, —OCHF₂, —OCCl₃, —OCF₃, —OCH₃, —CH₂NH₂, —NHCH₃, —N(CH₃)₂, and Ar¹.

In various aspects, R^{3c} is —CO₂H, —CH₂OH, or —CH₂NH₂ and R^{3a}, R^{3b}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹. In a further aspect, R^{3c} is —CO₂H, —CH₂OH, or —CH₂NH₂ and R^{3a}, R^{3b}, R^{3d}, and R^{3e} is independently selected from hydrogen, halogen, —CN, —NH₂, —OH, —NO₂, —CO₂H, —OC(O)CH₃, —OC(O)CH₂CH₃, —OC(O)CH(CH₃)₂, —OC(O)CH₂CH₂CH₃, methyl, ethyl, n-propyl, isopropyl, ethenyl, n-propenyl, isopropenyl, CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂Cl, —OCH₂F, —OCHCl₂, —OCHF₂, —OCCl₃, —OCF₃, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, and Ar¹. In a still further aspect, R^{3c} is —CO₂H, —CH₂OH, or —CH₂NH₂ and R^{3a}, R^{3b}, R^{3d}, and R^{3e} is independently selected from hydrogen, halogen, —CN, —NH₂, —OH, —NO₂, —CO₂H, —OC(O)CH₃, —OC(O)CH₂CH₃, methyl, ethyl, ethenyl, CH₂Cl, —CH₂F, —CH₂CH₂Cl, —CH₂CH₂F, —CH₂CN, —CH₂CH₂CN, —CH₂OH, —CH₂CH₂OH, —OCH₂Cl, —OCH₂F, —OCHCl₂, —OCHF₂, —OCCl₃, —OCF₃, —OCH₂CH₂Cl, —OCH₂CH₂F, —OCH₃, —OCH₂CH₃, —CH₂NH₂, —CH₂CH₂NH₂, —NHCH₃, —NHCH₂CH₃, —N(CH₃)₂, —N(CH₃)CH₂CH₃, and Ar¹. In yet a further aspect, R^{3c} is —CO₂H, —CH₂OH, or —CH₂NH₂ and R^{3a}, R^{3b}, R^{3d}, and R^{3e} is independently selected from hydrogen, halogen, —CN, —NH₂, —OH, —NO₂, —CO₂H, —OC(O)CH₃, —OC(O)CH₂CH₃, methyl, CH₂Cl, —CH₂F, —CH₂CN, —CH₂OH, —OCH₂Cl, —OCH₂F, —OCHCl₂, —OCHF₂, —OCCl₃, —OCF₃, —OCH₃, —CH₂NH₂, —NHCH₃, —N(CH₃)₂, and Ar¹.

In various aspects, one of R^{3a}, R^{3b}, R^{3c}, R^{3d}, and R^{3e} is —CH₂OH. In further aspects, three of R^{3a}, R^{3b}, R^{3c}, R^{3d}, and R^{3e} are hydrogen and one of R^{3a}, R^{3b}, R^{3c}, R^{3d}, and R^{3e} is —CH₂OH. In still further aspects, four of R^{3a}, R^{3b}, R^{3c}, R^{3d}, and R^{3e} are hydrogen and one of R^{3a}, R^{3b}, R^{3c}, R^{3d}, and R^{3e} is —CH₂OH. In further aspects, each of R^{3a}, R^{3b}, R^{3d}, and R^{3e} are hydrogen, and R^{3c} is —CH₂OH.

In various aspects, one of R^{3a}, R^{3b}, R^{3c}, R^{3d}, and R^{3e} is —CH₂NH₂. In further aspects, three of R^{3a}, R^{3b}, R^{3c}, R^{3d}, and R^{3e} are hydrogen and one of R^{3a}, R^{3b}, R^{3c}, R^{3d}, and R^{3e} is —CH₂NH₂. In still further aspects, four of R^{3a}, R^{3b}, R^{3c}, R^{3d}, and R^{3e} are hydrogen and one of R^{3a}, R^{3b}, R^{3c}, R^{3d}, and R^{3e} is —CH₂NH₂. In further aspects, each of R^{3a}, R^{3b}, R^{3d}, and R^{3e} are hydrogen, and R^{3c} is —CH₂NH₂.

In various aspects, each of R^{3a}, R^{3b}, R^{3d}, and R^{3e} are hydrogen.

In various aspects, R^{3c} is —CH₂OH.

In various aspects, R^{3c} is —CH₂NH₂.

e. R¹⁰ Groups

In one aspect, R¹⁰, when present, is selected from hydrogen, —OH, C1-C10 alkyl, C1-C10 alkoxy, C1-C10 alkylamino, and (C1-C10)(C1-C10) dialkylamino. In a further aspect R¹⁰, when present, is selected from hydrogen, —OH, C1-C8 alkyl, C1-C8 alkoxy, C1-C8 alkylamino, and (C1-C8)(C1-C8) dialkylamino. In a still further aspect, R¹⁰, when present, is selected from hydrogen, —OH, C1-C4 alkyl, C1-C4 alkoxy, C1-C4 alkylamino, and (C1-C4)(C1-C4) dialkylamino. In yet a further aspect, R¹⁰, when present, is selected from hydrogen, —OH, methyl, ethyl, n-propyl, isopropyl, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₂CH₃)CH₂CH₂CH₃. In an even further aspect, R¹⁰, when present, is selected from hydrogen, —OH, methyl, ethyl, —OCH₃, —OCH₂CH₃, —NHCH₃, —NHCH₂CH₃, —N(CH₃)₂, and —N(CH₃)CH₂CH₃. In a still further aspect, R¹⁰, when present, is selected from hydrogen, —OH, methyl, —OCH₃, —NHCH₃, and —N(CH₃)₂.

In one aspect, R¹⁰, when present, is selected from hydrogen, C1-C10 alkylamino, and (C1-C10)(C1-C10) dialkylamino. In a further aspect R¹⁰, when present, is selected from hydrogen, C1-C8 alkylamino, and (C1-C8)(C1-C8) dialkylamino. In a still further aspect, R¹⁰, when present, is selected from hydrogen, C1-C4 alkylamino, and (C1-C4)(C1-C4) dialkylamino. In yet a further aspect, R¹⁰, when present, is selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₂CH₃)CH₂CH₂CH₃. In an even further aspect, R¹⁰, when present, is selected from hydrogen, —NHCH₃, —NHCH₂CH₃, —N(CH₃)₂, and —N(CH₃)CH₂CH₃. In a still further aspect, R¹⁰, when present, is selected from hydrogen, —NHCH₃, and —N(CH₃)₂.

In one aspect, R¹⁰, when present, is selected from hydrogen, —OH, and C1-C10 alkoxy. In a further aspect R¹⁰, when present, is selected from hydrogen, —OH, and C1-C8 alkoxy. In a still further aspect, R¹⁰, when present, is selected from hydrogen, —OH, and C1-C4 alkoxy. In yet a further aspect, R¹⁰, when present, is selected from hydrogen, —OH, —OCH₃, —OCH₂CH₃, —OCH₂CH₂CH₃, and —OCH(CH₃)₂. In an even further aspect, R¹⁰, when present, is selected from hydrogen, —OH, —OCH₃, and —OCH₂CH₃. In a still further aspect, R¹⁰, when present, is selected from hydrogen, —OH, and —OCH₃.

In one aspect, R¹⁰, when present, is selected from hydrogen and C1-C10 alkyl. In a further aspect R¹⁰, when present, is selected from hydrogen and C1-C8 alkyl. In a still further aspect, R¹⁰, when present, is selected from hydrogen and C1-C4 alkyl. In yet a further aspect, R¹⁰, when present, is selected from hydrogen, methyl, ethyl, n-propyl, and isopropyl. In an even further aspect, R¹⁰, when present, is selected from hydrogen, methyl, and ethyl. In a still further aspect, R¹⁰, when present, is selected from hydrogen and methyl.

In various aspects, R¹⁰, when present, is selected from hydrogen and —OH. In a further aspect, R¹⁰, when present, is —OH. In a still further aspect, R¹⁰, when present, is hydrogen.

f. R¹¹ Groups

In one aspect, R¹¹, when present, is a carboxylate residue of a chemotherapeutic agent or a carbamide residue of a chemotherapeutic agent. Examples of chemotherapeutic agents include, but are not limited to, alkylating agents such as busulfan, cis-platin, mitomycin C, and carboplatin; antimitotic agents such as colchicine, vinblastine, paclitaxel (e.g., TAXOL®), and docetaxel; topoisomerase I inhibitors such as camptothecin and topotecan; topoisomerase II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, hydroxyurea, gemcitabine, capecitabine and thioguanine; antibodies such as HERCEPTIN® and RITUXAN®, as well as other known chemotherapeutics such as photofrin, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

In various aspects, the carboxylate or carbamide residue is selected from:

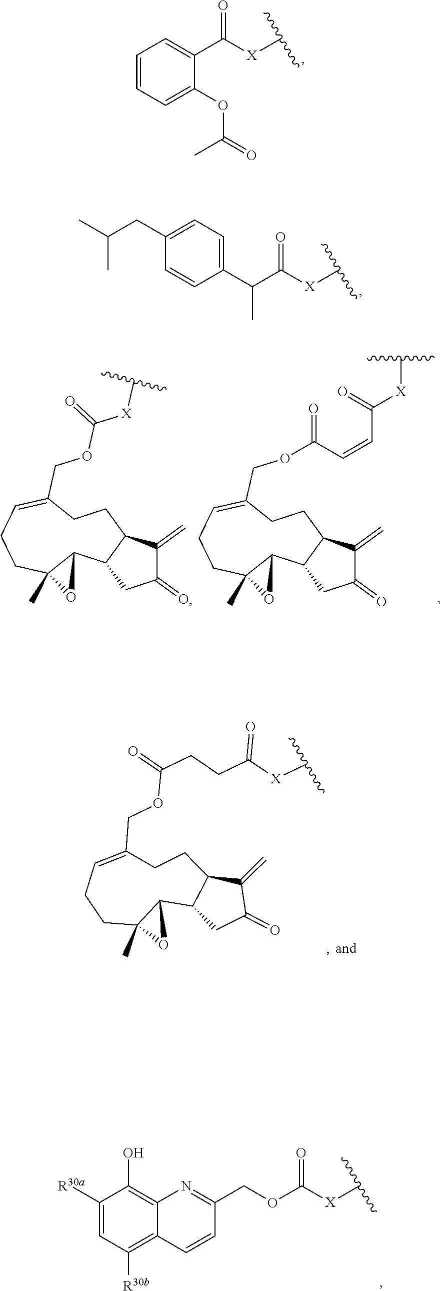

wherein X is selected from NH and O; and wherein each of R^{30a} and R^{30b}, when present, is independently selected from hydrogen, —Cl, —Br, and —I.

In various aspects, R¹¹, when present, is selected from:

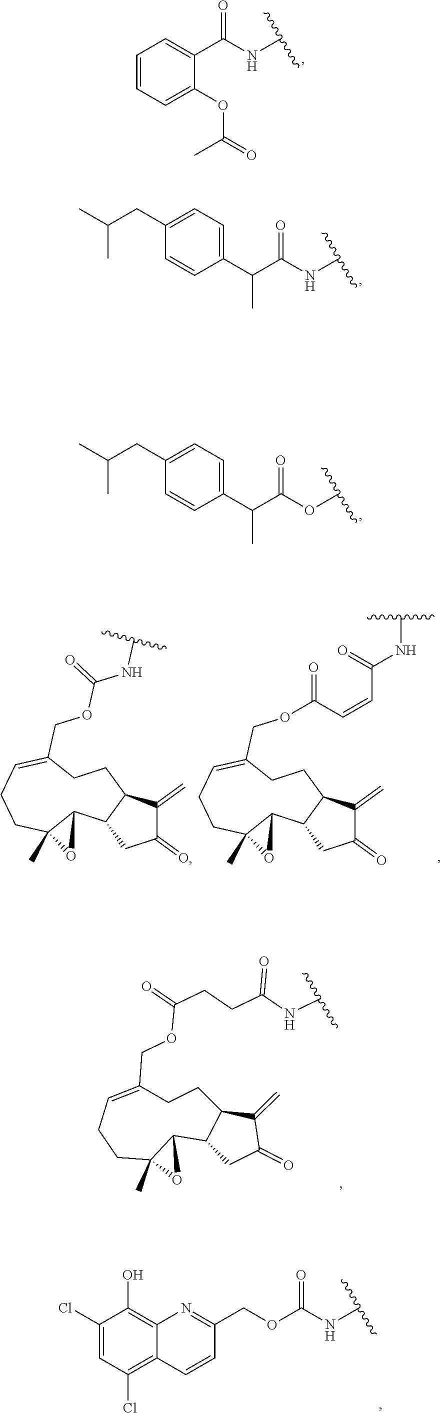

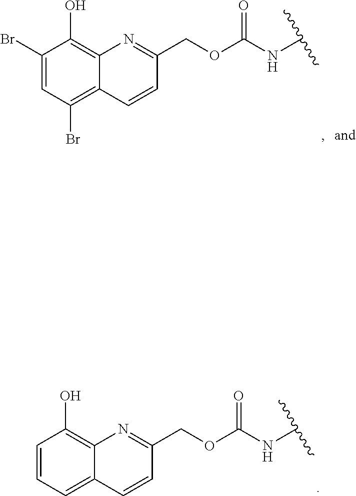 [SMILES: *NC(=O)OCc1ccc2c(Br)cc(Br)c(O)c2n1 ; *NC(=O)OCc1ccc2cccc(O)c2n1]

g. R²⁰ Groups

In one aspect, R²⁰, when present, is selected from hydrogen, —OH, C1-C10 alkyl, C1-C10 alkoxy, C1-C10 alkylamino, and (C1-C10)(C1-C10) dialkylamino. In a further aspect R¹⁰, when present, is selected from hydrogen, —OH, C1-C8 alkyl, C1-C8 alkoxy, C1-C8 alkylamino, and (C1-C8)(C1-C8) dialkylamino. In a still further aspect, R²⁰, when present, is selected from hydrogen, —OH, C1-C4 alkyl, C1-C4 alkoxy, C1-C4 alkylamino, and (C1-C4)(C1-C4) dialkylamino. In yet a further aspect, R²⁰, when present, is selected from hydrogen, —OH, methyl, ethyl, n-propyl, isopropyl, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₂CH₃)CH₂CH₂CH₃. In an even further aspect, R²⁰, when present, is selected from hydrogen, —OH, methyl, ethyl, —OCH₃, —OCH₂CH₃, —NHCH₃, —NHCH₂CH₃, —N(CH₃)₂, and —N(CH₃)CH₂CH₃. In a still further aspect, R²⁰, when present, is selected from hydrogen, —OH, methyl, —OCH₃, —NHCH₃, and —N(CH₃)₂.

In one aspect, R²⁰, when present, is selected from hydrogen, C1-C10 alkylamino, and (C1-C10)(C1-C10) dialkylamino. In a further aspect R²⁰, when present, is selected from hydrogen, C1-C8 alkylamino, and (C1-C8)(C1-C8) dialkylamino. In a still further aspect, R²⁰, when present, is selected from hydrogen, C1-C4 alkylamino, and (C1-C4)(C1-C4) dialkylamino. In yet a further aspect, R²⁰, when present, is selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, and —N(CH₂CH₃)CH₂CH₂CH₃. In an even further aspect, R²⁰, when present, is selected from hydrogen, —NHCH₃, —NHCH₂CH₃, —N(CH₃)₂, and —N(CH₃)CH₂CH₃. In a still further aspect, R²⁰, when present, is selected from hydrogen, —NHCH₃, and —N(CH₃)₂.

In one aspect, R²⁰, when present, is selected from hydrogen, —OH, and C1-C10 alkoxy. In a further aspect, R²⁰, when present, is selected from hydrogen, —OH, and C1-C8 alkoxy. In a still further aspect, R²⁰, when present, is selected from hydrogen, —OH, and C1-C4 alkoxy. In yet a further aspect, R²⁰, when present, is selected from hydrogen, —OH, —OCH₃, —OCH₂CH₃, —OCH₂CH₂CH₃, and —OCH(CH₃)₂. In an even further aspect, R²⁰, when present, is selected from hydrogen, —OH, —OCH₃, and —OCH₂CH₃. In a still further aspect, R²⁰, when present, is selected from hydrogen, —OH, and —OCH₃.

In one aspect, R²⁰, when present, is selected from hydrogen and C1-C10 alkyl. In a further aspect R²⁰, when present, is selected from hydrogen and C1-C8 alkyl. In a still further aspect, R²⁰, when present, is selected from hydrogen and C1-C4 alkyl. In yet a further aspect, R²⁰, when present, is selected from hydrogen, methyl, ethyl, n-propyl, and isopropyl. In an even further aspect, R²⁰, when present, is selected from hydrogen, methyl, and ethyl. In a still further aspect, R²⁰, when present, is selected from hydrogen and methyl.

In various aspects, R²⁰, when present, is selected from hydrogen and —OH. In a further aspect, R²⁰, when present, is —OH. In a still further aspect, R²⁰, when present, is hydrogen.

h. R^{30A} and R^{30b} Groups

In one aspect, each of R^{30a} and R^{30b}, when present, is independently selected from hydrogen, —Cl, —Br, and —I. In a still further aspect, each of R^{30a} and R^{30b}, when present, is independently selected from hydrogen, —Cl, and —Br. In yet a further aspect, each of R^{30a} and R^{30b}, when present, is independently selected from hydrogen and —Br. In an even further aspect, each of R^{30a} and R^{30b}, when present, is independently selected from hydrogen and —Cl.

In further aspects, each of R^{30a} and R^{30b}, when present, is hydrogen. In still further aspects, each of R^{30a} and R^{30b}, when present, is —Cl. In further aspects, each of R^{30a} and R^{30b}, when present, is —Br. In still further aspects, each of R^{30a} and R^{30b}, when present, is —I.

i. Cy¹ Groups

In one aspect, Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹, when present, is selected from cycloalkyl, heterocycloalkyl, aryl, and heteroaryl, and is substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is selected from cycloalkyl, heterocycloalkyl, aryl, and heteroaryl, and is substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is selected from cycloalkyl, heterocycloalkyl, aryl, and heteroaryl, and is monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is selected from cycloalkyl, heterocycloalkyl, aryl, and heteroaryl, and is unsubstituted.

In various aspects, Cy¹, when present, is selected from aryl and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹, when present, is selected from aryl and heteroaryl, and is substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is selected from aryl and heteroaryl, and is substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is selected from aryl and heteroaryl, and is monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is selected from aryl and heteroaryl, and is unsubstituted.

In various aspects, Cy¹, when present, is aryl substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthryl, and anthracenyl. In a further aspect, Cy¹, when present, is aryl substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is aryl substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is aryl monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted aryl.

In various aspects, Cy¹, when present, is C6 aryl substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹, when present, is C6 aryl substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is C6 aryl substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is C6 aryl monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted C6 aryl.

In various aspects, Cy¹, when present, is C6 aryl monosubstituted with a group selected from halogen, —CN, and C1-C4 alkoxy. In a further aspect, Cy¹, when present, is C6 aryl monosubstituted with a group selected from —Cl, —Br, —F, —CN, methoxy, ethoxy, n-propoxy, and isopropoxy. In a still further aspect, Cy¹, when present, is C6 aryl monosubstituted with a group selected from —Cl, —F, —CN, methoxy, and ethoxy. In yet a further aspect, Cy¹, when present, is C6 aryl monosubstituted with a group selected from —Cl, —F, —CN, and methoxy.

In various aspects, Cy¹, when present, is selected from:

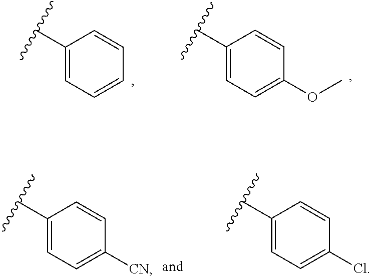

In various aspects, Cy¹, when present, is heteroaryl substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, pyrrole, furan, thiophene, pyridine, pyridazine, pyrimidine, pyrazine, triazine, purine, oxazole, benzo[d]oxazole, benzo[d]thiazole, indole, and isoxazole. In a further aspect, Cy¹, when present, is heteroaryl substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is heteroaryl substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is heteroaryl monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted heteroaryl.

In various aspects, Cy¹, when present, is selected from cycloalkyl and heterocycloalkyl, and is substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹, when present, is selected from cycloalkyl and heterocycloalkyl, and is substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is selected from cycloalkyl and heterocycloalkyl, and is substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is selected from cycloalkyl and heterocycloalkyl, and is monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is selected from cycloalkyl and heterocycloalkyl, and is unsubstituted.

In various aspects, Cy¹, when present, is cycloalkyl substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of cycloalkyls include, but are not limited to, cyclopropyl, cyclobutyl, cyclopentyl, cyclohexyl, and norbornyl. In a further aspect, Cy¹, when present, is cycloalkyl substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is cycloalkyl substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is cycloalkyl monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted cycloalkyl.

In various aspects, Cy¹, when present, is heterocycloalkyl substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heterocycloalkyls include, but are not limited to, aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. In a further aspect, Cy¹, when present, is heterocycloalkyl substituted with 0, 1, or 2 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is heterocycloalkyl substituted with 0 or 1 group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is heterocycloalkyl monosubstituted with a group selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted heterocycloalkyl.

j. Ar¹ Groups

In one aspect, Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar¹, when present, is selected from heteroaryl and aryl, and is substituted with 0, 1, or 2 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar¹, when present, is selected from heteroaryl and aryl, and is substituted with 0 or 1 group selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar¹, when present, is selected from heteroaryl and aryl, and is monosubstituted with a group selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar¹, when present, is selected from heteroaryl and aryl, and is unsubstituted.

In various aspects, Ar¹, when present, is heteroaryl substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further non-limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl. In a further aspect, Ar¹, when present, is heteroaryl substituted with 0, 1, or 2 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar¹, when present, is heteroaryl substituted with 0 or 1 group selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar¹, when present, is heteroaryl monosubstituted with a group selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar¹, when present, is unsubstituted heteroaryl.

In various aspects, Ar¹, when present, is aryl substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthryl, and anthracenyl. In a further aspect, Ar¹, when present, is aryl substituted with 0, 1, or 2 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar¹, when present, is aryl substituted with 0 or 1 group selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar¹, when present, is aryl monosubstituted with a group selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar¹, when present, is unsubstituted aryl.

In various aspects, Ar¹, when present, is aryl or heteroaryl substituted with 0, 1, 2, or 3 groups independently selected from hydrogen, —Br, —Cl, —F, —I, —OC(O)(CH₃), —OC(O)(CH₂CH₃), —OC(O)(CH(CH₃)₂), —OC(O)(CH₂CH₂CH₃), —OC(O)(CH(CH₂CH₃)CH₃), —OC(O)(CH₂CH₂CH₂CH₃), methyl, ethyl, n-propyl, isopropyl, butyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)₂Cl, —CH(CH₃)₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)₂OH, —CH₂OCH₂Cl, —CH₂OCH₂F, —CH₂OCH₂CH₂Cl, —CH₂OCH₂CH₂F, —CH₂OCH₂CH₂CH₂Cl, —CH₂OCH₂CH₂CH₂F, —CH₂OCH₃, —CH₂CH₂OCH₃, —CH₂OCH₂CH₃, —CH₂NH₂, —CH₂CH₂NH₂, —CH₂CH₂CH₂NH₂, —CH(CH₃)₂NH₂, —NHCH₃, —NHCH₂CH₃, —NHCH₂CH₂CH₃, —NHCH₂(CH₃)₂, —N(CH₃)₂, —N(CH₃)CH₂CH₃, —N(CH₃)CH₂CH₂CH₃, —N(CH₃)CH₂(CH₃)₂, —CH₂NHCH₃, —CH₂CH₂NHCH₃, —CH₂CH₂CH₂NHCH₃, and —CH₂CH₂CH₂NHCH₂CH₃. In a further aspect, Ar¹, when present, is aryl or heteroaryl substituted with 0, 1, or 2 groups independently selected from hydrogen, —Br, —Cl, —F, —I, —OC(O)(CH₃), —OC(O)(CH₂CH₃), —OC(O)(CH(CH₃)₂), —OC(O)(CH₂CH₂CH₃), —OC(O)(CH(CH₂CH₃)CH₃), —OC(O)(CH₂CH₂CH₂CH₃), methyl, ethyl, n-propyl, isopropyl, butyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)₂Cl, —CH(CH₃)₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)₂OH, —CH₂OCH₂Cl, —CH₂OCH₂F, —CH₂OCH₂CH₂Cl, —CH₂OCH₂CH₂F, —CH₂OCH₂CH₂CH₂Cl, —CH₂OCH₂CH₂CH₂F, —CH₂OCH₃, —CH₂CH₂OCH₃, —CH₂OCH₂CH₃, —CH₂NH₂, —CH₂CH₂NH₂, —CH₂CH₂CH₂NH₂, —CH(CH₃)₂NH₂, —NHCH₃, —NHCH₂CH₃, —NHCH₂CH₂CH₃, —NHCH₂(CH₃)₂, —N(CH₃)₂, —N(CH₃)CH₂CH₃, —N(CH₃)CH₂CH₂CH₃, —N(CH₃)CH₂(CH₃)₂, —CH₂NHCH₃, —CH₂CH₂NHCH₃, —CH₂CH₂CH₂NHCH₃, and —CH₂CH₂CH₂NHCH₂CH₃. In a still further aspect, Ar¹, when present, is aryl or heteroaryl substituted with 0 or 1 group selected from hydrogen, —Br, —Cl, —F, —I, —OC(O)(CH₃), —OC(O)(CH₂CH₃), —OC(O)(CH(CH₃)₂), —OC(O)(CH₂CH₂CH₃), —OC(O)(CH(CH₂CH₃)CH₃), —OC(O)(CH₂CH₂CH₂CH₃), methyl, ethyl, n-propyl, isopropyl, butyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)₂Cl, —CH(CH₃)₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)₂OH, —CH₂OCH₂Cl, —CH₂OCH₂F, —CH₂OCH₂CH₂Cl, —CH₂OCH₂CH₂F, —CH₂OCH₂CH₂CH₂Cl, —CH₂OCH₂CH₂CH₂F, —CH₂OCH₃, —CH₂CH₂OCH₃, —CH₂OCH₂CH₃, —CH₂NH₂, —CH₂CH₂NH₂, —CH₂CH₂CH₂NH₂, —CH(CH₃)₂NH₂, —NHCH₃, —NHCH₂CH₃, —NHCH₂CH₂CH₃, —NHCH₂(CH₃)₂, —N(CH₃)₂, —N(CH₃)CH₂CH₃, —N(CH₃)CH₂CH₂CH₃, —N(CH₃)CH₂(CH₃)₂, —CH₂NHCH₃, —CH₂CH₂NHCH₃, —CH₂CH₂CH₂NHCH₃, and —CH₂CH₂CH₂NHCH₂CH₃. In yet a further aspect, Ar¹, when present, is aryl or heteroaryl monosubstituted with a group selected from hydrogen, —Br, —Cl, —F, —I, —OC(O)(CH₃), —OC(O)(CH₂CH₃), —OC(O)(CH(CH₃)₂), —OC(O)(CH₂CH₂CH₃), —OC(O)(CH(CH₂CH₃)CH₃), —OC(O)(CH₂CH₂CH₂CH₃), methyl, ethyl, n-propyl, isopropyl, butyl, ethenyl, n-propenyl, isopropenyl, —CH₂Cl, —CH₂F, —CH₂CH₂Cl, —CH₂CH₂F, —CH₂CH₂CH₂Cl, —CH₂CH₂CH₂F, —CH(CH₃)₂Cl, —CH(CH₃)₂F, —CH₂CN, —CH₂CH₂CN, —CH₂CH₂CH₂CN, —CH(CH₃)₂CN, —CH₂OH, —CH₂CH₂OH, —CH₂CH₂CH₂OH, —CH(CH₃)₂OH, —CH₂OCH₂Cl, —CH₂OCH₂F, —CH₂OCH₂CH₂Cl, —CH₂OCH₂CH₂F, —CH₂OCH₂CH₂CH₂Cl, —CH₂OCH₂CH₂CH₂F, —CH₂OCH₃, —CH₂CH₂OCH₃, —CH₂OCH₂CH₃, —CH₂NH₂, —CH₂CH₂NH₂, —CH₂CH₂CH₂NH₂, —CH(CH₃)₂NH₂, —NHCH₃, —NHCH₂CH₃, —NHCH₂CH₂CH₃, —NHCH₂(CH₃)₂, —N(CH₃)₂, —N(CH₃)CH₂CH₃, —N(CH₃)CH₂CH₂CH₃, —N(CH₃)CH₂(CH₃)₂, —CH₂NHCH₃, —CH₂CH₂NHCH₃, —CH₂CH₂CH₂NHCH₃, and —CH₂CH₂CH₂NHCH₂CH₃.

In various aspects, Ar¹ is selected from naphthyl, furanyl, benzofuranyl, oxazolyl, isoxazolyl, oxadiazolyl, benzoxazolyl, benzoxadiazolyl, pyrrolyl, pyrazolyl, imidazolyl, quinolinyl, quinazolinyl, indazolyl, triazolyl, tetrazolyl, pyridinyl, pyrimidinyl, pyrazinyl, pyridazinyl, indolyl, isoindolyl, indolizinyl, benzimidazolyl, indazolyl, benzotriazolyl, tetrazolopyridazinyl, carbazolyl, purinyl, isoquinolinyl, and imidazopyridinyl.

### 2. Example Compounds

In one aspect, a compound can be present as:

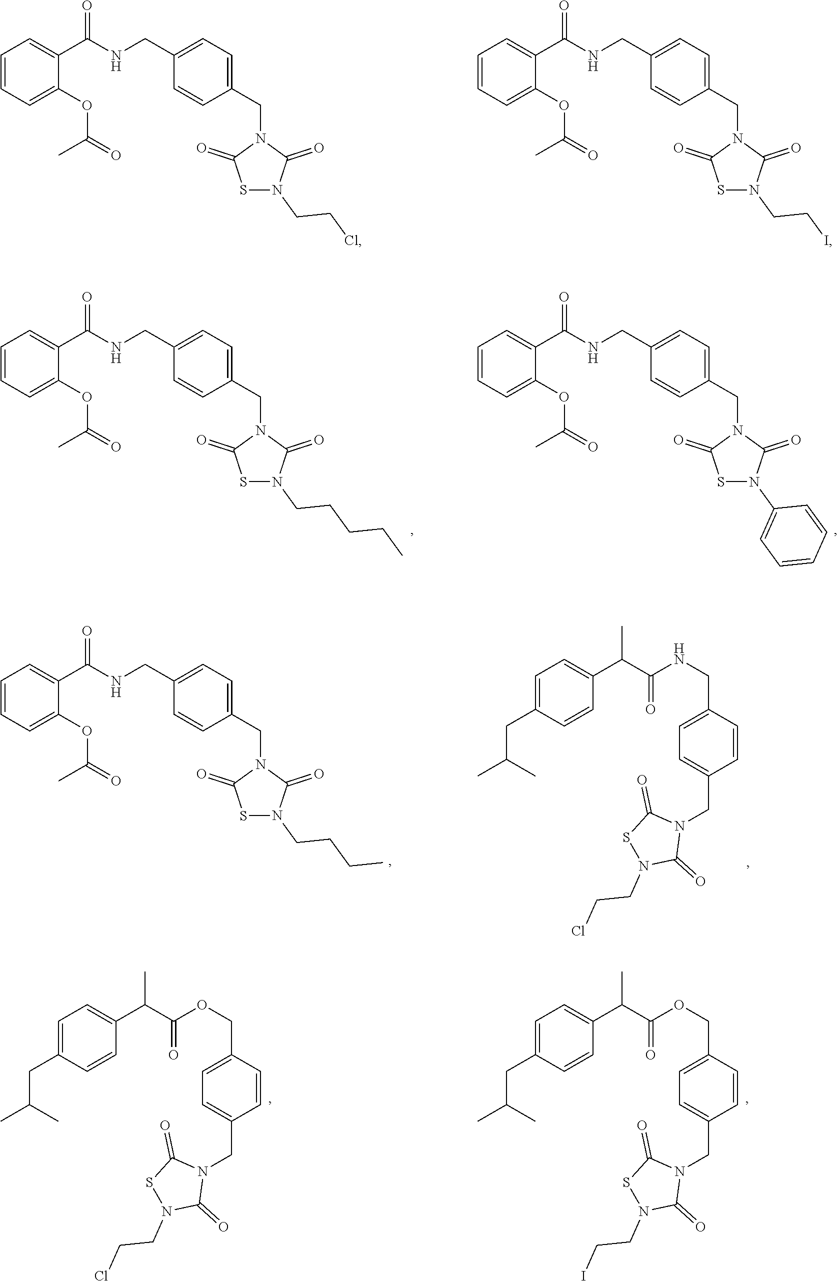

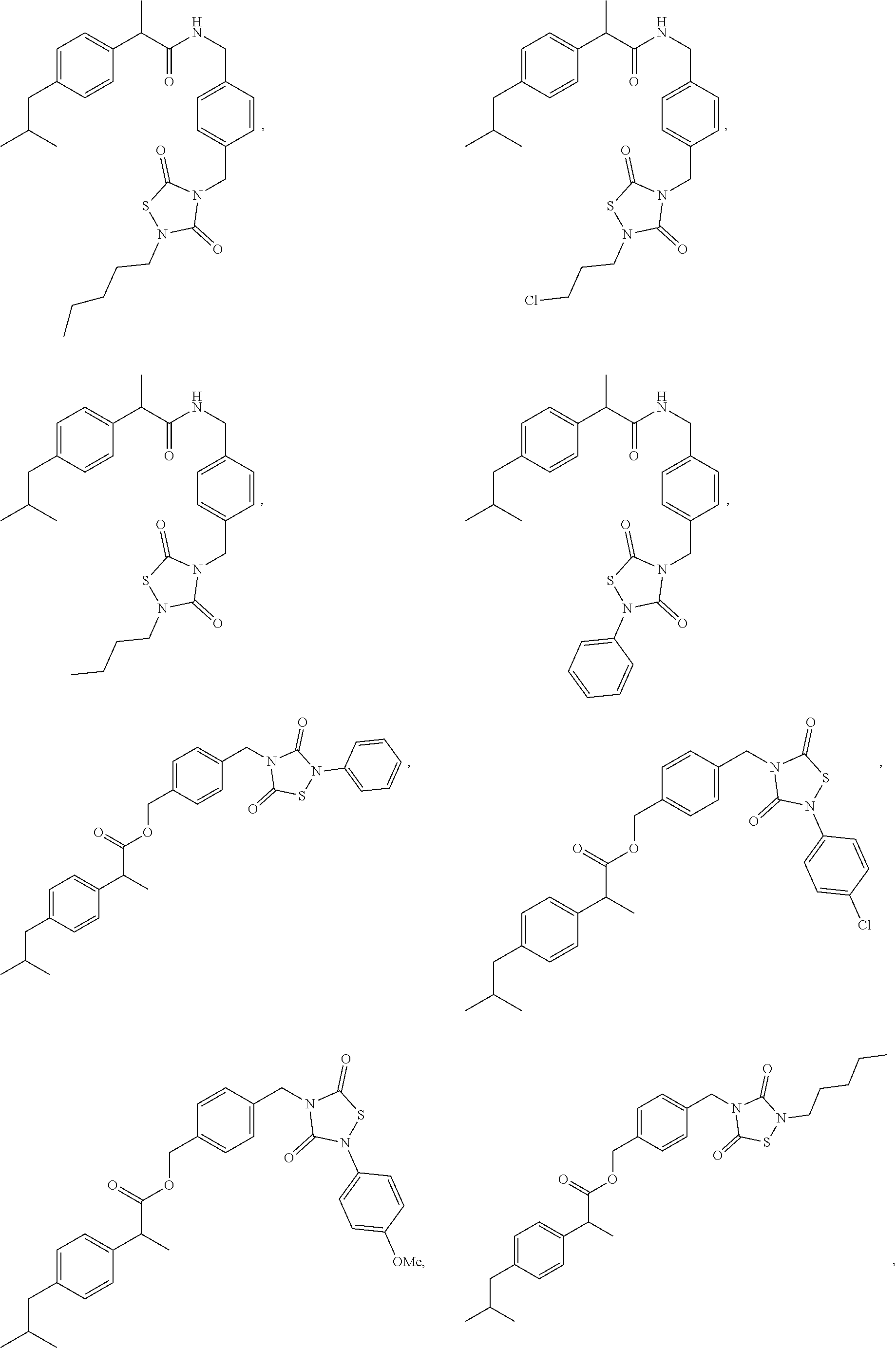

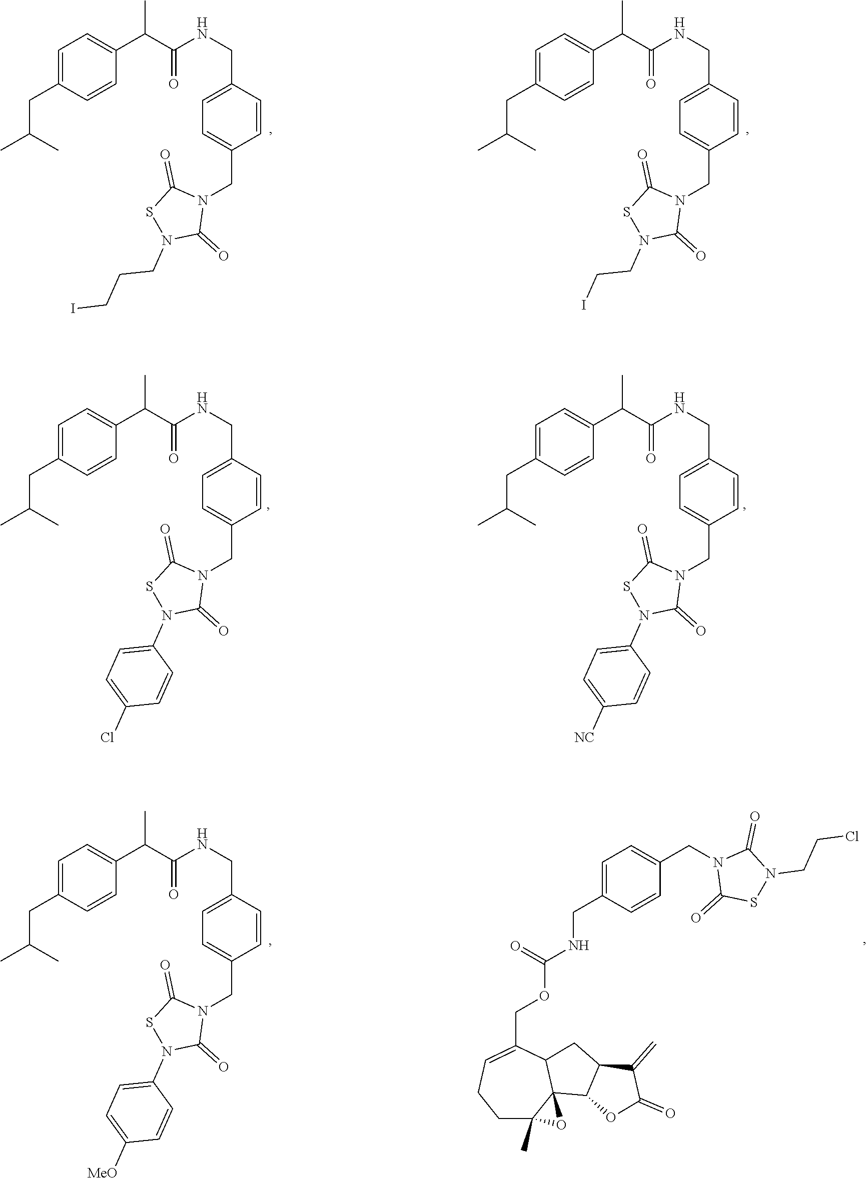 [SMILES: CC(C)Cc1ccc(C(C)C(=O)NCc2ccc(Cn3c(=O)sn(CCCI)c3=O)cc2)cc1 ; CC(C)Cc1ccc(C(C)C(=O)NCc2ccc(Cn3c(=O)sn(CCI)c3=O)cc2)cc1 ; CC(C)Cc1ccc(C(C)C(=O)NCc2ccc(Cn3c(=O)sn(-c4ccc(Cl)cc4)c3=O)cc2)cc1 ; CC(C)Cc1ccc(C(C)C(=O)NCc2ccc(Cn3c(=O)sn(-c4ccc(C#N)cc4)c3=O)cc2)cc1 ; COc1ccc(-n2sc(=O)n(Cc3ccc(CNC(=O)C(C)c4ccc(CC(C)C)cc4)cc3)c2=O)cc1 ; C=C1C(=O)O[C@H]2[C@H]1CC1C(COC(=O)NCc3ccc(Cn4c(=O)sn(CCCl)c4=O)cc3)=CCC[C@@]3(C)O[C@@]123]

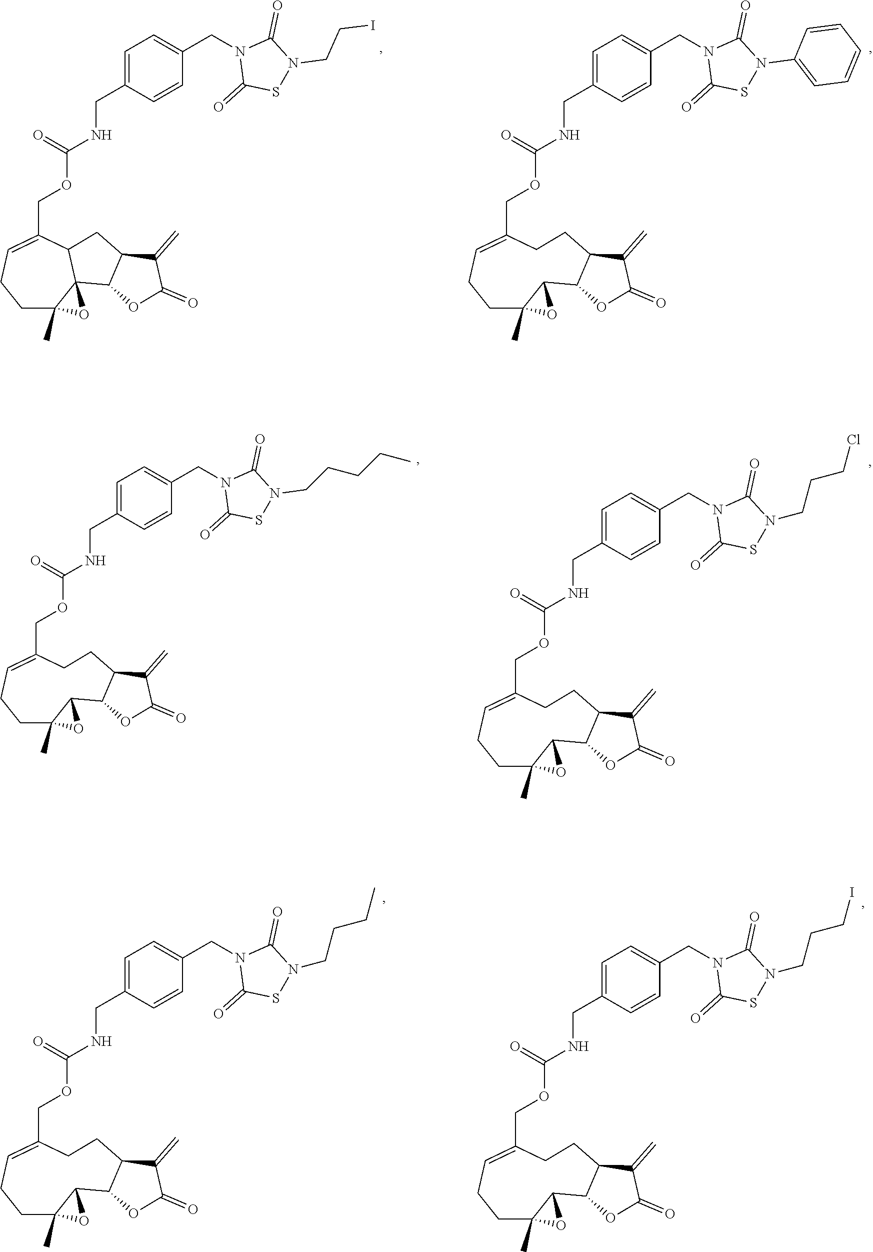

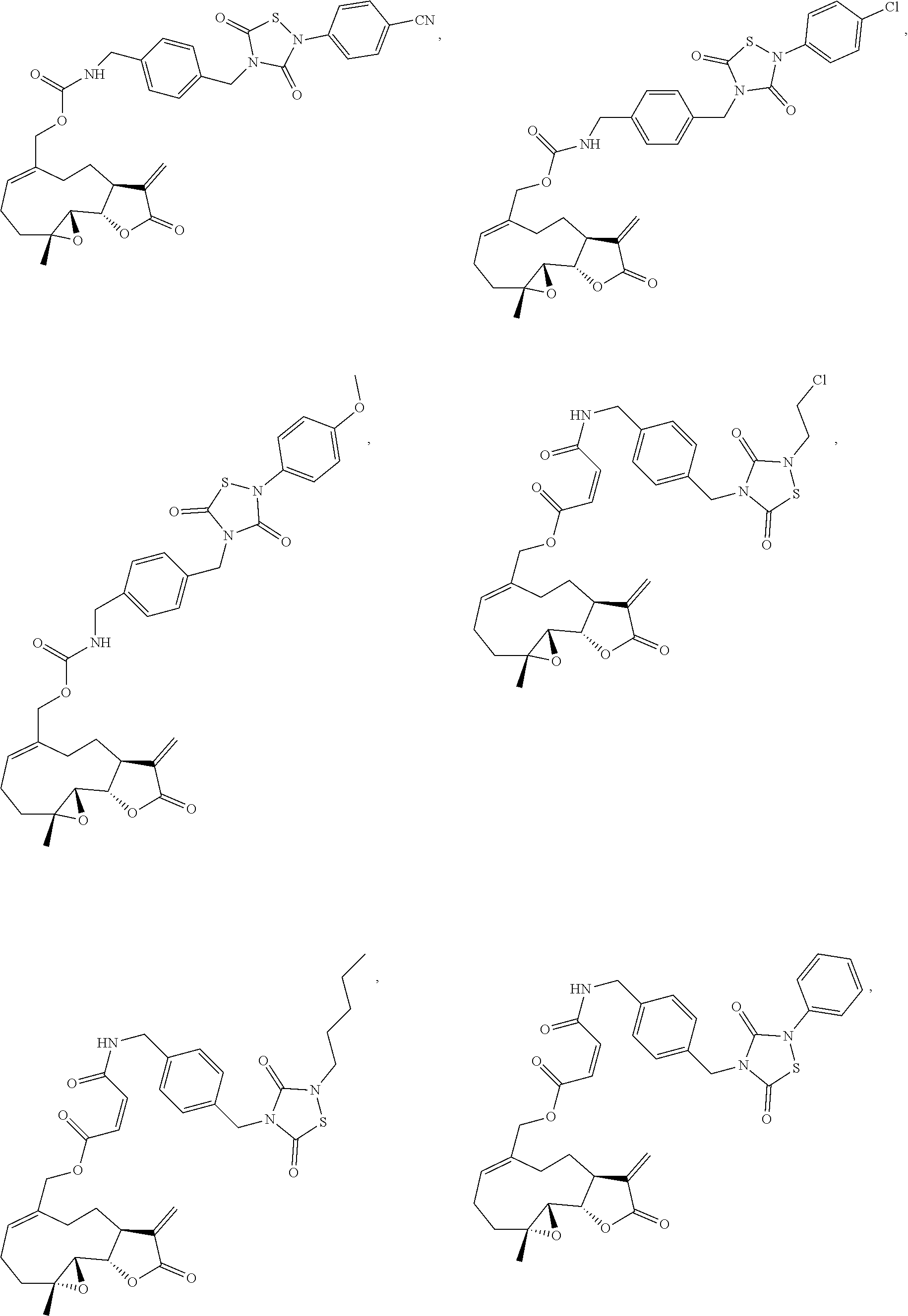

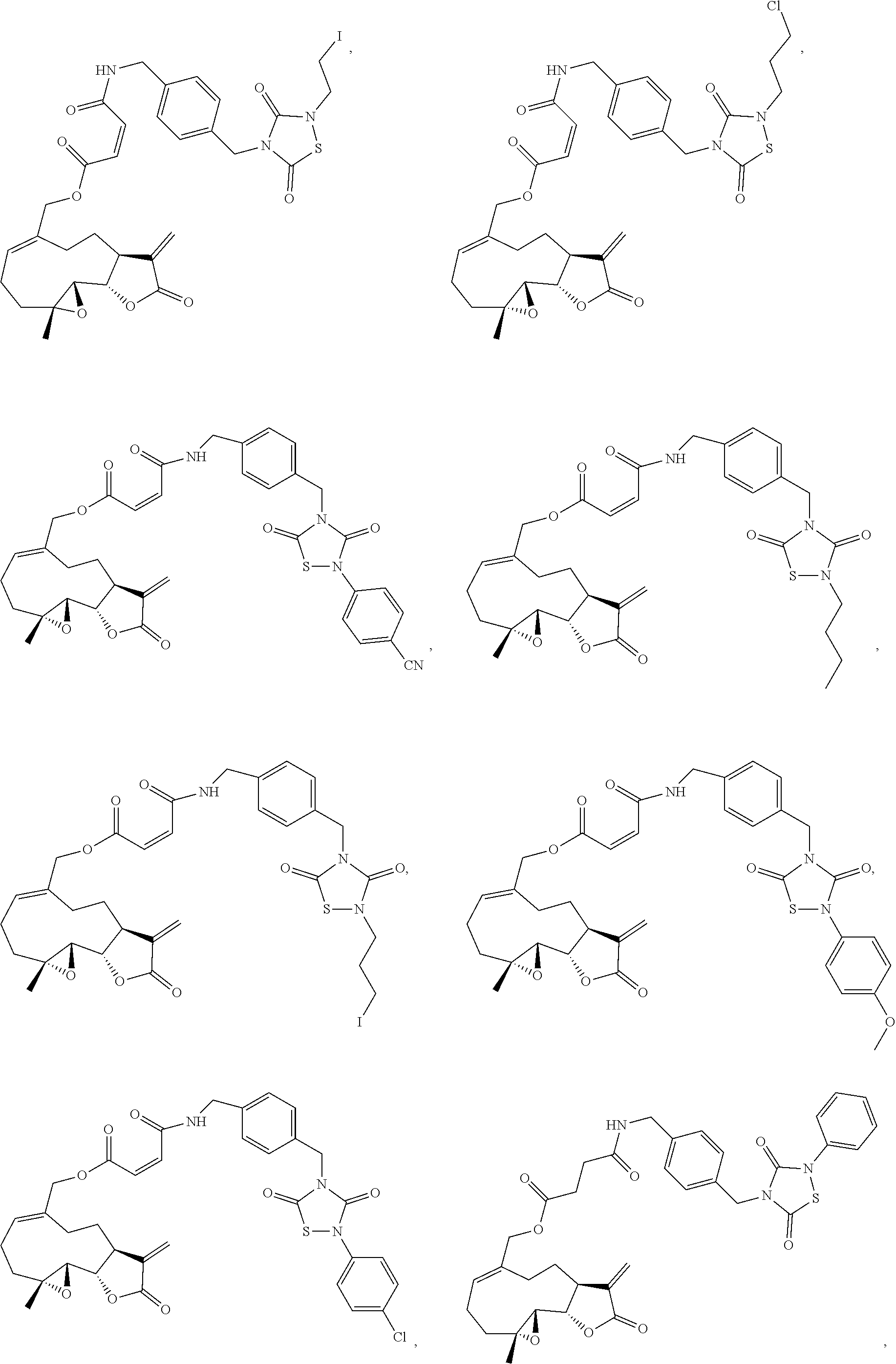

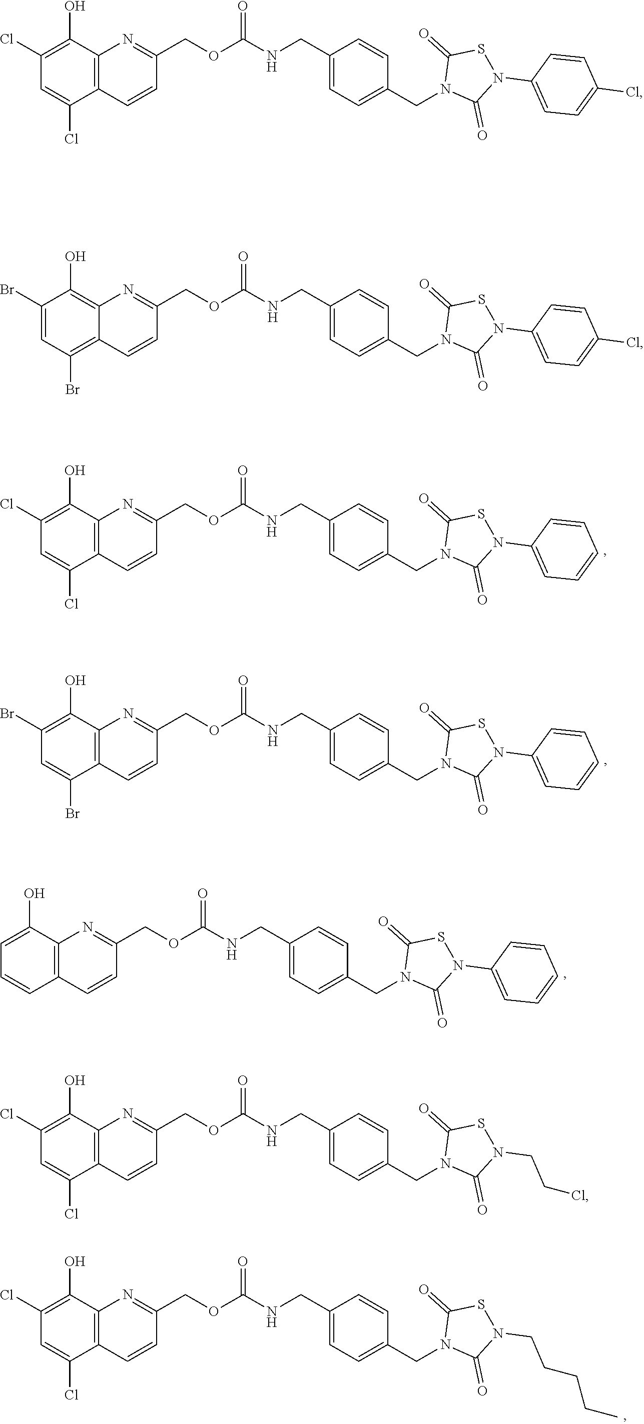

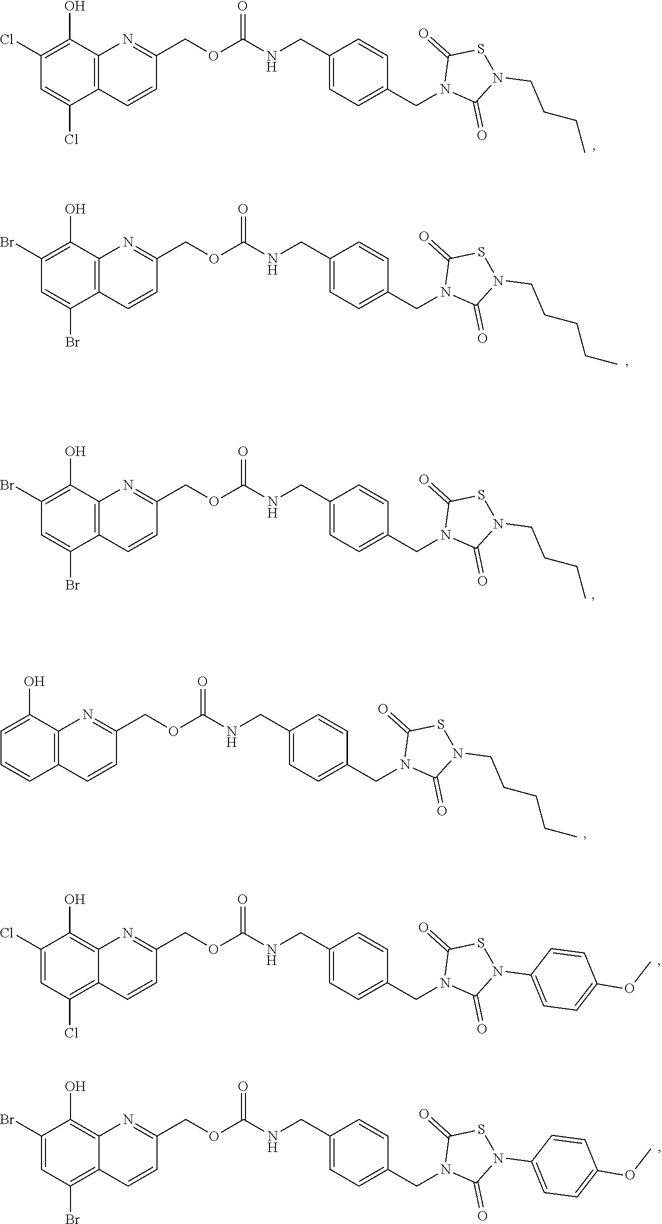 [SMILES: CCCCn1sc(=O)n(Cc2ccc(CNC(=O)OCc3ccc4c(Cl)cc(Cl)c(O)c4n3)cc2)c1=O ; CCCCCn1sc(=O)n(Cc2ccc(CNC(=O)OCc3ccc4c(Br)cc(Br)c(O)c4n3)cc2)c1=O ; CCCCn1sc(=O)n(Cc2ccc(CNC(=O)OCc3ccc4c(Br)cc(Br)c(O)c4n3)cc2)c1=O ; CCCCCn1sc(=O)n(Cc2ccc(CNC(=O)OCc3ccc4cccc(O)c4n3)cc2)c1=O ; COc1ccc(-n2sc(=O)n(Cc3ccc(CNC(=O)OCc4ccc5c(Cl)cc(Cl)c(O)c5n4)cc3)c2=O)cc1 ; COc1ccc(-n2sc(=O)n(Cc3ccc(CNC(=O)OCc4ccc5c(Br)cc(Br)c(O)c5n4)cc3)c2=O)cc1]

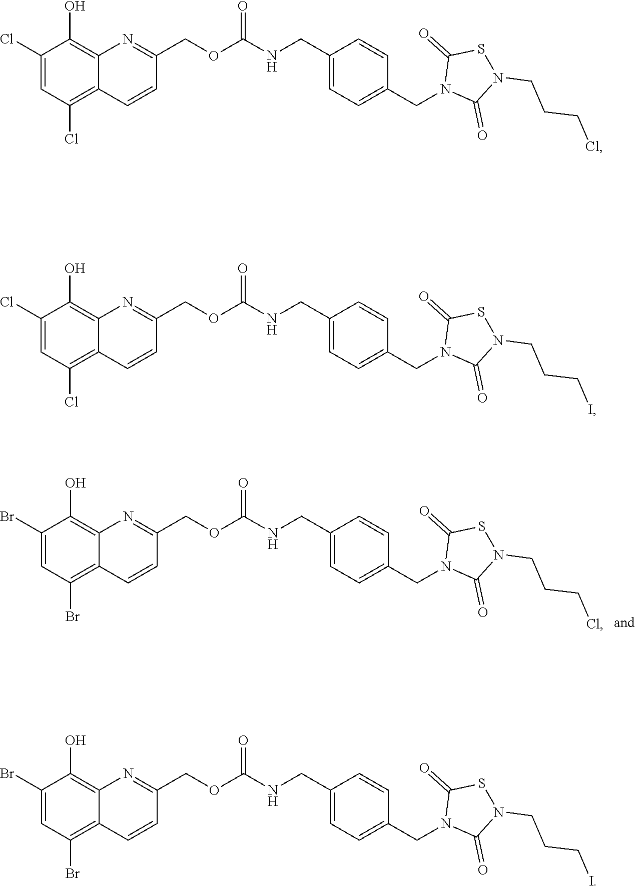 [SMILES: O=C(NCc1ccc(Cn2c(=O)sn(CCCCl)c2=O)cc1)OCc1ccc2c(Cl)cc(Cl)c(O)c2n1 ; O=C(NCc1ccc(Cn2c(=O)sn(CCCI)c2=O)cc1)OCc1ccc2c(Cl)cc(Cl)c(O)c2n1 ; O=C(NCc1ccc(Cn2c(=O)sn(CCCCl)c2=O)cc1)OCc1ccc2c(Br)cc(Br)c(O)c2n1 ; O=C(NCc1ccc(Cn2c(=O)sn(CCCI)c2=O)cc1)OCc1ccc2c(Br)cc(Br)c(O)c2n1]

or a pharmaceutically acceptable salt thereof.

In one aspect, a compound can be present as:

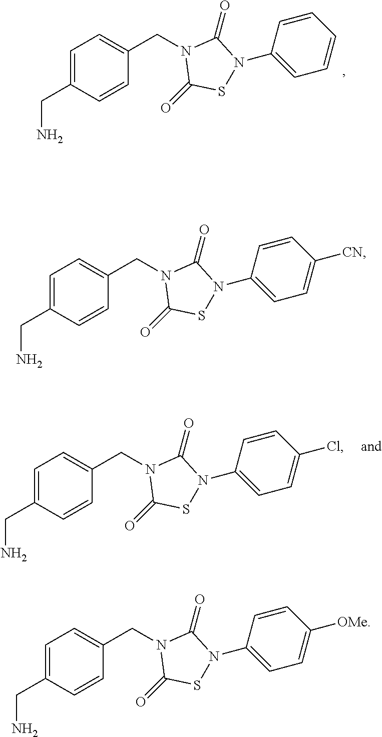 [SMILES: NCc1ccc(Cn2c(=O)sn(-c3ccccc3)c2=O)cc1 ; N#Cc1ccc(-n2sc(=O)n(Cc3ccc(CN)cc3)c2=O)cc1 ; NCc1ccc(Cn2c(=O)sn(-c3ccc(Cl)cc3)c2=O)cc1 ; COc1ccc(-n2sc(=O)n(Cc3ccc(CN)cc3)c2=O)cc1]

or a pharmaceutically acceptable salt thereof.

In one aspect, a compound can be present as:

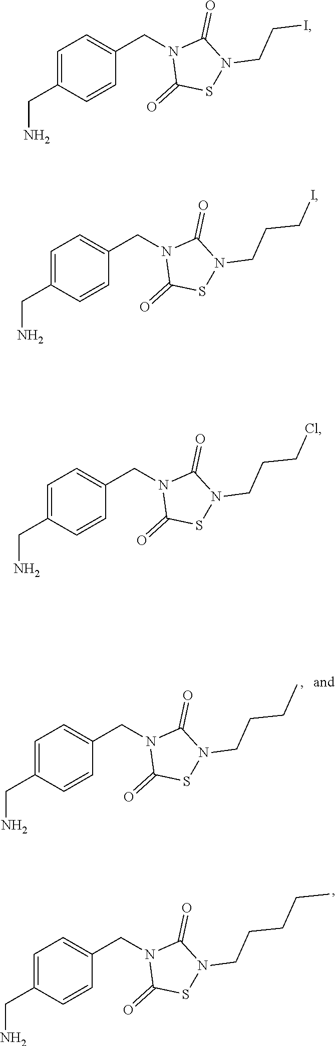 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof.

### C. PHARMACEUTICAL COMPOSITIONS

In one aspect, disclosed are pharmaceutical compositions comprising a disclosed compound, or a pharmaceutically acceptable salt thereof and a pharmaceutically acceptable carrier.

Thus, in one aspect, disclosed are pharmaceutical compositions comprising a therapeutically effective amount of a compound having a structure represented by a formula:

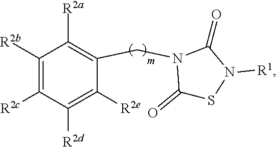

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

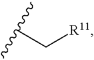

provided that one of R^{2a}, R^{2b}, R^{2c}, R^{2d}, and R^{2e} is Ar¹ or

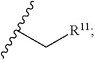

wherein R¹¹, when present, is a carboxylate residue of a chemotherapeutic agent or a carbamide residue of a chemotherapeutic agent; and wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R²⁰, when present, is selected from hydrogen, —OH, C1-C10 alkyl, C1-C10 alkoxy, C1-C10 alkylamino, and (C1-C10)(C1-C10) dialkylamino, provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is

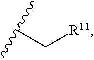

then R¹¹ is not —OC(O)₂(C1-C8 alkyl), —NHC(O)₂(C1-C8 alkyl), or —N(C1-C4 alkyl)C(O)₂(C1-C8 alkyl), or a pharmaceutically acceptable salt thereof, and a pharmaceutically acceptable carrier.

In one aspect, disclosed are pharmaceutical compositions comprising a therapeutically effective amount of a compound having a structure represented by a formula:

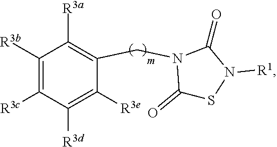

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH, or a pharmaceutically acceptable salt thereof, and a pharmaceutically acceptable carrier.

In one aspect, disclosed are pharmaceutical compositions comprising a therapeutically effective amount of a compound selected from:

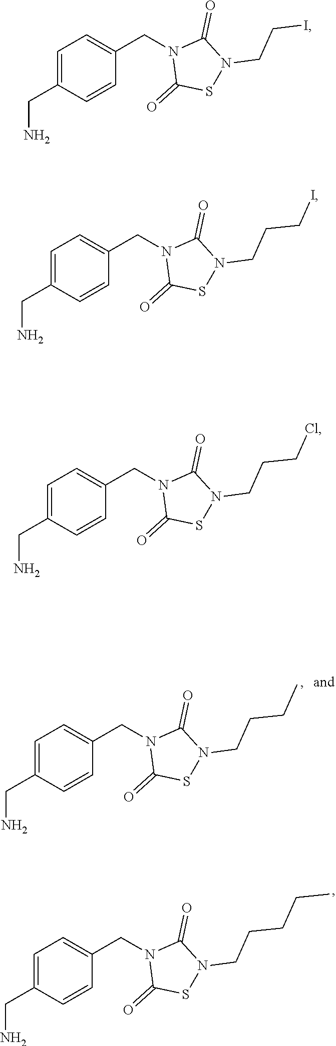 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof, and a pharmaceutically acceptable carrier.

In one aspect, disclosed are pharmaceutical compositions comprising a therapeutically effective amount of a compound:

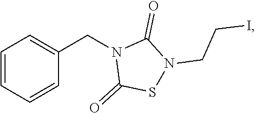 [SMILES: O=c1sn(CCI)c(=O)n1Cc1ccccc1]

or a pharmaceutically acceptable salt thereof, and a pharmaceutically acceptable carrier.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder of uncontrolled cellular proliferation such as, for example, cancers including, but not limited to, sarcomas, carcinomas, hematological cancers, solid tumors, breast cancer, cervical cancer, gastrointestinal cancer, colorectal cancer, brain cancer, skin cancer, prostate cancer, ovarian cancer, bladder cancer, thyroid cancer, testicular cancer, pancreatic cancer, endometrial cancer, melanomas, gliomas, leukemias, lymphomas, chronic myeloproliferative disorders, myelodysplastic syndromes, myeloproliferative neoplasms, and plasma cell neoplasms (myelomas).

It is understood that the disclosed compositions can be prepared from the disclosed compounds. It is also understood that the disclosed compositions can be employed in the disclosed methods of using.

### D. METHODS OF MAKING COMPOUNDS

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-IV, as described and exemplified below. The following examples are provided so that the invention might be more fully understood, are illustrative only, and should not be construed as limiting.

1. Route I

In one aspect, substituted TDZD analogs can be prepared as shown below.

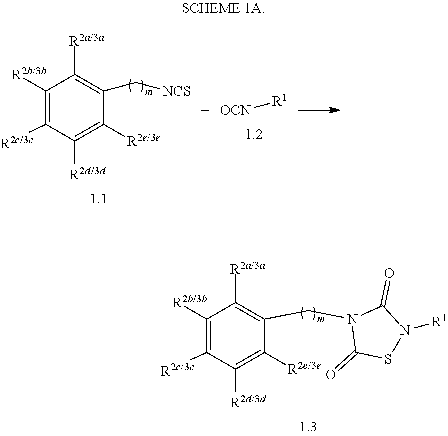

Compounds are represented in generic form, with substituents as noted in compound descriptions elsewhere herein. A more specific example is set forth below.

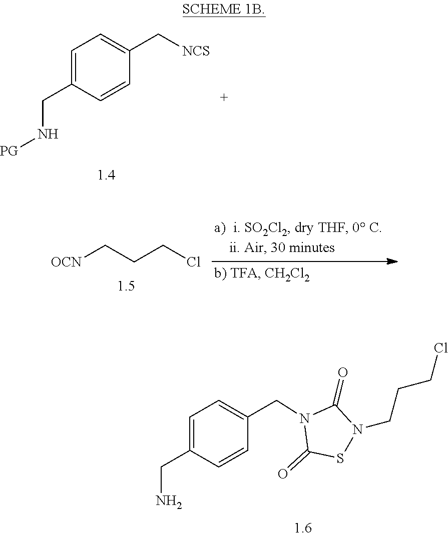

In one aspect, compounds of type 1.6 and similar compounds can be prepared according to reaction Scheme 1B above. Thus, compounds of type 1.6 can be prepared by reacting an isothiocyanate, e.g., 1.4 as shown above, with a corresponding isocyanate, e.g., 1.5 as shown above. Appropriate isothiocyanates and appropriate isocyanates are commercially available or prepared by methods known to one skilled in the art. The reaction is carried out in the presence of an appropriate chloride source, e.g., sulfuryl chloride as shown above, and an appropriate solvent, e.g., tetrahydrofuran (THF), at an appropriate temperature, e.g., 0° C., for an appropriate period of time, e.g., 30 minutes. For isothiocyanates with an amino functionality such as is present in 1.4, a protecting group (PG) can be used during the coupling reaction. In this case, an appropriate deprotecting agent, e.g., trifluoracetic acid, in an appropriate solvent, e.g., dichloromethane, can be used to yield the amine, e.g., 1.6. As can be appreciated by one skilled in the art, the above reaction provides an example of a generalized approach wherein compounds similar in structure to the specific reactants above (compounds similar to compounds of type 1.1 and 1.2) can be substituted in the reaction to provide 2,4-disubstituted thiadiazolidinone analogs similar to Formula 1.3 as shown in Scheme 1A above.

2. Route II

In one aspect, substituted TDZD analogs can be prepared as shown below.

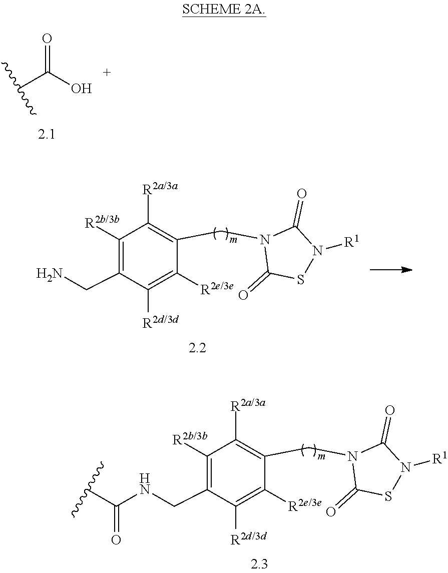

Compounds are represented in generic form, with substituents as noted in compound descriptions elsewhere herein. A more specific example is set forth below.

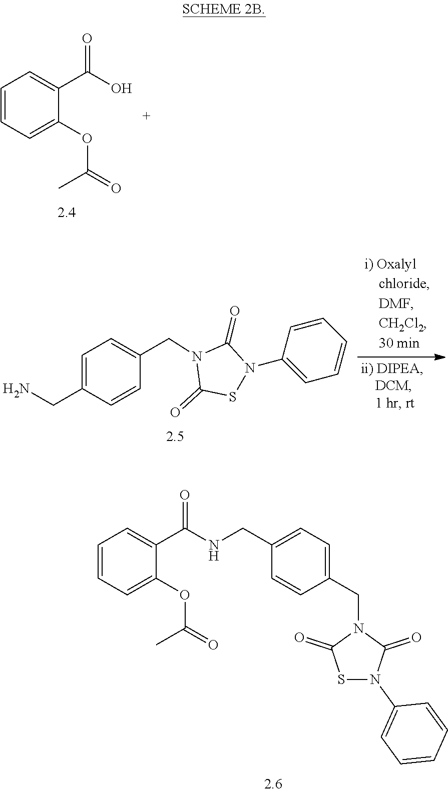 [SMILES: CC(=O)Oc1ccccc1C(=O)O ; NCc1ccc(Cn2c(=O)sn(-c3ccccc3)c2=O)cc1 ; CC(=O)Oc1ccccc1C(=O)NCc1ccc(Cn2c(=O)sn(-c3ccccc3)c2=O)cc1]

In one aspect, compounds of type 2.6 and similar compounds can be prepared according to reaction Scheme 2B above. Thus, compounds of type 2.6 can be prepared by a coupling reaction between an appropriate acid, e.g., 2-acetoxybenzoic acid 2.4 as shown above, and an appropriate amine, e.g., 2.5 as shown above. Appropriate acids and appropriate amines are commercially available or prepared by methods known to one skilled in the art. The reaction is carried out by converting the acid, e.g., 2.4, to its acid chloride using an appropriate chloride source, e.g., oxalyl chloride as shown above, in an appropriate solvent such as dichloromethane as shown above, for an appropriate time, e.g., 30 minutes. The acid chloride can then be coupled to an amine, e.g., 2.5, using an appropriate base, e.g., N,N-diisopropylethylamine (DIPEA), in an appropriate solvent, e.g., dichloromethane as shown above, for an appropriate time, e.g., one hour as shown above, to give the product, e.g., 2.6. As can be appreciated by one skilled in the art, the above reaction provides an example of a generalized approach wherein compounds similar in structure to the specific reactants above (compounds similar to compounds of type 2.1 and 2.2) can be substituted in the reaction to provide 2,4-disubstituted thiadiazolidinone analogs similar to Formula 2.3 as shown in Scheme 2A above.

3. Route III

In one aspect, substituted TDZD analogs can be prepared as shown below.

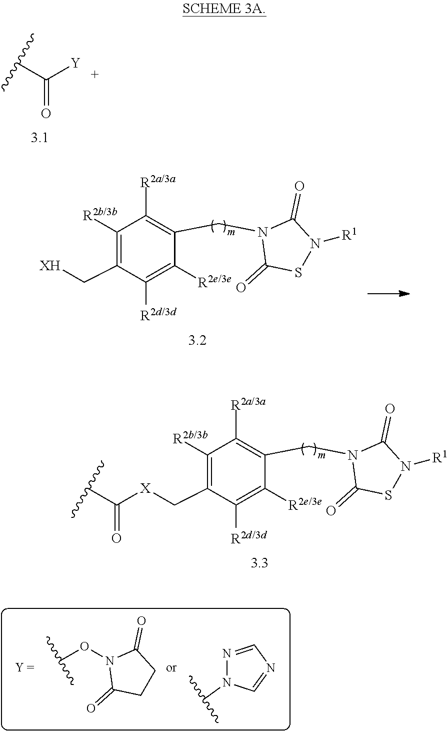

Compounds are represented in generic form, with X selected from —NH— and —O—, and other substituents as noted in compound descriptions elsewhere herein. A more specific example is set forth below.

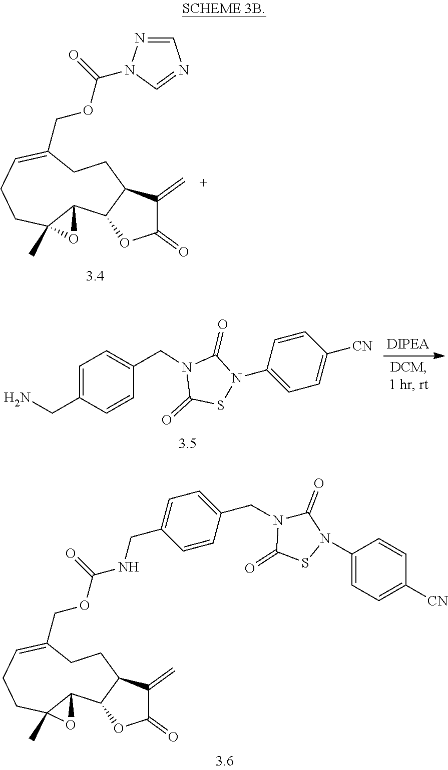 [SMILES: C=C1C(=O)O[C@H]2[C@H]1CC/C(COC(=O)n1cncn1)=C\CC[C@@]1(C)O[C@@H]21 ; N#Cc1ccc(-n2sc(=O)n(Cc3ccc(CN)cc3)c2=O)cc1 ; C=C1C(=O)O[C@H]2[C@H]1CC/C(COC(=O)NCc1ccc(Cn3c(=O)sn(-c4ccc(C#N)cc4)c3=O)cc1)=C\CC[C@@]1(C)O[C@@H]21]

In one aspect, compounds of type 3.6 and similar compounds can be prepared according to reaction Scheme 3B above. Thus, compounds of type 3.6 can be prepared by reacting a triazole, e.g., 3.4, with a corresponding alcohol or amine, e.g., 3.5. Appropriate triazoles, appropriate alcohols, and appropriate amines are commercially available or prepared by methods known to one skilled in the art. The coupling reaction can be carried out with a suitable base, e.g., N,N-diisopropylethylamine (DIPEA) as shown above, in an appropriate solvent, e.g., dichloromethane as shown above, for an appropriate time, e.g., one hour, to give the product, e.g., 3.6. As can be appreciated by one skilled in the art, the above reaction provides an example of a generalized approach wherein compounds similar in structure to the specific reactants above (compounds similar to compounds of type 3.1 and 3.2) can be substituted in the reaction to provide 2,4-disubstituted thiadiazolidinone analogs similar to Formula 3.3 as shown in Scheme 3A above.

4. Route IV

In one aspect, substituted TDZD analogs can be prepared as shown below.

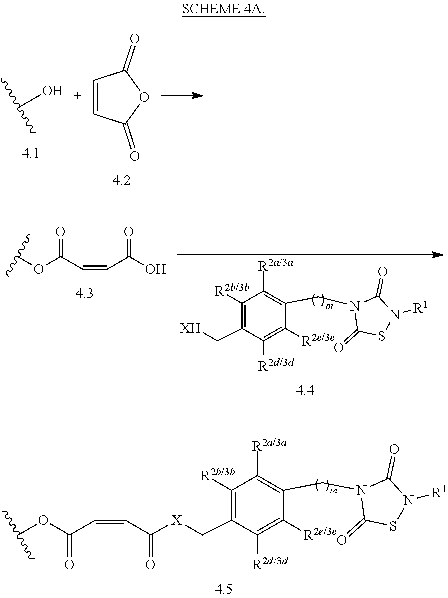

Compounds are represented in generic form, with X selected from —NH— and —O—, and other substituents as noted in compound descriptions elsewhere herein. A more specific example is set forth below.

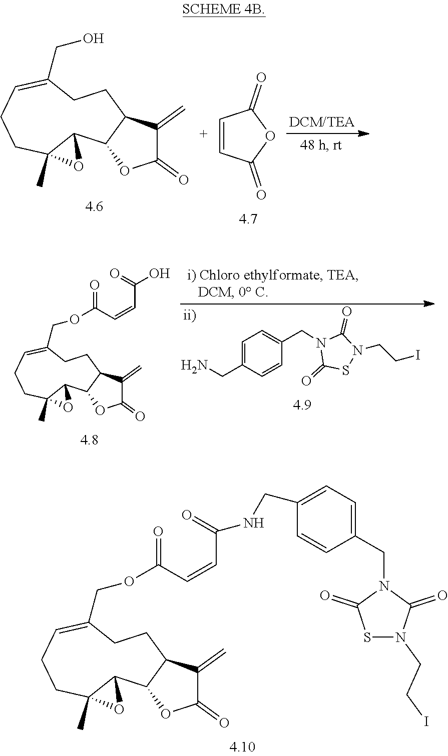

In one aspect, compounds of type 4.10 and similar compounds can be prepared according to reaction Scheme 4B above. Thus, compounds of type 4.8 can be prepared by reacting an alcohol, e.g., 4.6 as shown above, with maleic anhydride 4.7 to give the intermediate, e.g., 4.8. Appropriate alcohols are commercially available or prepared by methods known to one skilled in the art. The reaction can be carried out in a suitable solvent, e.g., dichloromethane as shown above, in the presence of a suitable base, e.g., triethylamine as shown above, for an appropriate time, e.g., 48 hours. Compounds of type 4.10 can be prepared by reacting the intermediate, e.g., 4.8, with a corresponding amine, e.g., 4.9, in the presence of a suitable activating agent, e.g., chloro ethylformate as shown above, and a suitable base, e.g., triethylamine, in a suitable solvent, e.g., dichloromethane, at a suitable temperature, e.g., 0° C. As can be appreciated by one skilled in the art, the above reaction provides an example of a generalized approach wherein compounds similar in structure to the specific reactants above (compounds similar to compounds of type 4.1, 4.2, 4.3, and 4.4) can be substituted in the reaction to provide 2,4-disubstituted thiadiazolidinone analogs similar to Formula 4.5 as shown in Scheme 4A above.

### E. TREATING DISORDERS OF UNCONTROLLED CELLULAR PROLIFERATION IN A SUBJECT

In one aspect, disclosed are methods of treating a disorder of uncontrolled cellular proliferation in a subject, the method comprising the step of administering to the subject an effective amount of at least one disclosed compound, or a pharmaceutically acceptable salt thereof.

Thus, in one aspect, disclosed are methods for treating a disorder of uncontrolled cellular proliferation in a subject, the method comprising administering to the subject an effective amount of a compound having a structure represented by a formula:

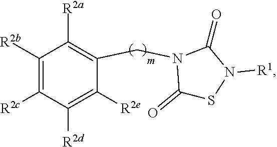

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

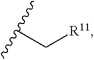

provided that one of R^{2a}, R^{2b}, R^{2c}, R^{2d}, and R^{2e} is Ar¹ or

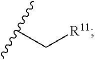

wherein R¹¹, when present, is a carboxylate residue of a chemotherapeutic agent or a carbamide residue of a chemotherapeutic agent; and wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R²⁰, when present, is selected from hydrogen, —OH, C1-C10 alkyl, C1-C10 alkoxy, C1-C10 alkylamino, and (C1-C10)(C1-C10) dialkylamino, provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is

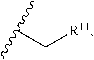

then R¹¹ is not —OC(O)₂(C1-C8 alkyl), —NHC(O)₂(C1-C8 alkyl), or —N(C1-C4 alkyl)C(O)₂(C1-C8 alkyl), or a pharmaceutically acceptable salt thereof.

In one aspect, disclosed are methods for treating a disorder of uncontrolled cellular proliferation in a subject, the method comprising administering to the subject an effective amount of a compound having a structure represented by a formula:

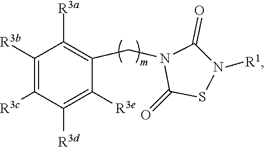

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH, or a pharmaceutically acceptable salt thereof, and methods of making and using same.

In one aspect, disclosed are methods for treating a disorder of uncontrolled cellular proliferation in a subject, the method comprising administering to the subject an effective amount of a compound selected from:

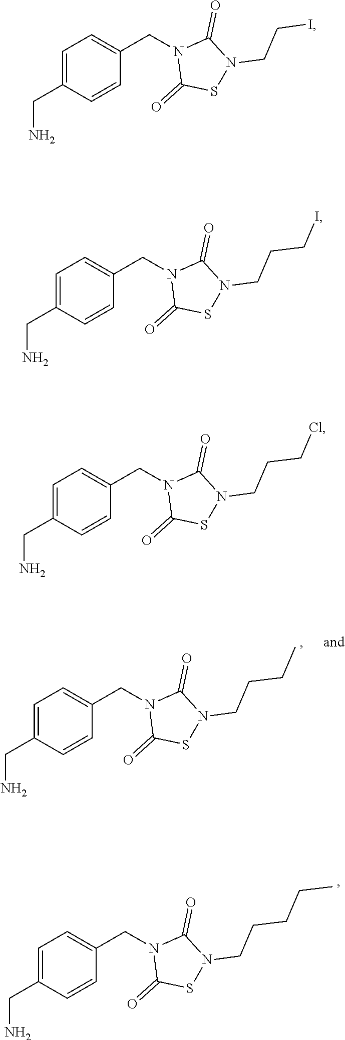 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof.

In one aspect, disclosed are methods for treating a disorder of uncontrolled cellular proliferation in a subject, the method comprising administering to the subject an effective amount of a compound:

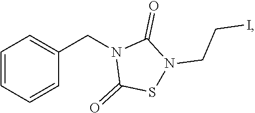 [SMILES: O=c1sn(CCI)c(=O)n1Cc1ccccc1]

or a pharmaceutically acceptable salt thereof.

In a further aspect, the effective amount is a therapeutically effective amount. In a still further aspect, the effective amount is a prophylactically effective amount.

In a further aspect, the subject is a mammal. In a still further aspect, the mammal is a human.

In a further aspect, the subject has been diagnosed with a need for treatment of the disorder prior to the administering step.

In a further aspect, the method further comprises the step of identifying a subject in need of treatment of the disorder.

In a further aspect, the disorder is a cancer. Examples of cancers include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

In a further aspect, the cancer is a hematological cancer. In a still further aspect, the hematological cancer is selected from leukemia, lymphoma, and multiple myeloma.

In a further aspect, the cancer is a solid tumor. In a still further aspect, the solid tumor is selected from lung cancer, liver cancer, pancreatic cancer, a central nervous system cancer, breast cancer, ovarian cancer, colon cancer, renal cancer, melanoma, prostate cancer, and head and neck cancer.

### F. TREATING A NEUROLOGICAL DISORDER IN A SUBJECT

In one aspect, disclosed are methods of treating a neurodegenerative disorder in a subject, the method comprising the step of administering to the subject an effective amount of at least one disclosed compound, or a pharmaceutically acceptable salt thereof.

Thus, in one aspect, disclosed are methods for treating a neurodegenerative disorder in a subject, the method comprising administering to the subject an effective amount of a compound having a structure represented by a formula:

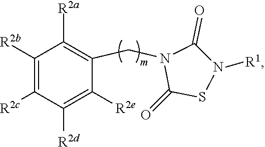

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

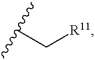

provided that one of R^{2a}, R^{2b}, R^{2c}, R^{2d}, and R^{2e} is Ar¹ or

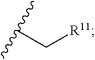

wherein R¹¹, when present, is a carboxylate residue of a chemotherapeutic agent or a carbamide residue of a chemotherapeutic agent; and wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R²⁰, when present, is selected from hydrogen, —OH, C1-C10 alkyl, C1-C10 alkoxy, C1-C10 alkylamino, and (C1-C10)(C1-C10) dialkylamino, provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is

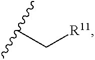

then R¹¹ is not —OC(O)₂(C1-C8 alkyl), —NHC(O)₂(C1-C8 alkyl), or —N(C1-C4 alkyl)C(O)₂(C1-C8 alkyl), or a pharmaceutically acceptable salt thereof.

In one aspect, disclosed are methods for treating a neurodegenerative disorder in a subject, the method comprising administering to the subject an effective amount of a compound having a structure represented by a formula:

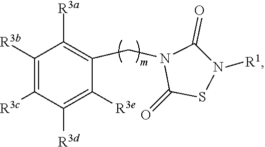

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH, or a pharmaceutically acceptable salt thereof, and methods of making and using same.

In one aspect, disclosed are methods for treating a neurodegenerative disorder in a subject, the method comprising administering to the subject an effective amount of a compound selected from:

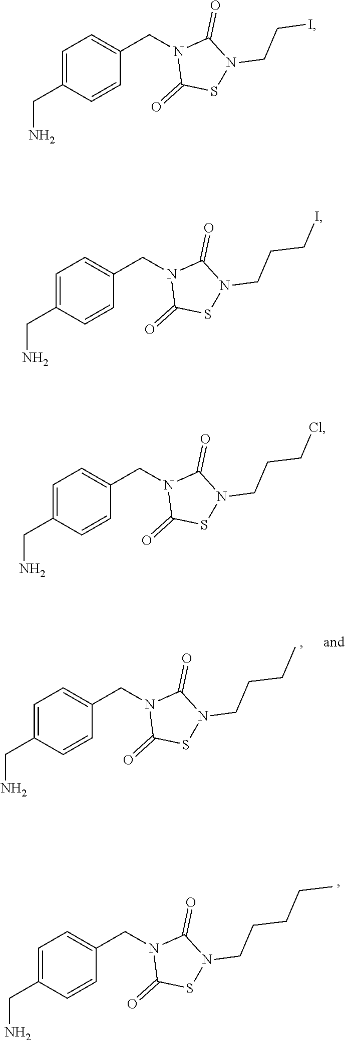 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof.

In one aspect, disclosed are methods for treating a neurological disorder in a subject, the method comprising administering to the subject an effective amount of a compound selected from:

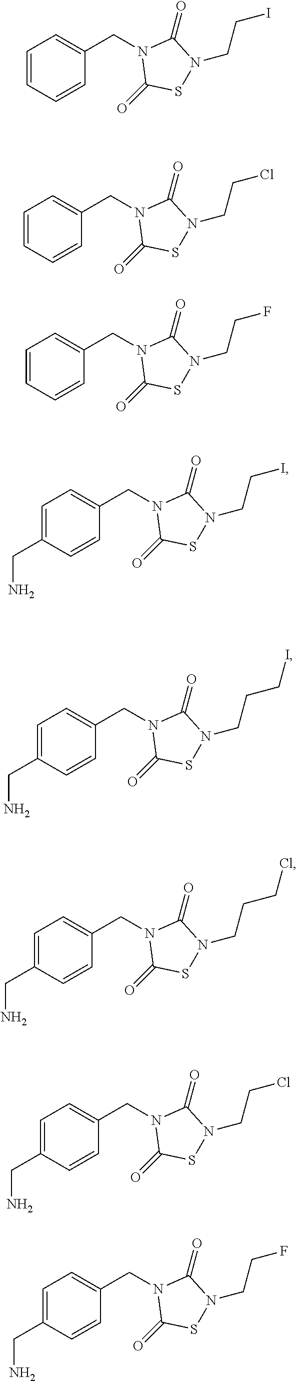

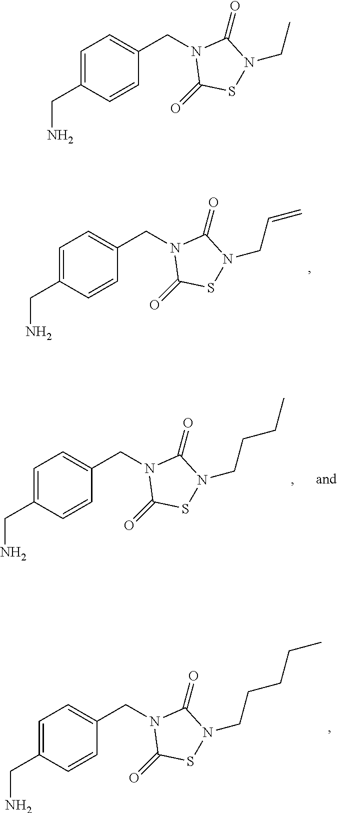 [SMILES: CCn1sc(=O)n(Cc2ccc(CN)cc2)c1=O ; C=CCn1sc(=O)n(Cc2ccc(CN)cc2)c1=O ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof.

In a further aspect, the effective amount is a therapeutically effective amount. In a still further aspect, the effective amount is a prophylactically effective amount.

In a further aspect, the subject is a mammal. In a still further aspect, the mammal is a human.

In a further aspect, the subject has been diagnosed with a need for treatment of the neurological disorder prior to the administering step.

In a further aspect, the method further comprises the step of identifying a subject in need of treatment of the neurological disorder.

In a further aspect, the neurological disorder is associated with age.

In a further aspect, the neurological disorder is selected from sarcopenia, supranuclear palsy, Alzheimer's disease, and dementia.

### G. ADDITIONAL METHODS OF USING THE COMPOUNDS

The compounds and pharmaceutical compositions of the invention are useful in treating or controlling neurodegenerative diseases and disorders of uncontrolled cellular proliferation such as, for example, cancer.

Examples of neurodegenerative diseases for which compounds and compositions can be useful in treating include, but are not limited to, sarcopenia, supranuclear palsy, Alzheimer's disease, dementia.

Examples of disorders of uncontrolled cellular proliferation for which the compounds and compositions can be useful in treating include, but are not limited to, cancers such as, for example, sarcomas, carcinomas, hematological cancers, solid tumors, breast cancer, cervical cancer, gastrointestinal cancer, colorectal cancer, brain cancer, skin cancer, prostate cancer, ovarian cancer, bladder cancer, thyroid cancer, testicular cancer, pancreatic cancer, endometrial cancer, melanomas, gliomas, leukemias, lymphomas, chronic myeloproliferative disorders, myelodysplastic syndromes, myeloproliferative neoplasms, and plasma cell neoplasms (myelomas).

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a disorder of uncontrolled cellular proliferation, such as cancer.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disorder of uncontrolled cellular proliferation, such as cancer.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Use of Compounds

In one aspect, the invention relates to the use of a disclosed compound or a product of a disclosed method. In a further aspect, a use relates to the manufacture of a medicament for the treatment of a disorder of uncontrolled cellular proliferation in a subject. In a still further aspect, a use relates to the manufacture of a medicament for the treatment of a neurodegenerative disease in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a disorder of uncontrolled cellular proliferation in a subject. In one aspect, the use is characterized in that the subject is a human. In one aspect, the use is characterized in that the disorder of uncontrolled cellular proliferation is a cancer.

In various aspects, the use relates to a treatment of a neurodegenerative disease in a subject. In one aspect, the use is characterized in that the subject is a human. In one aspect, the use is characterized in that the neurodegenerative disease is selected from sarcopenia, supranuclear palsy, Alzheimer's disease, and dementia.

In a further aspect, the use relates to the manufacture of a medicament for the treatment of a disorder of uncontrolled cellular proliferation in a subject.

In a further aspect, the use relates to the manufacture of a medicament for the treatment of a neurodegenerative disease in a subject.

It is understood that the disclosed uses can be employed in connection with the disclosed compounds, products of disclosed methods of making, methods, compositions, and kits.

In a further aspect, the invention relates to the use of a disclosed compound or a disclosed product in the manufacture of a medicament for the treatment of a disorder of uncontrolled cellular proliferation in a mammal. In a further aspect, the disorder of uncontrolled cellular proliferation is a cancer.

In a further aspect, the invention relates to the use of a disclosed compound or a disclosed product in the manufacture of a medicament for the treatment of a neurodegenerative disease in a mammal. In a further aspect, the neurodegenerative disease is selected from sarcopenia, supranuclear palsy, Alzheimer's disease, and dementia.

2. Manufacture of a Medicament

In one aspect, the invention relates to a method for the manufacture of a medicament for treating a disorder of uncontrolled cellular proliferation in a subject having the disorder, the method comprising combining a therapeutically effective amount of a disclosed compound or product of a disclosed method with a pharmaceutically acceptable carrier or diluent.

In one aspect, the invention relates to a method for the manufacture of a medicament for treating a neurodegenerative disease in a subject having the disorder, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a disorder of uncontrolled cellular proliferation. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

3. Kits

In one aspect, disclosed are kits comprising an effective amount of a disclosed compound, and one or more of: (a) at least one agent associated with the treatment of a disorder of uncontrolled cellular proliferation; (b) at least one agent associated with the treatment of a neurological disorder; (c) instructions for administering the compound in connection with treating a disorder of uncontrolled cellular proliferation; (d) instructions for administering the compound in connection with treating a neurological disorder; (e) instructions for treating a disorder of uncontrolled cellular proliferation; and (f) instructions for treating a neurological disorder.

Thus, in one aspect, disclosed are kits comprising an effective amount of a compound having a structure represented by a formula:

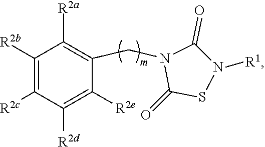

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{2a}, R^{2b}, R^{2e}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula:

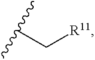

provided that one of R^{2a}, R^{2b}, R^{2c}, R^{2d}, and R^{2e} is Ar¹ or

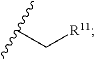

wherein R¹¹, when present, is a carboxylate residue of a chemotherapeutic agent or a carbamide residue of a chemotherapeutic agent; and wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R²⁰, when present, is selected from hydrogen, —OH, C1-C10 alkyl, C1-C10 alkoxy, C1-C10 alkylamino, and (C1-C10)(C1-C10) dialkylamino, provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is

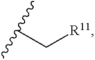

then R¹¹ is not —OC(O)₂(C1-C8 alkyl), —NHC(O)₂(C1-C8 alkyl), or —N(C1-C4 alkyl)C(O)₂(C1-C8 alkyl), or a pharmaceutically acceptable salt thereof, and one or more of: (a) at least one agent associated with the treatment of a disorder of uncontrolled cellular proliferation; (b) at least one agent associated with the treatment of a neurological disorder; (c) instructions for administering the compound in connection with treating a disorder of uncontrolled cellular proliferation; (d) instructions for administering the compound in connection with treating a neurological disorder; (e) instructions for treating a disorder of uncontrolled cellular proliferation; and (f) instructions for treating a neurological disorder.

Thus, in one aspect, disclosed are kits comprising an effective amount of a compound having a structure represented by a formula:

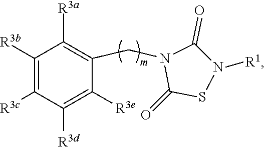

wherein m is 0, 1, 2, or 3; wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹; wherein R¹⁰, when present, is selected from hydrogen, —OH, C1-C10 alkyl, C1-C10 alkoxy, C1-C10 alkylamino, and (C1-C10)(C1-C10) dialkylamino; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH, or a pharmaceutically acceptable salt thereof, and one or more of: (a) at least one agent associated with the treatment of a disorder of uncontrolled cellular proliferation; (b) at least one agent associated with the treatment of a neurological disorder; (c) instructions for administering the compound in connection with treating a disorder of uncontrolled cellular proliferation; (d) instructions for administering the compound in connection with treating a neurological disorder; (e) instructions for treating a disorder of uncontrolled cellular proliferation; and (f) instructions for treating a neurological disorder.

In one aspect, disclosed are kits comprising an effective amount of a compound selected from:

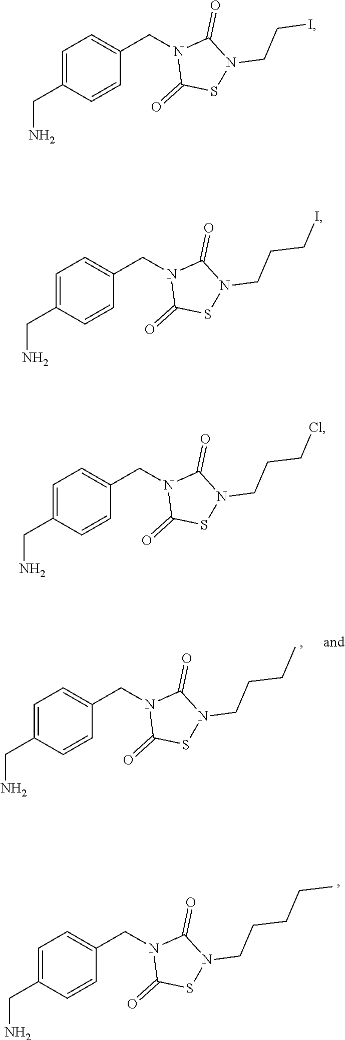 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]

or a pharmaceutically acceptable salt thereof, and one or more of: (a) at least one agent associated with the treatment of a disorder of uncontrolled cellular proliferation; (b) at least one agent associated with the treatment of a neurological disorder; (c) instructions for administering the compound in connection with treating a disorder of uncontrolled cellular proliferation; (d) instructions for administering the compound in connection with treating a neurological disorder; (e) instructions for treating a disorder of uncontrolled cellular proliferation; and (f) instructions for treating a neurological disorder.

In a further aspect, the agent associated with the treatment of a disorder of uncontrolled cellular proliferation is a chemotherapeutic agent. In a still further aspect, the chemotherapeutic agent is selected from an alkylating agent, an antimetabolite agent, an antineoplastic antibiotic agent, a mitotic inhibitor agent, and an mTor inhibitor agent.

In various aspects, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the mTor inhibitor agent is selected from everolimus, siroliumus, and temsirolimus, or a pharmaceutically acceptable salt, hydrate, solvate, or polymorph thereof.

In a further aspect, the agent associated with the treatment of a neurological disorder is selected from a cholinesterase inhibitor, an antidepressant, memantine, rilutek, radicava, levodopa, carbidopa, a dopamine agonist, a MAO-B inhibitor, a catechol-O-methyltransferase inhibitor, an anticholinergic, spinraza, tetrabenazine, an antipsychotic agent, levetiracetam, clonazepam, an antipsychotic agent, a mood-stabilizing agent, and amantadine.

In a further aspect, the disorder of uncontrolled cellular proliferation is a cancer.

In a further aspect, the neurological disorder is selected from sarcopenia, supranuclear palsy, Alzheimer's disease, and dementia.

In a further aspect, the compound and the agent are co-packaged.

In a further aspect, the compound and the agent are administered sequentially. In a still further aspect, the compound and the agent are administered simultaneously.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Chemistry Experimentals

a. Preparation of TDZD Analogues

In the literature, several methods have been reported for the preparation of TDZD analogues by reaction of isocyanates and isothiocyanates using a variety of reagents such as sulfuryl chloride (Slomczynska et al. (1984) Journal of Heterocyclic Chemistry. 21(1): 241-246), chlorine gas (Martinez et al. (2002) J Med Chem. 45(6): 1292-1299), N-chlorosuccinimide (NCS) (Nasim S et al. (2009) Tetrahedron Letters. 50(3): 257-259), etc. By comparing all these methods, it was found that sulfuryl chloride is the most suitable reagent for the synthesis of TDZD analogues (Structure II). The synthesis of TDZD analogues is shown in Scheme 1.

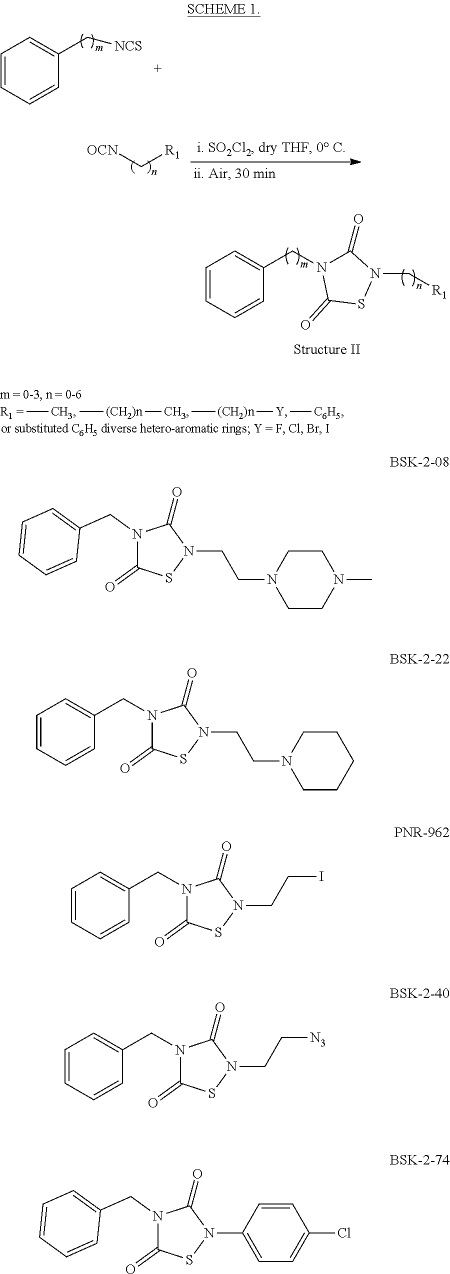

Initially, TDZD analogues with terminal amino or terminal hydroxyl groups (Structure III) were synthesized, as shown in Scheme 2.

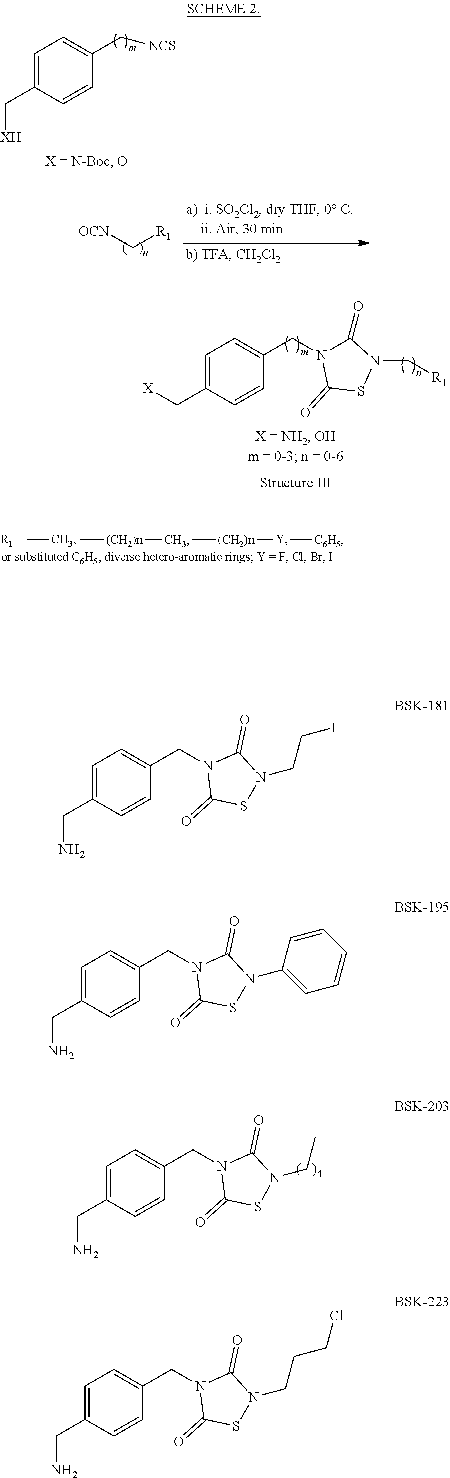

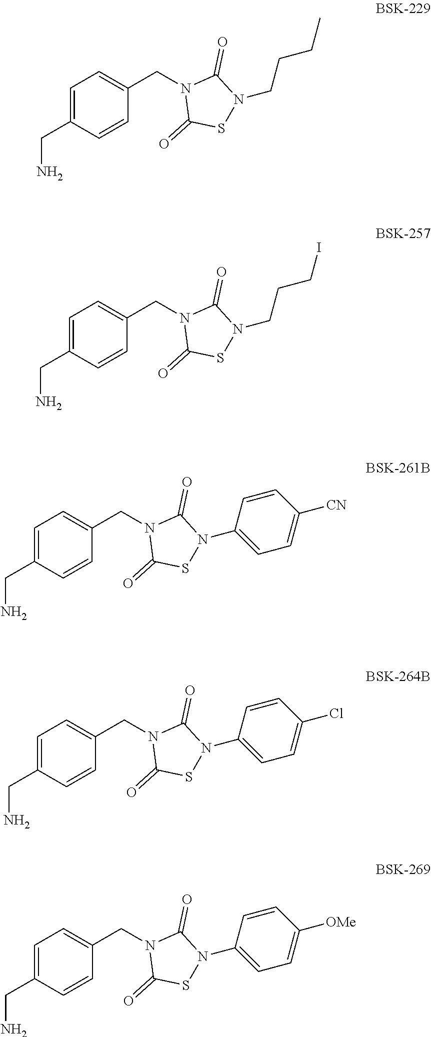 [SMILES: CCCCn1sc(=O)n(Cc2ccc(CN)cc2)c1=O ; NCc1ccc(Cn2c(=O)sn(CCCI)c2=O)cc1 ; N#Cc1ccc(-n2sc(=O)n(Cc3ccc(CN)cc3)c2=O)cc1 ; NCc1ccc(Cn2c(=O)sn(-c3ccc(Cl)cc3)c2=O)cc1 ; COc1ccc(-n2sc(=O)n(Cc3ccc(CN)cc3)c2=O)cc1]

In the next step, TDZD-aspirin analogs were synthesized by dissolving 2-acetoxybenzoic acid in dichloromethane and converting it to its acid chloride by reaction with oxalyl chloride followed by addition of a few drops of dimethyl formamide. After CO₂ gas evolution ceased, the mixture was concentrated and immediately reacted with simple and substituted 4-(4-(aminomethyl)benzyl)-2-(2-chloroethyl)-1,2,4-thiadiazolidine-3,5-diones and N,N-diisopropylethylamine (DIPEA) in dichloromethane, by drop-wise addition of crude 2-acetoxybenzoic acid chloride in dichloromethane (DCM). The resulting reaction mixture was stirred for 1 hr to obtain the appropriate TDZD-aspirin amide conjugate (Structure IV, Scheme 3).

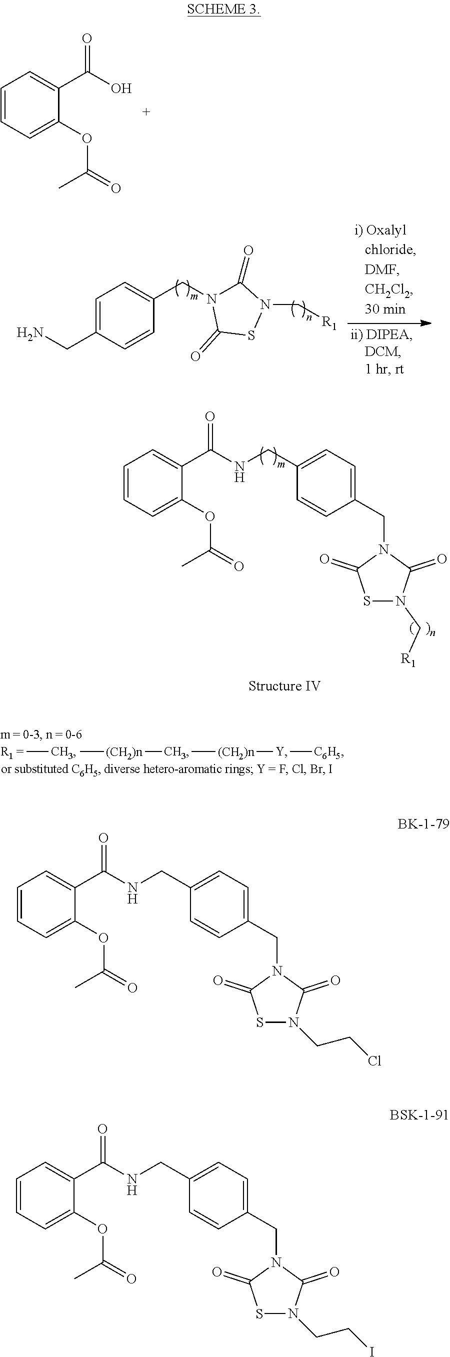

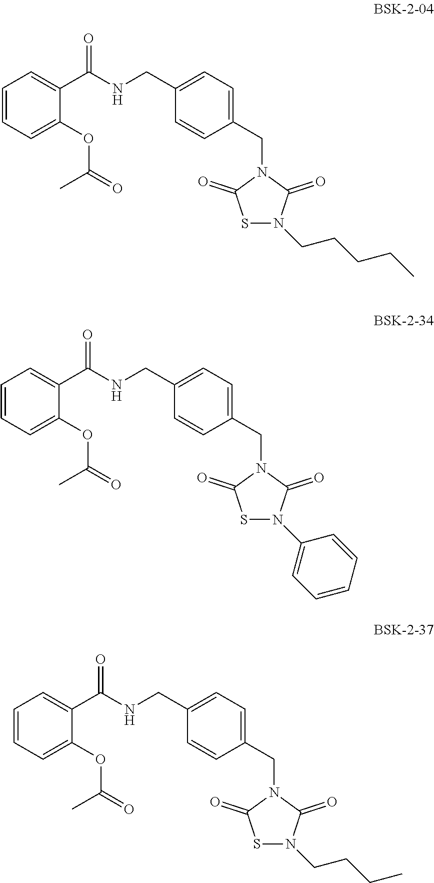 [SMILES: CCCCCn1sc(=O)n(Cc2ccc(CNC(=O)c3ccccc3OC(C)=O)cc2)c1=O ; CC(=O)Oc1ccccc1C(=O)NCc1ccc(Cn2c(=O)sn(-c3ccccc3)c2=O)cc1 ; CCCCn1sc(=O)n(Cc2ccc(CNC(=O)c3ccccc3OC(C)=O)cc2)c1=O]

In another iteration, a solution of 2-(4-isobutylphenyl)propanyl-N-hydroxy succinimide (ibuprofen succinimide) in dimethylformamide (DMF) was added dropwise to a mixture of compound III and N, N-diisopropylethylamine (DIPEA). The reaction mixture was stirred at room temperature for 4 hr to form the appropriate TDZD-ibuprofen amide or ester linked conjugate (Structure V, Scheme 4).

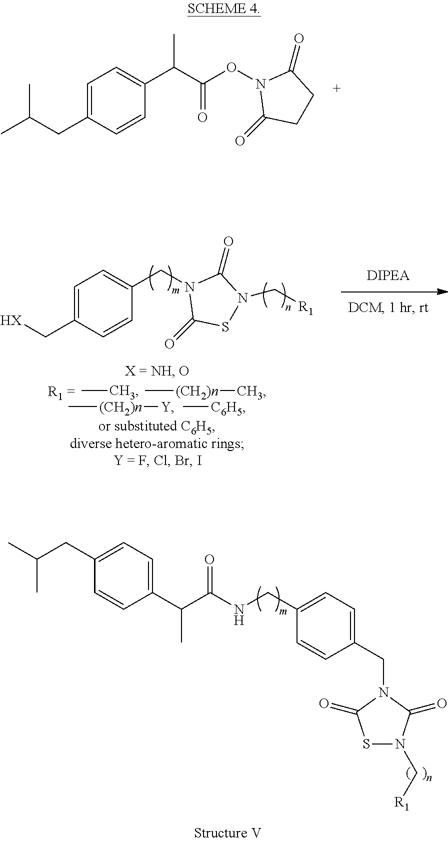

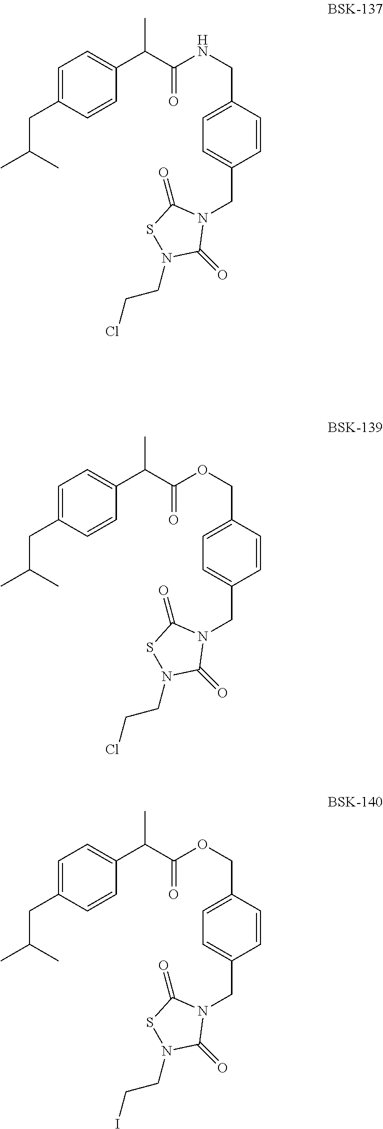 [SMILES: CC(C)Cc1ccc(C(C)C(=O)NCc2ccc(Cn3c(=O)sn(CCCl)c3=O)cc2)cc1 ; CC(C)Cc1ccc(C(C)C(=O)OCc2ccc(Cn3c(=O)sn(CCCl)c3=O)cc2)cc1 ; CC(C)Cc1ccc(C(C)C(=O)OCc2ccc(Cn3c(=O)sn(CCI)c3=O)cc2)cc1]

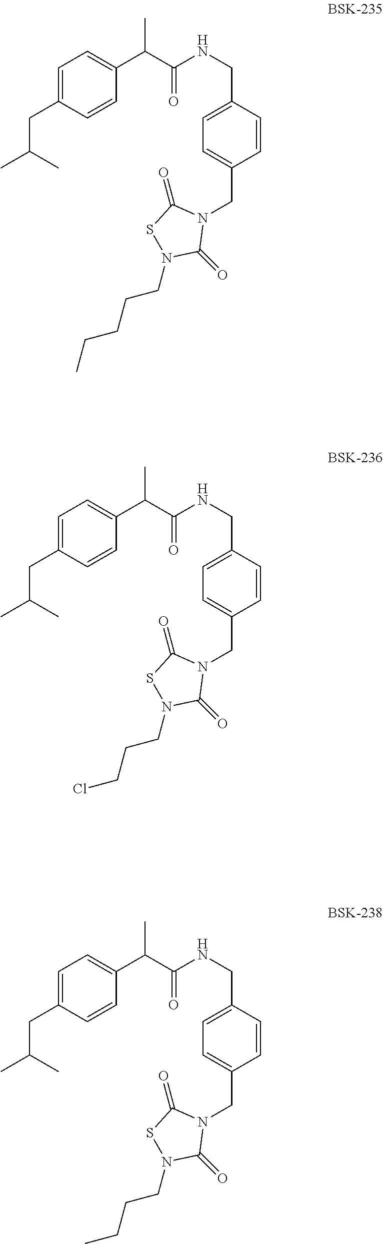 [SMILES: CCCCCn1sc(=O)n(Cc2ccc(CNC(=O)C(C)c3ccc(CC(C)C)cc3)cc2)c1=O ; CC(C)Cc1ccc(C(C)C(=O)NCc2ccc(Cn3c(=O)sn(CCCCl)c3=O)cc2)cc1 ; CCCCn1sc(=O)n(Cc2ccc(CNC(=O)C(C)c3ccc(CC(C)C)cc3)cc2)c1=O]

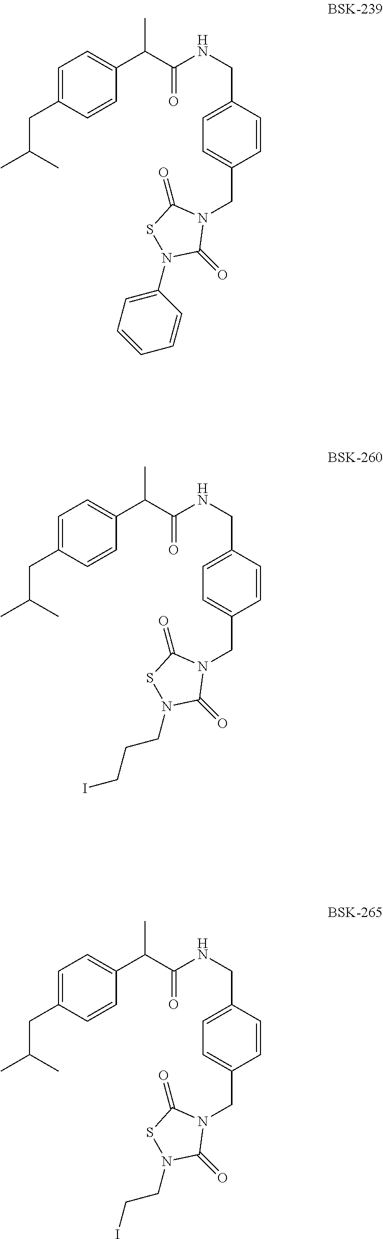 [SMILES: CC(C)Cc1ccc(C(C)C(=O)NCc2ccc(Cn3c(=O)sn(-c4ccccc4)c3=O)cc2)cc1 ; CC(C)Cc1ccc(C(C)C(=O)NCc2ccc(Cn3c(=O)sn(CCCI)c3=O)cc2)cc1 ; CC(C)Cc1ccc(C(C)C(=O)NCc2ccc(Cn3c(=O)sn(CCI)c3=O)cc2)cc1]

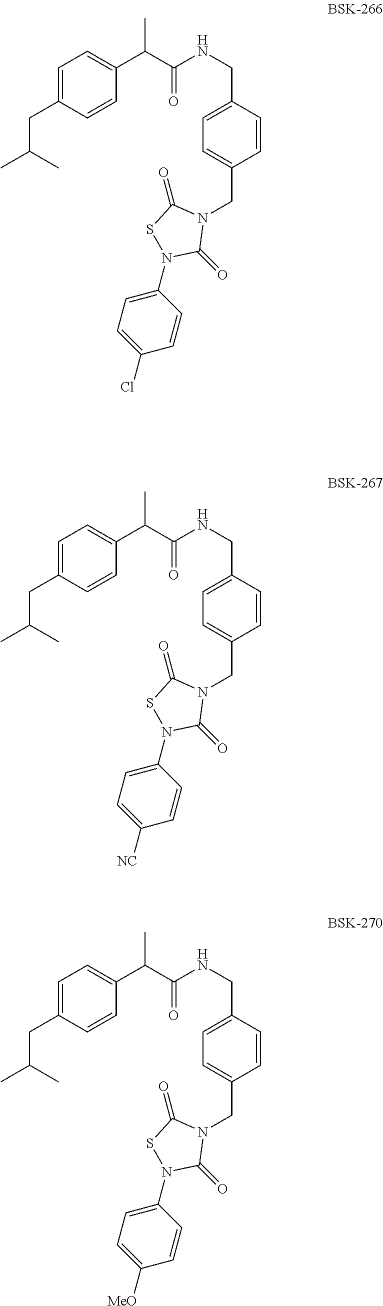 [SMILES: CC(C)Cc1ccc(C(C)C(=O)NCc2ccc(Cn3c(=O)sn(-c4ccc(Cl)cc4)c3=O)cc2)cc1 ; CC(C)Cc1ccc(C(C)C(=O)NCc2ccc(Cn3c(=O)sn(-c4ccc(C#N)cc4)c3=O)cc2)cc1 ; COc1ccc(-n2sc(=O)n(Cc3ccc(CNC(=O)C(C)c4ccc(CC(C)C)cc4)cc3)c2=O)cc1]

In another reaction, the triazole intermediate of MMB was reacted with various TDZD amines or alcohols (Structure I) in the presence of base to form carbamate or carbonate analogues of MMB-TDZD, respectively (Structure VI, Scheme 5).

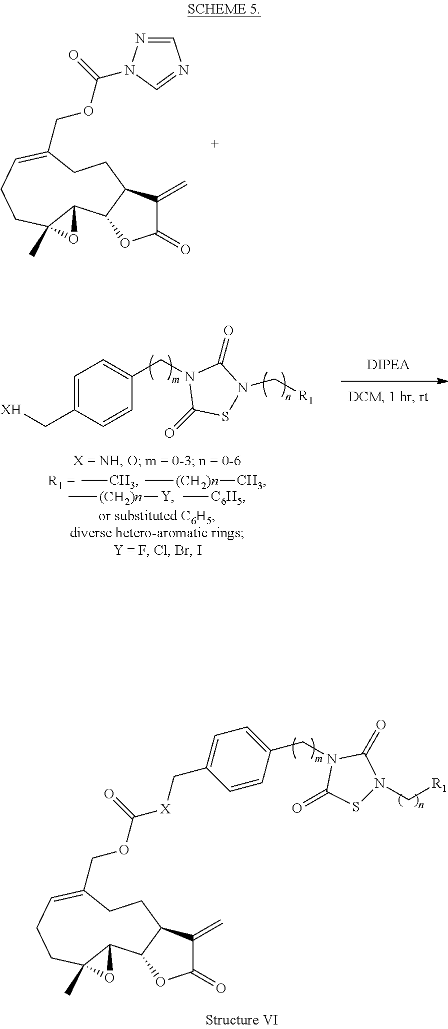

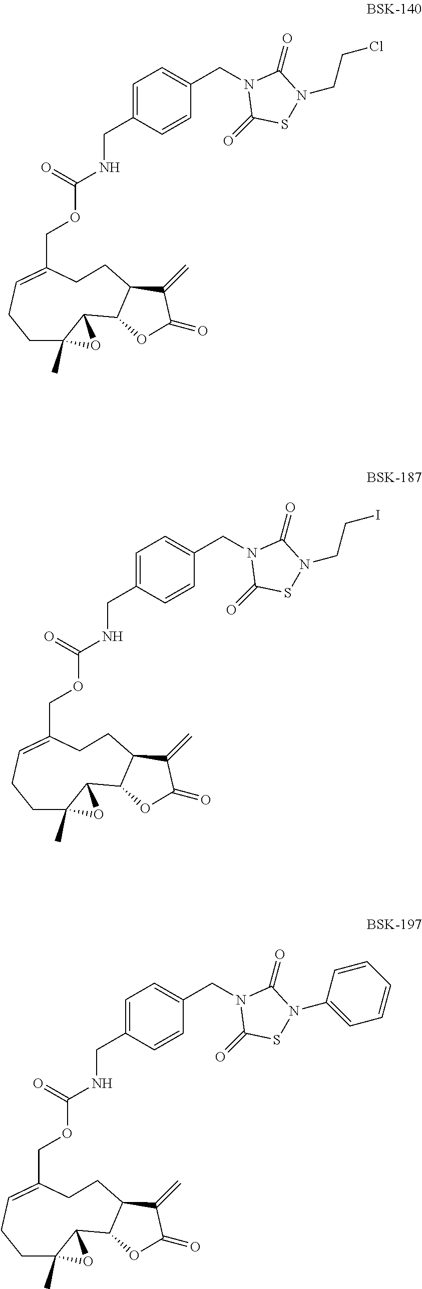 [SMILES: C=C1C(=O)O[C@H]2[C@H]1CC/C(COC(=O)NCc1ccc(Cn3c(=O)sn(CCCl)c3=O)cc1)=C\CC[C@@]1(C)O[C@@H]21 ; C=C1C(=O)O[C@H]2[C@H]1CC/C(COC(=O)NCc1ccc(Cn3c(=O)sn(CCI)c3=O)cc1)=C\CC[C@@]1(C)O[C@@H]21 ; C=C1C(=O)O[C@H]2[C@H]1CC/C(COC(=O)NCc1ccc(Cn3c(=O)sn(-c4ccccc4)c3=O)cc1)=C\CC[C@@]1(C)O[C@@H]21]

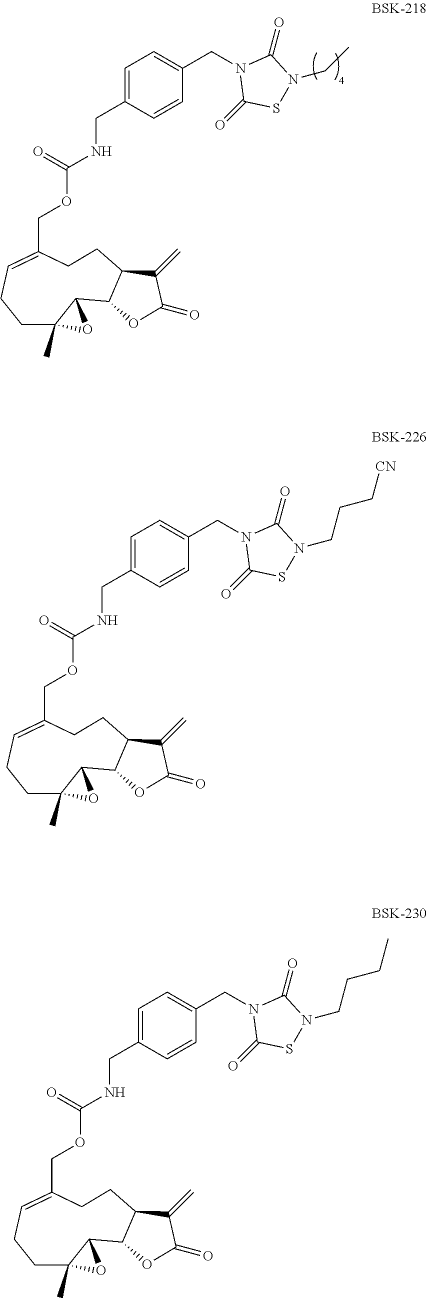

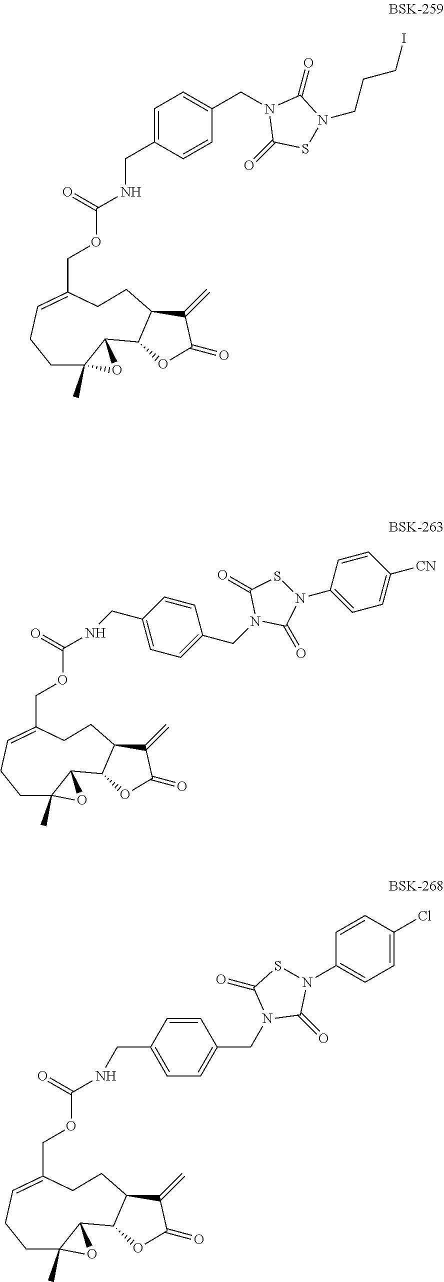 [SMILES: C=C1C(=O)O[C@H]2[C@H]1CC/C(COC(=O)NCc1ccc(Cn3c(=O)sn(CCCI)c3=O)cc1)=C\CC[C@@]1(C)O[C@@H]21 ; C=C1C(=O)O[C@H]2[C@H]1CC/C(COC(=O)NCc1ccc(Cn3c(=O)sn(-c4ccc(C#N)cc4)c3=O)cc1)=C\CC[C@@]1(C)O[C@@H]21 ; C=C1C(=O)O[C@H]2[C@H]1CC/C(COC(=O)NCc1ccc(Cn3c(=O)sn(-c4ccc(Cl)cc4)c3=O)cc1)=C\CC[C@@]1(C)O[C@@H]21]

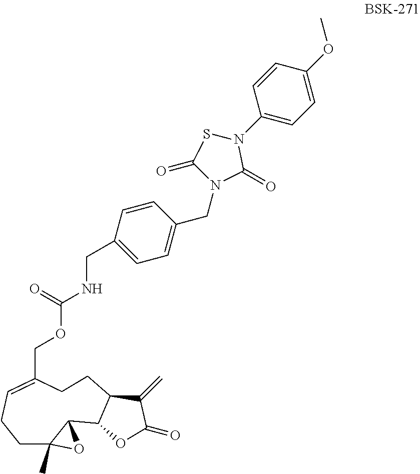 [SMILES: C=C1C(=O)O[C@H]2[C@H]1CC/C(COC(=O)NCc1ccc(Cn3c(=O)sn(-c4ccc(OC)cc4)c3=O)cc1)=C\CC[C@@]1(C)O[C@@H]21]

In another iteration, MMB was reacted with maleic anhydride in the presence of triethylamine in dichloromethane (DCM) as a solvent at ambient temperature for 48 h to form MMB-carboxylic acid. The obtained MMB-carboxylic acid intermediate was further reacted with ethyl chloroformate in the presence of triethylamine in dry tetrahydrofuran (THF), followed by reaction with TDZD amine to obtain MMB-TDZD amide and ester conjugated derivatives (Structure VII, Scheme 6).

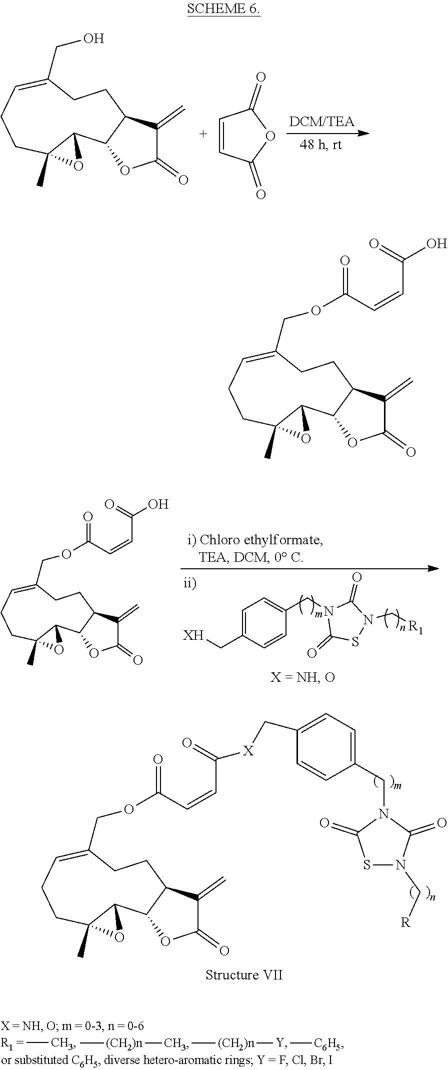

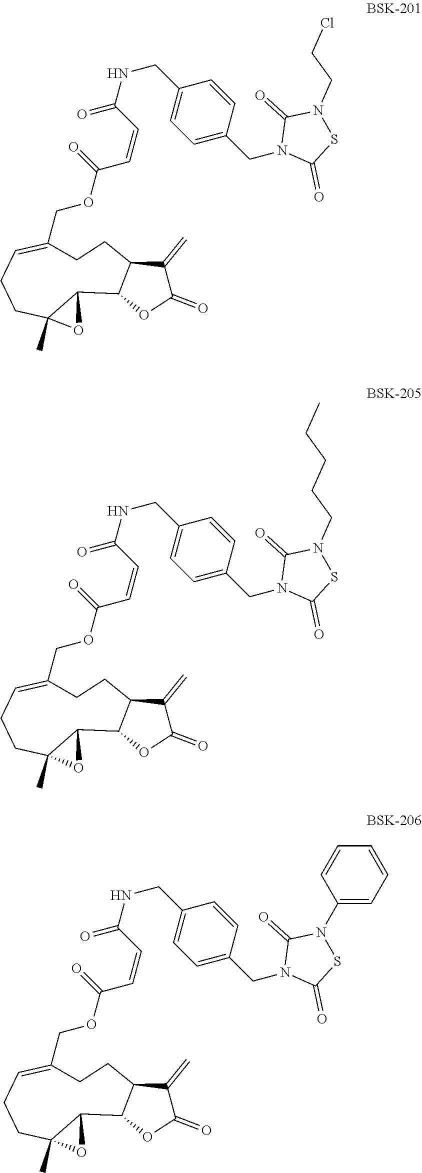 [SMILES: C=C1C(=O)O[C@H]2[C@H]1CC/C(COC(=O)/C=C\C(=O)NCc1ccc(Cn3c(=O)sn(CCCl)c3=O)cc1)=C\CC[C@@]1(C)O[C@@H]21 ; C=C1C(=O)O[C@H]2[C@H]1CC/C(COC(=O)/C=C\C(=O)NCc1ccc(Cn3c(=O)sn(CCCCC)c3=O)cc1)=C\CC[C@@]1(C)O[C@@H]21 ; C=C1C(=O)O[C@H]2[C@H]1CC/C(COC(=O)/C=C\C(=O)NCc1ccc(Cn3c(=O)sn(-c4ccccc4)c3=O)cc1)=C\CC[C@@]1(C)O[C@@H]21]

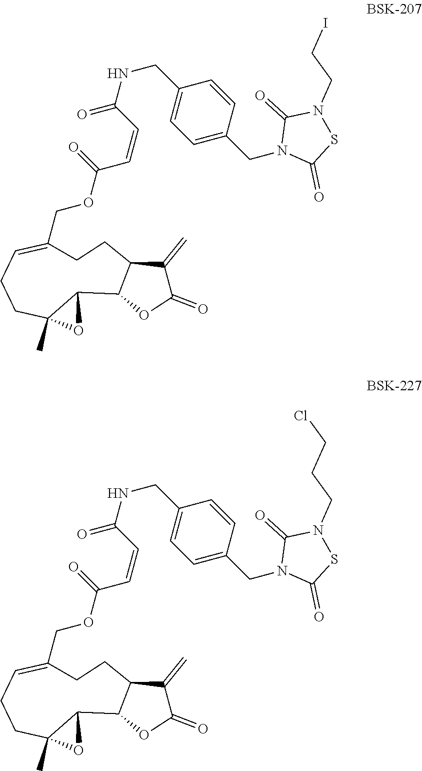 [SMILES: C=C1C(=O)O[C@H]2[C@H]1CC/C(COC(=O)/C=C\C(=O)NCc1ccc(Cn3c(=O)sn(CCI)c3=O)cc1)=C\CC[C@@]1(C)O[C@@H]21 ; C=C1C(=O)O[C@H]2[C@H]1CC/C(COC(=O)/C=C\C(=O)NCc1ccc(Cn3c(=O)sn(CCCCl)c3=O)cc1)=C\CC[C@@]1(C)O[C@@H]21]

In another disclosure, MMB was reacted with fumaric acid in the presence of N-(3-dimethylaminopropyl)-N′-ethylcarbodiimide hydrochloride (EDC), 1-hydroxybenzotriazole (HOBt), triethylamine in dry dimethylformamide at 0° C. to RT over 12 h to afford MMB-fumarate. The MMB-fumarate intermediate was further reacted with ethyl chloroformate in the presence of triethylamine in dry THF, followed by reaction with amino TDZD or hydroxyl TDZD to afford MMB-TDZD fumaric acid amide or ester conjugates (Structure VIII).

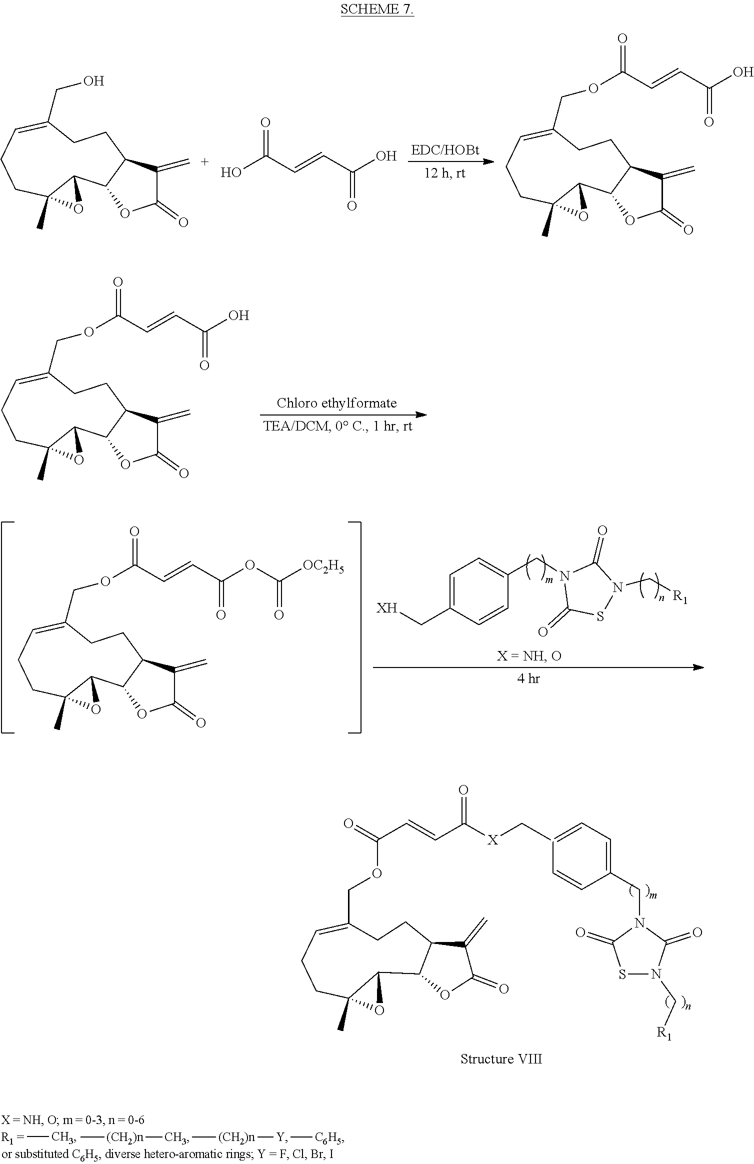

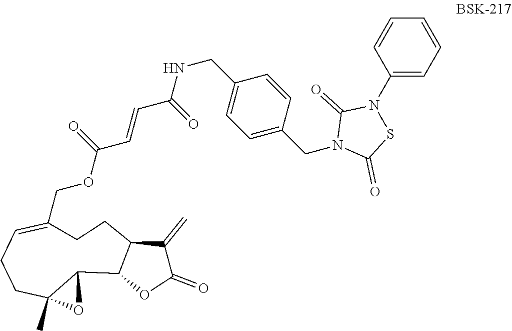 [SMILES: C=C1C(=O)O[C@H]2[C@H]1CC/C(COC(=O)/C=C/C(=O)NCc1ccc(Cn3c(=O)sn(-c4ccccc4)c3=O)cc1)=C\CC[C@@]1(C)O[C@@H]21]

In the next step, synthesis of hydroxyquinoline-TDZD carbamate (Structure-IX) analogs were carried out by the reaction of simple (8-((tert-butoxycarbonyl)oxy)-quinolin-2-yl)methyl 1H-1,2,4-triazole-1-carboxylate or (8-((tert-butoxycarbonyl)oxy)-5,7-dihaloquinolin-2-yl)methyl 1H-1,2,4-triazole-1-carboxylate, amino-TDZD or hydroxy-TDZD and triethylamine in dichloromethane. The resulting reaction mixture was stirred for 1h at rt to obtain the Boc-hydroxyquinoline-TDZD carbamate derivatives, which were then dissolved in the mixture of dichloromethane (DCM) and trifluoroacetic acid (TFA), stirred for 6h to obtain the hydroxyquinoline-TDZD carbamate derivatives. (Scheme 8).

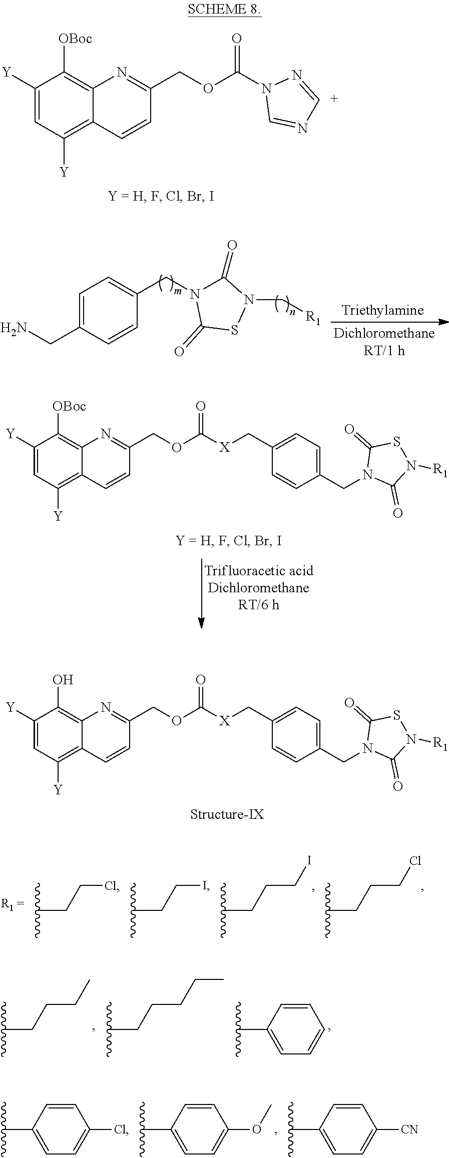

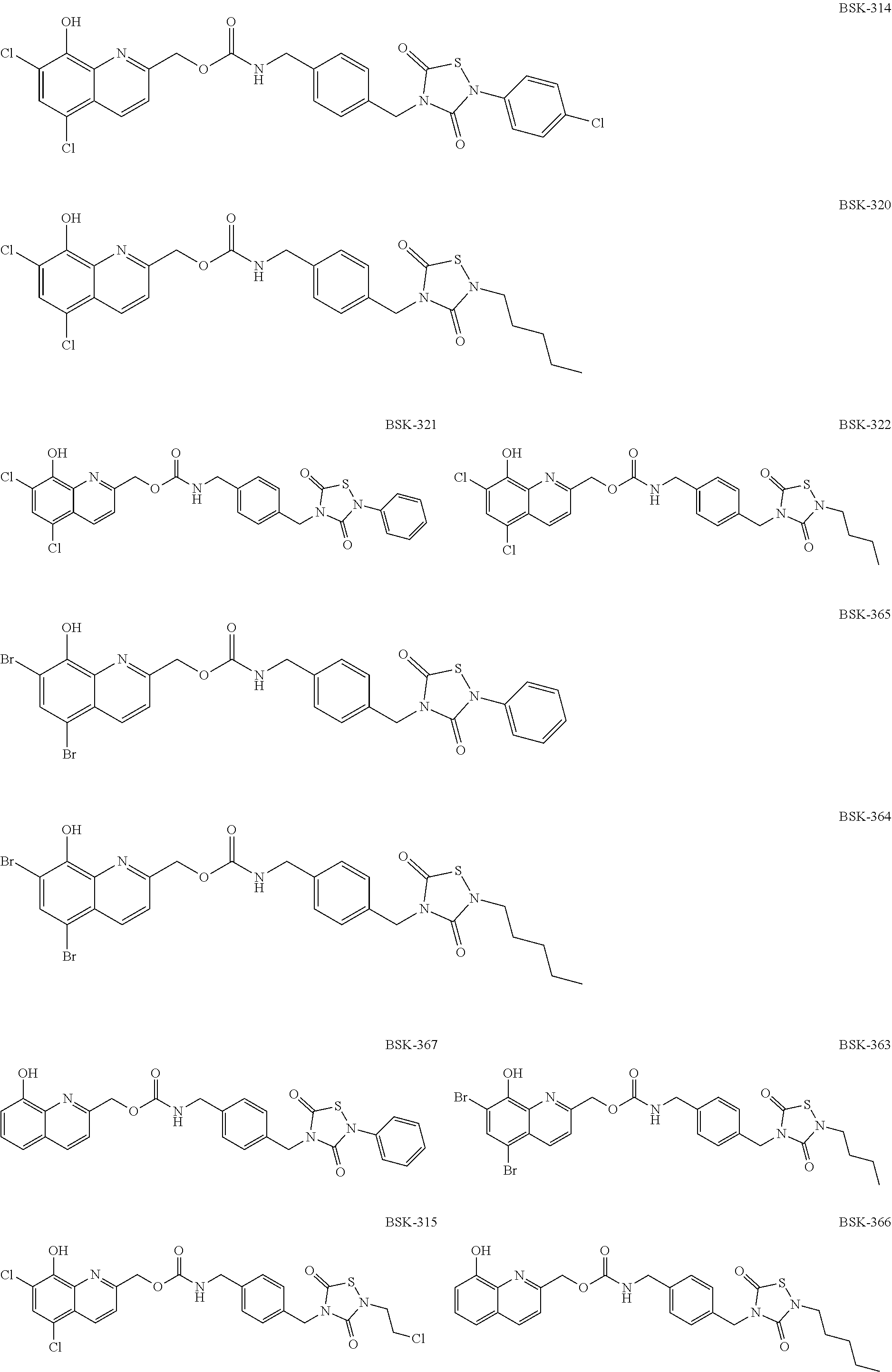

Without wishing to be bound by theory, in a general procedure for the synthesis of novel TDZD analogues, the use of various solvents such as, for example, chloroform, dichloromethane (DCM), diethyl ether, acetonitrile, tetrahydrofuran (THF), 1,4-dioxane, and mixtures thereof, is envisioned.

The temperature for the formation of TDZD compounds can range from about 0° C. to reflux temperatures, but the optimal temperature range is 0 to 5° C.

b. Synthesis and Analytical Data

### i. 2-(4-((2-(2-chloroethyl)-3,5-dioxo-1,2,4-thiadiazolidin-4-yl)methyl)benzylcarbamoyl)phenyl acetate

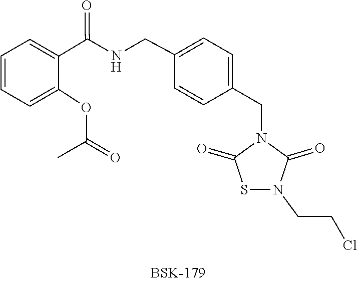 [SMILES: CC(=O)Oc1ccccc1C(=O)NCc1ccc(Cn2c(=O)sn(CCCl)c2=O)cc1]

To a solution of 2-acetoxybenzoic acid (0.2 g, 1.1 mmol) in dichloromethane (10 ml), was added oxalyl chloride (0.17 g, 1.3 mmol) followed by two drops of dimethylformamide. After gas evolution ceased, the mixture was concentrated and used immediately for the next step.

To a solution of 4-(4-(aminomethyl)benzyl)-2-(2-chloroethyl)-1,2,4-thiadiazolidine-3,5-dione (0.3 g, 1 mmol) and N, N-diisopropylethylamine (0.18 g, 1.3 mmol) in dichloromethane (10 ml), was added crude 2-acetoxybenzoic acid chloride in dichloromethane (10 mL) dropwise. The resulting reaction mixture was stirred 1 hr. and then diluted with water and extracted with dichloromethane. The combined organic phases were dried over magnesium sulfate, filtered, and then concentrated. Flash chromatography (silicagel, 2% methanolic dichloromethane) was used to purify the crude reaction mixture to yield 2-(4-((2-(2-chloroethyl)-3,5-dioxo-1,2,4-thiadiazolidin-4-yl)methyl)benzylcarbamoyl)phenyl acetate, a white solid. Yield: 76%. ¹H NMR (MeOH-d₄, 400 MHz): δ 7.57 (d, J=7.2 Hz, 1H), 7.45 (t, J=8.0 Hz, 1H), 7.30-7.33 (m, 5H), 7.11 (d, J=8.0 Hz, 1H), 4.79 (s, 2H), 4.46 (s, 2H), 3.94 (1, J=5.6 Hz, 2H), 3.74 (1, J=6.0 Hz, 2H), 2.01 (s, 3H); ppm; ¹³C NMR (MeOH-d₄, 100 MHz): 169.37, 166.19, 153.49, 148.03, 138.67, 134.59, 131.27, 129.13, 128.50, 128.22, 128.14, 127.70, 127.65, 125.73, 122.85, 46.20, 44.98, 42.57, 41.53, 19.29 ppm.

### ii. 2-((4-((3,5-dioxo-2-phenyl-1,2,4-thiadiazolidin-4-yl)methyl)benzyl)carbamoyl)phenyl acetate

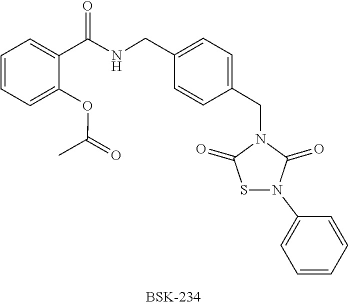 [SMILES: CC(=O)Oc1ccccc1C(=O)NCc1ccc(Cn2c(=O)sn(-c3ccccc3)c2=O)cc1]

To a solution of 2-acetoxybenzoic acid (0.2 g, 1.1 mmol) in dichloromethane (10 ml), was added oxalyl chloride (0.17 g, 1.3 mmol) followed by two drops of dimethylformamide. After gas evolution ceased, the mixture was concentrated and used immediately for the next step.

To a solution of 4-(4-(aminomethyl)benzyl)-2-phenyl-1,2,4-thiadiazolidine-3,5-dione (0.31 g, 1 mmol) and N, N-diisopropylethylamine (0.18 g, 1.3 mmol) in dichloromethane (10 ml), was added crude 2-acetoxybenzoic acid chloride in dichloromethane (10 mL) dropwise. The resulting reaction mixture was stirred 1 hr. and then diluted with water and extracted with dichloromethane. The combined organic phases were dried over magnesium sulfate, filtered, and then concentrated. Flash chromatography (silicagel, 2% methanolic dichloromethane) was used to purify the crude reaction mixture to yield 2-((4-((3,5-dioxo-2-phenyl-1,2,4-thiadiazolidin-4-yl)methyl)benzyl)carbamoyl)phenyl acetate, a white solid. Yield: 75%. ¹H NMR (CDCl₃, 400 MHz): δ 7.73 (d, J=8.0 Hz, 1H), 7.36-7.47 (m, 7H), 7.23-7.31 (m, 4H), 7.06 (d, J=8.4 Hz, 1H), 6.57 (brs, 1H), 4.86 (s, 2H), 4.53 (d, J=6.0 Hz, 2H), 2.06 (s, 3H) ppm; ¹³C NMR (CDCl₃, 100 MHz): 169.1, 165.45, 164.96, 150.87, 147.87, 138.37, 135.67, 134.48, 131.83, 139.73, 129.51, 128.32, 128.19, 127.04, 126.3, 123.37123.18, 45.73, 43.57, 20.77

### iii. 2-((4-((2-butyl-3,5-dioxo-1,2,4-thiadiazolidin-4-yl)methyl)benzyl)carbamoyl)phenyl acetate

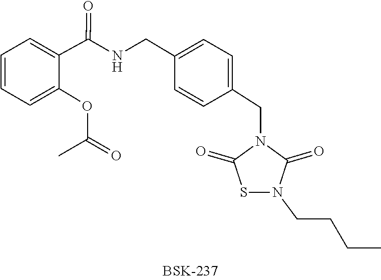 [SMILES: CCCCn1sc(=O)n(Cc2ccc(CNC(=O)c3ccccc3OC(C)=O)cc2)c1=O]

To a solution of 2-acetoxybenzoic acid (0.2 g, 1.1 mmol) in dichloromethane (10 ml), was added oxalyl chloride (0.17 g, 1.3 mmol) followed by two drops of dimethylformamide. After gas evolution ceased, the mixture was concentrated and used immediately for the next step.

To a solution of 4-(4-(aminomethyl)benzyl)-2-butyl-1,2,4-thiadiazolidine-3,5-dione (0.29 g, 1 mmol) and N, N-diisopropylethylamine (0.18 g, 1.3 mmol) in dichloromethane (10 ml), was added crude 2-acetoxybenzoic acid chloride in dichloromethane (10 mL) dropwise. The resulting reaction mixture was stirred 1 hr. and then diluted with water and extracted with dichloromethane. The combined organic phases were dried over magnesium sulfate, filtered, and then concentrated. Flash chromatography (silicagel, 2% methanolic dichloromethane) was used to purify the crude reaction mixture to yield 2-((4-((2-butyl-3,5-dioxo-1,2,4-thiadiazolidin-4-yl)methyl)benzyl)carbamoyl)phenyl acetate, a white solid. Yield: 76%: ¹H NMR (CDCl₃, 400 MHz): δ 7.73 (d, J=7.6 Hz, 1H), 7.38-7.45 (m, 3H), 7.23-7.29 (m, 3H), 7.06 (d, J=8.4 Hz, 1H), 6.51 (brs, 1H), 4.78 (s, 2H), 4.54 (d, J=5.6 Hz, 2H), 3.61 (1, J=7.2 Hz, 2H), 2.05 (s, 3H), 1.54-1.61 (m, 2H), 1.30-1.36 (m, 2H), 0.9 (1, J=7.2 Hz, 3H)ppm; ¹³C NMR (CDCl₃, 100 MHz): 169.1, 165.93, 165.40, 152.81, 147.83, 138.12, 134.79, 131.84, 129.77, 129.28, 128.29, 128.17, 126.33, 123.17, 45.5, 44.68, 43.61, 30.63, 29.66, 20.74, 19.46, 13.48

### iv. N-(4-((2-(2-chloroethyl)-3,5-dioxo-1,2,4-thiadiazolidin-4-yl)methyl)benzyl)-2-(4-isobutylphen-yl)propanamide

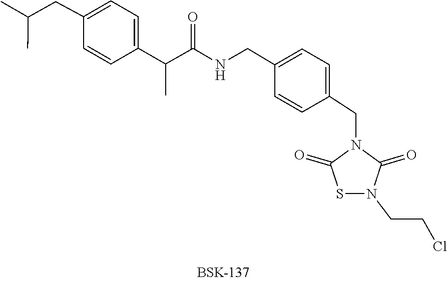 [SMILES: CC(C)Cc1ccc(C(C)C(=O)NCc2ccc(Cn3c(=O)sn(CCCl)c3=O)cc2)cc1]

A solution of 2-(4-isobutylphenyl)propanyl-NHS (50 mg, 0.16 mmol) in dichloromethane (3 mL) was added dropwise to a solution of 4-(4-(aminomethyl)benzyl)-2-(2-chloroethyl)-1,2,4-thiadiazolidine-3,5-dione (48 mg, 0.16 mmol) and N, N-diisopropylethylamine (41 mg, 0.32 mmol) in dichloromethane (2 mL). The resulting solution was stirred at room temperature for 1 hr. after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (CDCl₃, 400 MHz): δ 7.33 (d, J=8.0 Hz, 2H), 7.18 (d, J=8.4 Hz, 2H), 7.11 (t, J=6.8 Hz, 4H), 5.57 (brs, 1H), 4.77 (s, 2H), 4.36 (d, J=6.0 Hz, 2H), 3.93 (t, J=5.6 Hz, 2H), 3.70 (t, J=5.6 Hz, 2H), 3.56 (q, J=6.8 Hz, 1H), 2.44 (d, J=7.4 Hz, 2H), 1.85 (m, 1H), 1.53 (d, J=6.4 Hz, 3H), 0.89 (d, J=6.8 Hz, 6H) ppm; ¹³C NMR (CDCl₃, 100 MHz): δ 174.35, 165.75, 153.10, 140.82, 138.66, 138. 36, 134.01, 129.68, 129.14, 127.66, 127.33, 46.75, 45.66, 44.96, 43.11, 41.87, 30.14, 22.35, 18.40 ppm.

### v. N-(4-((2-(2-iodoethyl)-3,5-dioxo-1,2,4-thiadiazolidin-4-yl)methyl)benzyl)-2-(4-isobutylphen-yl)propanamide

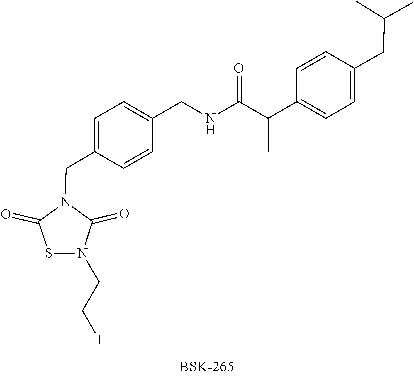 [SMILES: CC(C)Cc1ccc(C(C)C(=O)NCc2ccc(Cn3c(=O)sn(CCI)c3=O)cc2)cc1]

A solution of 2-(4-isobutylphenyl)propanyl-NHS (49 mg, 0.16 mmol) in dichloromethane (3 mL) was added dropwise to a solution of 4-(4-(aminomethyl)benzyl)-2-(2-iodoethyl)-1,2,4-thiadiazolidine-3,5-dione (64.5 mg, 0.16 mmol) and N, N-diisopropylethylamine (41 mg, 0.32 mmol) in dichloromethane (2 mL). The resulting solution was stirred at room temperature for 1 hr. after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (CDCl₃, 400 MHz): δ 7.33 (d, J=8.0 Hz, 2H), 7.18 (d, J=8.0 Hz, 2H), 7.1 (t, J=7.6 Hz, 4H), 5.59 (m, 1H), 4.76 (s, 2H), 4.35 (d, J=4.4 Hz, 2H), 3.96 (t, J=6.6 Hz, 2H), 3.57 (q, J=7.2 Hz, 1H), 3.29 (t, J=6.8 Hz, 2H), 2.44 (d, J=7.2 Hz, 2H), 1.79-1.86 (m, 1H), 1.52 (d, J=7.2 Hz, 3H), 0.88 (d, J=6.8 Hz, 6H) ppm; ¹³C NMR (CDCl₃, 100 MHz): δ 174.34, 165.29, 152.77, 140.85, 138.64, 138.33, 133.97, 129.7, 129.17, 127.83, 127.66, 127.34, 47.14, 46.76, 45.72, 44.97, 43.11, 30.15, 22.36, 18.40, −0.019 ppm.

### vi. N-(4-((2-(4-cyanophenyl)-3,5-dioxo-1,2,4-thiadiazolidin-4-yl)methyl)benzyl)-2-(4-isobutyl-phenyl)propanamide

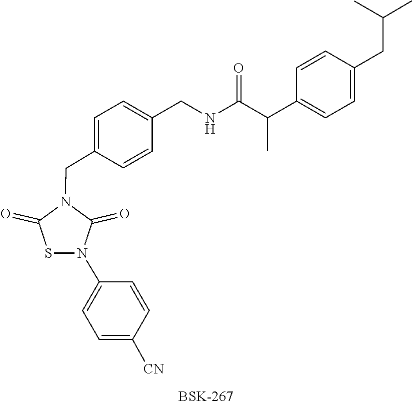 [SMILES: CC(C)Cc1ccc(C(C)C(=O)NCc2ccc(Cn3c(=O)sn(-c4ccc(C#N)cc4)c3=O)cc2)cc1]

A solution of 2-(4-isobutylphenyl)propanyl-NHS (49 mg, 0.16 mmol) in dichloromethane (3 mL) was added dropwise to a solution of 4-(4-(aminomethyl)benzyl)-2-(4-cyanophenyl)-1,2,4-thiadiazolidine-3,5-dione (54.5 mg, 0.16 mmol) and N, N-diisopropylethylamine (41 mg, 0.32 mmol) in dichloromethane (2 mL). The resulting solution was stirred at room temperature for 1 hr. after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (DMSO-d₆, 400 MHz): δ 8.37 (t, J=6.0 Hz, 1H), 7.63 (d, J=8.4 Hz, 2H), 7.54 (d, J=8.8 Hz, 2H), 7.19 (d, J=8.0 Hz, 2H), 7.14 (d, J=8.0 Hz, 2H), 7.04 (t, J=7.6 Hz, 4H), 4.21 (s, 2H), 4.18 (d, J=6.0 Hz, 2H), 3.59 (q, J=7.2 Hz, 1H), 2.36 (d, J=7.2 Hz, 2H), 1.71-1.77 (m, 1H), 1.29 (d, J=7.2 Hz, 3H), 0.8 (d, J=6.8 Hz, 6H) ppm; ¹³C NMR (DMSO-d₆, 100 MHz): δ 173.8, 155.02, 154.94, 145.35, 145.23, 139.96, 139.68, 138.74, 138.56, 133.61, 129.17, 127.41, 127.39, 119.87, 117.93, 117.84, 102.84, 45.19, 44.65, 42.86, 42.11, 30.07, 22.56, 18.86 ppm.

### vii. 2-(4-isobutylphenyl)-n-(4-((2-(4-methoxyphenyl)-3,5-dioxo-1,2,4-thiadiazolidin-4-yl)meth-yl)benzyl)propanamide

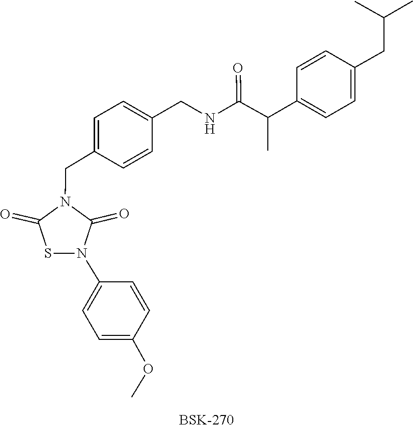 [SMILES: COc1ccc(-n2sc(=O)n(Cc3ccc(CNC(=O)C(C)c4ccc(CC(C)C)cc4)cc3)c2=O)cc1]

A solution of 2-(4-isobutylphenyl)propanyl-NHS (49 mg, 0.16 mmol) in dichloromethane (3 mL) was added dropwise to a solution of 4-(4-(aminomethyl)benzyl)-2-(4-methoxyphenyl)-1,2,4-thiadiazolidine-3,5-dione (55 mg, 0.16 mmol) and N, N-diisopropylethylamine (41 mg, 0.32 mmol) in dichloromethane (2 mL). The resulting solution was stirred at room temperature for 1 hr, after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (CDCl₃, 400 MHz): δ 7.38 (d, J=8.0 Hz, 2H), 7.34 (d, J=8.8 Hz, 2H), 7.18 (d, J=8.0 Hz, 2H), 7.10 (d, J=8.4 Hz, 4H), 6.9 (d, J=8.8 Hz, 2H), 5.66 (brs, 1H), 4.82 (s, 2H), 4.35 (d, J=5.6 Hz, 2H), 3.79 (s, 3H), 3.56 (q, J=6.8 Hz, 1H), 2.44 (d, J=7.6 Hz, 2H), 1.81-1.84 (m, 1H), 1.52 (d, J=7.6 Hz, 3H), 0.88 (d, J=6.8 Hz, 6H) ppm; ¹³C NMR (CDCl₃, 100 MHz): δ 174.38, 165.32, 158.8, 151.33, 140.82, 138.64, 138.36, 134.13, 129.68, 129.55, 129.39, 127.96, 127.66, 127.34, 126.18, 114.69, 55.55, 46.74, 45.76, 44.97, 43.12, 30.14, 22.34, 18.40 ppm.

### viii. ((1aR,7aS,10aS,10bS,E)-1a-methyl-8-methylene-9-oxo-1a,2,3,6,7,7a,8,9,10a,10b-decahydro-oxireno[2′,3′:9,10]cyclodeca[1,2-b]furan-5-yl)methyl(4-((3,5-dioxo-2-phenyl-1,2,4-thiadia-zolidin-4-yl)methyl)benzyl)carbamate

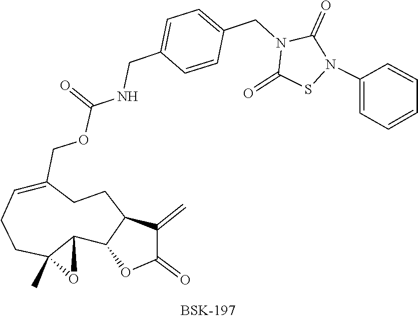 [SMILES: C=C1C(=O)O[C@H]2[C@H]1CC/C(COC(=O)NCc1ccc(Cn3c(=O)sn(-c4ccccc4)c3=O)cc1)=C\CC[C@@]1(C)O[C@@H]21]

To MMB triazole (50 mg, 0.14 mmol) in dichloromethane (2 mL), 4-(4-(aminomethyl)benzyl)-2-phenyl-1,2,4-thiadiazolidine-3,5-dione (44 mg, 0.14 mmol) and triethylamine (28 mg 0.28 mmol) was added at it Resulting reaction mixture was stirred for 1 hr at rt, after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (CDCl₃, 400 MHz): δ 7.47-7.37 (m, 6H), 7.25-7.22 (m, 3H), 6.17 (s, 1H), 5.64 (brs, 1H), 5.47 (s, 1H), 5.15 (bs, 1H), 4.85 (s, 2H), 4.61 (d, J=12.0 Hz, 1H), 4.47 (d, J=12.0 Hz, 1H), 4.31 (d, J=5.2 Hz, 2H), 3.81 (t, 9.6 Hz, 1H), 2.87-2.79 (m, 2H), 2.37-2.10 (m, 6H), 1.60 (t, J=10.4 Hz, 1H), 1.49 (s, 3H), 1.02 (t, 12.0 Hz, 1H); ¹³C NMR (CDCl₃, 100 MHz): δ 169.43, 164.99, 156.15, 150.89, 138.72, 135.66, 134.32, 130.16 (d), 129.50 (d), 127.86, 27.04, 123.38, 120.26, 81.04, 67.30, 63.22, 59.94, 45.73, 44.72, 42.58, 36.59, 25.74, 24.50, 23.75, 17.95, 15.24.

### ix. ((1aR,7aS,10aS,10bS,E)-1a-methyl-8-methylene-9-oxo-1a,2,3,6,7,7a,8,9,10a,10b-decahydro-oxireno[2′,3′:9,10]cyclodeca[1,2-b]furan-5-yl)methyl(4-((2-butyl-3,5-dioxo-1,2,4-thiadiazoli-din-4-yl)methyl)benzyl)carbamate

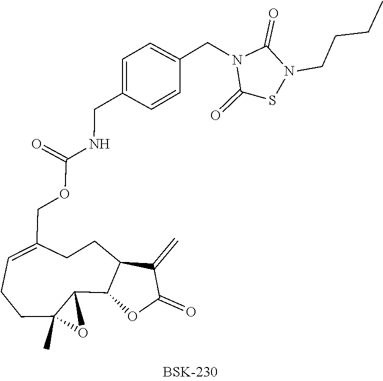 [SMILES: C=C1C(=O)O[C@H]2[C@H]1CC/C(COC(=O)NCc1ccc(Cn3c(=O)sn(CCCC)c3=O)cc1)=C\CC[C@@]1(C)O[C@@H]21]

To MMB triazole (50 mg, 0.14 mmol) in dichloromethane (2 mL), 4-(4-(aminomethyl)benzyl)-2-butyl-1,2,4-thiadiazolidine-3,5-dione (42 mg, 0.14 mmol) and triethylamine (28 mg 0.28 mmol) was added at it Resulting reaction mixture was stirred for 1 hr at rt, after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (CDCl₃, 400 MHz): δ 7.36 (d, J=8.0 Hz, 2H), 7.20 (d, J=8.0 Hz, 2H), 6.15 (s, 1H), 5.63 (t, J=8.0 Hz, 1H), 5.46 (s, 1H), 5.17 (br-s, 1H), 4.74 (s, 2H), 4.60 (d, J=12.0 Hz, 1H), 4.46 (d, J=12.4 Hz, 1H), 4.29 (d, J=5.6 Hz, 2H), 3.81 (t, J=9.6 Hz, 1H), 3.59 (t, J=7.2 Hz, 2H), 2.86-2.79 (m, 2H), 2.36-2.07 (m, 6H), 1.66-1.52 (m, 3H), 1.49 (s, 3H), 1.36-1.26 (m, 2H). 1.07 (t, J=12.4 Hz, 1H), 0.91 (t, J=7.6 Hz, 3H). ¹³C NMR (CDCl₃, 100 MHz): δ 169.42, 165.97, 156.15, 152.82, 138.72 (d), 135.40, 134.56, 130.13, 129.15, 127.78, 120.24, 81.04, 67.28, 63.21, 59.94, 45.49, 44.66, 42.57, 36.59, 30.61, 25.73, 24.50, 23.75, 19.54, 17.95, 13.49.

### x. ((1aR,7aS,10aS,10bS,E)-1a-methyl-8-methylene-9-oxo-1a,2,3,6,7,7a,8,9,10a,10b-decahydro-oxtreno[2′,3′:9,10]cyclodeca[1,2-b]furan-5-yl)methyl (4-((2-(3-iodopropyl)-3,5-dioxo-1,2,4-thiadiazolidin-4-yl)methyl)benzyl)carbamate

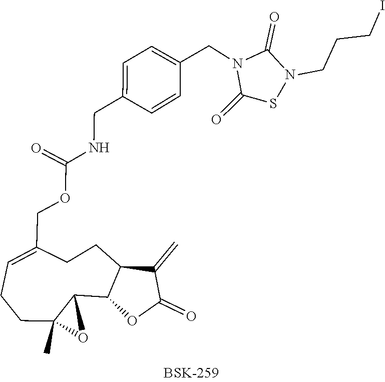 [SMILES: C=C1C(=O)O[C@H]2[C@H]1CC/C(COC(=O)NCc1ccc(Cn3c(=O)sn(CCCI)c3=O)cc1)=C\CC[C@@]1(C)O[C@@H]21]

To MMB triazole (50 mg, 0.14 mmol) in dichloromethane (2 mL), 4-(4-(aminomethyl)benzyl)-2-(3-iodopropyl)-1,2,4-thiadiazolidine-3,5-dione (57 mg, 0.14 mmol) and triethylamine (28 mg 0.28 mmol) was added at it Resulting reaction mixture was stirred for 1 hr at rt, after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (CDCl₃, 400 MHz): δ 7.36 (d, 2H, J=8.0 Hz), 7.22 (d, J=8.0 Hz, 2H), 6.17 (s, 1H), 5.66 (t, J=8.0 Hz, 1H), 5.48 (s, 1H), 5.12 (bs, 1H), 4.76 (s, 2H), 4.62 (d, J=12.4 Hz, 1H), 4.48 (d, J=12.4 Hz, 1H), 4.31 (d, J=6.0 Hz, 2H), 3.82 (t, J 9.6 Hz, 1H), 3.70 (t, J=6.8 Hz, 2H), 3.16 (t, J 6.8 Hz, 2H), 2.87-2.80 (m, 2H), 2.51-2.08 (m, 7H), 1.74 (s, 1H), 1.64 (t, J=10.0 Hz, 1H), 1.50 (s, 3H), 1.09 (t, J=12 Hz, 1H). 13C NMR (CDCl₃, 100 MHz): δ 169.41, 165.65, 156.14, 152.96, 138.73 (d), 135.38, 34.41, 130.18, 129.28, 127.82, 120.26, 81.04, 67.31, 63.22, 59.95, 45.66, 45.62, 44.71, 42.58, 36.60, 32.06, 25.76, 24.53, 23.77, 17.98, 0.998 ppm.

### xi. ((1aR,7aS,10aS,10bS,E)-1a-methyl-8-methylene-9-oxo-1a,2,3,6,7,7a,8,9,10a,10b-decahydro-oxireno [2′,3′:9,10]cyclodeca[1,2-b]furan-5-yl)methyl(4-((2-(4-chlorophenyl)-3,5-dioxo-1,2,4-thiadiazolidin-4-yl)methyl)benzyl)carbamate

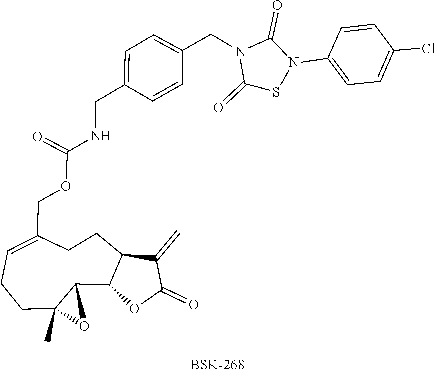 [SMILES: C=C1C(=O)O[C@H]2[C@H]1CC/C(COC(=O)NCc1ccc(Cn3c(=O)sn(-c4ccc(Cl)cc4)c3=O)cc1)=C\CC[C@@]1(C)O[C@@H]21]

To MMB triazole (50 mg, 0.14 mmol) in dichloromethane (2 mL), 4-(4-(aminomethyl)benzyl)-2-(4-chlorophenyl)-1,2,4-thiadiazolidine-3,5-dione (48.5 mg, 0.14 mmol) and triethylamine (28 mg 0.28 mmol) was added at it Resulting reaction mixture was stirred for 1 hr at rt, after completion of reaction (monitored by TLC); water was added and the aqueous mixture was extracted with dichloromethane. The organic layer was washed with brine solution (30% NaCl aq.solution), separated organic layer was dried over anhydrous Na₂SO₄ and concentrated to yield a crude product, which was purified by column chromatography (silica gel, 3% methanol in dichloromethane) to yield the compound as a white solid. ¹H NMR (CDCl₃, 400 MHz): δ 7.43 (m, 4H), 7.36 (d, J=8.0 Hz, 2H), 7.51 (d, J=8.0 Hz, 2H), 6.17 (s, 1H), 5.67 (t, J=7.6 Hz, 1H), 5.48 (s, 1H), 5.08 (bs, 1H), 4.85 (s, 2H), 4.63 (d, J=12.4 Hz, 1H), 4.49 (d, J=12.4 Hz, 1H), 4.31 (d, J=5.6 Hz, 2H), 3.82 (t, J=9.6 Hz, 1H), 2.90-2.80 (m, 2H), 2.42-2.09 (m, 6H), 1.64 (t, J=10.4 Hz, 1H), 1.50 (s, 3H), 1.09 (t, J=12.0 Hz, 1H). ¹³C NMR (CDCl₃, 100 MHz): δ 169.38, 164.54, 156.11, 150.82, 138.74, 135.37, 134.25, 134.18, 132.51, 130.22, 129.58, 129.49, 127.88, 124.51. 120.23, 81.03, 67.33, 63.23, 59.91, 45.85, 44.73, 42.59, 36.60, 25.77, 24.53, 23.77, 17.96.

### xii. ((1aR,7aS,10aS,10bS,E)-1a-methyl-8-methylene-9-oxo-1a,2,3,6,7,7a,8,9,10a,10b-decahydro-oxireno[2′,3′:9,10]cyclodeca[1,2-b]furan-5-yl)methyl (z)-4-((4-((2-(2-chloroethyl)-3,5-dioxo-1,2,4-thiadiazolidin-4-yl)methyl)benzyl)amino)-4-oxobut-2-enoate

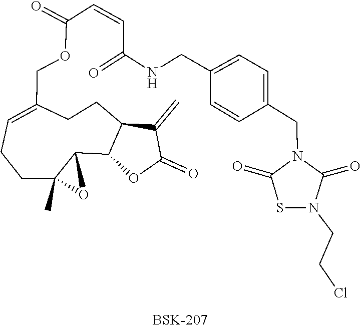 [SMILES: C=C1C(=O)O[C@H]2[C@H]1CC/C(COC(=O)/C=C\C(=O)NCc1ccc(Cn3c(=O)sn(CCCl)c3=O)cc1)=C\CC[C@@]1(C)O[C@@H]21]

To the reaction mixture of MMB-carboxylic acid, (65 mg, 0.18 mmol), ethyl chloroformate (20 mg, 0.18 mmol), and triethylamine (18 mg, 0.18 mmol) in dry THE (5 ml) was stirred at 0° C. for 1 hr., then 4-(4-(aminomethyl)benzyl)-2-(2-chloroethyl)-1,2,4-thiadiazolidine-3,5-dione (53 mg, 0.18 mmol) was added. The resulting reaction mixture was stirred at ambient temperature for 4 hr. after reaction was complete (as monitored by TLC); added water and extracted with dichloromethane. The organic layer was dried and concentrated to get crude compound, which was further purified by column chromatography (silica gel, 3% methanol in dichloromethane) to afford pure product as a white solid. ¹H NMR (CDCl₃, 400 MHz): δ 7.90 (br-s, 1H), 7.31-7.34 (m, 2H), 7.22-7.24 (m, 2H), 6.25 (d, J=12.8 Hz, 1H), 6.14 (s, 1H), 6.06 (d, J=12.8 Hz, 1H), 5.66 (t, J=8.4 Hz, 1H), 5.47 (s, 1H), 4.75 (s, 2H), 4.68 (d, J=12.0 Hz, 1H), 4.49 (d, J=12.0 Hz, 1H), 4.43 (br-s, 2H), 3.90 (t, J=6.0 Hz, 2H), 3.77 (t, 0.1=9.2 Hz, 1H), 3.64 (t, J=6.0 Hz, 2H), 2.84-2.78 (m, 2H), 2.40-2.09 (m, 6H), 1.63 (t, J=11.2 Hz, 1H), 1.49 (s, 3H), 1.08 (t, J=12.4 Hz, 1H). ¹³C NMR (CDCl₃, 100 MHz): δ 169.41, 165.97, 165.81, 163.79, 153.10, 138.60, 137.95, 136.53, 136.27, 134.33, 134.24, 131.81, 129.05, 129.01, 128.07, 126.21, 126.08, 120.42, 80.99, 67.99, 63.21, 59.98, 53.49, 46.69, 45.62, 43.24, 42.65, 41.94, 36.49, 25.56, 24.41, 23.83, 17.94 ppm.

### xiii. ((1aR,7aS,10aS,10bS,E)-1a-methyl-8-methylene-9-oxo-1a,2,3,6,7,7a,8,9,10a,10b-decahydro-oxireno[2′,3′:9,10]cyclodeca[1,2-b]furan-5-yl)methyl (4-((2-(2-iodoethyl)-3,5-dioxo-1,2,4-thiadiazolidin-4-yl)methyl)benzyl)carbamate

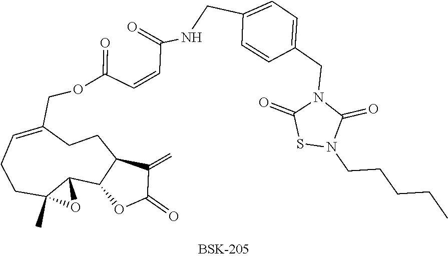 [SMILES: C=C1C(=O)O[C@H]2[C@H]1CC/C(COC(=O)/C=C\C(=O)NCc1ccc(Cn3c(=O)sn(CCCCC)c3=O)cc1)=C\CC[C@@]1(C)O[C@@H]21]

To the reaction mixture of MMB-carboxylic acid, (65 mg, 0.18 mmol), ethyl chloroformate (20 mg, 0.18 mmol), and triethylamine (18 mg, 0.18 mmol) in dry THE (5 ml) was stirred at 0° C. for 1 hr., then 4-(4-(aminomethyl)benzyl)-2-pentyl-1,2,4-thiadiazolidine-3,5-dione (56 mg, 0.18 mmol) was added. The resulting reaction mixture was stirred at ambient temperature for 4 hr. after reaction was complete (as monitored by TLC); added water and extracted with dichloromethane. The organic layer was dried and concentrated to get crude compound, which was further purified by column chromatography (silica gel, 3% methanol in dichloromethane) to afford pure product as a white solid. ¹H NMR (CDCl₃, 400 MHz): δ 7.88 (br-s, 1H), 7.31 (d, 2H, J=8.0 Hz), 7.20 (d, J=8.0 Hz, 2H), 6.25 (d, J=12.4 Hz, 1H), 6.05 (d, J=12.4 Hz, 1H), 6.13 (s, 1H), 5.65 (t, J=8.4 Hz, 1H), 5.47 (s, 1H), 4.72 (s, 2H), 4.68 (d, J=12.4 Hz, 1H), 4.48 (d, J=12.4 Hz, 1H), 4.42 (br-s, 2H), 3.77 (t, J=9.2 Hz, 1H), 3.56 (t, J 6.8 Hz, 2H), 2.83-2.78 (m, 2H), 2.40-2.09 (m, 6H), 1.62-1.52 (m, 3H), 1.48 (s, 3H), 1.31-1.20 (m, 4H), 1.01 (t, J=12.4 Hz, 1H), 0.82 (t, J 6.4 Hz, 3H). ¹³C NMR (CDCl₃, 100 MHz): δ 169.42, 165.99, 165.97, 163.8, 152.81, 138.6, 137.83, 136.27, 134.49, 134.36, 131.79, 129.02, 128.03, 126.19, 120.41, 80.98, 67.99, 63.21, 59.98, 45.47, 44.9, 43.23, 42.65, 36.49, 28.37, 28.28, 25.54, 24.41, 23.82, 22.07, 17.92, 13.86.

### xiv. ((1aR,7aS,10aS,10bS,E)-1a-methyl-8-methylene-9-oxo-1a,2,3,6,7,7a,8,9,10a,10b-decahydro-oxireno[2′,3′:9,10]cyclodeca[1,2-b]furan-5-yl)methyl (4-((2-(2-iodoethyl)-3,5-dioxo-1,2,4-thiadiazolidin-4-yl)methyl)benzyl)carbamate

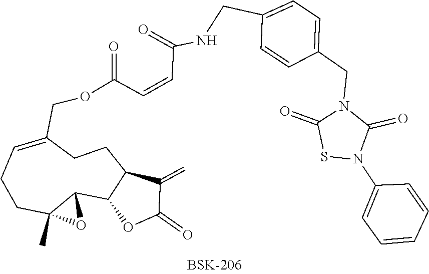 [SMILES: C=C1C(=O)O[C@H]2[C@H]1CC/C(COC(=O)/C=C\C(=O)NCc1ccc(Cn3c(=O)sn(-c4ccccc4)c3=O)cc1)=C\CC[C@@]1(C)O[C@@H]21]

To the reaction mixture of MMB-carboxylic acid, (65 mg, 0.18 mmol), ethyl chloroformate (20 mg, 0.18 mmol), and triethylamine (18 mg, 0.18 mmol) in dry THE (5 ml) was stirred at 0° C. for 1 hr., then 4-(4-(aminomethyl)benzyl)-2-phenyl-1,2,4-thiadiazolidine-3,5-dione (56.5 mg, 0.18 mmol) was added. The resulting reaction mixture was stirred at ambient temperature for 4 hr. after reaction was complete (as monitored by TLC); added water and extracted with dichloromethane. The organic layer was dried and concentrated to get crude compound, which was further purified by column chromatography (silica gel, 3% methanol in dichloromethane) to afford pure product as a white solid. ¹H NMR (CDCl₃, 400 MHz): δ 7.93 (br-s, 1H), 7.46-7.35 (m, 6H), δ 7.27-7.22 (m, 3H), 6.28 (d, J=12.8 Hz, 1H), 6.17 (s, 1H), 6.07 (d, J=12.8 Hz, 1H), 5.67 (t, J=8.0 Hz, 1H), 5.49 (s, 1H), 4.84 (s, 2H), 4.69 (d, J=12.4 Hz, 1H), 4.50 (d, J=12.4 Hz, 1H), 4.46 (br-s, 2H), 3.77 (t, J=9.2 Hz, 1H), 2.84-2.79 (m, 2H), 2.42-2.10 (m, 6H), 1.62 (t, J=12.0 Hz, 1H), 1.49 (s, 3H), 1.03 (t, J=12.0 Hz, 1H). ¹³C NMR (CDCl₃, 100 MHz): δ 169.38, 165.94, 164.99, 163.77, 152.77, 138.61, 138.01, 136.79, 135.65, 134.3, 134.26, 131.86, 129.51, 129.36, 128.13, 127.06, 125.96, 123.38, 120.41, 80.97, 68.0, 63.22, 59.95, 45.74, 43.3, 42.66, 36.5, 25.57, 24.42, 23.84, 17.93.

### xv. ((1aR,7aS,10aS,10bS,E)-1a-methyl-8-methylene-9-oxo-1a,2,3,6,7,7a,8,9,10a,10b-decahydro-oxireno[2′,3′:9,10]cyclodeca[1,2-b]furan-5-yl)methyl(4-((2-(2-iodoethyl)-3,5-dioxo-1,2,4-thiadiazolidin-4-yl)methyl)benzyl)carbamate

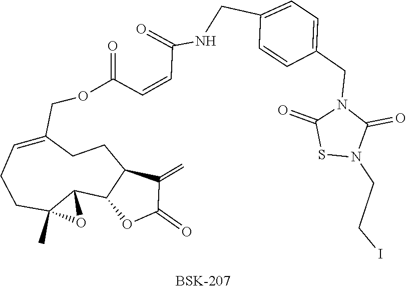 [SMILES: C=C1C(=O)O[C@H]2[C@H]1CC/C(COC(=O)/C=C\C(=O)NCc1ccc(Cn3c(=O)sn(CCI)c3=O)cc1)=C\CC[C@@]1(C)O[C@@H]21]

To the reaction mixture of MMB-carboxylic acid, (65 mg, 0.18 mmol), ethyl chloroformate (20 mg, 0.18 mmol), and triethylamine (18 mg, 0.18 mmol) in dry THE (5 ml) was stirred at 0° C. for 1 hr., then 4-(4-(aminomethyl)benzyl)-2-(2-iodoethyl)-1,2,4-thiadiazolidine-3,5-dione (70.5 mg, 0.18 mmol) was added. The resulting reaction mixture was stirred at ambient temperature for 4 hr. after reaction was complete (as monitored by TLC); added water and extracted with dichloromethane. The organic layer was dried and concentrated to get crude compound, which was further purified by column chromatography (silica gel, 3% methanol in dichloromethane) to afford pure product as a white solid. ¹H NMR (CDCl₃, 400 MHz): δ 7.93 (br-s, 1H), 7.36 (d, 2H, J=8.0 Hz), 7.24 (d, J=8.0 Hz, 2H), 6.31 (d, J=12.4 Hz, 1H), 6.7 (s, 1H), 6.08 (d, J=12.4 Hz, 1H), 5.67 (t, J=8.0 Hz, 1H), 5.48 (s, 1H), 4.77 (s, 2H), 4.69 (d, J=12.4 Hz, 1H), 4.50 (d, J=12.4 Hz, 1H), 4.45 (br-s, 2H), 3.91 (t, J 6.8 Hz, 2H), 3.79 (t, J 9.6 Hz, 1H), 3.25 (t, J 6.4 Hz, 2H), 2.82-2.80 (m, 2H), 2.45-2.10 (m, 6H), 1.59 (t, J=10.4 Hz, 1H), 1.51 (s, 3H), 1.10 (t, J=12.4 Hz, 1H). ¹³C NMR (CDCl₃, 100 MHz): δ 169.37, 165.94, 165.36, 163.75, 152.78, 138.71, 138.61, 137.93, 136.88, 134.29, 134.21, 131.87, 129.12, 128.09, 125.93, 120.4, 103.91, 80.97, 80.91, 68.01, 63.22, 59.94, 47.07, 45.69, 43.3, 42.6, 36.5, 25.58, 24.42, 23.84, 17.96, −0.14.

### xvi. ((1aR,7aS,10aS,10bS,E)-1a-methyl-8-methylene-9-oxo-1a,2,3,6,7,7a,8,9,10a,10b-decahydro-oxireno[2′,3′:9,10]cyclodeca[1,2-b]furan-5-yl)methyl (4-((2-(2-iodoethyl)-3,5-dioxo-1,2,4-thiadiazolidin-4-yl)methyl)benzyl)carbamate

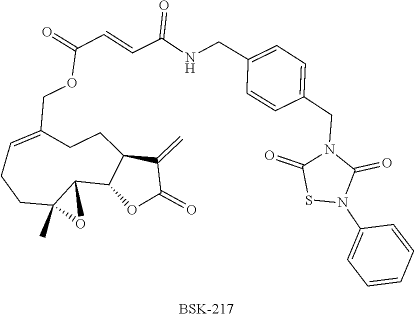 [SMILES: C=C1C(=O)O[C@H]2[C@H]1CC/C(COC(=O)/C=C/C(=O)NCc1ccc(Cn3c(=O)sn(-c4ccccc4)c3=O)cc1)=C\CC[C@@]1(C)O[C@@H]21]

To the reaction mixture of MMB-carboxylic acid, (65 mg, 0.18 mmol), ethyl chloroformate (20 mg, 0.18 mmol), and triethylamine (18 mg, 0.18 mmol) in dry THE (5 ml) was stirred at 0° C. for 1 hr., then 4-(4-(aminomethyl)benzyl)-2-phenyl-1,2,4-thiadiazolidine-3,5-dione (56.5 mg, 0.18 mmol) was added. The resulting reaction mixture was stirred at ambient temperature for 4 hr. after reaction was complete (as monitored by TLC); added water and extracted with dichloromethane. The organic layer was dried and concentrated to get crude compound, which was further purified by column chromatography (silica gel, 3% methanol in dichloromethane) to afford pure product as a white solid. ¹H NMR (CDCl₃, 400 MHz): δ 7.46 7.36 (m, 6H), δ 7.25-7.20 (m, 3H), 6.86 (d, J=15.6 Hz, 1H), 6.79 (d, J=15.6 Hz, 1H), 6.40 (br-s, 1H), 6.19 (s, 1H), 5.64 (t, J=8.0 Hz, 1H), 5.50 (s, 1H), 4.71 (s, 2H), 4.68 (d, J=12.4 Hz, 1H), 4.46 (d, J=12.4 Hz, 1H), 4.46 (br-s, 2H), 3.79 (t, J=9.2 Hz, 1H), 2.87-2.79 (m, 2H), 2.45-2.11 (m, 6H), 1.62 (t, J=12.0 Hz, 1H), 1.51 (s, 3H), 1.03 (t, J=12.0 Hz, 1H). ¹³C NMR (CDCl₃, 100 MHz): 169.37, 165.1, 165.01, 163.2, 150.89, 138.58, 137.7, 136.7, 135.62, 134.54, 134.36, 130.77, 130.07, 129.51, 129.5, 128.32, 127.09, 123.4, 120.44, 80.99, 67.21, 63.21, 45.72, 43.61, 42.6, 36.53, 25.54, 24.2, 23.77, 17.93 ppm.

### xvii. (5,7-dichloro-8-hydroxyquinolin-2-yl)methyl (4-((2-(4-chlorophenyl)-3,5-dioxo-1,2,4-thiadi-azolidin-4-yl) methyl)benzyl)carbamate

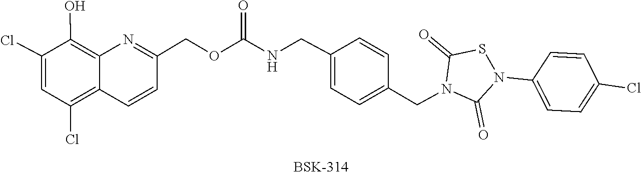 [SMILES: O=C(NCc1ccc(Cn2c(=O)sn(-c3ccc(Cl)cc3)c2=O)cc1)OCc1ccc2c(Cl)cc(Cl)c(O)c2n1]

To the compound (8-((tert-butoxycarbonyl)oxy)-5,7-dichloroquinolin-2-yl)methyl 1H-1,2,4-triazole-1-carboxylate (77 mg, 0.17 mmol) in dichloromethane (2 mL), 4-(4-(aminomethyl)ben-zyl)-2-(4-chlorophenyl)-1,2,4-thiadiazolidine-3,5-dione (62 mg, 0.17 m mol) and trimethylamine (34 mg, 0.34 m mol) were added at RT. The reaction mixture was stirred for 1h, after completion of reaction (monitored by TLC), added water and the aqueous mixture was extracted with dichloromethane. The separated organic layer was washed with water, followed by brine solution, dried over anhydrous Na₂SO₄ and concentrated to afford the crude O-Boc-protected BSK-314. This crude product was purified by column chromatography (silica gel, 2% methanol in dichloromethane) to afford pure O-Boc-protected BSK 314, as a white solid.

The above obtained O-Boc-protected BSK 314 compound was treated with trifluoroacetic acid (0.5 mL) in dichloromethane (5 mL) and stirred for 6 h until the O-Boc-deprotection reaction is completed. Saturated NaHCO₃ solution (5 mL) was added to the reaction mixture and extracted with dichloromethane. The separated organic layer was washed with water, followed by brine solution and dried over anhydrous Na₂SO₄, concentrated to afford compound BSK 314 as a white solid.

¹H NMR (400 MHz, DMSO-d₆) δ 10.53 (brs, 1H), 8.52 (d, J=6.4 Hz, 1H), 8.02 (brs, 1H), 7.76 (s, 1H), 7.69 (d, J=8.8 Hz, 1H), 7.56-7.49 (m, 4H), 7.28-7.25 (m, 4H), 5.31 (s, 2H), 4.77 (s, 2H), 4.18 (s, 2H), ¹³C NMR (100 MHz, DMSO-d₆) 165.16, 158.07, 156.49, 151.17, 149.18, 139.72, 135.29, 134.5, 134.35, 131.31, 129.84, 128.29, 128.11, 127.82, 125.67, 124.49, 121.3, 119.56, 116.42, 66.75.

### xviii. 4-(4-(aminomethyl)benzyl)-2-(4-methoxyphenyl)-1,2,4-thiadiazolidine-3,5-dione

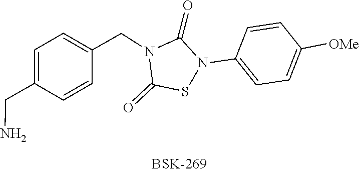 [SMILES: COc1ccc(-n2sc(=O)n(Cc3ccc(CN)cc3)c2=O)cc1]

Sulfuryl chloride (47 mg, 0.36 mmol) was added to the mixture of tert-butyl (4-(isothiocyanatomethyl)-benzyl)carbamate (100 mg, 0.36 mmol) and 4-methoxyphenyl isocyanate (54 mg, 0.36 mmol) in dry THE at 0° C. under N₂ atmosphere. The mixture was stirred for 12h at room temperature. Then, water was added and stirring was continued for another 30 min. Solvent was evaporated and extracted with ethyl acetate. The combined organic layers were washed with brine, dried, and concentrated. The residue was purified by flash chromatography (25% EtOAc in hexanes) to give N-Boc-protected BSK-269.

N-Boc-protected BSK-269 compound was treated with trifluoroacetic acid (2 eq) in dichloromethane and stirred for 6 h. Then, Saturated NaHCO₃ solution was added to the reaction mixture and extracted with dichloromethane. The separated organic layer was washed with water, followed by brine solution and dried over anhydrous Na₂SO₄, concentrated to afford pure BSK-269 analog as white solid.

¹H NMR (DMSO-d₆, 400 MHz): δ 8.16 (br-s, 2H), 7.43 (d, J=8.8 Hz, 2H), 7.38 (d, J=8.4 Hz, 4H), 7.0 (d, J=8.8 Hz, 2H), 4.8 (s, 2H), 3.99 (br-s, 2H), 3.74 (s, 3H); ¹³C NMR (DMSO-d₆, 100 MHz): δ 165.67, 158.83, 151.51, 136.41, 134.03, 129.64, 128.49, 128.33, 127.12, 115.11, 55.92, 45.52, 42.39.

2. Protocols for Screenings and Computational Analysis

a. Screening and Computational Analysis

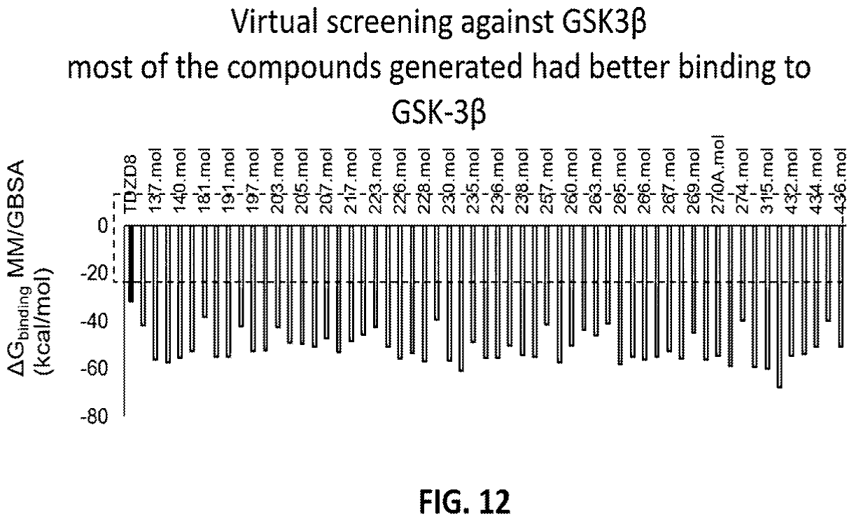

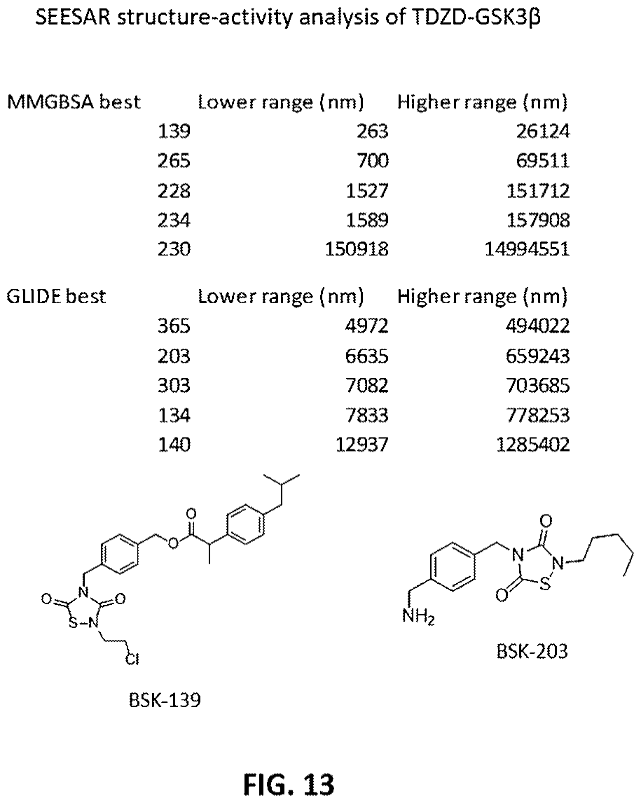

Representative images and data of TDZD lead optimization, virtual screening for stable binding to GSK3β, and SeeSAR structure-activity analysis of TDZD-GSK3β are shown in FIG. 12 and FIG. 13. Data regarding protein aggregation reduction and IC₅₀ values from the structure-activity analysis of TDZD-GSK3β are shown in Table 1 below. Note that “Log” refers to the logarithm with base 10.

TABLE 1

| Compounds | IC₅₀ (M) | Log(IC₅₀) |
|---|---|---|
| 139 | 1.0E−09 | −8.715795 |
| 265 | 10.7 | 1.027757 |
| 230 | 3.36 | 0.526081 |
| 228 | 0.00 | −7.98E+36 |
| 203 | 64.4 | 1.808684 |
| 234 | 0.06 | −1.211478 |
| 140 | 88.25 | 1.945715 |
| 134 | 971.8 | 2.987577 |

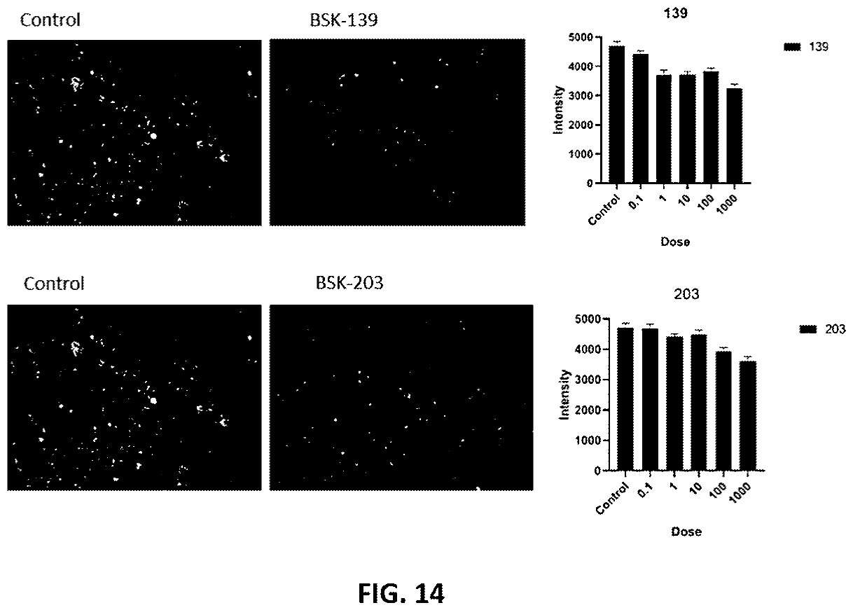

In vitro screening of representative compounds in HEK-TAU cells (thioflavin-T staining) was performed. See FIG. 14.

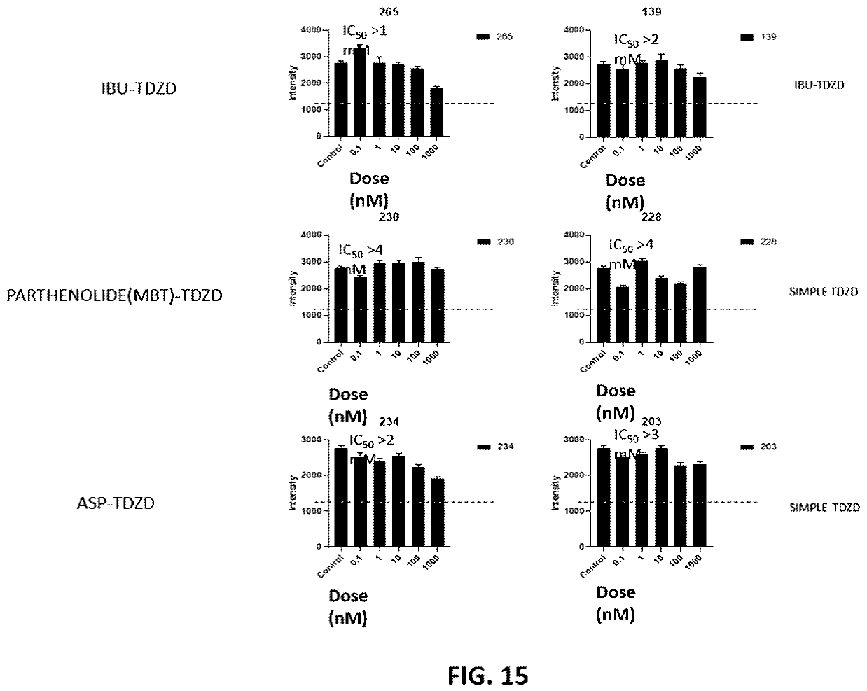

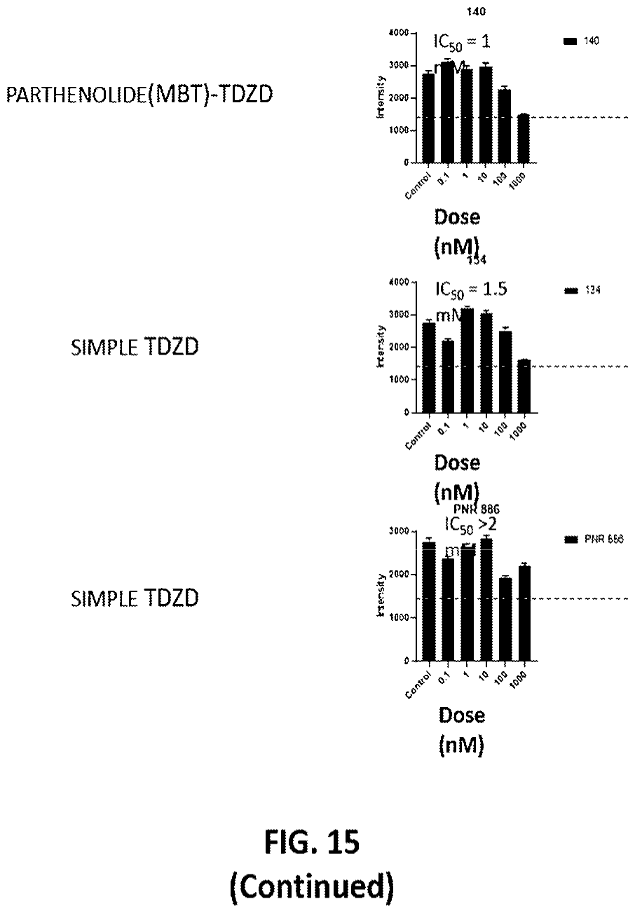

Data from a screen conducted using SY5Y-APP are shown in FIG. 15.

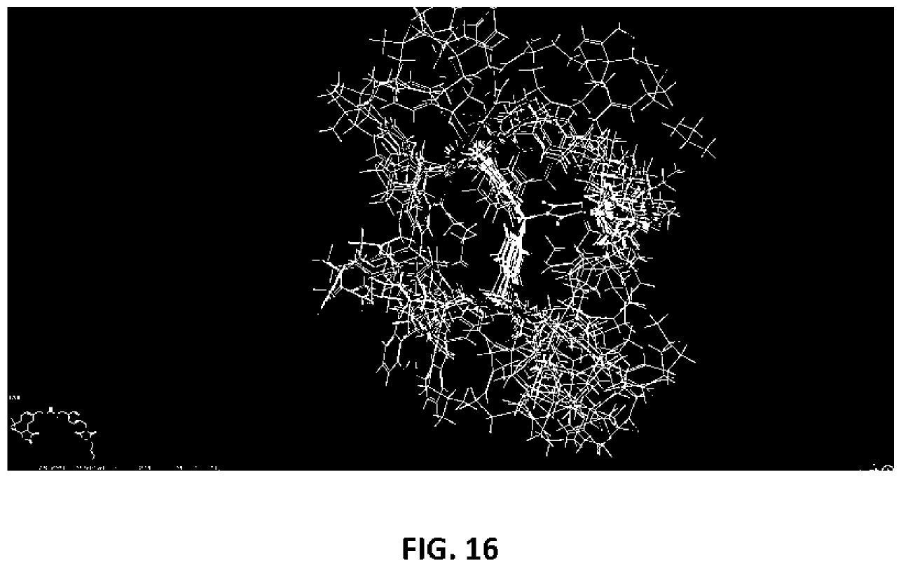

Alignment and molecular descriptors achieved using Maestro are shown in FIG. 16.

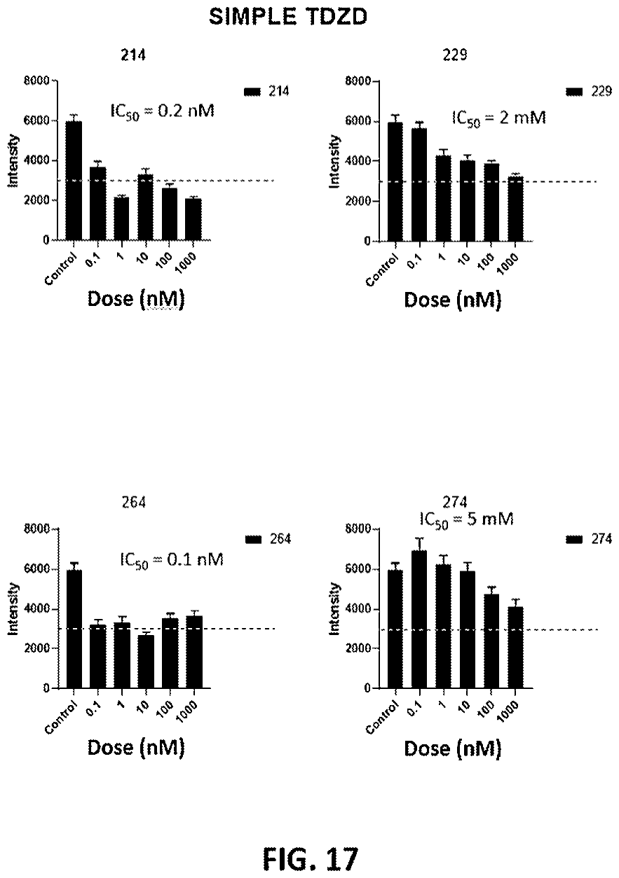

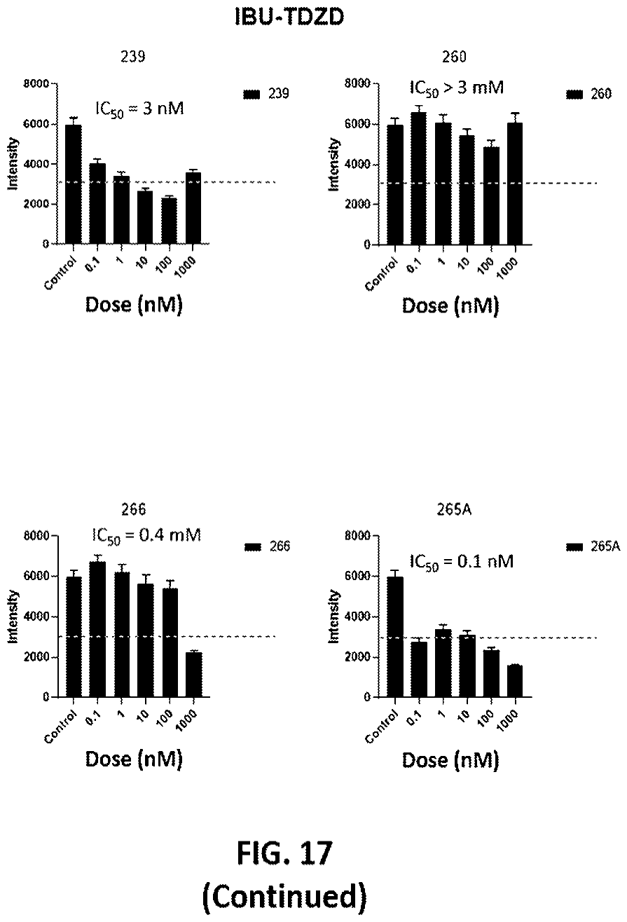

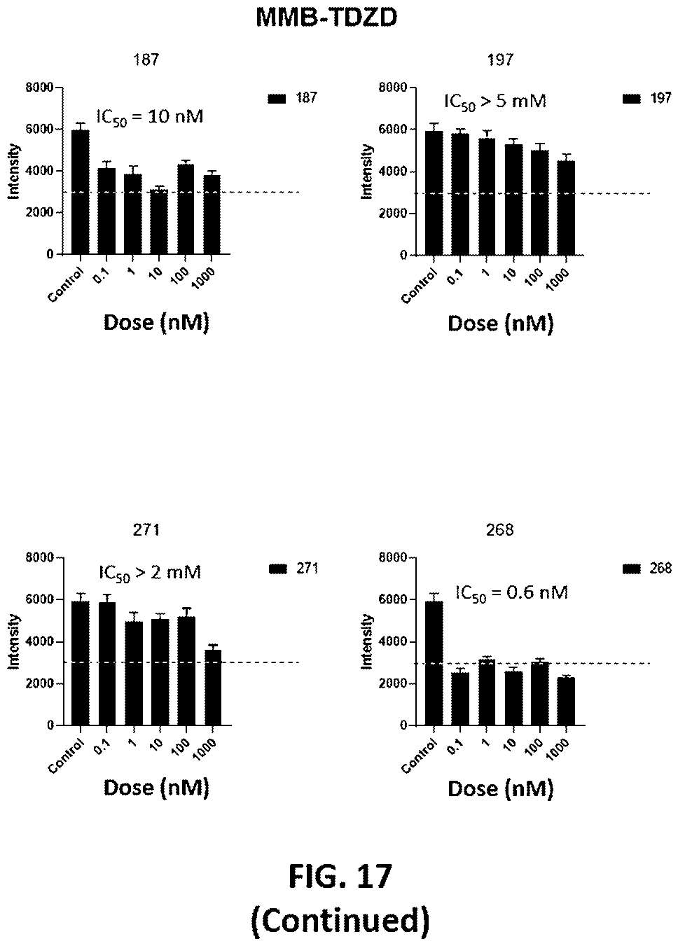

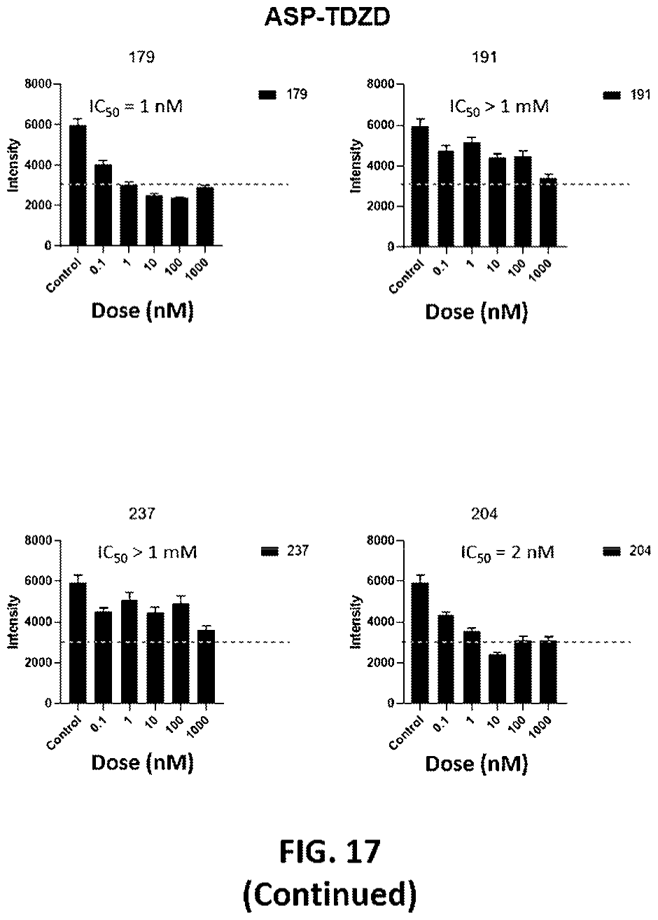

Data from a screen conducted on 16 compounds in SY5Y-APP are shown in FIG. 17.

b. Screening of TDZD-MMB, TDZD-Aspirin, and TDZD-Ibuprofen Conjugates Against Protein Aggregation

Several TDZD-conjugated drugs (putative GSK-3 inhibitors) were evaluated for their ability to reduce accumulation of protein aggregates, and for protection against aggregation-associated end-points in the following model systems: (i.) SH-SY5Y-APP_{Sw}, a neuroblastoma cell line expressing the Swedish mutation of Amyloid Precursor Protein (APP_{Sw}), and thus predisposed to form β-amyloid deposits; (ii.) C. elegans strain CL4176, a model of Alzheimer-like amyloidopathy that can be induced to express human Aβ₁₋₄₂ in muscle, or may express it at a lower level without induction; (iii.) C. elegans strain AM141, a model of Huntington-like polyglutamine aggregation that constitutively expresses Q40::YFP (a tract of 40 glutamines fused in-frame to yellow fluorescent protein) in muscle cells and forms aggregates progressively with adult aging. The TDZD analog PNR-962 (a putative GSK-3 inhibitor) and other related compounds (not shown) were protective in all aggregation assays.

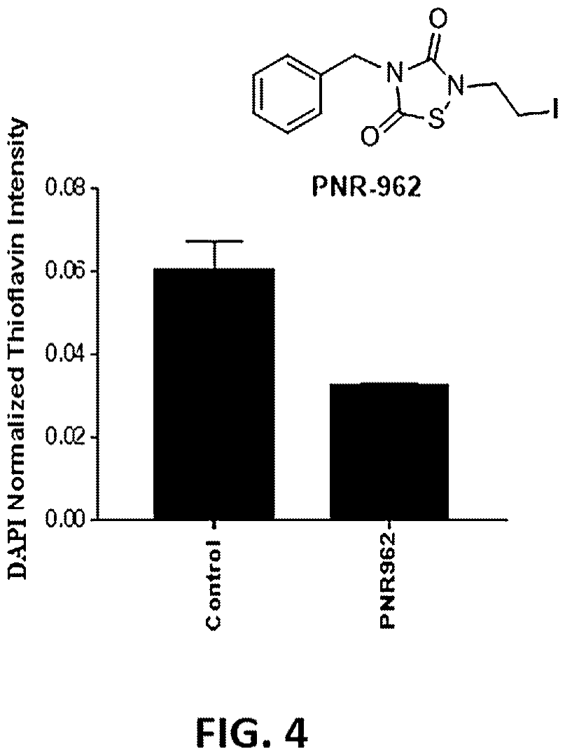

First, the effects of TDZD analogues were tested on the human neuroblastoma cell line SH-SY5Y-APP_{Sw}, a model of Alzheimer-like amyloid aggregation expressing the “Sw” mutant form of Amyloid Precursor Protein, APP. These drugs all produced significant reductions in amyloid-like aggregates, by 30-50% as quantified by thioflavin-T staining intensity (data for PNR-962 are shown in FIG. 4). Modified TDZD-8 analogues PNR-962 and BSK-179 are more anti-aggregative than TDZD-8, lowering total amyloid-staining intensity by a further 14% (data not shown).

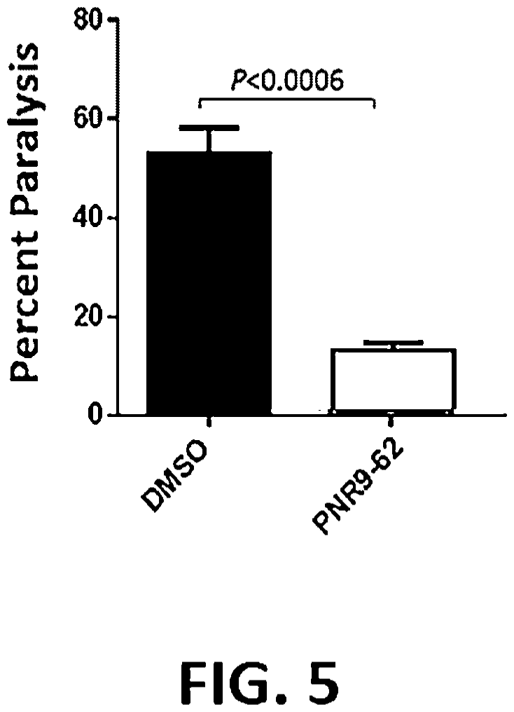

These analogues were even more effective in opposing amyloid-associated paralysis in C. elegans strain CL4176, a model of Alzheimer-like amyloidopathy in which human Aβ₁₋₄₂ is expressed (and is cytotoxic) in muscle. These worms become progressively paralyzed, 24-42 h after induction, but >75% of paralysis is rescued by exposure to PNR-962 (FIG. 5).

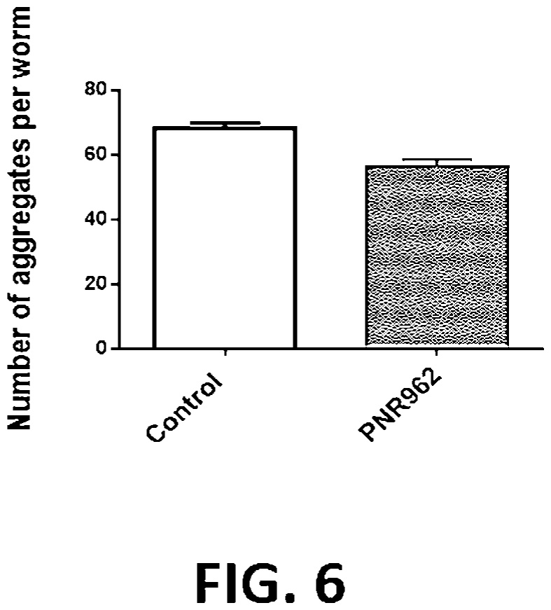

Exposure of C. elegans strain AM141 to PNR-962 reduced the number of aggregates significantly (FIG. 6); it also reduced aggregate intensity, i.e., content of Q40-YFP, to a similar extent (not shown).

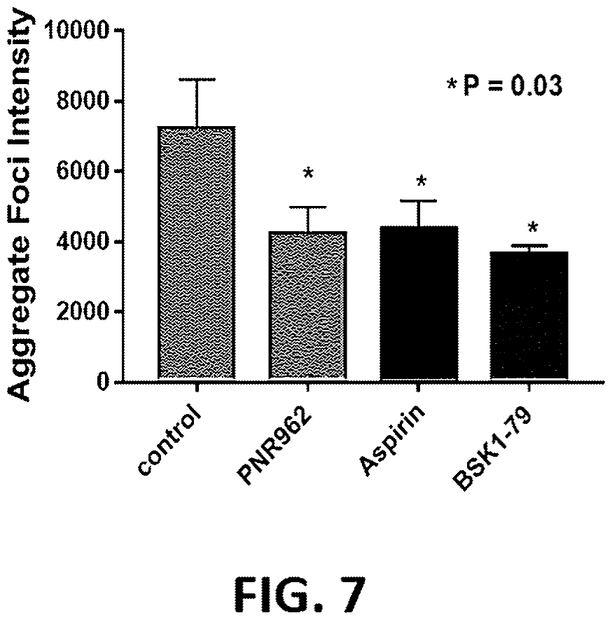

BSK-179 comprises an acetyl donor (similar to aspirin), coupled to PNR-962. It reduced the total intensity of Q40::YFP aggregates by >2-fold, a substantially greater protection than afforded by PNR-962 or aspirin alone (FIG. 7).

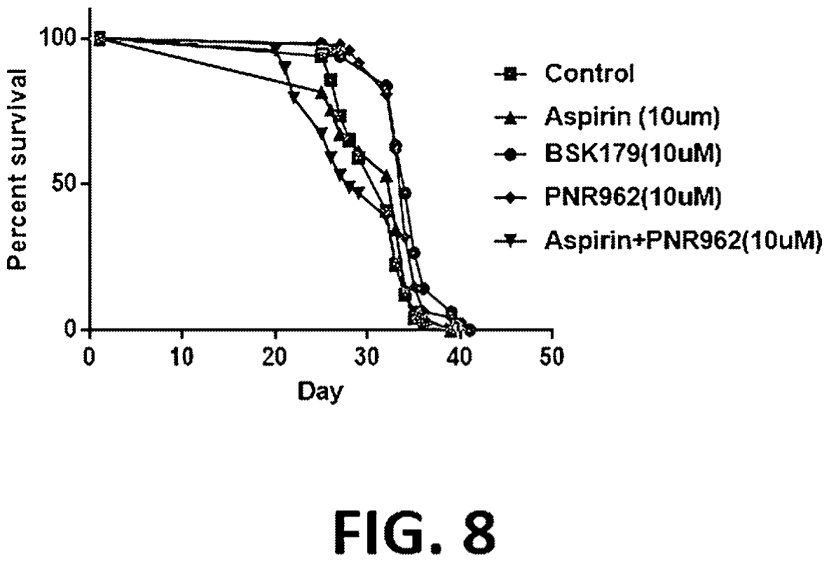

It was previously shown that several interventions that improve age-associated phenotypes also modulate lifespan (Ayyadevara et al. (2017) Antioxid Redox Signal. 27(17): 1383-1396; Ayyadevara et al. (2005) Aging Cell. 4(5): 257-271). The lifespan was assessed in C. elegans, either mock-treated or exposed to TDZD analogues. PNR-962 and BSK-179 extended the lifespan of wild-type nematodes to an identical degree, increasing the mean by roughly 25-30% (P<0.001; FIG. 8).

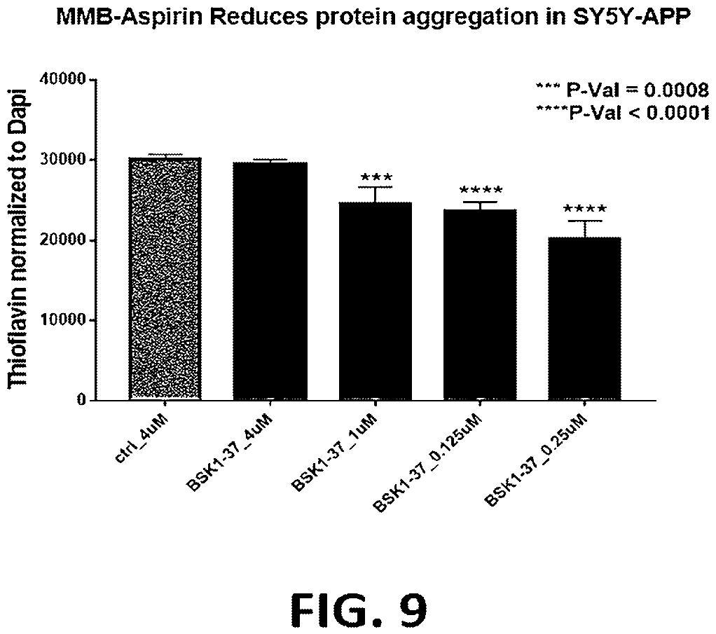

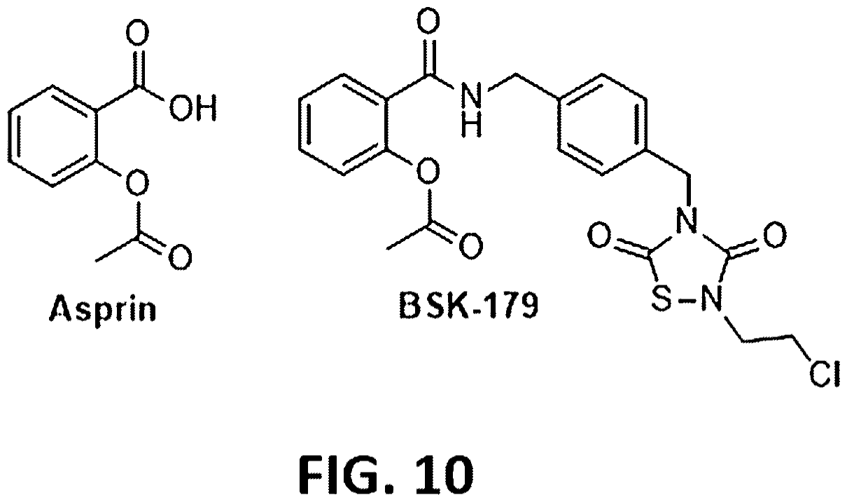

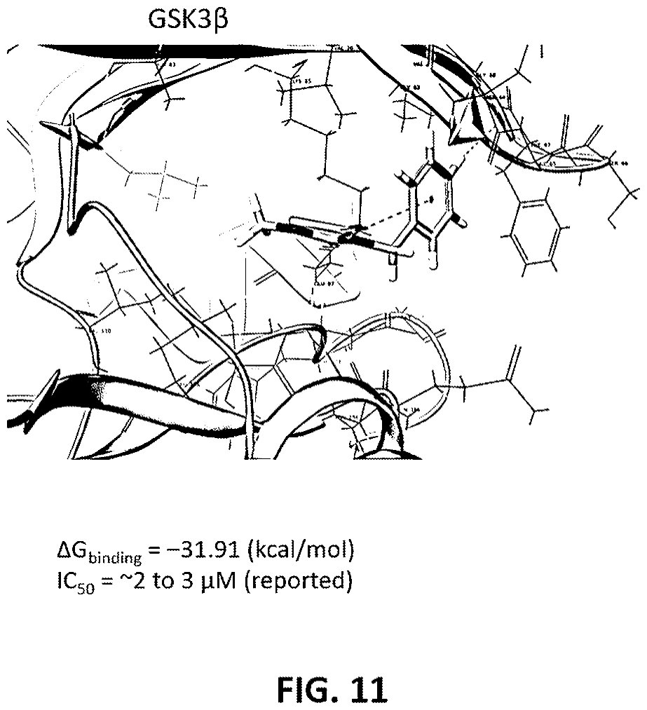

A TDZD-ibuprofen analog (BSK-137), a drug combining the GSK-3 inhibitor with Ibuprofen, was synthesized and its effect on protein aggregation was tested in neuronal cells. The combination drug BSK-137 reduced aggregate intensity by 34% as evidenced by reduced Thioflavin—T staining (FIG. 9).

Without wishing to be bound by theory, this provides a distinct advance over life-extension, and reduction in disease risks, observed with previous NSAIDs such as aspirin and ibuprofen (Ayyadevara et al. (2013) Antioxid Redox Signal. 18(5): 481-490; Lou et al. (2016) Lipids Health Dis. 15: 106; Shebl et al. (2012) Br J Cancer. 107(1): 207-214), and over previous inhibitors of GSK3β.

As illustrated in FIG. 3-9, above, these drugs are potent in reducing aggregates and extending survival; it is also noted that these analogues have very low toxicity in both human cells and in intact animals (C. elegans). In each case, lethality was 0% at doses of 0.1-1 mM (data not shown). Diverse applications of these novel drugs are given below.

c. Methods for Qsar Modeling

A mini-scale high throughput screening of the TDZD family of small molecules was initially performed, and some lead analogs were characterized. The initial screen was assessed using machine-learning algorithms to predict effective small molecules, often referred to as “computer-aided drug discovery” (Ain, et al. (2020) TLR4-Targeting Therapeutics: Structural Basis and Computer-Aided Drug Discovery Approaches. Molecules 25: 627; Ain, et al. (2020) TLR4-Targeting Therapeutics: Structural Basis and Computer-Aided Drug Discovery Approaches. Molecules 25; Pereira, C. A. et al. (2020) Computational approaches for drug discovery against trypanosomatid-caused diseases. Parasitology 147: 611-633; Batool, et al. (2019) A Structure-Based Drug Discovery Paradigm. Int J Mol Sci 20). Illustratively, most previous methods relied on linear relationships that have been useful in the past, but often require data transformation, and may fail for non-linear relationships for which complex algorithms like NN, KNN, SVM and others might be more effective. Here, machine learning algorithms were applied in quantitative structure-activity relationship (QSAR) modeling to discover what structural or physical properties of molecules in a small-molecule library might be improved to increase the potency of a given drug (Rajkumar, S. (2020) The high cost of prescription drugs: causes and solutions. Blood Cancer Journal 10; Gronde, et al. (2017) Addressing the challenge of high-priced prescription drugs in the era of precision medicine: A systematic review of drug life cycles, therapeutic drug markets and regulatory frameworks. PLoS One 12, e0182613; Jatto and Okhamafe (2002) An Overview of Pharmaceutical Validation and Process Controls in Drug Development. Tropical Journal of Pharmaceutical Research 1: 115; Kaitin, K. I. (2010) Deconstructing the Drug Development Process: The New Face of Innovation. Clinical Pharmacology & Therapeutics 87, 356-361). QSAR modeling approaches have been used to study many physiochemical properties of small molecules including drug toxicity, anti-aggregation properties, solubility, electronic factors, and hydrophobicity, among others (Ponzoni, I. et al. (2017) Hybridizing Feature Selection and Feature Learning Approaches in QSAR Modeling for Drug Discovery. Sci Rep 7, 2403; Tropsha, A. (2010) Best Practices for QSAR Model Development, Validation, and Exploitation. Molecular Informatics 29, 476-488; McKenzie, et al. (2006) Can pharmaceutical process development become high tech? AIChE Journal 52: 3990-3994; Gramatica, P. (2007) Principles of QSAR models validation: internal and external. QSAR & Combinatorial Science 26: 694-701; Wang, H. et al. (2017) Design of cinnamaldehyde amino acid Schiff base compounds based on the quantitative structure-activity relationship. R Soc Open Sci 4: 170516).

Having shown dose-dependent anti-aggregative activity for some analogs of the thiadiazolidines (TDZDs), it is sought to use aggregation inhibiting activity as an endpoint, and to explore the power of predictive modeling with QSAR machine-learning algorithms to define the molecular properties that contribute most to anti-aggregative effects of compounds. The small-molecule library was modeled, the ligands prepared, structures superimposed by a common ring or side-chain of interest (TDZD ring), and molecular spreadsheets were subsequently generated in Maestro covering over 400 molecular descriptors. The molecular descriptors generated included 1-dimensional (e.g., atom count, molecular weight, and number of bonds), 2-dimensional (e.g., topological, structural, functional-group count), 3-dimensional (e.g. electronic, spatial, and geometric indices) and 4-dimensional (4D, i.e., time-dependent) features.

A robust QSAR pipeline was designed, tested, and validated using other endpoints like log(IC₅₀) of anti-aggregative activity, and 7 highly optimized machine learning algorithms were employed, namely, k-nearest neighbors (KNN), neural network (NN), partial least squares (PLS), support vector machine (SVM), bagging (B), random forest (RF), and decision tree (DT), for predictive QSAR modeling for the TDZD family of small molecules.

By this approach, linear and non-linear relationships between the physiochemical properties of the small molecules and their associated anti-aggregative activity were found. This predictive power ultimately helps to design better anti-aggregative drugs to target AD and other age-associated neurodegenerative diseases. A virtual screening for the entire TDZD library was performed using the most predictive well-tuned algorithm followed by ranking and testing for the activity to assess the correlation (agreement) between predicted vs. observed activity. Subsequently, the relative importance of the major molecular descriptors useful in predicting aggregation inhibition activity of TDZD analogs in the small-molecule library will be estimated. By this approach, the synthesis of the most active candidate compounds in the library to treat AD and associated neurodegenerative diseases can be guided. The compounds predicted to be most active will be synthesized and then characterized in human cell-culture and nematode models of AD, for aggregation inhibition, age-progressive paralysis, and life span studies, among others.

d. Structural Modeling of TDZD Analogs and GSK3β

The inactive conformation of GSK3β from previous work (Balasubramaniam, et al. (2020) Structural modeling of GSK3β implicates the inactive (DFG-out) conformation as the target bound by TDZD analogs. Scientific Reports, 10(1)) was converted to Autodock format. The structure of NR962 were converted to simplified molecular-input line-entry system (SMILES) format using ChemDraw and subsequently converted to SYBYL mol2 format. SMILES uses ASCII strings for line notation to represent the structures of chemical species. Because the previously derived structure of the inactive conformation protein omits several loops, those gaps (i.e., missing hydrogens, side chains, and loops) were filled by template modelling using the in-built protein preparation wizard in the Maestro Prime module (Schrodinger, Inc.). All subsequent computational docking and simulations studies used the preprocessed inactive conformation of the GSK3β protein template just described.

e. Docking of Tdzd Analogs to Inactive Conformation of GSK3β.

Computational modeling, docking, and simulation approach was followed as described in previous studies, using Glide docking and MMBGSA assay, which demonstrated that TDZD-8 binds in the allosteric hydrophobic pocket of GSK3β (Balasubramaniam, et al., (2018) Structural modeling of GSK3β implicates the inactive (DFG-out) conformation as the target bound by TDZD analogs. Scientific Reports, 10(1)), which is only present in the inactive conformation of the protein. To examine the drug-binding modes of the novel TDZD analogue PNR962 to GSK313, the modelled full-length GSK3β in the inactive conformation was used, and its interaction with PNR962 simulated. Unbiased docking specifically requires enclosing the full-length protein in a grid box, thereby allowing the ligand (PNR962) to search globally for a preferred binding site to protein (GSK3β in the inactive conformation) as previously described (Bommagani, et al., (2019) A novel tetrazole analogue of resveratrol is a potent anticancer agent. Bioorg Med Chem Lett, 29(2): 172-178; Janganati, et al. (2018) MMB triazole analogs are potent NF-κB inhibitors and anti-cancer agents against both hematological and solid tumor cells. Eur J Med Chem, 157: 562-581). Unbiased docking of ligands to the allosteric pocket in the GSK3β inactive conformation was performed using Autodock-Vina with Raccoon interface via a Linux-based server. The grid box was created using the Receptor Grid Generator Wizard in Maestro. Docking computations were performed in standard precision mode, which ensures flexible ligand sampling. Visualization and analysis of results were all carried out in Maestro Viewer and Discovery Studio Visualizer. Next, the predicted binding site for the inactive conformation of the GSK3β allosteric pocket (obtained by unbiased Autodock-Vina docking studies) was used in Glide docking interfaced with Maestro 2017-2 Suite (Schrodinger) on a commercial license and assessed by the MMBGSA method for a protein-ligand targeted docking.

f. Preparation and Running Simulation

The stability of a protein-ligand complex (after docking) is of great pharmaco logical interest. Regular protein-ligand simulations were performed to ascertain the stability of the molecular complex. To achieve this, the prepared ligand-protein molecular complexes (PNR962-GSK3β, and TDZD-8-GSK3β) were each enclosed in an orthorhombic box, ensuring that all edges of the box are at least 10-Å from the protein. Solvation and neutralization of the boxed protein were accomplished with Simple Point Charge (SPC) water and Na⁺, Cl⁻ counter-ions, respectively. NaCl was added at 0.15 M to ensure the appropriate physiological salt concentration. Following NVT procedure with a Nose-Hoover chain thermostat, the system was equilibrated (at 300° K) prior to simulation. A secondary equilibration was performed following NPT procedure, after which the Molecular Dynamic (MD) simulation was run as described in computational approaches (Balasubramaniam, et al. (2020) Structural modeling of GSK3β implicates the inactive (DFG-out) conformation as the target bound by TDZD analogs. Scientific Reports, 10(1); Balasubramaniam, et al. (2019) Interleukin-1β drives NEDD8 nuclear-to-cytoplasmic translocation, fostering parkin activation via NEDD8 binding to the P-ubiquitin activating site. Journal of Neuroinflammation, 16(1); Lakkaniga, et al. (2019) Structural Characterization of the Aurora Kinase B “DFG-flip” Using Metadynamics. Aaps Journal, 22(1)). With collective variables (CVs) performing significant aspects in simulations (Park, et al. (2016) Molecular Dynamics Analysis of Binding of Kinase Inhibitors to WT EGFR and the T790M Mutant. Journal of Chemical Theory and Computation, 12(4): 2066-2078), a Guassian distance of 0.05 Å was ensured for the Phe 201-Ser168 distance in the inactive conformation of the GSK3β structure. Simulations were performed under standard conditions (temperature, T=300° K and pressure, p=1.0 bar, with RESPA integrator) using Desmond v2018.1 enhanced on an in-house GPU cluster (NVIDIA Quadro P5000). The Simulation Interaction Diagram Generator module in Desmond-Maestro was used to view and analyze the resulting simulation trajectories for the protein-ligand simulated complex.

g. Binding Energy (ΔG_{Binding}) Computation for MM/GBSA

Glide docking poses served as the starting entries (inputs) for computing the solvent-based free energy (ΔG_{binding}) for each molecular complex in retrospect (i.e., PNR962-GSK3β, PNR962-GSK3β, and TDZD-GSK3β). In estimating the binding free energy of the individual ligands binding to GSK313, the in-built Prime module from Schrodinger Suite was employed for the MM-GBSA procedure.

3. Biological Protocols

a. Anti-Leukemic Activity of TDZD Analogs

The synthesized compounds were evaluated for anti-leukemic activity against the MV4-11 cell line (myelomonocytic leukemia), utilizing PTL and TDZD-8 as reference positive-control drugs in all assays. The results from the MV4-11 cell assay indicated that the MMB-TDZD analogs (BSK-140 to BSK-271) were cytotoxic to MV4-11 cells with equal or greater potency to that of parthenolide (PTL) or TDZD-8. Two of these analogues, BSK-259 and BSK-230, were the most potent antileukemic agents examined, with LD₅₀ values of 10 nM and 20 nM, respectively. Thus, BSK-259 is 273-fold more cytotoxic than PTL and 329-fold more cytotoxic than TDZD-8. Other compounds of interest were BSK-2-68, BSK-271, BSK-218 and BSK-197, which exhibited LD₅₀ values 130 nM, 680 nM, 760 nM and 980 nM respectively. See Table 2, which shows the LD₅₀ (μM) values of MMB-TDZD analogs against cultured MV-411 cells after 24-h.

TABLE 2

| | 7AAD (μM) | YOPRO (μM) | Alamar blue (μM) |
|---|---|---|---|
| Compd. | 24 h | 24 h | 24 h |
| TDZD-8 | 3.61 | 3.29 | 4.5 |
| PTL | 2.27 | 2.56 | 2.7 |
| BSK-140 | 4.42 | 2.88 | 1.50 |
| BSK-187 | 2.37 | 1.21 | 1.0 |
| BSK-197 | 1.26 | 1.17 | 2.02 |
| BSK-218 | 1.3 | 1.07 | 0.8 |
| BSK-226 | 0.75 | 0.37 | 0.45 |
| BSK-230 | 0.01 | 0.02 | 0.30 |
| BSK-259 | 0.01 | 0.01 | 0.30 |
| BSK-263 | 0.23 | 0.13 | 0.75 |
| BSK-268 | 0.55 | 0.68 | 0.63 |
| BSK-271 | 1.74 | 2.07 | 1.20 |

The synthesized Ibuprofen-TDZD combination drugs were evaluated for anti-leukemic activity against MV4-11 cell lines by utilizing TDZD-8 as a positive control in all assays. The results from the MV4-11 cell assay indicated that the Ibuprofen-TDZD analogs (BSK-137 to BSK-270) exhibited cytotoxic potency equal or greater than 2.3-fold that of TDZD-8. Compounds BSK-260 and BSK-236 were the most potent antileukemic agents examined with LD₅₀ values of 1.42 μM and 1.75 μM, respectively. Other compounds of interest were BSK-235, BSK-265 and BSK-137, which exhibited LD₅₀ values 2.54 μM, 2.71 μM and 3.39 μM respectively. See Table 3, LD₅₀ (μM) values of Ibuprofen-TDZD analogs against cultured MV-411 cells after 24h.

TABLE 3

| | 7AAD (μM) | YOPRO (μM) | Alamar blue (μM) |
|---|---|---|---|
| Compd. | 24 h | 24 h | 24 h |
| TDZD-8 | 3.61 | 3.29 | 4.5 |
| BSK-137 | 3.39 | 3.28 | 7.00 |
| BSK-235 | 2.54 | 3.2 | 3.50 |
| BSK-236 | 1.75 | 1.77 | 2.50 |
| BSK-238 | 11.2 | 10.9 | 10.00 |
| BSK-239 | 4.91 | 4.24 | 4.70 |
| BSK-260 | 1.42 | 1.48 | 1.49 |
| BSK-265 | 2.71 | 2.87 | 2.50 |
| BSK-266 | 10.7 | 10.6 | 12.00 |
| BSK-267 | 14.8 | 17.3 | 12.00 |
| BSK-270 | 8.21 | 6.14 | 9.00 |

b. In Vitro Growth Inhibition and Cytotoxicity Against a NCI-60 Human Cancer Cell Panel

In a primary screen, all the synthesized TDZD conjugate compounds were evaluated for cytotoxic potency at the National Cancer Institute (NCI). From the preliminary 60 cell-line screen, the 8 compounds that showed ≥60% growth inhibition in at least eight of the cancer cell lines screened were selected for complete dose-response curves comprising 5 concentrations per drug (10⁻⁴ M, 10⁻⁵M, 10⁻⁶ M, 10⁻⁷ M and 10⁻⁸ M). Cytotoxic potencies are summarized in Table 4 as GI₅₀ values (molar drug concentration at 50% growth inhibition); a measure of drug cytotoxicity, LD₅₀, was also determined (data not shown). GI₅₀ values <1.0 are emphasized in bold font as strongly indicative of high potency. We note that BSK-259 displayed high potency against all 6 leukemia cell lines in the NCI panel, while BSK-226 was comparably potent against 4 out of 5 leukemia cell lines. BSK-230, the second most potent drug against the MV-411 cell line, was highly potent against 3 of the 6 leukemia cell lines in the NCI panel. These 3 drugs are thus the most promising candidates as anti-leukemic agents.

TABLE 4

| | BSK-140 | BSK-187 | BSK-197 | BSK-226 | BSK-218 | BSK-230 | BSK-259 | BSK-271 |
|---|---|---|---|---|---|---|---|---|
| Panel/ | GI₅₀^{a} | GI₅₀^{a} | GI₅₀^{a} | GI₅₀^{a} | GI₅₀^{a} | GI₅₀^{a} | GI₅₀^{a} | GI₅₀^{a} |
| Cell line | (μM) | (μM) | (μM) | (μM) | (μM) | (μM) | (μM) | (μM) |
| Leukemia | | | | | | | | |
| CCRF-CEM | 1.73 | 0.50 | 0.83 | 0.57 | 0.36 | 0.65 | 0.31 | 0.56 |
| HL-60(TB) | 1.91 | ND | ND | 0.96 | 1.10 | 1.81 | 0.30 | 2.12 |
| K-562 | 2.07 | 0.65 | 1.80 | 0.72 | NA | 0.89 | 0.34 | ND |
| MOLT-4 | 1.85 | 1.35 | 2.20 | 1.50 | 5.90 | 1.55 | 0.29 | 1.93 |
| RPMI-8226 | 2.37 | 1.98 | 2.03 | 0.76 | 1.12 | 1.48 | 0.90 | 2.01 |
| SR | 1.48 | ND | ND | ND | 2.99 | 0.40 | 0.28 | 0.53 |
| Non-Small-Cell | | | | | | | | |
| Lung Cancer | | | | | | | | |
| EKVX | 1.99 | 2.11 | 1.85 | 2.82 | 1.85 | 3.32 | 2.09 | 3.5 |
| HOP-92 | 1.50 | 1.38 | 1.82 | 1.10 | 1.38 | 1.29 | 1.22 | 1.61 |
| NCI-H23 | 2.18 | 3.46 | 3.55 | 2.33 | 1.94 | 4.19 | 2.11 | 2.97 |
| NCI-H460 | 5.10 | 6.42 | 3.67 | 11.3 | 3.42 | 5.92 | 3.20 | 3.7 |
| NCI-H522 | 1.38 | 1.72 | 1.74 | 0.53 | 1.59 | 1.19 | 1.07 | 1.67 |
| Colon Cancer | | | | | | | | |
| COLO 205 | 1.84 | 1.86 | 1.77 | 2.04 | 1.78 | 2.00 | 1.95 | 1.85 |
| HCT-116 | 1.62 | 1.76 | 1.52 | 0.56 | 1.73 | 1.36 | 1.05 | 1.93 |
| HCT-15 | 1.85 | 1.91 | 1.94 | 1.94 | 1.66 | 2.02 | 1.89 | 1.68 |
| HT29 | 2.16 | 2.10 | 2.15 | 2.46 | 2.09 | 2.08 | 1.74 | 1.96 |
| SW-620 | 1.17 | 1.66 | 1.91 | 1.23 | 1.31 | 1.51 | 1.40 | 1.77 |
| CNS Cancer | | | | | | | | |
| SF-268 | 3.73 | 2.41 | 2.21 | 2.63 | 1.88 | 4.28 | 2.54 | 3.14 |
| SF-539 | 1.82 | 1.72 | 1.57 | 1.73 | 1.64 | 1.68 | 1.71 | 1.96 |
| SNB-19 | 11.9 | 6.88 | 4.26 | 4.84 | 5.06 | 6.63 | 3.12 | 11.7 |
| SNB-75 | 7.65 | ND | 2.37 | 2.03 | 1.59 | ND | ND | 3.44 |
| U251 | 6.79 | 4.67 | ND | 5.05 | 2.20 | 41.5 | 1.75 | 4.11 |
| Melanoma | | | | | | | | |
| LOX IMVI | 1.69 | 1.69 | 1.62 | 1.58 | 1.59 | 1.65 | 1.40 | 1.73 |
| MALME-3M | 1.72 | 1.68 | 2.09 | 1.34 | 1.61 | 1.60 | 1.39 | 1.81 |
| M14 | 1.82 | 1.82 | 1.80 | 1.93 | 1.89 | 1.45 | 1.74 | 2.19 |
| MDA-MB-435 | 1.74 | 2.45 | 2.07 | 1.80 | 1.71 | 1.65 | 1.68 | 1.83 |
| SK-MEL-2 | 10.6 | 11.7 | 2.39 | 4.83 | 2.42 | 4.53 | 2.42 | 3.19 |
| SK-MEL-28 | 1.74 | 2.06 | 1.70 | 1.69 | 1.85 | 1.80 | 1.92 | 1.97 |
| SK-MEL-5 | 1.70 | 5.37 | 3.24 | 3.72 | 3.92 | 3.18 | 1.91 | 12.0 |
| UACC-257 | 1.85 | 2.24 | 1.86 | 1.61 | 1.57 | 2.03 | 1.79 | 2.03 |
| UACC-62 | 1.86 | 1.98 | 2.04 | 1.44 | 1.59 | 1.62 | 1.41 | 1.86 |
| Ovarian Cancer | | | | | | | | |
| IGROV1 | 2.50 | 2.08 | 2.09 | 1.78 | 2.11 | 1.83 | 1.75 | 2.85 |
| OVCAR-3 | 1.53 | 1.64 | 1.68 | 0.77 | 1.41 | 1.59 | 1.19 | 1.61 |
| OVCAR-4 | 1.92 | 2.02 | 2.74 | 1.67 | 1.62 | 1.52 | 1.46 | 1.64 |
| OVCAR-5 | 1.97 | 3.28 | 2.51 | 2.64 | 2.01 | 1.93 | 1.85 | 2.06 |
| OVCAR-8 | 2.04 | 4.55 | 2.83 | 2.32 | 2.26 | 3.64 | 3.04 | 2.37 |
| Renal Cancer | | | | | | | | |
| 786-0 | 1.79 | 1.82 | 1.81 | 1.48 | 1.98 | 2.00 | 1.68 | 2.05 |
| A498 | 11.5 | 4.44 | 3.91 | 2.83 | 2.91 | 3.28 | 4.85 | 5.65 |
| ACHN | 1.54 | 1.89 | 1.64 | 1.66 | 1.68 | 1.67 | 1.70 | 1.73 |
| CAKI-1 | 1.76 | 3.79 | 2.49 | 2.05 | 2.84 | 1.64 | 1.70 | 2.34 |
| RXF 393 | 1.18 | 1.61 | 1.75 | 1.35 | ND | 1.57 | 1.52 | ND |
| SN12C | 2.01 | 2.16 | 2.13 | 1.36 | 1.71 | 1.73 | 1.57 | 2.14 |
| TK-10 | 1.77 | 1.91 | 2.21 | 1.75 | 1.96 | 1.81 | 1.71 | 1.91 |
| UO-31 | 1.65 | 1.65 | 1.68 | 1.40 | 1.46 | 1.45 | 1.52 | 1.56 |
| Prostate Cancer | | | | | | | | |
| PC-3 | 8.09 | 1.75 | 2.36 | 2.13 | 1.92 | 1.75 | 1.94 | 2.19 |
| DU-145 | 1.72 | 1.82 | 1.71 | 1.64 | 1.68 | 1.81 | 1.73 | 1.81 |
| Breast Cancer | | | | | | | | |
| MCF7 | 1.91 | 1.71 | 1.59 | 1.71 | 1.79 | 1.87 | 1.41 | 1.77 |
| MDA-MB- | 1.89 | 1.77 | 1.71 | 1.72 | 1.72 | 1.80 | 1.75 | 1.97 |
| 231/ATCC | | | | | | | | |
| HS 578T | 12.1 | 7.29 | 4.14 | 5.64 | 2.79 | 3.24 | 3.17 | 2.92 |
| BT-549 | 1.45 | 2.40 | 2.14 | 1.48 | 1.91 | 1.56 | 1.38 | 4.37 |
| T-47D | 1.97 | 1.32 | 1.98 | 1.97 | 1.75 | 2.04 | 1.79 | 1.86 |
| MDA-MB-468 | 1.78 | 1.25 | 1.17 | 1.53 | 1.19 | 1.73 | 1.54 | 1.53 |

In conclusion, in the present study, a series of novel combination drugs (TDZD-aspirin, TDZD-ibuprofen, and TDZD-MMB analogs) were prepared and evaluated for their inhibition of protein aggregation in human cells and in nematodes, for anticancer activity against the MV4-11 leukemia cell line, and against a large panel of human tumor cell lines derived from nine different cancer types. The iodo-TDZD analog (PNR-962) and TDZD-aspirin analog (BSK-179) extended the lifespan of wild-type nematodes to an identical degree, increasing the mean by roughly 25-30%. The TDZD-ibuprofen analog (BSK-137), reduced aggregate intensity by 34%.

Three of the hybrid drugs (BSK-259, BSK-226 and BSK-230) exhibited the most potent growth inhibition, with GI₅₀ values in the range 280-900 nM against human leukemia cell lines. Compound BSK-226 also had GI₅₀ values 530 nM and 560 nM against cell lines NCI-H₅₂₂ (non-small-cell lung cancer) and HCT-116 (colon cancer), respectively. Compound BSK-259 was the most potent compound against the MV4-11 cell line with an LD₅₀ value (50% lethality) of 10 nM. The results from this study clearly show that several analogs of MMB-TDZD provide significantly improved anticancer activity over previous cytotoxic TDZD drugs. Thus, both BSK-259 and BSK-226 are considered potential lead molecules in the search for new anticancer agents that can be used as treatments for both hematopoetic and solid tumors.

c. Kinase Assay Suggests TDZD Analogs and PNR962 Inhibit GSK3β

The GSK3β activity assay kit (BPS Bioscience) was used, with modification of the manufacturer's protocol. The assay was done in three replicates at three doses (0.01, 0.1, and 1 μM) of each inhibitor (TDZD8, PNR962) in a 96-well plate, using 1% DMSO (final solvent concentration) as the drug-free control. The kinase assay reaction was incubated for 45 minutes at 30′C and Kinase-Glo Max Assay (Promega) was then added and incubated for 15 minutes at room temperature. A negative control contained all the reagents except the test inhibitors and GSK3β enzyme and positive control contained all reagents except the test inhibitor. SpectraMax M3 (Molecular Devices, LLC) was used as a microplate reader. The luminescence negative control reading was used as 0% activity, positive control reading was used as 100% activity and kinase assay was based on the fact that all the reading in wells with test inhibitor, should lie between these values of negative control and positive controls. GSK3β inhibitions were calculated after subtracting negative control values from all the wells and graphs were generated using GraphPad Prism.

d. Synthetic Protocol of TDZD-Aspirin Analogs

TDZD-aspirin analogs were synthesized by dissolving 2-acetoxybenzoic acid in dichloromethane and converting it to its acid chloride by reaction with oxalyl chloride followed by addition of a few drops of dimethyl formamide. After CO₂ gas evolution ceased, the mixture was concentrated and immediately reacted with simple and substituted 4-(4-(aminomethyl)benzyl)-2-(2-chloroethyl)-1,2,4-thiadiazolidine-3,5-diones and N,N-diisopropylethylamine (DIPEA) in dichloromethane, by drop-wise addition of crude 2-acetoxybenzoic acid chloride in dichloromethane (DCM). The resulting reaction mixture was stirred for 1 hr to obtain the appropriate TDZD-aspirin amide conjugate.

e. Effects of PNR962 on Protein Aggregation in Human Cells

Following previously described experimental procedures (Liu, et al. (2005). S100B-induced microglial and neuronal IL-1 expression is mediated by cell type-specific transcription factors. Journal of Neurochemistry. 92(3): 546-553), SH-SY5Y-APP_{Sw} neuronal cells expressing an aggregation-prone double mutant of amyloid precursor protein (APP_{Sw}) were cultured in DMEM plus 10% (v/v) fetal bovine serum (FBS) at 37° C. Cells were suspended in trypsin/EDTA and rinsed in PBS prior to re-plating or harvesting. Prior to assay, cells were grown 48 h in the presence of TDZD analogues at 4 doses (0.001, 0.01, 0.1, and 1 μM) dissolved in DMSO (diluted in culture medium to 0.02% final DMSO concentration) or the same final concentration of DMSO for control cells. Human neuroblastoma cells were treated with PNR962, either simultaneous with sAPPα or beginning 1 h prior to sAPPα addition (pre-treatment) in order to ascertain the protective benefits of PNR962 on protein aggregation in human neuroblastoma cell lines. A similar experimental procedure was followed to examine the effect of treatment with these analogs on non-neuronal human cells, exposing human embryonic kidney cells (HEK) that express tau-like aggregates (found in AD-associated diseases) to 0.001, 0.01, and 1-μM doses of TDZD-8 and its analogues.

f. Thioflavin-T and Antibody Staining of Amyloid and Tau in Human-Cultured Cells

To observe the protective effects of the novel TDZD analogs on protein aggregation in SY5Y-APP_{Sw} cells, using DMEM medium containing 10% fetal calf serum in T98G flasks, cells were cultured at 37′C for 26 hrs allowing cells to approximately double in number. After 48 h of cell incubation in the presence of specified concentrations of drug or vehicle, cells were fixed 15 min in formaldehyde (4% v/v), washed, and stained 20 min at ˜22° C. in a dark container with 0.1% w/v Thioflavin T mixed with DAPI (1 μg/ml; Life Technologies, Grand Island, N.Y., USA). After four washes with PBS, cells were covered with Antifade and their fluorescent images captured on a Nikon DS-Fi2 camera mounted on a Nikon C2 inverted microscope with motorized stage for automated well-by-well imaging, using the appropriate filters, DAPI/blue and Thioflavin T/green (the latter at an excitation of 358 nm and emission of 461 nm). Methodology for immunohistochemistry has been outlined in previous work (Balasubramaniam, et al. (2018) Structural insights into pro-aggregation effects of C. elegans CRAM-1 and its human ortholog SERF2. Scientific Reports, 8). The intensity of aggregate/thioflavin-T fluorescence per field was quantified via an Image J plug-in developed in-house, and normalized to the number of DAPI⁺ nuclei counted per field, to obtain an average intensity of aggregates per cell for each treatment.

g. Nematode C. elegans Strains

All nematode strains used in this research were obtained from the Caenorhabditis Genetics Center (CGC; Minneapolis, Minn., USA): wild-type Bristol-N2 [DRM stock]; CL4176, [smg-1^{ts}; myo-3p:Aβ₁₋₄₂::let-851 3′-UTR; rol-6(su1006)] which expresses human Aβ₁₋₄₂ in body wall muscle and AM141 that expresses polyglutamine [Q40]fused in-frame to YFP [Q40::YFP] in muscle cells. C. elegans strains were maintained at 20° C. on 2% (w/v) agar plates in nematode growth medium (NGM), seeded in the center with E. coli strain OP50.

h. Effects of Novel TDZD Analogs on Protein Aggregation in C. elegans strain AM141

Fresh agar plates supporting bacterial lawns, which serve as food for C. elegans, were prepared at least 1 day ahead of use. C. elegans AM141 (forming Q40::YFP aggregates in muscle, progressively in early adulthood) were placed on these 100-mm plates. Day-4 adult worms were lysed with alkaline hypochlorite solution to obtain eggs from worms, a procedure that permits synchronization of worms in a cohort. Eggs were placed on plates spotted with different concentrations of TDZD analogues after allowing the compounds to be evenly distributed on the plates (˜1 hour after adding drugs to plates). Vehicle-only (DMSO) controls are included in each experiment, as a baseline for drug effects in experimental groups. Young-adult AM141 (day-3 adults) were transferred on alternate days onto fresh plates that contained the same doses of compounds, with fresh E. coli to prevent starvation of worms. Equal samples (n=25) were randomly picked from experimental groups and control group for microscopic imaging. Using an in-house plugin for ImageJ software (NIH), several images of AM141 were processed for aggregate count per worm. The fluorescence intensity of each aggregate (indicating the size/content of protein aggregates) was also measured. Each drug treatment was repeated multiple times to validate the results. Data were displayed as bar charts of counts or fluorescence intensities (mean±SEM) per worm for each treatment group.

i. Age-Progressive Paralysis and Chemotaxis Assays in Ab-Transgenic Nematode Strain CL4176

Paralysis assay was carried out in C. elegans strains carrying an Aβ transgene, and capable of induction to express Aβ₁₋₄₂ in muscle (CL4176) or in neurons (CL2355). Worms were maintained at 20° C. with ample E. coli (0P50) bacteria, and lysed at day 3.5 post-hatch (adult day 1), releasing unlaid eggs to generate a synchronized cohort. Eggs were plated on 100-mm Petri dishes containing NGM-agar seeded in a central area with OP50 bacteria plus drug or vehicle (to a final concentration of 0.02% v/v DMSO). Worms were either upshifted to 25.5° C. at the L3-L4 transition to induce expression of the human Aβ1.42 transgene and assayed after a further 48 hr., or were aged without induction and assayed at a series of later times. Paralysis and chemotaxis assays (Dostal and Link, 2010) were performed as described previously (Ayyadevara, et al. (2016) Proteins that mediate protein aggregation and cytotoxicity distinguish Alzheimer's hippocampus from normal controls. Aging Cell, 15(5): 924-939).

j. Lifespan Studies on the Nematode C. elegans Wild-Type Bristol-N2 DRM

To obtain synchronized eggs for lifespan studies, worms were maintained for 2 generations, free of contamination and starvation. Healthy, well-fed adult worms from the 2^{nd} generation were then lysed, and eggs placed on plates with appropriate doses of TDZD analogue PNR962, or DMSO vehicle alone (for a final concentration of 0.02% v/v DMSO). At the L4 larval stage, worms were transferred daily to fresh plates for 7 days, and thereafter on alternate days until all the worms are dead. C. elegans that moved spontaneously or responded to gentle prodding were scored as alive. For the mortality computation, worms that were lost for reasons other than natural death were censored. The methods described were adopted from previously described experimental procedures (Ayyadevara, et al. (2016) PIP3-binding proteins promote age-dependent protein aggregation and limit survival in C. elegans. Oncotarget, 7(31): 48870-48886; Bharill, et al. (2013) Extreme Depletion of PIP3 Accompanies the Increased Life Span and Stress Tolerance of PI3K-null C. elegans Mutants. Frontiers in Genetics, 4).

4. Results

a. Computational Modeling and Docking Studies

Since TDZD-8 is effective in binding and inhibiting GSK-3, simple TDZD-8 analogues and dual-drug combinations with TDZD were screened alongside other effective anti-aggregative molecules including aspirin, ibruprofen, and quinolones. The combination drugs were tested for their ability to bind and inhibit GSK-3. Previous computational studies using Glide docking and MMBGSA assay have demonstrated that the lead TDZD analog, TDZD-8, binds to the allosteric hydrophobic pocket of the inactive conformation of GSK3β. Here, the predicted binding site of TDZD-8 was used (i.e., allosteric pocket in GSK3β) and previously described computational modeling and simulation studies of novel analogs of TDZD (chiefly PNR-886 and PNR-962) were followed.

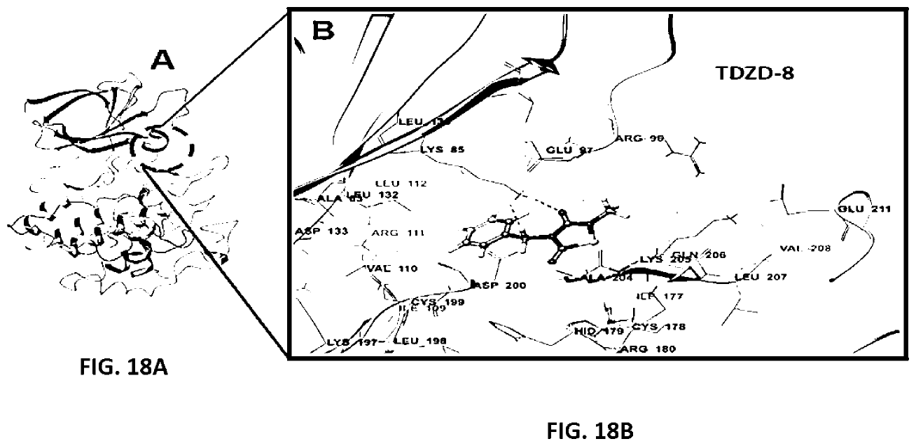

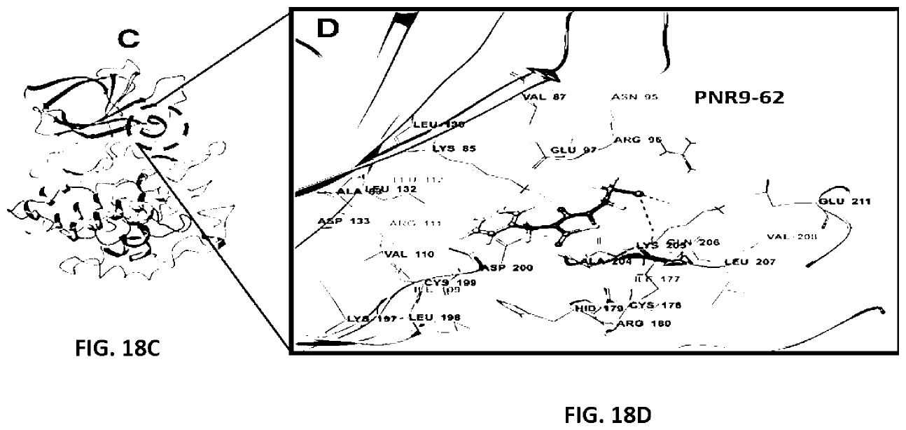

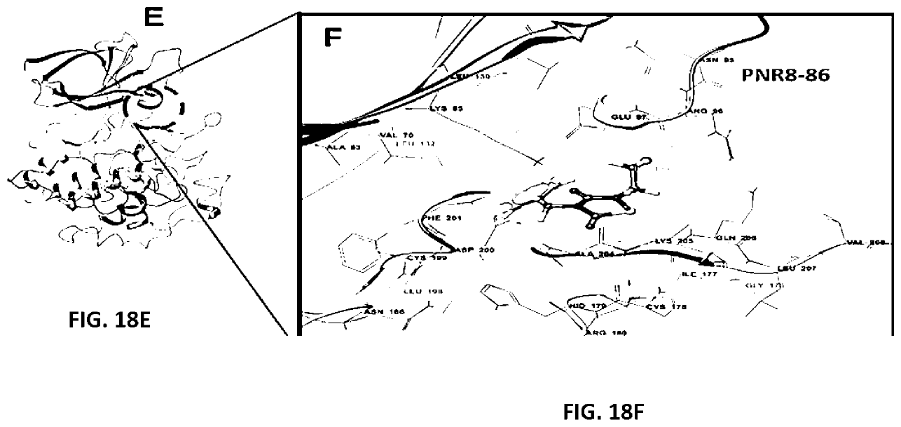

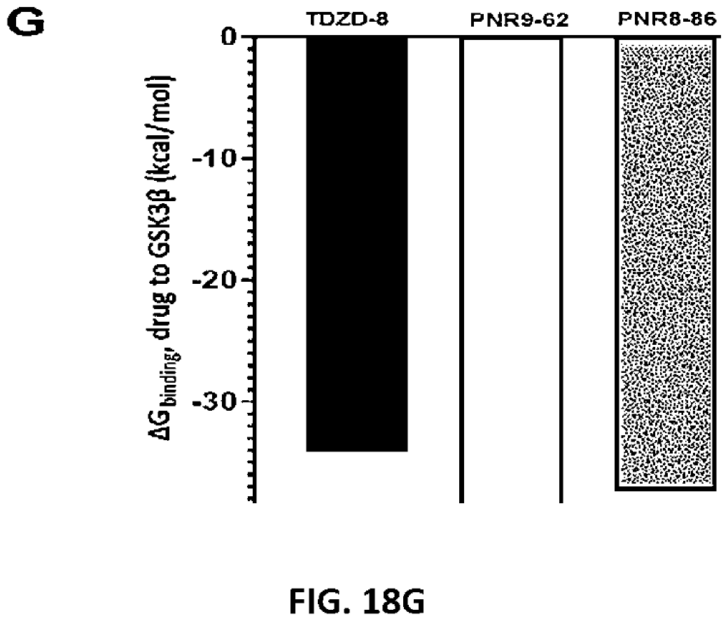

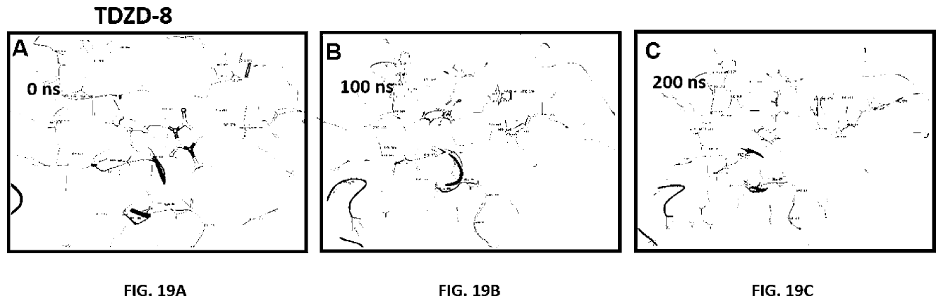

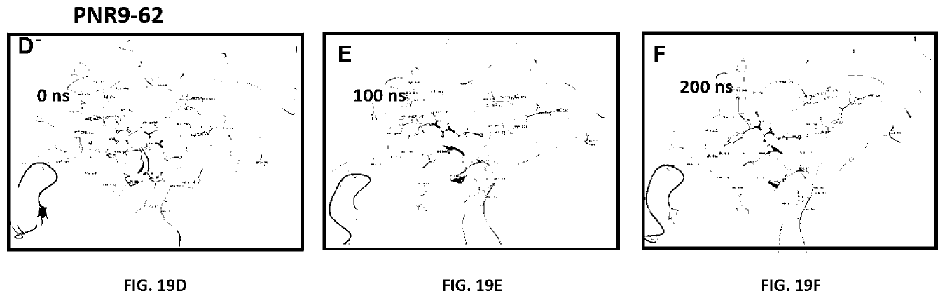

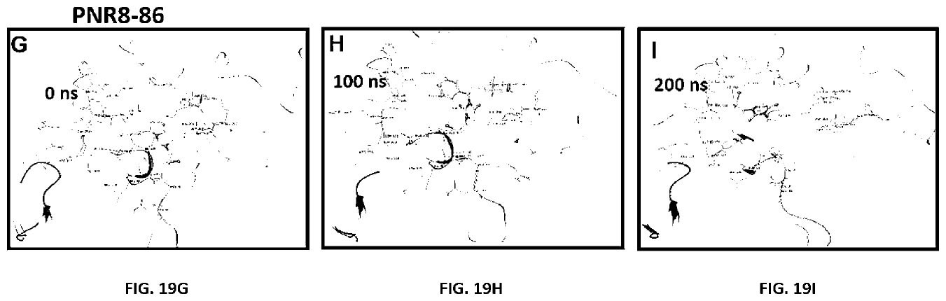

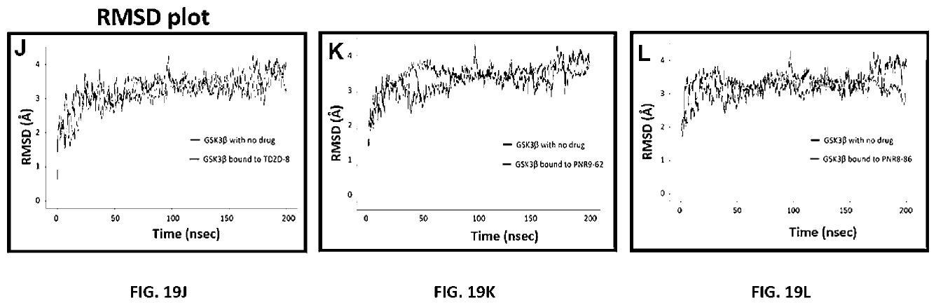

Computational modeling and docking studies predicted that PNR886 and PNR962 bind to the same allosteric hydrophobic pocket in GSK3β shown in FIG. 18A-F. Compared to TDZD-8, both PNR-886 and PNR-962 have greatly improved binding affinity for GSK3β. The Gibbs Free Energy of binding (ΔG_{binding}) calculated by the MM/GBSA method for PNR886 and PNR962 predicts that both drugs have high binding affinity for GSK3β in the inactive conformation (FIG. 18G). Snapshots were taken from 0.5-μs simulations of full-length GSK3β bound to TDZD-8 and its analogs PNR886 and PNR962, at 0-ns, 100-ns and 200-ns (FIG. 19A-I). Root Mean Square Deviation (RMSD) of protein-ligand complexes, during 200-ns simulations of GSK3β binding to TDZD-8, PNR962, and PNR886, predict stable binding of these compounds to the allosteric hydrophobic pocket of the inactive GSK3β conformation (FIG. 19J-L). Schrödinger Maestro 11.4 (https://www.schrodinger.com/) was used to depict the docked molecular structures.

b. Analysis of Drug Docking with GSK-3β

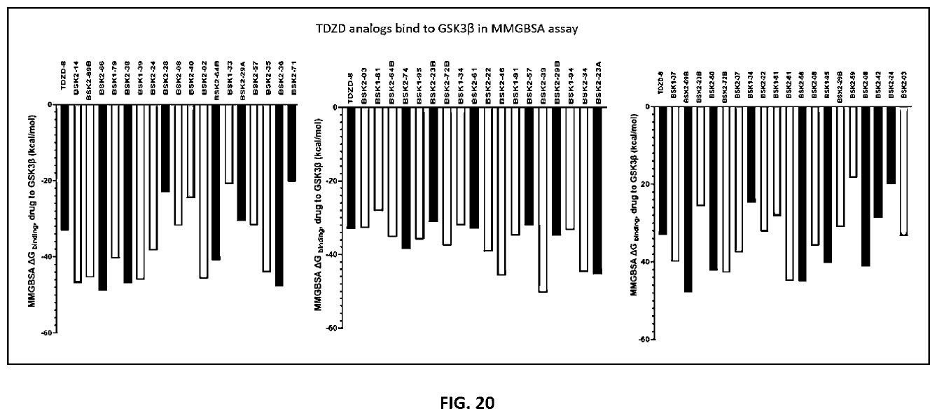

Drug docking with GSK-3β (by SeeSAR) was analyzed to identify GSK3β inhibitors effective in the nM range. As illustrated in FIG. 20, virtual screening of TDZD analogs, including dual-drug compounds, was performed to assess stable binding to GSK3β. A total of 55 compounds are shown, of which 28 (51%) are predicted to bind GSK3β with greater affinity than the parent drug, TDZD8, using the MM/GBSA protocol.

c. Analysis of Best Predicted Inhibitors

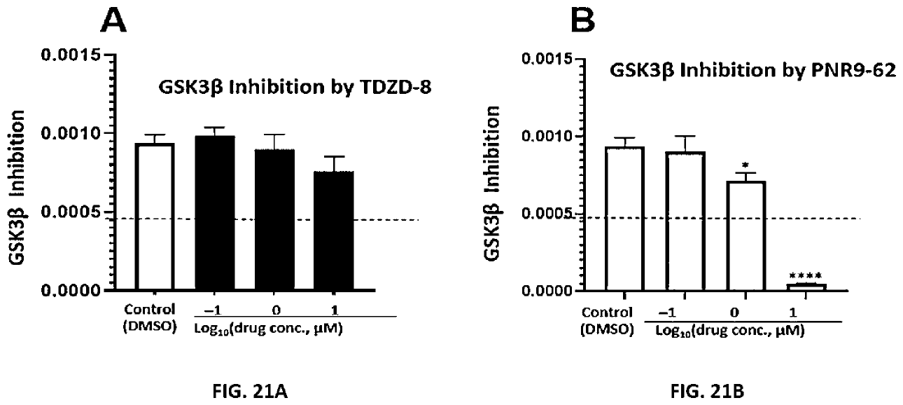

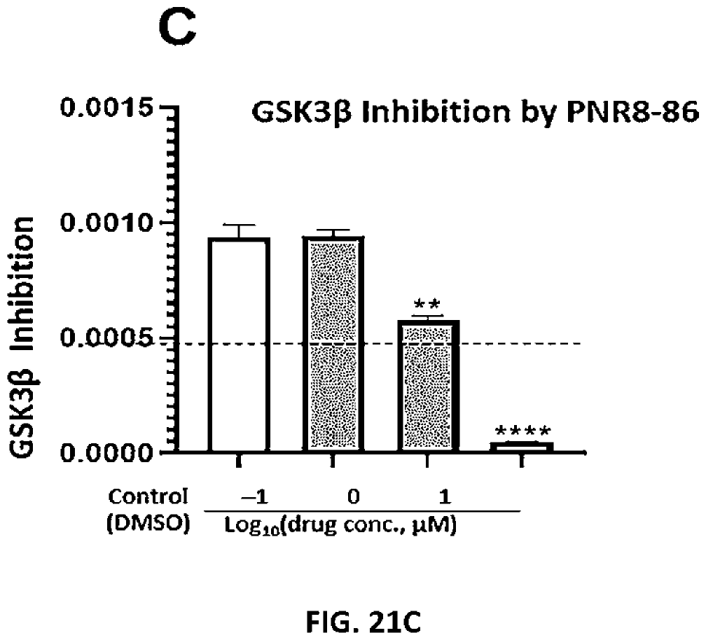

The best predicted inhibitors were synthesized, and screened in both human neuronal cells, and in C. elegans models of neurodegeneration. These compounds were also tested for their ability to inhibit GSK3β activity in vitro. Here, data is shown for PNR962 and PNR886, both of which were markedly superior to TDZD8 in their ability to inhibit GSK3β activity in vitro (FIG. 21A-C), as well as in several in vivo models (not shown).

d. Qsar Studies

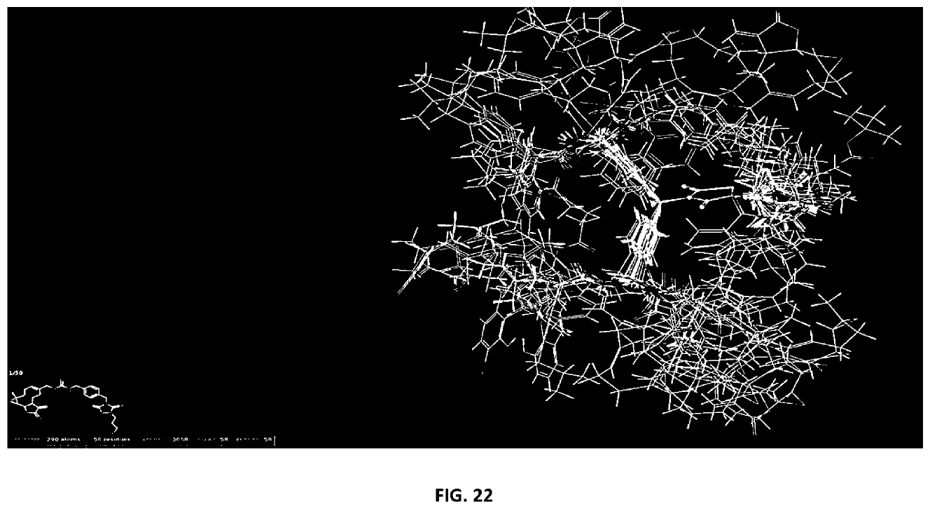

QSAR (quantitative structure-activity relationship) studies were performed based on dose-response curves in cultured human neuronal cells (SY5Y-APP_{Sw}). See FIG. 22.

e. Impact on Tau Hyperphosphorylation

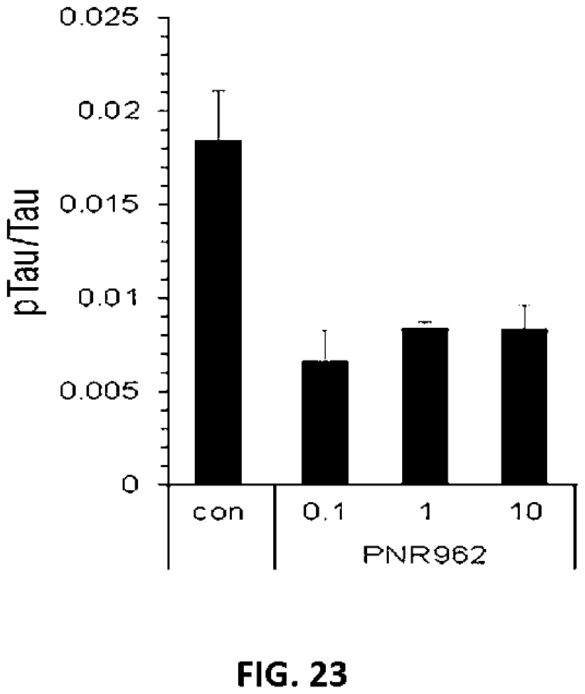

Microtubule assembly involves stable tau binding to other proteins in the complex, including tubulin. Hyperphosphorylated tau is readily sequestered into aggregates and generates intracellular neurofibrillary tangles (“tau tangles”). GSK3β was shown to phosphorylate tau at serine 202 and threonine 205, initiating hyperphosphorylation of tau at other sites. The top drugs were tested for impact on tau hyperphosphorylation in rat cortical cells (FIG. 23).

f. Mouse Testing

Mice are now being treated with PNR962, one of the lead compounds in this screen, for its efficacy in reducing aggregation and improving cognition (assessed by novel-object recognition and Morris water-maze tests). For this assessment, BRI-Aβ transgenic mice, a model of age-dependent, cerebral amyloid deposition, are used.

g. Neuronal Cultures

Primary cultures were established from cerebral cortex of E18 rats; under serum-free conditions (Neurobasal/B27) these cultures contain ˜85% neurons, with the remainder comprising astrocytes (˜12% and microglia ˜3%). At the 8^{th} population doubling, triplicate cultures were exposed to the test compounds at the indicated concentrations; a separate triplicate was exposed to vehicle (0.5% DMSO final concentration). After 24 h, cultures were washed once with ice-cold PBS and then scraped in RIPA buffer containing inhibitors of proteases and phosphatases. Lysates were centrifuged at 14,000 g at 4° C. for 7 min. Supernatants were removed and stored at −80° C. after removal of small aliquots for determination of protein concentration by BCA. Samples equilibrated for total protein were analyzed by Wes™ capillary-gel immunodetection (ProteinSimple) using the AT8 clone of anti-phosphoTau (Invitrogen MN 1020) or total Tau (ABclonal A0002). Detected antigen was calculated by peak area (automated peak fit), and values are reported as AT8 normalized to total Tau.

5. Prospective Risk Reduction and/or Delay of Onset, for Alzheimer's, Parkinson's, and/or Huntington's Disease and/or Other Dementias or Neurodegenerative Diseases

a) Risk reduction, or delay of onset, for progression from mild cognitive impairment to Alzheimer's disease, Parkinson's disease, or Huntington's disease, and/or other dementias or neurodegenerative diseases

b) Risk reduction, or delay of onset, of diagnosis for Alzheimer's disease, Parkinson's disease, Huntington's disease, and/or other neurodegenerative diseases or dementias, for those individuals identified as being at high risk, due to (a.) family history of such diseases; (b.) traumatic brain injury; (c.) recurrent head trauma; or (d.) Down Syndrome, or partial trisomy of chromosome 21 which might constitute a mild form of Down Syndrome.

c) Reduction in the rate of progression, or possibly reversal, of Alzheimer's disease, Parkinson's disease, Huntington's disease, and/or other neurodegenerative diseases or dementias, following clinical diagnosis.

d) Risk reduction, or delay in onset and/or progression of, other diseases and disease-predisposing conditions that feature protein aggregation and/or aggregation-associated inflammation, including but not limited to: diabetes, insulin resistance, cardiovascular disease, hypertension, peripheral artery disease, kidney disease or insufficiency, sarcopenia, cachexia, rheumatism, rheumatoid arthritis, and osteoarthritis.

### I. REFERENCES

Martinez A., Fernandez E., Castro A., et al. N-Benzylpiperidine derivatives of 1,2,4-thiadiazolidinone as new acetylcholinesterase inhibitors. Eur J Med Chem. 2000; 35(10): 913-922.
Martinez A., Castro A., Dorronsoro I., Alonso M. Glycogen synthase kinase 3 (GSK-3) inhibitors as new promising drugs for diabetes, neurodegeneration, cancer, and inflammation. Med Res Rev. 2002; 22(4): 373-384.
Martnez A., Alonso M., Castro A., Perez C., Moreno F. J. First non-ATP competitive glycogen synthase kinase 3 beta (GSK-3beta) inhibitors: thiadiazolidinones (TDZD) as potential drugs for the treatment of Alzheimer's disease. J Med Chem. 2002; 45(6): 1292-1299.
Martinez A., Alonso M., Castro A., et al. SAR and 3D-QSAR studies on thiadiazolidinone derivatives: exploration of structural requirements for glycogen synthase kinase 3 inhibitors. J Med Chem. 2005; 48(23): 7103-7112.
Martinez A., Castro A., Cardelús I., Llenas J., Palacios J. Arylimino-1,2,4-thiadiazolidinones: A new family of potassium channel openers. Bioorganic & Medicinal Chemistry. 1997; 5(7): 1275-1283.
Martinez A., Alonso D., Castro A., et al. Synthesis and potential muscarinic receptor binding and antioxidant properties of 3-(thiadiazolyl)pyridine 1-oxide compounds. Arch Pharm (Weinheim). 1999; 332(6): 191-194.
Dominguez J. M., Fuertes A., Orozco L., del Monte-Millan M., Delgado E., Medina M. Evidence for irreversible inhibition of glycogen synthase kinase-3beta by tideglusib. J Biol Chem. 2012; 287(2): 893-904.
Bartolucci A. A., Tendera M., Howard G. Meta-analysis of multiple primary prevention trials of cardiovascular events using aspirin. Am J Cardiol. 2011; 107(12): 1796-1801.
Ong G., Davis T. M., Davis W. A. Aspirin is associated with reduced cardiovascular and all-cause mortality in type 2 diabetes in a primary prevention setting: the Fremantle Diabetes study. Diabetes Care. 2010; 33(2): 317-321.
Szekely C. A., Green R. C., Breitner J. C., et al. No advantage of A beta 42-lowering NSAIDs for prevention of Alzheimer dementia in six pooled cohort studies. Neurology. 2008; 70(24): 2291-2298.
Ayyadevara S., Balasubramaniam M., Kakraba S., Alla R., Mehta J. L., Shmookler Reis R. J. Aspirin-Mediated Acetylation Protects Against Multiple Neurodegenerative Pathologies by Impeding Protein Aggregation. Antioxid Redox Signal. 2017; 27(17): 1383-1396.
Taylor J. L., Brown J. H. Inhibition of protein aggregation by vitamin E and selenium. Proc Soc Exp Biol Med. 1974; 145(1): 32-36.
Halicka H. D., Zhao H., Li J., et al. Potential anti-aging agents suppress the level of constitutive mTOR- and DNA damage-signaling. Aging (Albany N.Y.). 2012; 4(12): 952-965.
Pomponi M. F., Gambassi G., Pomponi M., Di Gioia A., Masullo C. Why docosahexaenoic acid and aspirin supplementation could be useful in women as a primary prevention therapy against Alzheimer's disease? Ageing Res Rev. 2011; 10(1): 124-131.
Ayyadevara S., Bharill P., Dandapat A., et al. Aspirin inhibits oxidant stress, reduces age-associated functional declines, and extends lifespan of Caenorhabditis elegans. Antioxid Redox Signal. 2013; 18(5): 481-490.
Strong R., Miller R. A., Astle C. M., et al. Nordihydroguaiaretic acid and aspirin increase lifespan of genetically heterogeneous male mice. Aging Cell. 2008; 7(5): 641-650.
Pignone M., Alberts M. J., Colwell J. A., et al. Aspirin for primary prevention of cardiovascular events in people with diabetes: a position statement of the American Diabetes Association, a scientific statement of the American Heart Association, and an expert consensus document of the American College of Cardiology Foundation. Diabetes Care. 2010; 33(6): 1395-1402.
Ching T. T., Chiang W. C., Chen C. S., Hsu A. L. Celecoxib extends C. elegans lifespan via inhibition of insulin-like signaling but not cyclooxygenase-2 activity. Aging Cell. 2011; 10(3): 506-519.
Guzman M. L., Li X., Corbett C. A., et al. Rapid and selective death of leukemia stem and progenitor cells induced by the compound 4-benzyl, 2-methyl, 1,2,4-thiadiazolidine, 3,5 dione (TDZD-8). Blood. 2007; 110(13): 4436-4444.
Nasim S., Guzman M. L., Jordan C. T., Crooks P. A. Discovery of 1,2,4-thiadiazolidine-3,5-dione analogs that exhibit unusual and selective rapid cell death kinetics against acute myelogenous leukemia cells in culture. Bioorg Med Chem Lett. 2011; 21(16): 4879-4883.
Chaturvedi D. Sesquiterpene lactones: structural diversity and their biological activities, In-Opportunity, Challanges and Scope of Natural Products in Medicinal Chemistry. ISBN: 978-81-308-0448-4, Research Signpost, Trivandrum. 2011: 313-334.
Chadwick M., Trewin H., Gawthrop F., Wagstaff C. Sesquiterpenoids lactones: benefits to plants and people. Int J Mol Sci. 2013; 14(6): 12780-12805.
Irmgard M. Perspectives on Sesquiterpene Lactones in Inflammation and Cancer. Current Drug Targets. 2011; 12(11): 1560-1573.
Orofino Kreuger M. R., Grootjans S., Biavatti M. W., Vandenabeele P., D'Herde K. Sesquiterpene lactones as drugs with multiple targets in cancer treatment: focus on parthenolide. Anti-Cancer Drugs. 2012; 23(9): 883-896.
Knight D. W. Feverfew: chemistry and biological activity. Nat Prod Rep. 1995; 12(3): 271-276.
Bork P. M., Schmitz M. L., Kuhnt M., Escher C., Heinrich M. Sesquiterpene lactone containing Mexican Indian medicinal plants and pure sesquiterpene lactones as potent inhibitors of transcription factor NF-kappaB. FEBS Lett. 1997; 402(1): 85-90.
Wen J., You K. R., Lee S. Y., Song C. H., Kim D. G. Oxidative stress-mediated apoptosis. The anticancer effect of the sesquiterpene lactone parthenolide. J Biol Chem. 2002; 277(41): 38954-38964.
Helmer S. P., Heinrich M., Bork P. M., et al. Sesquiterpene lactones specifically inhibit activation of NF-kappa B by preventing the degradation of I kappa B-alpha and I kappa B-beta. J Biol Chem. 1998; 273(3): 1288-1297.
Sweeney C., Li L., Shanmugam R., et al. Nuclear factor-kappaB is constitutively activated in prostate cancer in vitro and is overexpressed in prostatic intraepithelial neoplasia and adenocarcinoma of the prostate. Clin Cancer Res. 2004; 10(16): 5501-5507.
Yip-Schneider M. T., Nakshatri H., Sweeney C. J., Marshall M. S., Wiebke E. A., Schmidt C. M. Parthenolide and sulindac cooperate to mediate growth suppression and inhibit the nuclear factor-kappa B pathway in pancreatic carcinoma cells. Mol Cancer Ther. 2005; 4(4): 587-594.
Nozaki S., Sledge G. W., Jr., Nakshatri H. Repression of GADD153/CHOP by NF-kappaB: a possible cellular defense against endoplasmic reticulum stress-induced cell death. Oncogene. 2001; 20(17): 2178-2185.
Guzman M. L., Rossi R. M., Kamischky L., et al. The sesquiterpene lactone parthenolide induces apoptosis of human acute myelogenous leukemia stem and progenitor cells. Blood. 2005; 105(11): 4163-4169.
Guzman M. L., Jordan C. T. Feverfew: weeding out the root of leukaemia. Expert Opin Biol Ther. 2005; 5(9): 1147-1152.
Dai Y., Guzman M. L., Chen S., et al. The NF (Nuclear factor)-kappaB inhibitor parthenolide interacts with histone deacetylase inhibitors to induce MKK7/JNK1-dependent apoptosis in human acute myeloid leukaemia cells. Br J Haematol. 2010; 151(1): 70-83.
Kim Y. R., Eom J. I., Kim S. J., et al. Myeloperoxidase Expression as a Potential Determinant of Parthenolide-Induced Apoptosis in Leukemia Bulk and Leukemia Stem Cells. Journal of Pharmacology and Experimental Therapeutics. 2010; 335(2): 389-400.
Deschler B., Lubbert M. Acute myeloid leukemia: epidemiology and etiology. Cancer. 2006; 107(9): 2099-2107.
Estey E., Milner H. Acute myeloid leukaemia. The Lancet. 2006; 368(9550): 1894-1907.
Löwenberg B., Suciu S., Archimbaud E., et al. Mitoxantrone versus daunorubicin in induction-consolidation chemotherapy—the value of low-dose cytarabine for maintenance of remission, and an assessment of prognostic factors in acute myeloid leukemia in the elderly: final report. European Organization for the Research and Treatment of Cancer and the Dutch-Belgian Hemato-Oncology Cooperative Hovon Group. Journal of Clinical Oncology. 1998; 16(3): 872-881.
Tazzari P. L., Cappellini A., Ricci F., et al. Multidrug resistance-associated protein 1 expression is under the control of the phosphoinositide 3 kinase/Akt signal transduction network in human acute myelogenous leukemia blasts. Leukemia. 2007; 21(3): 427-438.
Nasim S., Crooks P. A. Antileukemic activity of aminoparthenolide analogs. Bioorganic & Medicinal Chemistry Letters. 2008; 18(14): 3870-3873.
S. El-Feraly F. Melampolides from Magnolia grandiflora. Phytochemistry. 1984; 23(10): 2372-2374.
Macias F. A., Galindo J. C. G., Massanet G. M. Potential allelopathic activity of several sesquiterpene lactone models. Phytochemistry. 1992; 31(6): 1969-1977.
Nasim S., Pei S., Hagen F. K., Jordan C. T., Crooks P. A. Melampomagnolide B: a new antileukemic sesquiterpene. Bioorg Med Chem. 2011; 19(4): 1515-1519.
Janganati V., Penthala N. R., Madadi N. R., Chen Z., Crooks P. A. Anti-cancer activity of carbamate derivatives of melampomagnolide B. Bioorg Med Chem Lett. 2014; 24(15): 3499-3502.
Janganati V., Ponder J., Jordan C. T., Borrelli M. J., Penthala N. R., Crooks P. A. Dimers of Melampomagnolide B Exhibit Potent Anticancer Activity against Hematological and Solid Tumor Cells. J Med Chem. 2015; 58(22): 8896-8906.
Albayati Z. A. F., Janganati V., Chen Z., et al. Identification of a melampomagnolide B analog as a potential lead molecule for treatment of acute myelogenous leukemia. Bioorg Med Chem. 2017; 25(3): 1235-1241.
Bommagani S., Ponder J., Penthala N. R., et al. Indole carboxylic acid esters of melampomagnolide B are potent anticancer agents against both hematological and solid tumor cells. Eur J Med Chem. 2017; 136: 393-405.
Janganati V., Ponder J., Thakkar S., Jordan C. T., Crooks P. A. Succinamide derivatives of melampomagnolide B and their anti-cancer activities. Bioorg Med Chem. 2017; 25(14): 3694-3705.
Janganati V., Ponder J., Balasubramaniam M., et al. MMB triazole analogs are potent NF-kappaB inhibitors and anti-cancer agents against both hematological and solid tumor cells. Eur J Med Chem. 2018; 157: 562-581.
Slomczyfiska U., Barany G. Efficient synthesis of 1,2,4-dithiazolidine-3,5-diones (dithiasuccinoyl-amines) and observations on formation of 1,2,4-thiadiazolidine-3,5-diones by related chemistry. Journal of Heterocyclic Chemistry. 1984; 21(1): 241-246.
Nasim S., Crooks P. A. N-Chlorosuccinimide is a convenient oxidant for the synthesis of 2,4-disubstituted 1,2,4-thiadiazolidine-3,5-diones. Tetrahedron Letters. 2009; 50(3): 257-259.
Ayyadevara S., Engle M. R., Singh S. P., et al. Lifespan and stress resistance of Caenorhabditis elegans are increased by expression of glutathione transferases capable of metabolizing the lipid peroxidation product 4-hydroxynonenal. Aging Cell. 2005; 4(5): 257-271.
Lou G., Chen J., Xia Y. Effects of low-dose aspirin in subjects with dyslipidemia. Lipids Health Dis. 2016; 15: 106.
Shebl F. M., Sakoda L. C., Black A., et al. Aspirin but not ibuprofen use is associated with reduced risk of prostate cancer: a PLCO study. Br J Cancer. 2012; 107(1): 207-214.
Ayyadevara, S., Balasubramaniam, M., Johnson, J., Alla, R., Mackintosh, S. G., & Shmookler Reis, R. J. (2016). PIP3-binding proteins promote age-dependent protein aggregation and limit survival in C. elegans. Oncotarget, 7(31), 48870-48886.
Ayyadevara, S., Balasubramaniam, M., Parton, P. A., Barger, S. W., Griffin, W. S. T., Alla, R., . . . Shmookler Reis, R. J. (2016). Proteins that mediate protein aggregation and cytotoxicity distinguish Alzheimer's hippocampus from normal controls. Aging Cell, 15(5), 924-939.
Balasubramaniam, M., Ayyadevara, S., & Reis, R. J. S. (2018). Structural insights into pro-aggregation effects of C. elegans CRAM-1 and its human ortholog SERF2. Scientific Reports, 8.
Balasubramaniam, M., Mainali, N., Bowroju, S. K., Atluri, P., Penthala, N. R., Ayyadevera, S., . . . Shmookler Reis, R. J. (2020). Structural modeling of GSK3β implicates the inactive (DFG-out) conformation as the target bound by TDZD analogs. Scientific Reports, 10(1).
Balasubramaniam, M., Parcon, P. A., Bose, C., Liu, L., Jones, R. A., Farlow, M. R., . . . Griffin, W. S. T. (2019). Interleukin-1β drives NEDD8 nuclear-to-cytoplasmic translocation, fostering parkin activation via NEDD8 binding to the P-ubiquitin activating site. Journal of Neuroinflammation. 16(1).
Bharill, P., Ayyadevara, S., Alla, R., & Shmookler Reis, R. J. (2013). Extreme Depletion of PIP3 Accompanies the Increased Life Span and Stress Tolerance of PI3K-null C. elegans Mutants. Frontiers in Genetics. 4.
Bommagani, S., Penthala, N. R., Balasubramaniam, M., Kuravi, S., Caldas-Lopes, E., Guzman, M. L., . . . Crooks, P. A. (2019). A novel tetrazole analogue of resveratrol is a potent anticancer agent. Bioorg Med Chem Lett, 29(2), 172-178.
Dosanjh, L. E., Brown, M. K., Rao, G., Link, C. D., & Luo, Y. (2010). Behavioral phenotyping of a transgenic Caenorhabditis elegans expressing neuronal amyloid-beta. J Alzheimers Dis, 19(2), 681-690.
Dostal, V., & Link, C. D. (2010). Assaying &beta; -amyloid Toxicity using a Transgenic <em>C. elegans</em> Model. Journal of Visualized Experiments (44).
Janganati, V., Ponder, J., Balasubramaniam, M., Bhat-Nakshatri, P., Bar, E. E., Nakshatri, H., . . . Crooks, P. A. (2018). MMB triazole analogs are potent NF-κB inhibitors and anti-cancer agents against both hematological and solid tumor cells. Eur J Med Chem, 157, 562-581.
Lakkaniga, N. R., Balasubramaniam, M., Zhang, S. X., Frett, B., & Li, H. Y. (2019). Structural Characterization of the Aurora Kinase B “DFG-flip” Using Metadynamics. Aaps Journal, 22(1).
Liu, L., Li, Y. K., Van Eldik, L. J., Griffin, W. S. T., & Barger, S. W. (2005). S100B-induced microglial and neuronal IL-1 expression is mediated by cell type-specific transcription factors. Journal of Neurochemistry, 92(3), 546-553.
Park, J., McDonald, J. J., Petter, R. C., & Houk, K. N. (2016). Molecular Dynamics Analysis of Binding of Kinase Inhibitors to WT EGFR and the T790M Mutant. Journal of Chemical Theory and Computation, 12(4), 2066-2078.
Ain, Q. U., Batool, M. & Choi, S. TLR4-Targeting Therapeutics: Structural Basis and Computer-Aided Drug Discovery Approaches. Molecules 25, 627, doi:10.3390/molecules25030627 (2020).
Ain, Q. U., Batool, M. & Choi, S. TLR4-Targeting Therapeutics: Structural Basis and Computer-Aided Drug Discovery Approaches. Molecules 25, doi:10.3390/molecules25030627 (2020).
Pereira, C. A. et al. Computational approaches for drug discovery against trypanosomatid-caused diseases. Parasitology 147, 611-633, doi:10.1017/s0031182020000207 (2020).
Batool, M., Ahmad, B. & Choi, S. A Structure-Based Drug Discovery Paradigm. Int J Mol Sci 20, doi:10.3390/ijms20112783 (2019).
Vincent Rajkumar, S. The high cost of prescription drugs: causes and solutions. Blood Cancer Journal 10, doi:10.1038/s41408-020-0338-x (2020).
Gronde, T. V., Uyl-de Groot, C. A. & Pieters, T. Addressing the challenge of high-priced prescription drugs in the era of precision medicine: A systematic review of drug life cycles, therapeutic drug markets and regulatory frameworks. PLoS One 12, e0182613, doi:10.1371/joumal.pone.0182613 (2017).
Jatto, E. & Okhamafe, A. An Overview of Pharmaceutical Validation and Process Controls in Drug Development. Tropical Journal of Pharmaceutical Research 1, 115, doi:10.4314/tjpr.v1i2.14592 (2002).
Kaitin, K. I. Deconstructing the Drug Development Process: The New Face of Innovation. Clinical Pharmacology & Therapeutics 87, 356-361, doi:10.1038/clpt.2009.293 (2010).
Ponzoni, I. et al. Hybridizing Feature Selection and Feature Learning Approaches in QSAR Modeling for Drug Discovery. Sci Rep 7, 2403, doi:10.1038/s41598-017-02114-3 (2017).
Tropsha, A. Best Practices for QSAR Model Development, Validation, and Exploitation. Molecular Informatics 29, 476-488, doi:10.1002/minf.201000061 (2010).
McKenzie, P., Kiang, S., Tom, J., Rubin, A. E. & Futran, M. Can pharmaceutical process development become high tech? AIChE Journal 52, 3990-3994, doi:10.100²/aic.11022 (2006).
Gramatica, P. Principles of QSAR models validation: internal and external. QSAR & Combinatorial Science 26, 694-701, doi:10.1002/qsar.200610151 (2007).
Wang, H. et al. Design of cinnamaldehyde amino acid Schiff base compounds based on the quantitative structure-activity relationship. R Soc Open Sci 4, 170516, doi:10.1098/rsos.170516 (2017).

### J. ASPECTS

A first aspect of the present disclosure provides design and synthesis of the compounds of given general structure (Structure I), and evaluation of the compounds of general structure (Structure I), as inhibitors of protein aggregation, which are expected to delay, prevent, or reverse age-associated diseases including but not limited to sarcopenia; Alzheimer's disease and other dementias; hematologic cancers such as leukemia, lymphoma and multiple myeloma; and solid tumors such as lung cancer, liver cancer, pancreatic cancer, CNS cancers, breast cancer, ovarian cancer, colon cancer, renal cancer, melanoma, prostate cancer and head and neck cancers.

The second aspect of the present disclosure is formulation of novel TDZD analogues utilizing pharmaceutically acceptable salts, or by employing nanoparticle drug-delivery formulations.

In the third aspect, the compound comprising structure-I may be a free form or a salt. The compound salt is preferably a pharmaceutically acceptable salt. Pharmaceutically acceptable salts may include, without limitation, hydrochloride, hydrobromide, phosphate, sulfate, methane-sulfonate, acetate, formate, tartaric acid, bitartrate, stearate, phthalate, hydroiodide, lactate, monohydrate, mucate, nitrate, phosphate, salicylate, phenylpropionate, isobutyrate, hypophosphite, maleic acid, malic acid, citrate, isocitrate, succinate, lactate, gluconate, glucuronate, pyruvate, oxalate, fumarate, propionate, aspartate, glutamate, benzoate, terephthalate, and the like. In other embodiments, the pharmaceutical acceptable salt includes an alkaline or alkaline earth metal ion salt. In particular, sodium, potassium or other pharma-ceutically acceptable inorganic salts are used.

The fourth aspect of the present disclosure provides a composition of the general structures that may be chosen from (E) or (Z)-isomers, or R- and S-isomers for chiral molecules.

The fifth aspect of the present disclosure is route of administration (drug delivery) of all the aforementioned novel TDZD analogues, which may include (without limitation) oral, intravenous, intraperitoneal, intramuscular, intrathecal, intranasal, transdermal, subdermal (depot), inhalation, or buccal.

The sixth aspect of the present disclosure is that all the aforementioned novel TDZD analogues may be polymorphic in form, including amorphous or crystalline composition, or any other physical state in formulations that may enhance the pharmacokinetic properties of the molecule. The salt forms may be amorphous or in various polymeric forms including hydrates, or solvates with alcohols or other solvents.

Pharmaceutical Compositions: The disclosure also provides a pharmaceutical composition comprising the compound comprising structure I and at least one pharmaceutically acceptable excipient. One or more of the compounds described in this disclosure may be combined with at least one pharmaceutically acceptable excipient.

A pharmaceutical composition of the disclosure comprises at least one pharmaceutically acceptable excipient. Non-limiting examples of suitable excipients may include diluents, binders, fillers, buffering agents, pH modifying agents, disintegrants, dispersing agents, stabilizers, preservatives, and coloring agents. The amount and types of excipients may be selected according to known principles of pharmaceutical science.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 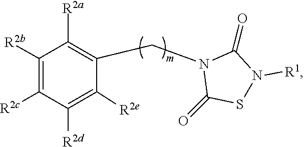
wherein m is 0, 1, 2, or 3;
wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹;
wherein R¹⁰, when present, is selected from hydrogen, —OH, C1-C10 alkyl, C1-C10 alkoxy, C1-C10 alkylamino, and (C1-C10)(C1-C10) dialkylamino;
wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and
wherein each of R^{2a}, R^{2b}, R^{2c}, R^{2d}, and R^{2e} is independently selected from hydrogen, halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, Ar¹, and a structure having a formula: 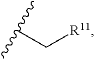
provided that one of R^{2a}, R^{2b}, R^{2c}, R^{2d}, and R^{2e} is Ar¹ or 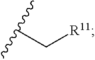
wherein R¹¹, when present, is a carboxylate residue of a chemotherapeutic agent or a carbamide residue of a chemotherapeutic agent; and
wherein Ar¹, when present, is selected from heteroaryl and aryl, and is substituted with 0, 1, 2, or 3 groups independently selected from halogen, —CN, —NH₂, —OH, —NO₂, —CO₂R²⁰, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and
wherein R²⁰, when present, is selected from hydrogen, —OH, C1-C10 alkyl, C1-C10 alkoxy, C1-C10 alkylamino, and (C1-C10)(C1-C10) dialkylamino,
provided that when m is 1, R¹ is C1-C10 alkyl, C2-C10 alkenyl, or C1-C10 haloalkyl, and one of R^{2a}, R^{2b}, R^{2c}, R^{2d}, and R^{2e} is 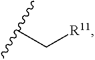
then R¹¹ is not —OC(O)₂(C1-C8 alkyl), —NHC(O)₂(C1-C8 alkyl), or —N(C1-C4 alkyl)C(O)₂(C1-C8 alkyl),
or a pharmaceutically acceptable salt thereof.

2. The compound of claim 1, wherein the carboxylate or carbamide residue is selected from: 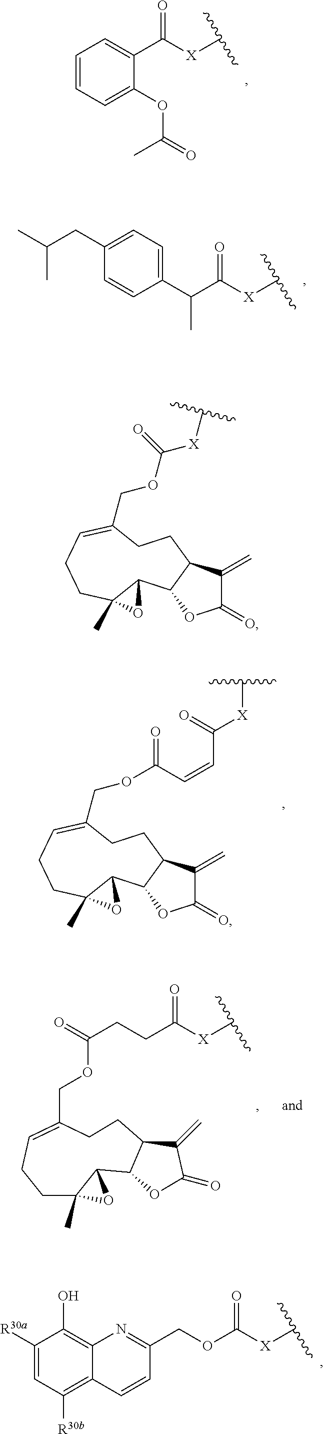
wherein X is selected from NH and O; and
wherein each of R^{30a} and R^{30b}, when present, is independently selected from hydrogen, —Cl, —Br, and —I.

3. The compound of claim 1, wherein m is 1.

4. (canceled)

5. The compound of claim 1, wherein R¹ is selected from C1-C4 alkyl, C2-C4 alkenyl, C1-C4 haloalkyl, C1-C4 cyanoalkyl, C1-C4 nitroalkyl, C1-C4 hydroxyalkyl, C1-C4 alkoxy, C1-C4 alkenoxy, C1-C4 thioalkyl, C1-C4 alkylthiol, C1-C4 alkylamino, (C1-C4)(C1-C4) dialkylamino, C1-C4 aminoalkyl, —(C1-C4 alkyl)-O—(C1-C4 alkyl), —(C1-C4 alkyl)C(O)R¹⁰, —(C1-C4 alkyl)OC(O)(C1-C4 alkyl), —(C1-C4 alkyl)NHC(O)(C1-C4 alkyl), —(C1-C4 alkyl)N(C1-C4 alkyl)C(O)(C1-C4 alkyl), —(C1-C4)Cy¹, and Cy¹.

6. The compound of claim 1, wherein R¹ is selected from C1-C10 alkyl, C1-C10 haloalkyl, and Cy¹.

7-9. (canceled)

10. The compound of claim 1, wherein Cy¹, when present, is C6 aryl monosubstituted with a group selected from halogen, —CN, and C1-C4 alkoxy or wherein Cy¹, when present, is unsubstituted C6 aryl.

11-12. (canceled)

13. The compound of claim 1, wherein three of R^{2a}, R^{2b}, R^{2c}, R^{2d}, and R^{2e} are hydrogen and one of R^{2a}, R^{2b}, R^{2c}, R^{2d}, and R^{2e} is selected from Ar¹ and

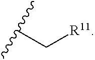

14-16. (canceled)

17. The compound of claim 13, wherein R¹¹ is selected from: 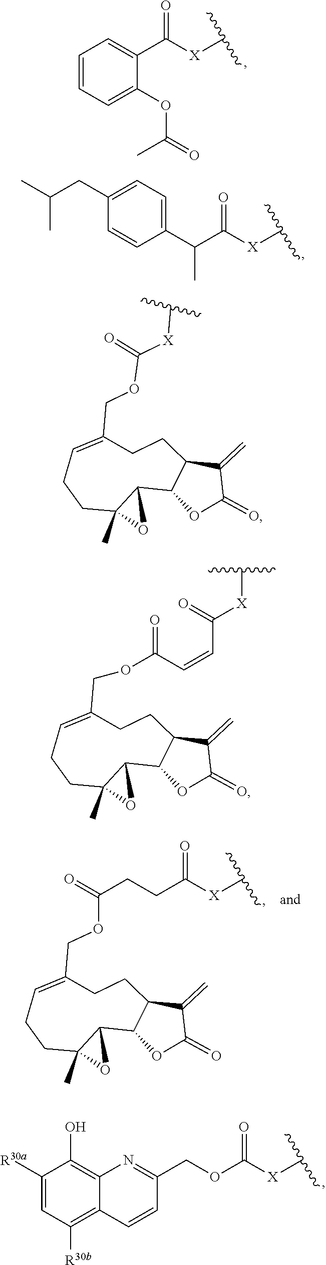
wherein X is selected from NH and O; and
wherein each of R^{30a} and R^{30b}, when present, is independently selected from hydrogen, —Cl, —Br, and —I.

18. The compound of claim 1, wherein one of R^{2a}, R^{2b}, R^{2c}, R^{2d}, and R^{2e} is Ar¹.

19-20. (canceled)

21. The compound of claim 1, wherein the compound has a structure represented by a formula selected from: 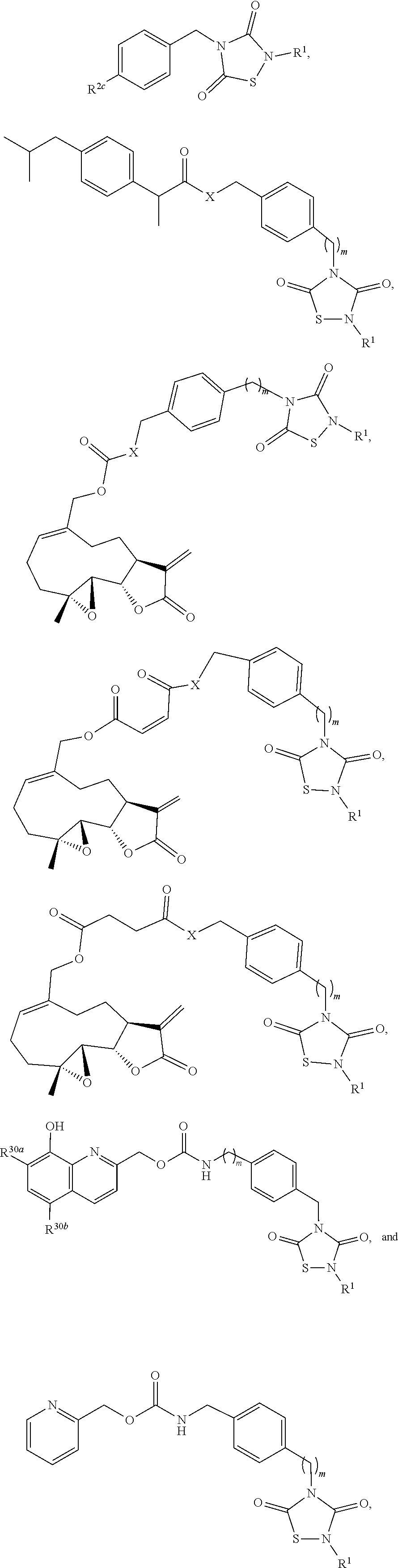
wherein X is NH or O,
wherein each of R^{30a} and R^{30b} is independently selected from hydrogen and halogen.

22-36. (canceled)

37. The compound of claim 31, wherein the compound has a structure represented by a formula: 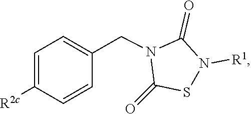
wherein R¹ is selected from C1-C10 alkyl, C1-C10 haloalkyl, and Cy¹;
wherein R^{2c} is 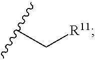
and
wherein R¹¹ is selected from: 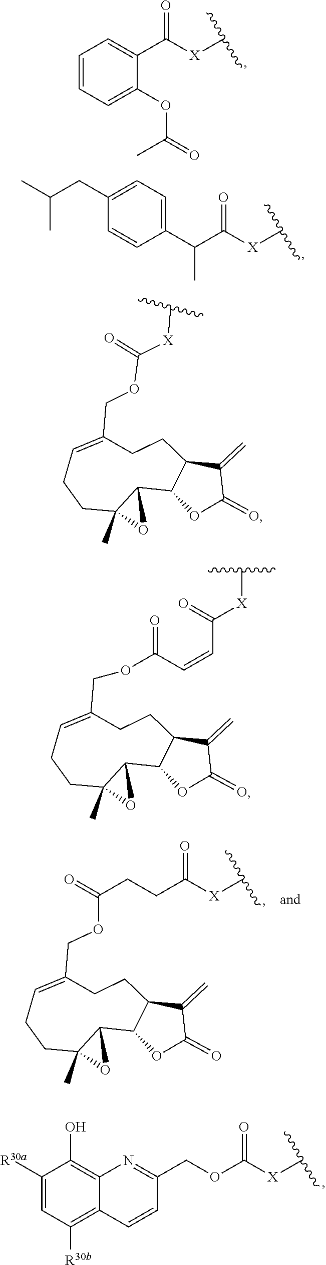
wherein X is selected from NH and O;
wherein each of R^{30a} and R^{30b} is independently selected from hydrogen and halogen.

38. The compound of claim 1, wherein the compound is selected from:

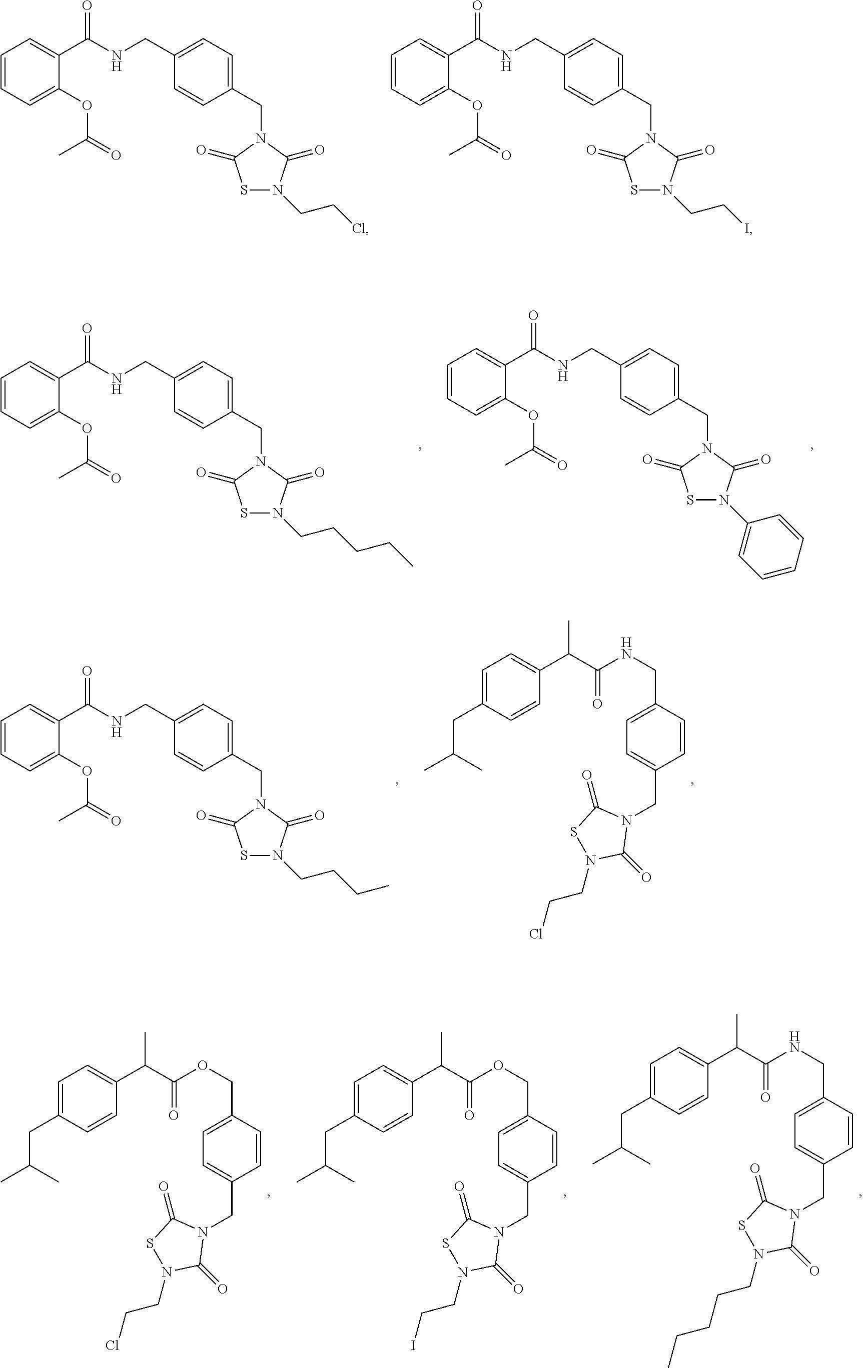

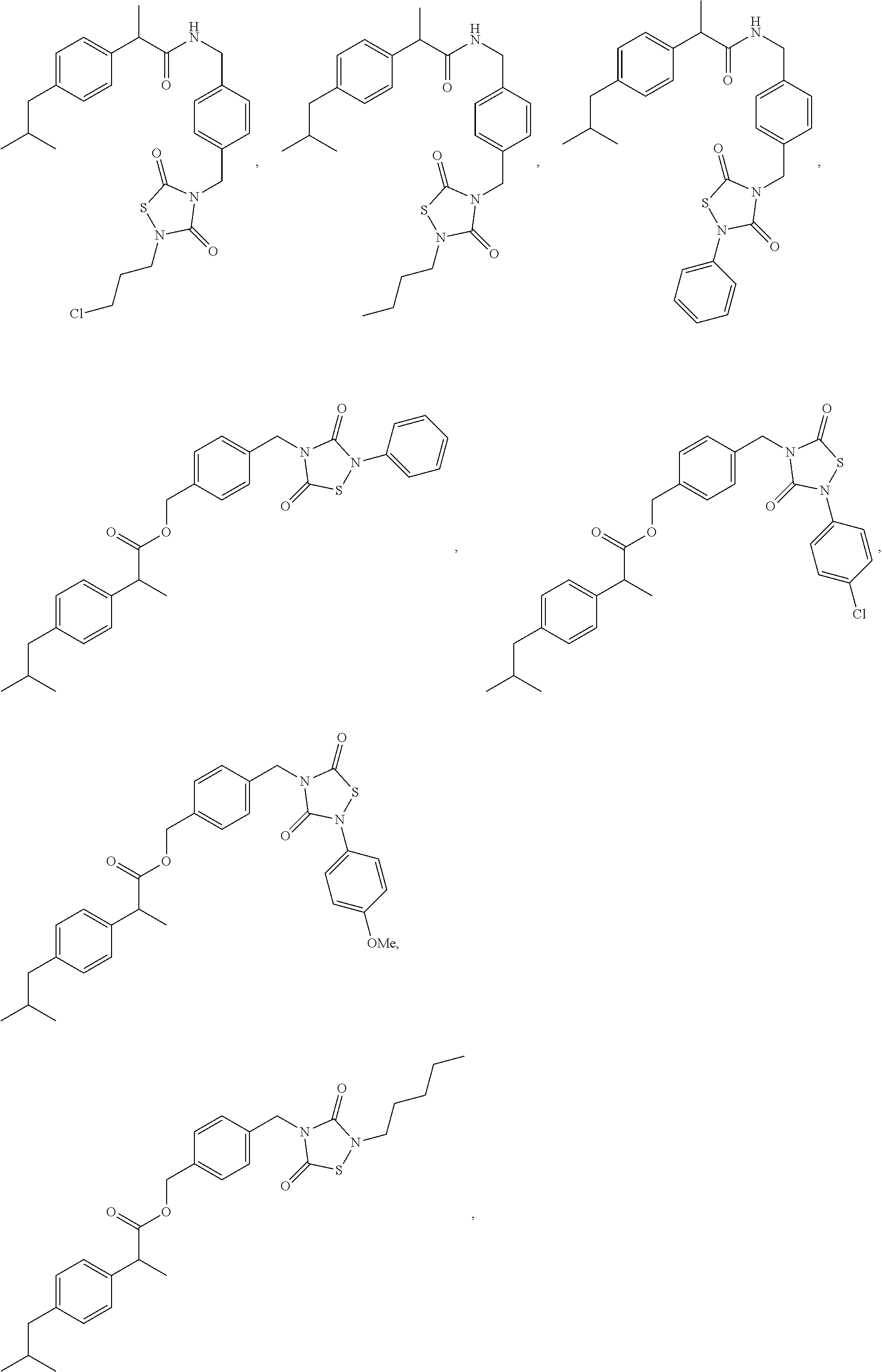

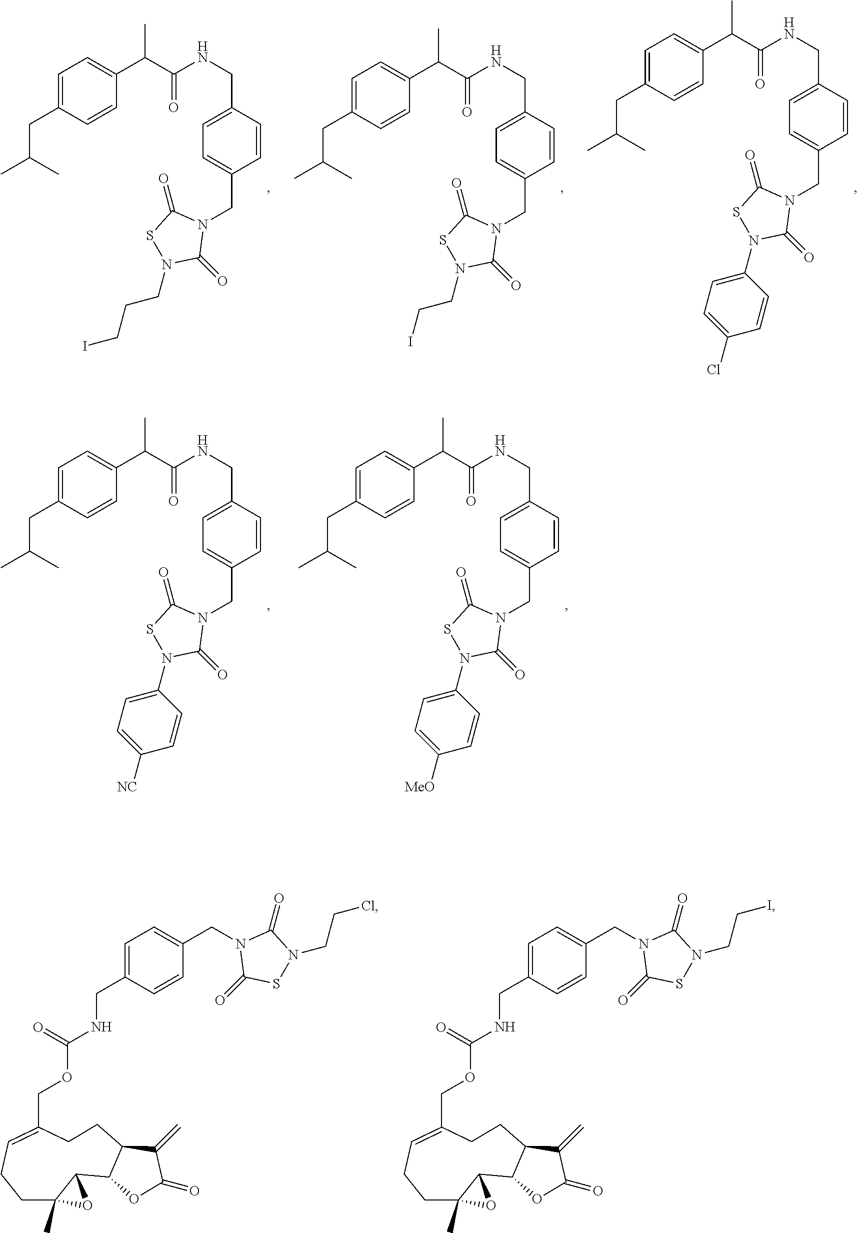

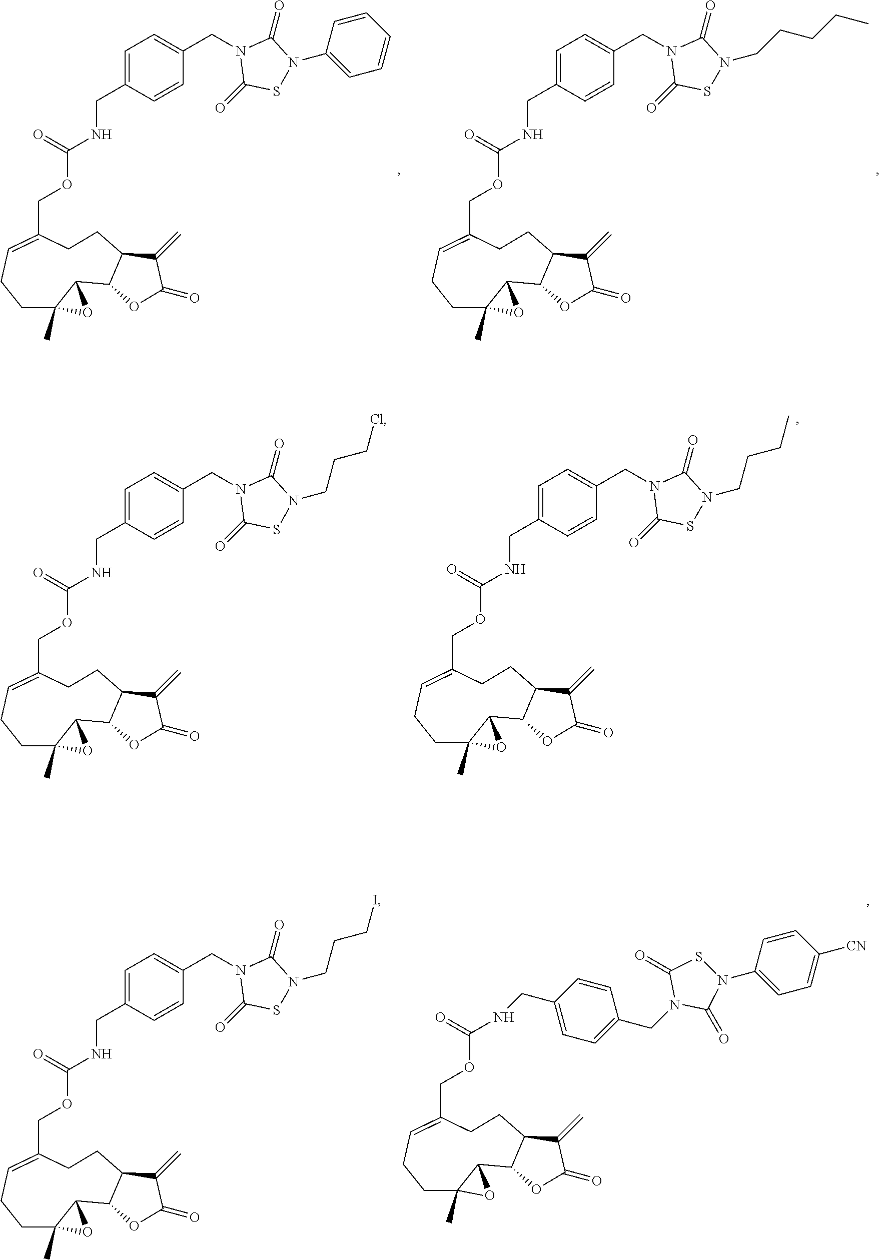

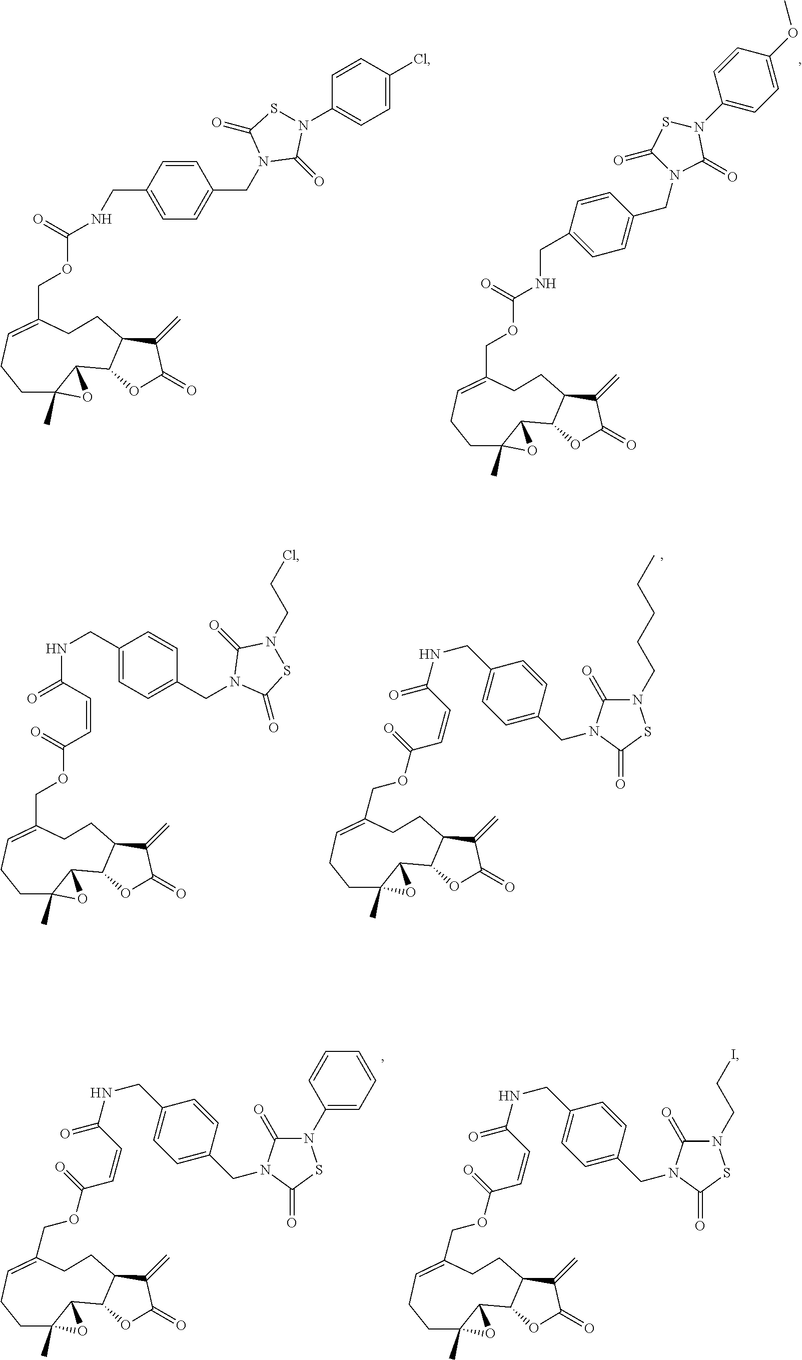

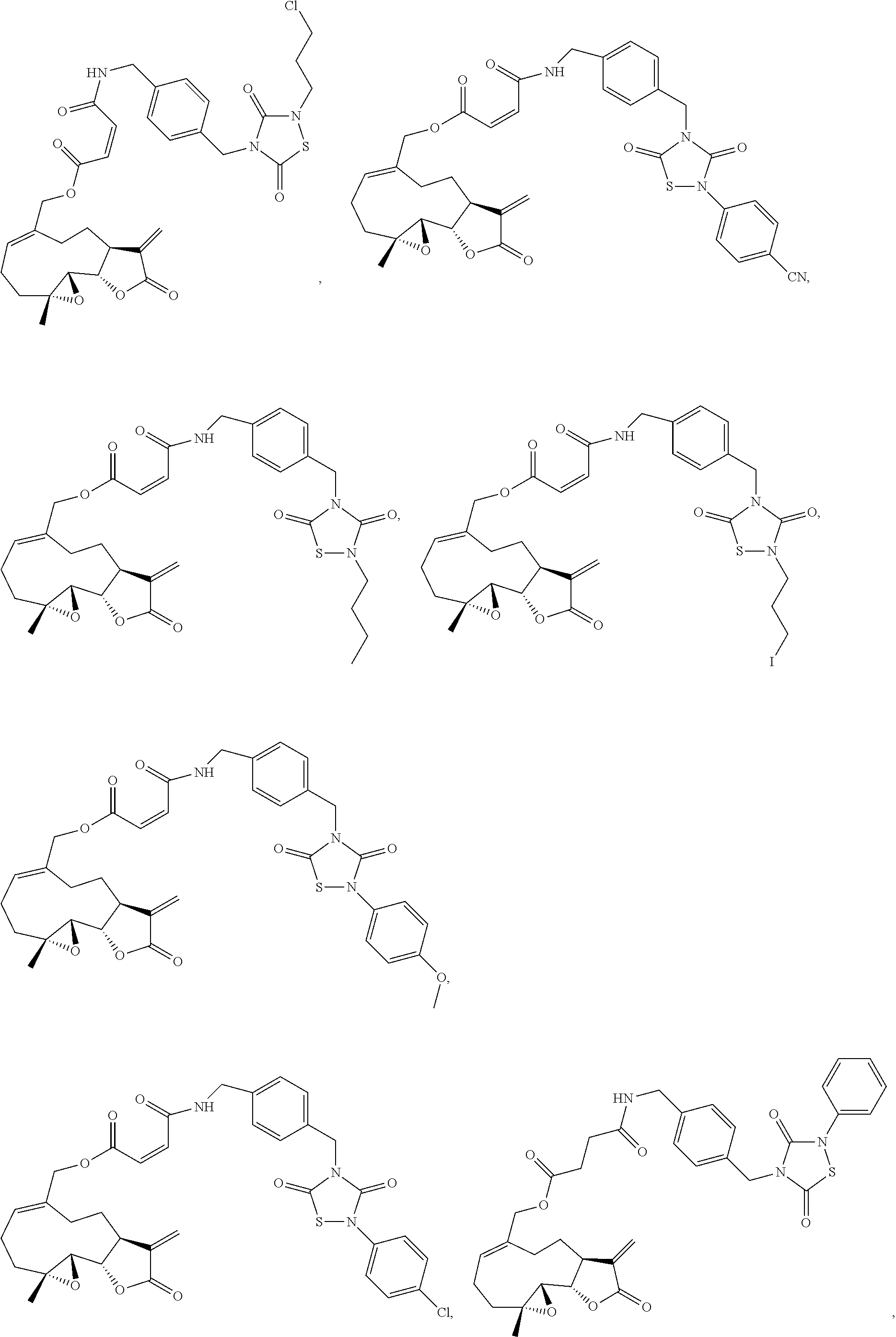

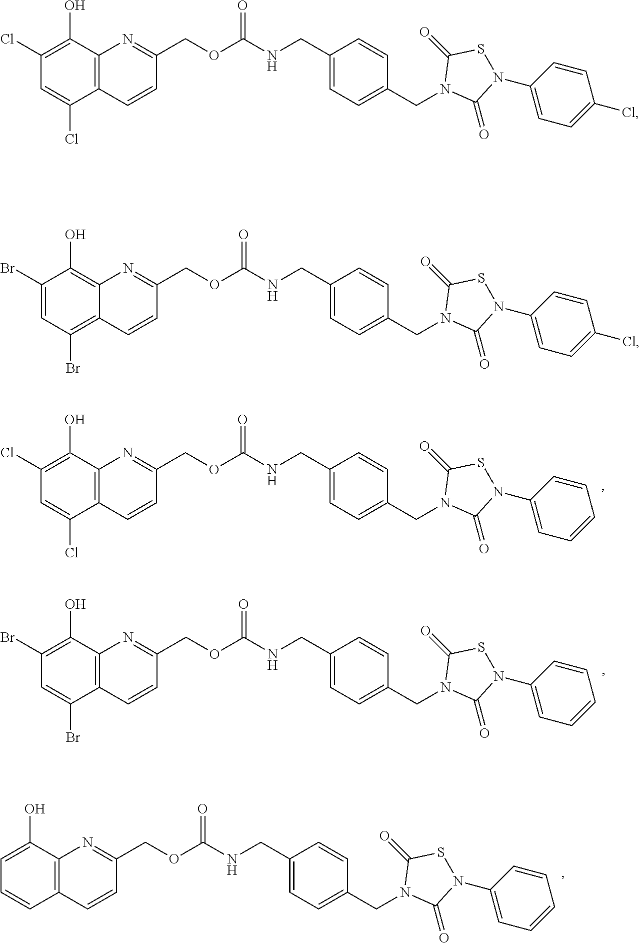 [SMILES: O=C(NCc1ccc(Cn2c(=O)sn(-c3ccc(Cl)cc3)c2=O)cc1)OCc1ccc2c(Cl)cc(Cl)c(O)c2n1 ; O=C(NCc1ccc(Cn2c(=O)sn(-c3ccc(Cl)cc3)c2=O)cc1)OCc1ccc2c(Br)cc(Br)c(O)c2n1 ; O=C(NCc1ccc(Cn2c(=O)sn(-c3ccccc3)c2=O)cc1)OCc1ccc2c(Cl)cc(Cl)c(O)c2n1 ; O=C(NCc1ccc(Cn2c(=O)sn(-c3ccccc3)c2=O)cc1)OCc1ccc2c(Br)cc(Br)c(O)c2n1 ; O=C(NCc1ccc(Cn2c(=O)sn(-c3ccccc3)c2=O)cc1)OCc1ccc2cccc(O)c2n1]

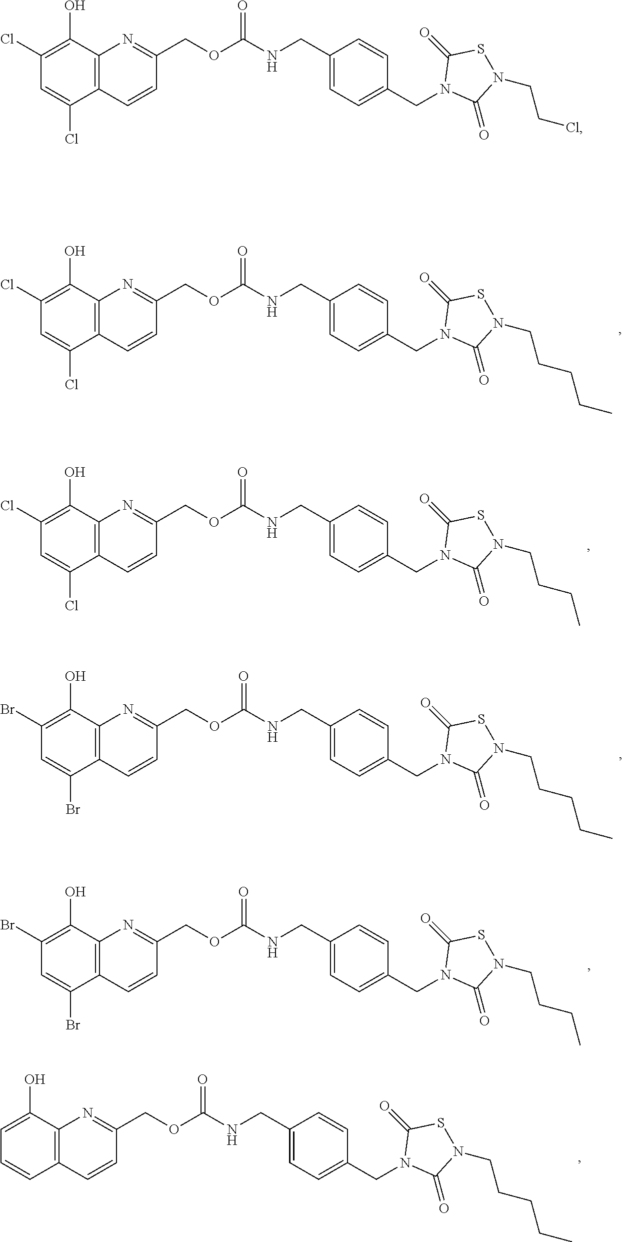 [SMILES: O=C(NCc1ccc(Cn2c(=O)sn(CCCl)c2=O)cc1)OCc1ccc2c(Cl)cc(Cl)c(O)c2n1 ; CCCCCn1sc(=O)n(Cc2ccc(CNC(=O)OCc3ccc4c(Cl)cc(Cl)c(O)c4n3)cc2)c1=O ; CCCCn1sc(=O)n(Cc2ccc(CNC(=O)OCc3ccc4c(Cl)cc(Cl)c(O)c4n3)cc2)c1=O ; CCCCCn1sc(=O)n(Cc2ccc(CNC(=O)OCc3ccc4c(Br)cc(Br)c(O)c4n3)cc2)c1=O ; CCCCn1sc(=O)n(Cc2ccc(CNC(=O)OCc3ccc4c(Br)cc(Br)c(O)c4n3)cc2)c1=O ; CCCCCn1sc(=O)n(Cc2ccc(CNC(=O)OCc3ccc4cccc(O)c4n3)cc2)c1=O]

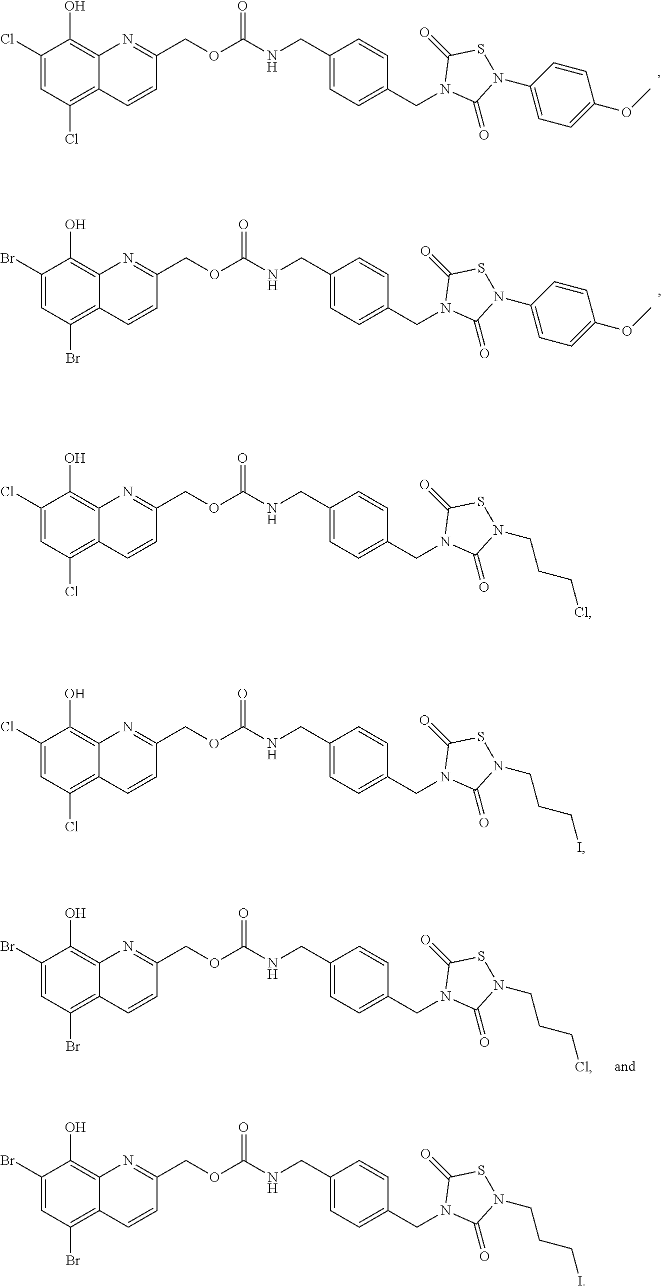 [SMILES: COc1ccc(-n2sc(=O)n(Cc3ccc(CNC(=O)OCc4ccc5c(Cl)cc(Cl)c(O)c5n4)cc3)c2=O)cc1 ; COc1ccc(-n2sc(=O)n(Cc3ccc(CNC(=O)OCc4ccc5c(Br)cc(Br)c(O)c5n4)cc3)c2=O)cc1 ; O=C(NCc1ccc(Cn2c(=O)sn(CCCCl)c2=O)cc1)OCc1ccc2c(Cl)cc(Cl)c(O)c2n1 ; O=C(NCc1ccc(Cn2c(=O)sn(CCCI)c2=O)cc1)OCc1ccc2c(Cl)cc(Cl)c(O)c2n1 ; O=C(NCc1ccc(Cn2c(=O)sn(CCCCl)c2=O)cc1)OCc1ccc2c(Br)cc(Br)c(O)c2n1 ; O=C(NCc1ccc(Cn2c(=O)sn(CCCI)c2=O)cc1)OCc1ccc2c(Br)cc(Br)c(O)c2n1]

39. A pharmaceutical composition comprising a therapeutically effective amount of the compound of claim 1 and a pharmaceutically acceptable carrier.

40. (canceled)

41. A method for treating a disorder of uncontrolled cellular proliferation in a subject, the method comprising administering to the subject an effective amount of the compound of claim 1.

42-47. (canceled)

48. The method of claim 41, wherein the disorder is a cancer selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

49-53. (canceled)

54. A method for treating a neurological disorder in a subject, the method comprising administering to the subject an effective amount of the compound of claim 1.

55-61. (canceled)

62. The method of claim 54, wherein the neurological disorder is selected from sarcopenia, supranuclear palsy, Alzheimer's disease, and dementia.

63-76. (canceled)

77. A compound having a structure represented by a formula: 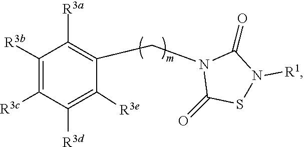
wherein m is 0, 1, 2, or 3;
wherein R¹ is selected from C1-C10 alkyl, C2-C10 alkenyl, C1-C10 haloalkyl, C1-C10 cyanoalkyl, C1-C10 nitroalkyl, C1-C10 hydroxyalkyl, C1-C10 alkoxy, C1-C10 alkenoxy, C1-C10 thioalkyl, C1-C10 alkylthiol, C1-C10 alkylamino, (C1-C10)(C1-C10) dialkylamino, C1-C10 aminoalkyl, —(C1-C10 alkyl)-O—(C1-C10 alkyl), —(C1-C10 alkyl)C(O)R¹⁰, —(C1-C10 alkyl)OC(O)(C1-C10 alkyl), —(C1-C10 alkyl)NHC(O)(C1-C10 alkyl), —(C1-C10 alkyl)N(C1-C10 alkyl)C(O)(C1-C10 alkyl), —(C1-C10)Cy¹, and Cy¹;
wherein R¹⁰, when present, is selected from hydrogen, —OH, C1-C10 alkyl, C1-C10 alkoxy, C1-C10 alkylamino, and (C1-C10)(C1-C10) dialkylamino;
wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, —NO₂, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and
wherein each of R^{3a}, R^{3b}, R^{3c}, R^{3d}, and R^{3e} is independently selected from hydrogen, halogen, —CN, —NH₂, —OH, —NO₂, —CO₂H, —OC(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar¹, provided that one of R^{3a}, R^{3b}, R^{3c}, R^{3d}, and R^{3e} is —CO₂H, —CH₂OH, or —CH₂NH₂, and
provided that when R¹ is C1-C10 alkyl, C2-C10 alkenyl, or C1-C10 haloalkyl, then one of R^{3a}, R^{3b}, R^{3c}, R^{3d}, and R^{3e} is —CO₂H or —CH₂OH,
or a pharmaceutically acceptable salt thereof.

78-86. (canceled)

87. The compound of claim 77, wherein the compound is selected from:

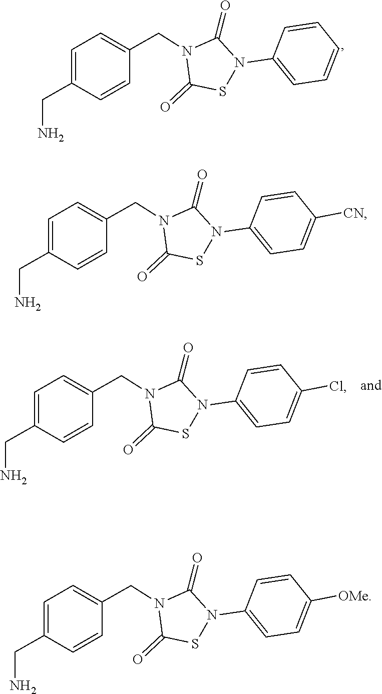 [SMILES: NCc1ccc(Cn2c(=O)sn(-c3ccccc3)c2=O)cc1 ; N#Cc1ccc(-n2sc(=O)n(Cc3ccc(CN)cc3)c2=O)cc1 ; NCc1ccc(Cn2c(=O)sn(-c3ccc(Cl)cc3)c2=O)cc1 ; COc1ccc(-n2sc(=O)n(Cc3ccc(CN)cc3)c2=O)cc1]

88. A compound selected from: 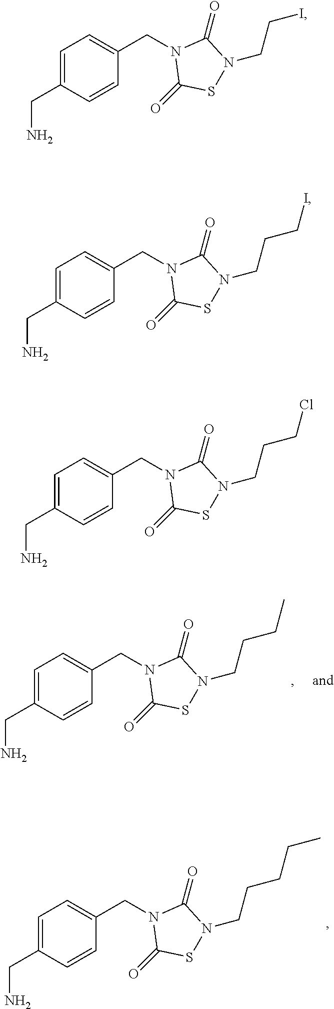 [SMILES: NCc1ccc(Cn2c(=O)sn(CCI)c2=O)cc1 ; NCc1ccc(Cn2c(=O)sn(CCCI)c2=O)cc1 ; NCc1ccc(Cn2c(=O)sn(CCCCl)c2=O)cc1 ; CCCCn1sc(=O)n(Cc2ccc(CN)cc2)c1=O ; CCCCCn1sc(=O)n(Cc2ccc(CN)cc2)c1=O]
or a pharmaceutically acceptable salt thereof.

89-91. (canceled)
